SEQUENCE LISTING (777 sequences)

SEQ ID NO: 1 - protein, 1368 aa, synthetic construct
  REGION 1..1368: note = Synthetic Polypeptide
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 2 - DNA, 4104 bp, Streptococcus pyogenes
  source 1..4104: mol_type = genomic DNA
atggataagaaatactcaataggcttagatatcggcacaaatagcgtcggatgggcggtgatcactgatgattataaggttccgtctaaaaagttcaaggttctgggaaatacagaccgccacagtatcaaaaaaaatcttataggggctcttttatttggcagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggcagattctactgataaagcggatttgcgcttaatctatttggccttagcgcatatgattaagtttcgtggtcattttttgattgagggagatttaaatcctgataatagtgatgtggacaaactatttatccagttggtacaaatctacaatcaattatttgaagaaaaccctattaacgcaagtagagtagatgctaaagcgattctttctgcacgattgagtaaatcaagacgattagaaaatctcattgctcagctccccggtgagaagagaaatggcttgtttgggaatctcattgctttgtcattgggattgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatagtgaaataactaaggctcccctatcagcttcaatgattaagcgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgagggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggcgcctaccatgatttgctaaaaattattaaagataaagattttttggataatgaagaaaatgaagatatcttagaggatattgttttaacattgaccttatttgaagatagggggatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagatattcaaaaagcacaggtgtctggacaaggccatagtttacatgaacagattgctaacttagctggcagtcctgctattaaaaaaggtattttacagactgtaaaaattgttgatgaactggtcaaagtaatggggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctacaaaatggaagagacatgtatgtggaccaagaattagatattaatcgtttaagtgattatgatgtcgatcacattgttccacaaagtttcattaaagacgattcaatagacaataaggtactaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgactga

SEQ ID NO: 3 - protein, 1367 aa, Streptococcus pyogenes
MDKKYSIGLDIGTNSVGWAVITDDYKVPSKKFKVLGNTDRHSIKKNLIGALLFGSGETAEATRLKRTARRRYTRRKNRICYLQEIFSNEMAKVDDSFFHRLEESFLVEEDKKHERHPIFGNIVDEVAYHEKYPTIYHLRKKLADSTDKADLRLIYLALAHMIKFRGHFLIEGDLNPDNSDVDKLFIQLVQIYNQLFEENPINASRVDAKAILSARLSKSRRLENLIAQLPGEKRNGLFGNLIALSLGLTPNFKSNFDLAEDAKLQLSKDTYDDDLDNLLAQIGDQYADLFLAAKNLSDAILLSDILRVNSEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGAYHDLLKIIKDKDFLDNEENEDILEDIVLTLTLFEDRGMIEERLKTYAHLFDDKVMKQLKRRRYTGWGRLSRKLINGIRDKQSGKTILDFLKSDGFANRNFMQLIHDDSLTFKEDIQKAQVSGQGHSLHEQIANLAGSPAIKKGILQTVKIVDELVKVMGHKPENIVIEMARENQTTQKGQKNSRERMKRIEEGIKELGSQILKEHPVENTQLQNEKLYLYYLQNGRDMYVDQELDINRLSDYDVDHIVPQSFIKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 4 - DNA, 4212 bp, synthetic construct
  misc_feature 1..4212: note = Synthetic Polynucleotide
  source 1..4212: mol_type = other DNA
atggataaaaagtattctattggtttagacatcggcactaattccgttggatgggctgtcataaccgatgaatacaaagtaccttcaaagaaatttaaggtgttggggaacacagaccgtcattcgattaaaaagaatcttatcggtgccctcctattcgatagtggcgaaacggcagaggcgactcgcctgaaacgaaccgctcggagaaggtatacacgtcgcaagaaccgaatatgttacttacaagaaatttttagcaatgagatggccaaagttgacgattctttctttcaccgtttggaagagtccttccttgtcgaagaggacaagaaacatgaacggcaccccatctttggaaacatagtagatgaggtggcatatcatgaaaagtacccaacgatttatcacctcagaaaaaagctagttgactcaactgataaagcggacctgaggttaatctacttggctcttgcccatatgataaagttccgtgggcactttctcattgagggtgatctaaatccggacaactcggatgtcgacaaactgttcatccagttagtacaaacctataatcagttgtttgaagagaaccctataaatgcaagtggcgtggatgcgaaggctattcttagcgcccgcctctctaaatcccgacggctagaaaacctgatcgcacaattacccggagagaagaaaaatgggttgttcggtaaccttatagcgctctcactaggcctgacaccaaattttaagtcgaacttcgacttagctgaagatgccaaattgcagcttagtaaggacacgtacgatgacgatctcgacaatctactggcacaaattggagatcagtatgcggacttatttttggctgccaaaaaccttagcgatgcaatcctcctatctgacatactgagagttaatactgagattaccaaggcgccgttatccgcttcaatgatcaaaaggtacgatgaacatcaccaagacttgacacttctcaaggccctagtccgtcagcaactgcctgagaaatataaggaaatattctttgatcagtcgaaaaacgggtacgcaggttatattgacggcggagcgagtcaagaggaattctacaagtttatcaaacccatattagagaagatggatgggacggaagagttgcttgtaaaactcaatcgcgaagatctactgcgaaagcagcggactttcgacaacggtagcattccacatcaaatccacttaggcgaattgcatgctatacttagaaggcaggaggatttttatccgttcctcaaagacaatcgtgaaaagattgagaaaatcctaacctttcgcataccttactatgtgggacccctggcccgagggaactctcggttcgcatggatgacaagaaagtccgaagaaacgattactccatggaattttgaggaagttgtcgataaaggtgcgtcagctcaatcgttcatcgagaggatgaccaactttgacaagaatttaccgaacgaaaaagtattgcctaagcacagtttactttacgagtatttcacagtgtacaatgaactcacgaaagttaagtatgtcactgagggcatgcgtaaacccgcctttctaagcggagaacagaagaaagcaatagtagatctgttattcaagaccaaccgcaaagtgacagttaagcaattgaaagaggactactttaagaaaattgaatgcttcgattctgtcgagatctccggggtagaagatcgatttaatgcgtcacttggtacgtatcatgacctcctaaagataattaaagataaggacttcctggataacgaagagaatgaagatatcttagaagatatagtgttgactcttaccctctttgaagatcgggaaatgattgaggaaagactaaaaacatacgctcacctgttcgacgataaggttatgaaacagttaaagaggcgtcgctatacgggctggggacgattgtcgcggaaacttatcaacgggataagagacaagcaaagtggtaaaactattctcgattttctaaagagcgacggcttcgccaataggaactttatgcagctgatccatgatgactctttaaccttcaaagaggatatacaaaaggcacaggtttccggacaaggggactcattgcacgaacatattgcgaatcttgctggttcgccagccatcaaaaagggcatactccagacagtcaaagtagtggatgagctagttaaggtcatgggacgtcacaaaccggaaaacattgtaatcgagatggcacgcgaaaatcaaacgactcagaaggggcaaaaaaacagtcgagagcggatgaagagaatagaagagggtattaaagaactgggcagccagatcttaaaggagcatcctgtggaaaatacccaattgcagaacgagaaactttacctctattacctacaaaatggaagggacatgtatgttgatcaggaactggacataaaccgtttatctgattacgacgtcgatcacattgtaccccaatcctttttgaaggacgattcaatcgacaataaagtgcttacacgctcggataagaaccgagggaaaagtgacaatgttccaagcgaggaagtcgtaaagaaaatgaagaactattggcggcagctcctaaatgcgaaactgataacgcaaagaaagttcgataacttaactaaagctgagaggggtggcttgtctgaacttgacaaggccggatttattaaacgtcagctcgtggaaacccgccaaatcacaaagcatgttgcacagatactagattcccgaatgaatacgaaatacgacgagaacgataagctgattcgggaagtcaaagtaatcactttaaagtcaaaattggtgtcggacttcagaaaggattttcaattctataaagttagggagataaataactaccaccatgcgcacgacgcttatcttaatgccgtcgtagggaccgcactcattaagaaatacccgaagctagaaagtgagtttgtgtatggtgattacaaagtttatgacgtccgtaagatgatcgcgaaaagcgaacaggagataggcaaggctacagccaaatacttcttttattctaacattatgaatttctttaagacggaaatcactctggcaaacggagagatacgcaaacgacctttaattgaaaccaatggggagacaggtgaaatcgtatgggataagggccgggacttcgcgacggtgagaaaagttttgtccatgccccaagtcaacatagtaaagaaaactgaggtgcagaccggagggttttcaaaggaatcgattcttccaaaaaggaatagtgataagctcatcgctcgtaaaaaggactgggacccgaaaaagtacggtggcttcgatagccctacagttgcctattctgtcctagtagtggcaaaagttgagaagggaaaatccaagaaactgaagtcagtcaaagaattattggggataacgattatggagcgctcgtcttttgaaaagaaccccatcgacttccttgaggcgaaaggttacaaggaagtaaaaaaggatctcataattaaactaccaaagtatagtctgtttgagttagaaaatggccgaaaacggatgttggctagcgccggagagcttcaaaaggggaacgaactcgcactaccgtctaaatacgtgaatttcctgtatttagcgtcccattacgagaagttgaaaggttcacctgaagataacgaacagaagcaactttttgttgagcagcacaaacattatctcgacgaaatcatagagcaaatttcggaattcagtaagagagtcatcctagctgatgccaatctggacaaagtattaagcgcatacaacaagcacagggataaacccatacgtgagcaggcggaaaatattatccatttgtttactcttaccaacctcggcgctccagccgcattcaagtattttgacacaacgatagatcgcaaacgatacacttctaccaaggaggtgctagacgcgacactgattcaccaatccatcacgggattatatgaaactcggatagatttgtcacagcttgggggtgacggatcccccaagaagaagaggaaagtctcgagcgactacaaagaccatgacggtgattataaagatcatgacatcgattacaaggatgacgatgacaaggctgcagga

SEQ ID NO: 5 - protein, 1368 aa, synthetic construct
  REGION 1..1368: note = Synthetic Polypeptide
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 6 - protein, 18 aa, synthetic construct
  REGION 1..18: note = Synthetic Polypeptide
GGSGGSGGSGGSGGSGGS

SEQ ID NO: 7 - protein, 16 aa, synthetic construct
  REGION 1..16: note = Synthetic Polypeptide
SGSETPGTSESATPES

SEQ ID NO: 8 - protein, 12 aa, synthetic construct
  REGION 1..12: note = Synthetic Polypeptide
SGSETPGTSESA

SEQ ID NO: 9 - protein, 21 aa, synthetic construct
  REGION 1..21: note = Synthetic Polypeptide
SGSETPGTSESATPEGGSGGS

SEQ ID NO: 10 - protein, 15 aa, synthetic construct
  REGION 1..15: note = Synthetic Polypeptide
VPFLLEPDNINGKTC

SEQ ID NO: 11 - protein, 12 aa, synthetic construct
  REGION 1..12: note = Synthetic Polypeptide
GSAGSAAGSGEF

SEQ ID NO: 12 - protein, 12 aa, synthetic construct
  REGION 1..12: note = Synthetic Polypeptide
SIVAQLSRPDPA

SEQ ID NO: 13 - protein, 10 aa, synthetic construct
  REGION 1..10: note = Synthetic Polypeptide
MKIIEQLPSA

SEQ ID NO: 14 - protein, 10 aa, synthetic construct
  REGION 1..10: note = Synthetic Polypeptide
VRHKLKRVGS

SEQ ID NO: 15 - protein, 12 aa, synthetic construct
  REGION 1..12: note = Synthetic Polypeptide
GHGTGSTGSGSS

SEQ ID NO: 16 - protein, 7 aa, synthetic construct
  REGION 1..7: note = Synthetic Polypeptide
MSRPDPA

SEQ ID NO: 17 - protein, 4 aa, synthetic construct
  REGION 1..4: note = Synthetic Polypeptide
GGSM

SEQ ID NO: 18-19 - skipped (2 entries)

SEQ ID NO: 20 - DNA, 22 bp, synthetic construct
  misc_feature 1..22: note = Synthetic Polynucleotide
  source 1..22: mol_type = other DNA
ggtgtttcgtcctttccacaag

SEQ ID NO: 21 - DNA, 41 bp, synthetic construct
  misc_feature 1..41: note = Synthetic Polynucleotide
  source 1..41: mol_type = other DNA
gcacactagttagggataacagttttagagctagaaatagc

SEQ ID NO: 22 - DNA, 40 bp, synthetic construct
  misc_feature 1..40: note = Synthetic Polynucleotide
  source 1..40: mol_type = other DNA
gcccatgacccttctcctctgttttagagctagaaatagc

SEQ ID NO: 23 - DNA, 41 bp, synthetic construct
  misc_feature 1..41: note = Synthetic Polynucleotide
  source 1..41: mol_type = other DNA
gctcagggcctgtgatgggaggttttagagctagaaatagc

SEQ ID NO: 24 - DNA, 40 bp, synthetic construct
  misc_feature 1..40: note = Synthetic Polynucleotide
  source 1..40: mol_type = other DNA
ggcccatgacccttctcctcgttttagagctagaaatagc

SEQ ID NO: 25 - DNA, 41 bp, synthetic construct
  misc_feature 1..41: note = Synthetic Polynucleotide
  source 1..41: mol_type = other DNA
gcctcagggcctgtgatgggagttttagagctagaaatagc

SEQ ID NO: 26 - DNA, 40 bp, synthetic construct
  misc_feature 1..40: note = Synthetic Polynucleotide
  source 1..40: mol_type = other DNA
gacttgaaacactctttttcgttttagagctagaaatagc

SEQ ID NO: 27 - DNA, 41 bp, synthetic construct
  misc_feature 1..41: note = Synthetic Polynucleotide
  source 1..41: mol_type = other DNA
gagttgaagacacacaacacagttttagagctagaaatagc

SEQ ID NO: 28 - DNA, 40 bp, synthetic construct
  misc_feature 1..40: note = Synthetic Polynucleotide
  source 1..40: mol_type = other DNA
ggaactcatgtgattaactggttttagagctagaaatagc

SEQ ID NO: 29 - DNA, 41 bp, synthetic construct
  misc_feature 1..41: note = Synthetic Polynucleotide
  source 1..41: mol_type = other DNA
gtctacctctcatgagccggtgttttagagctagaaatagc

SEQ ID NO: 30 - DNA, 41 bp, synthetic construct
  misc_feature 1..41: note = Synthetic Polynucleotide
  source 1..41: mol_type = other DNA
gtttcccgcaggatgtgggatgttttagagctagaaatagc

SEQ ID NO: 31 - DNA, 41 bp, synthetic construct
  misc_feature 1..41: note = Synthetic Polynucleotide
  source 1..41: mol_type = other DNA
gcctggggatttatgttcttagttttagagctagaaatagc

SEQ ID NO: 32 - DNA, 41 bp, synthetic construct
  misc_feature 1..41: note = Synthetic Polynucleotide
  source 1..41: mol_type = other DNA
gaaatagcacaatgaatggaagttttagagctagaaatagc

SEQ ID NO: 33 - DNA, 41 bp, synthetic construct
  misc_feature 1..41: note = Synthetic Polynucleotide
  source 1..41: mol_type = other DNA
gactttttgggggagagggaggttttagagctagaaatagc

SEQ ID NO: 34 - DNA, 40 bp, synthetic construct
  misc_feature 1..40: note = Synthetic Polynucleotide
  source 1..40: mol_type = other DNA
ggagacttaagtccaaaaccgttttagagctagaaatagc

SEQ ID NO: 35 - DNA, 41 bp, synthetic construct
  misc_feature 1..41: note = Synthetic Polynucleotide
  source 1..41: mol_type = other DNA
gtcagctatgatcacttccctgttttagagctagaaatagc

SEQ ID NO: 36 - DNA, 56 bp, synthetic construct
  misc_feature 1..56: note = Synthetic Polynucleotide
  source 1..56: mol_type = other DNA
tcgtctcggcgtccccaattttcccaaacagaggtctgtaaaccgaggtgagacgg

SEQ ID NO: 37 - DNA, 56 bp, synthetic construct
  misc_feature 1..56: note = Synthetic Polynucleotide
  source 1..56: mol_type = other DNA
ccgtctcacctcggtttacagacctctgtttgggaaaattggggacgccgagacga

SEQ ID NO: 38 - DNA, 57 bp, synthetic construct
  misc_feature 1..57: note = Synthetic Polynucleotide
  source 1..57: mol_type = other DNA
tcgtctcggcgtccccaattttcccaaacagaggttctgtaaaccgaggtgagacgg

SEQ ID NO: 39 - DNA, 57 bp, synthetic construct
  misc_feature 1..57: note = Synthetic Polynucleotide
  source 1..57: mol_type = other DNA
ccgtctcacctcggtttacagaacctctgtttgggaaaattggggacgccgagacga

SEQ ID NO: 40 - DNA, 58 bp, synthetic construct
  misc_feature 1..58: note = Synthetic Polynucleotide
  source 1..58: mol_type = other DNA
tcgtctcggcgtccccaattttcccaaacagaggtatctgtaaaccgaggtgagacgg

SEQ ID NO: 41 - DNA, 58 bp, synthetic construct
  misc_feature 1..58: note = Synthetic Polynucleotide
  source 1..58: mol_type = other DNA
ccgtctcacctcggtttacagatacctctgtttgggaaaattggggacgccgagacga

SEQ ID NO: 42 - DNA, 59 bp, synthetic construct
  misc_feature 1..59: note = Synthetic Polynucleotide
  source 1..59: mol_type = other DNA
tcgtctcggcgtccccaattttcccaaacagaggtaatctgtaaaccgaggtgagacgg

SEQ ID NO: 43 - DNA, 59 bp, synthetic construct
  misc_feature 1..59: note = Synthetic Polynucleotide
  source 1..59: mol_type = other DNA
ccgtctcacctcggtttacagattacctctgtttgggaaaattggggacgccgagacga

SEQ ID NO: 44 - DNA, 60 bp, synthetic construct
  misc_feature 1..60: note = Synthetic Polynucleotide
  source 1..60: mol_type = other DNA
tcgtctcggcgtccccaattttcccaaacagaggtaaatctgtaaaccgaggtgagacgg

SEQ ID NO: 45 - DNA, 60 bp, synthetic construct
  misc_feature 1..60: note = Synthetic Polynucleotide
  source 1..60: mol_type = other DNA
ccgtctcacctcggtttacagatttacctctgtttgggaaaattggggacgccgagacga

SEQ ID NO: 46 - DNA, 61 bp, synthetic construct
  misc_feature 1..61: note = Synthetic Polynucleotide
  source 1..61: mol_type = other DNA
tcgtctcggcgtccccaattttcccaaacagaggtgaaatctgtaaaccgaggtgagacgg

SEQ ID NO: 47 - DNA, 61 bp, synthetic construct
  misc_feature 1..61: note = Synthetic Polynucleotide
  source 1..61: mol_type = other DNA
ccgtctcacctcggtttacagatttcacctctgtttgggaaaattggggacgccgagacga

SEQ ID NO: 48 - DNA, 62 bp, synthetic construct
  misc_feature 1..62: note = Synthetic Polynucleotide
  source 1..62: mol_type = other DNA
tcgtctcggcgtccccaattttcccaaacagaggtcgaaatctgtaaaccgaggtgagacgg

SEQ ID NO: 49 - DNA, 62 bp, synthetic construct
  misc_feature 1..62: note = Synthetic Polynucleotide
  source 1..62: mol_type = other DNA
ccgtctcacctcggtttacagatttcgacctctgtttgggaaaattggggacgccgagacga

SEQ ID NO: 50 - DNA, 63 bp, synthetic construct
  misc_feature 1..63: note = Synthetic Polynucleotide
  source 1..63: mol_type = other DNA
tcgtctcggcgtccccaattttcccaaacagaggttcgaaatctgtaaaccgaggtgagacgg

SEQ ID NO: 51 - DNA, 63 bp, synthetic construct
  misc_feature 1..63: note = Synthetic Polynucleotide
  source 1..63: mol_type = other DNA
ccgtctcacctcggtttacagatttcgaacctctgtttgggaaaattggggacgccgagacga

SEQ ID NO: 52 - DNA, 54 bp, synthetic construct
  misc_feature 1..54: note = Synthetic Polynucleotide
  source 1..54: mol_type = other DNA
tcgtctcggaggttttggaacctctgtttgggaaaattggggagtctgagacgg

SEQ ID NO: 53 - DNA, 54 bp, synthetic construct
  misc_feature 1..54: note = Synthetic Polynucleotide
  source 1..54: mol_type = other DNA
ccgtctcagactccccaattttcccaaacagaggttccaaaacctccgagacga

SEQ ID NO: 54 - DNA, 55 bp, synthetic construct
  misc_feature 1..55: note = Synthetic Polynucleotide
  source 1..55: mol_type = other DNA
tcgtctcggaggttttggacacctctgtttgggaaaattggggagtctgagacgg

SEQ ID NO: 55 - DNA, 55 bp, synthetic construct
  misc_feature 1..55: note = Synthetic Polynucleotide
  source 1..55: mol_type = other DNA
ccgtctcagactccccaattttcccaaacagaggtgtccaaaacctccgagacga

SEQ ID NO: 56 - DNA, 56 bp, synthetic construct
  misc_feature 1..56: note = Synthetic Polynucleotide
  source 1..56: mol_type = other DNA
tcgtctcggaggttttggactacctctgtttgggaaaattggggagtctgagacgg

SEQ ID NO: 57 - DNA, 56 bp, synthetic construct
  misc_feature 1..56: note = Synthetic Polynucleotide
  source 1..56: mol_type = other DNA
ccgtctcagactccccaattttcccaaacagaggtagtccaaaacctccgagacga

SEQ ID NO: 58 - DNA, 57 bp, synthetic construct
  misc_feature 1..57: note = Synthetic Polynucleotide
  source 1..57: mol_type = other DNA
tcgtctcggaggttttggacttacctctgtttgggaaaattggggagtctgagacgg

SEQ ID NO: 59 - DNA, 57 bp, synthetic construct
  misc_feature 1..57: note = Synthetic Polynucleotide
  source 1..57: mol_type = other DNA
ccgtctcagactccccaattttcccaaacagaggtaagtccaaaacctccgagacga

SEQ ID NO: 60 - DNA, 58 bp, synthetic construct
  misc_feature 1..58: note = Synthetic Polynucleotide
  source 1..58: mol_type = other DNA
tcgtctcggaggttttggacttaacctctgtttgggaaaattggggagtctgagacgg

SEQ ID NO: 61 - DNA, 58 bp, synthetic construct
  misc_feature 1..58: note = Synthetic Polynucleotide
  source 1..58: mol_type = other DNA
ccgtctcagactccccaattttcccaaacagaggttaagtccaaaacctccgagacga

SEQ ID NO: 62 - DNA, 59 bp, synthetic construct
  misc_feature 1..59: note = Synthetic Polynucleotide
  source 1..59: mol_type = other DNA
tcgtctcggaggttttggacttagacctctgtttgggaaaattggggagtctgagacgg

SEQ ID NO: 63 - DNA, 59 bp, synthetic construct
  misc_feature 1..59: note = Synthetic Polynucleotide
  source 1..59: mol_type = other DNA
ccgtctcagactccccaattttcccaaacagaggtctaagtccaaaacctccgagacga

SEQ ID NO: 64 - DNA, 60 bp, synthetic construct
  misc_feature 1..60: note = Synthetic Polynucleotide
  source 1..60: mol_type = other DNA
tcgtctcggaggttttggacttagcacctctgtttgggaaaattggggagtctgagacgg

SEQ ID NO: 65 - DNA, 60 bp, synthetic construct
  misc_feature 1..60: note = Synthetic Polynucleotide
  source 1..60: mol_type = other DNA
ccgtctcagactccccaattttcccaaacagaggtgctaagtccaaaacctccgagacga

SEQ ID NO: 66 - DNA, 61 bp, synthetic construct
  misc_feature 1..61: note = Synthetic Polynucleotide
  source 1..61: mol_type = other DNA
tcgtctcggaggttttggacttagctacctctgtttgggaaaattggggagtctgagacgg

SEQ ID NO: 67 - DNA, 61 bp, synthetic construct
  misc_feature 1..61: note = Synthetic Polynucleotide
  source 1..61: mol_type = other DNA
ccgtctcagactccccaattttcccaaacagaggtagctaagtccaaaacctccgagacga

SEQ ID NO: 68 - DNA, 54 bp, synthetic construct
  misc_feature 1..54: note = Synthetic Polynucleotide
  source 1..54: mol_type = other DNA
tcgtctctgcacccccaattttcccaaacagaggtctgtaaaccgatgagacgg

SEQ ID NO: 69 - DNA, 54 bp, synthetic construct
  misc_feature 1..54: note = Synthetic Polynucleotide
  source 1..54: mol_type = other DNA
ccgtctcatcggtttacagacctctgtttgggaaaattgggggtgcagagacga

SEQ ID NO: 70 - DNA, 55 bp, synthetic construct
  misc_feature 1..55: note = Synthetic Polynucleotide
  source 1..55: mol_type = other DNA
tcgtctctgcacccccaattttcccaaacagaggttctgtaaaccgatgagacgg

SEQ ID NO: 71 - DNA, 55 bp, synthetic construct
  misc_feature 1..55: note = Synthetic Polynucleotide
  source 1..55: mol_type = other DNA
ccgtctcatcggtttacagaacctctgtttgggaaaattgggggtgcagagacga

SEQ ID NO: 72 - DNA, 56 bp, synthetic construct
  misc_feature 1..56: note = Synthetic Polynucleotide
  source 1..56: mol_type = other DNA
tcgtctctgcacccccaattttcccaaacagaggtatctgtaaaccgatgagacgg

SEQ ID NO: 73 - DNA, 56 bp, synthetic construct
  misc_feature 1..56: note = Synthetic Polynucleotide
  source 1..56: mol_type = other DNA
ccgtctcatcggtttacagatacctctgtttgggaaaattgggggtgcagagacga

SEQ ID NO: 74 - DNA, 57 bp, synthetic construct
  misc_feature 1..57: note = Synthetic Polynucleotide
  source 1..57: mol_type = other DNA
tcgtctctgcacccccaattttcccaaacagaggtaatctgtaaaccgatgagacgg

SEQ ID NO: 75 - DNA, 57 bp, synthetic construct
  misc_feature 1..57: note = Synthetic Polynucleotide
  source 1..57: mol_type = other DNA
ccgtctcatcggtttacagattacctctgtttgggaaaattgggggtgcagagacga

SEQ ID NO: 76 - DNA, 58 bp, synthetic construct
  misc_feature 1..58: note = Synthetic Polynucleotide
  source 1..58: mol_type = other DNA
tcgtctctgcacccccaattttcccaaacagaggtaaatctgtaaaccgatgagacgg

SEQ ID NO: 77 - DNA, 58 bp, synthetic construct
  misc_feature 1..58: note = Synthetic Polynucleotide
  source 1..58: mol_type = other DNA
ccgtctcatcggtttacagatttacctctgtttgggaaaattgggggtgcagagacga

SEQ ID NO: 78 - DNA, 59 bp, synthetic construct
  misc_feature 1..59: note = Synthetic Polynucleotide
  source 1..59: mol_type = other DNA
tcgtctctgcacccccaattttcccaaacagaggtgaaatctgtaaaccgatgagacgg

SEQ ID NO: 79 - DNA, 59 bp, synthetic construct
  misc_feature 1..59: note = Synthetic Polynucleotide
  source 1..59: mol_type = other DNA
ccgtctcatcggtttacagatttcacctctgtttgggaaaattgggggtgcagagacga

SEQ ID NO: 80 - DNA, 60 bp, synthetic construct
  misc_feature 1..60: note = Synthetic Polynucleotide
  source 1..60: mol_type = other DNA
tcgtctctgcacccccaattttcccaaacagaggtcgaaatctgtaaaccgatgagacgg

SEQ ID NO: 81 - DNA, 60 bp, synthetic construct
  misc_feature 1..60: note = Synthetic Polynucleotide
  source 1..60: mol_type = other DNA
ccgtctcatcggtttacagatttcgacctctgtttgggaaaattgggggtgcagagacga

SEQ ID NO: 82 - DNA, 61 bp, synthetic construct
  misc_feature 1..61: note = Synthetic Polynucleotide
  source 1..61: mol_type = other DNA
tcgtctctgcacccccaattttcccaaacagaggttcgaaatctgtaaaccgatgagacgg

SEQ ID NO: 83 - DNA, 61 bp, synthetic construct
  misc_feature 1..61: note = Synthetic Polynucleotide
  source 1..61: mol_type = other DNA
ccgtctcatcggtttacagatttcgaacctctgtttgggaaaattgggggtgcagagacga

SEQ ID NO: 84 - DNA, 56 bp, synthetic construct
  misc_feature 1..56: note = Synthetic Polynucleotide
  source 1..56: mol_type = other DNA
tcgtctcgccgaggttttggaacctctgtttgggaaaattggggctcgtgagacgg

SEQ ID NO: 85 - DNA, 56 bp, synthetic construct
  misc_feature 1..56: note = Synthetic Polynucleotide
  source 1..56: mol_type = other DNA
ccgtctcacgagccccaattttcccaaacagaggttccaaaacctcggcgagacga

SEQ ID NO: 86 - DNA, 57 bp, synthetic construct
  misc_feature 1..57: note = Synthetic Polynucleotide
  source 1..57: mol_type = other DNA
tcgtctcgccgaggttttggacacctctgtttgggaaaattggggctcgtgagacgg

SEQ ID NO: 87 - DNA, 57 bp, synthetic construct
  misc_feature 1..57: note = Synthetic Polynucleotide
  source 1..57: mol_type = other DNA
ccgtctcacgagccccaattttcccaaacagaggtgtccaaaacctcggcgagacga

SEQ ID NO: 88 - DNA, 58 bp, synthetic construct
  misc_feature 1..58: note = Synthetic Polynucleotide
  source 1..58: mol_type = other DNA
tcgtctcgccgaggttttggactacctctgtttgggaaaattggggctcgtgagacgg

SEQ ID NO: 89 - DNA, 58 bp, synthetic construct
  misc_feature 1..58: note = Synthetic Polynucleotide
  source 1..58: mol_type = other DNA
ccgtctcacgagccccaattttcccaaacagaggtagtccaaaacctcggcgagacga

SEQ ID NO: 90 - DNA, 59 bp, synthetic construct
  misc_feature 1..59: note = Synthetic Polynucleotide
  source 1..59: mol_type = other DNA
tcgtctcgccgaggttttggacttacctctgtttgggaaaattggggctcgtgagacgg

SEQ ID NO: 91 - DNA, 59 bp, synthetic construct
  misc_feature 1..59: note = Synthetic Polynucleotide
  source 1..59: mol_type = other DNA
ccgtctcacgagccccaattttcccaaacagaggtaagtccaaaacctcggcgagacga

SEQ ID NO: 92 - DNA, 60 bp, synthetic construct
  misc_feature 1..60: note = Synthetic Polynucleotide
  source 1..60: mol_type = other DNA
tcgtctcgccgaggttttggacttaacctctgtttgggaaaattggggctcgtgagacgg

SEQ ID NO: 93 - DNA, 60 bp, synthetic construct
  misc_feature 1..60: note = Synthetic Polynucleotide
  source 1..60: mol_type = other DNA
ccgtctcacgagccccaattttcccaaacagaggttaagtccaaaacctcggcgagacga

SEQ ID NO: 94 - DNA, 61 bp, synthetic construct
  misc_feature 1..61: note = Synthetic Polynucleotide
  source 1..61: mol_type = other DNA
tcgtctcgccgaggttttggacttagacctctgtttgggaaaattggggctcgtgagacgg

SEQ ID NO: 95 - DNA, 61 bp, synthetic construct
  misc_feature 1..61: note = Synthetic Polynucleotide
  source 1..61: mol_type = other DNA
ccgtctcacgagccccaattttcccaaacagaggtctaagtccaaaacctcggcgagacga

SEQ ID NO: 96 - DNA, 62 bp, synthetic construct
  misc_feature 1..62: note = Synthetic Polynucleotide
  source 1..62: mol_type = other DNA
tcgtctcgccgaggttttggacttagcacctctgtttgggaaaattggggctcgtgagacgg

SEQ ID NO: 97 - DNA, 62 bp, synthetic construct
  misc_feature 1..62: note = Synthetic Polynucleotide
  source 1..62: mol_type = other DNA
ccgtctcacgagccccaattttcccaaacagaggtgctaagtccaaaacctcggcgagacga

SEQ ID NO: 98 - DNA, 63 bp, synthetic construct
  misc_feature 1..63: note = Synthetic Polynucleotide
  source 1..63: mol_type = other DNA
tcgtctcgccgaggttttggacttagctacctctgtttgggaaaattggggctcgtgagacgg

SEQ ID NO: 99 - DNA, 63 bp, synthetic construct
  misc_feature 1..63: note = Synthetic Polynucleotide
  source 1..63: mol_type = other DNA
ccgtctcacgagccccaattttcccaaacagaggtagctaagtccaaaacctcggcgagacga

SEQ ID NO: 100 - DNA, 58 bp, synthetic construct
  misc_feature 1..58: note = Synthetic Polynucleotide
  source 1..58: mol_type = other DNA
tcgtctcggcgtcccctcccatcacaggccctgaggtttaagagaaaacctgagacgg

SEQ ID NO: 101 - DNA, 58 bp, synthetic construct
  misc_feature 1..58: note = Synthetic Polynucleotide
  source 1..58: mol_type = other DNA
ccgtctcaggttttctcttaaacctcagggcctgtgatgggaggggacgccgagacga

SEQ ID NO: 102 - DNA, 64 bp, synthetic construct
  misc_feature 1..64: note = Synthetic Polynucleotide
  source 1..64: mol_type = other DNA
tcgtctcgaaccatggttttgtgggccaggcccatgacccttctcctctgggagtctgagacgg

SEQ ID NO: 103 - DNA, 64 bp, synthetic construct
  misc_feature 1..64: note = Synthetic Polynucleotide
  source 1..64: mol_type = other DNA
ccgtctcagactcccagaggagaagggtcatgggcctggcccacaaaaccatggttcgagacga

SEQ ID NO: 104 - DNA, 60 bp, synthetic construct
  misc_feature 1..60: note = Synthetic Polynucleotide
  source 1..60: mol_type = other DNA
tcgtctctgcaccccctcccatcacaggccctgaggtttaagagaaaaccattgagacgg

SEQ ID NO: 105 - DNA, 60 bp, synthetic construct
  misc_feature 1..60: note = Synthetic Polynucleotide
  source 1..60: mol_type = other DNA
ccgtctcaatggttttctcttaaacctcagggcctgtgatgggagggggtgcagagacga

SEQ ID NO: 106 - DNA, 62 bp, synthetic construct
  misc_feature 1..62: note = Synthetic Polynucleotide
  source 1..62: mol_type = other DNA
tcgtctcgccatggttttgtgggccaggcccatgacccttctcctctgggctcgtgagacgg

SEQ ID NO: 107 - DNA, 62 bp, synthetic construct
  misc_feature 1..62: note = Synthetic Polynucleotide
  source 1..62: mol_type = other DNA
ccgtctcacgagcccagaggagaagggtcatgggcctggcccacaaaaccatggcgagacga

SEQ ID NO: 108 - DNA, 43 bp, synthetic construct
  misc_feature 1..43: note = Synthetic Polynucleotide
  source 1..43: mol_type = other DNA
atccgtctccagtcgagtcggatttgatctgatcaagagacag

SEQ ID NO: 109 - DNA, 45 bp, synthetic construct
  misc_feature 1..45: note = Synthetic Polynucleotide
  source 1..45: mol_type = other DNA
aaccgtctcggtgcgttcggatttgatccagacatgataagatac

SEQ ID NO: 110 - DNA, 30 bp, synthetic construct
  misc_feature 1..30: note = Synthetic Polynucleotide
  source 1..30: mol_type = other DNA
hscgcgttgagacgctgccatccgtctcgc

SEQ ID NO: 111 - DNA, 30 bp, synthetic construct
  misc_feature 1..30: note = Synthetic Polynucleotide
  source 1..30: mol_type = other DNA
hstcgagcgagacggatggcagcgtctcaa

SEQ ID NO: 112 - DNA, 130 bp, synthetic construct
  misc_feature 1..130: note = Synthetic Polynucleotide
  source 1..130: mol_type = other DNA
gttgttcgtctcggcgtccttgtgttgtgtgtcttcaactcacagagttaaacgatgctttacacagagtagacttgaaacactctttttctggagtctgagacggttctgttttggtgtgattagttat

SEQ ID NO: 113 - DNA, 130 bp, synthetic construct
  misc_feature 1..130: note = Synthetic Polynucleotide
  source 1..130: mol_type = other DNA
gttggtcgtctctgcacccttgtgttgtgtgtcttcaactcacagagttaaacgatgctttacacagagtagacttgaaacactctttttctggctcgtgagacggttctgttttggtgtgattagttat

SEQ ID NO: 114 - DNA, 131 bp, synthetic construct
  misc_feature 1..131: note = Synthetic Polynucleotide
  source 1..131: mol_type = other DNA
gttgttcgtctcggcgtcccaccggctcatgagaggtagagctaaggtccaaacctaggtttatctgagaccggaactcatgtgattaactgtggagtctgagacggttctgttttggtgtgattagttat

SEQ ID NO: 115 - DNA, 131 bp, synthetic construct
  misc_feature 1..131: note = Synthetic Polynucleotide
  source 1..131: mol_type = other DNA
gttggtcgtctctgcaccccaccggctcatgagaggtagagctaaggtccaaacctaggtttatctgagaccggaactcatgtgattaactgtggctcgtgagacggttctgttttggtgtgattagttat

SEQ ID NO: 116 - DNA, 130 bp, synthetic construct
  misc_feature 1..130: note = Synthetic Polynucleotide
  source 1..130: mol_type = other DNA
gttgttcgtctcggcgtccttaagaacataaatccccaggaattcacagaaaccttggtttgagctttggatttcccgcaggatgtgggataggagtctgagacggttctgttttggtgtgattagttat

SEQ ID NO: 117 - DNA, 130 bp, synthetic construct
  misc_feature 1..130: note = Synthetic Polynucleotide
  source 1..130: mol_type = other DNA
gttggtcgtctctgcacccttaagaacataaatccccaggaattcacagaaaccttggtttgagctttggatttcccgcaggatgtgggataggctcgtgagacggttctgttttggtgtgattagttat

SEQ ID NO: 118 - DNA, 129 bp, synthetic construct
  misc_feature 1..129: note = Synthetic Polynucleotide
  source 1..129: mol_type = other DNA
gttgttcgtctcggcgtccactccctctcccccaaaaagtaaaggtagaaaaccaaggtttacaggcaacaaatagcacaatgaatggaatggagtctgagacggttctgttttggtgtgattagttat

SEQ ID NO: 119 - DNA, 129 bp, synthetic construct
  misc_feature 1..129: note = Synthetic Polynucleotide
  source 1..129: mol_type = other DNA
gttggtcgtctctgcacccactccctctcccccaaaaagtaaaggtagaaaaccaaggtttacaggcaacaaatagcacaatgaatggaatggctcgtgagacggttctgttttggtgtgattagttat

SEQ ID NO: 120 - DNA, 130 bp, synthetic construct
  misc_feature 1..130: note = Synthetic Polynucleotide
  source 1..130: mol_type = other DNA
gttgttcgtctcggcgtcctagggaagtgatcatagctgagtttctggaaaaacctaggttttaaagttgaggagacttaagtccaaaacctggagtctgagacggttctgttttggtgtgattagttat

SEQ ID NO: 121 - DNA, 130 bp, synthetic construct
  misc_feature 1..130: note = Synthetic Polynucleotide
  source 1..130: mol_type = other DNA
gttggtcgtctctgcaccctagggaagtgatcatagctgagtttctggaaaaacctaggttttaaagttgaggagacttaagtccaaaacctggctcgtgagacggttctgttttggtgtgattagttat

SEQ ID NO: 122 - DNA, 47 bp, synthetic construct
  misc_feature 1..47: note = Synthetic Polynucleotide
  source 1..47: mol_type = other DNA
ttcatcggatccgataaaaagtattctattggtttagctatcggcac

SEQ ID NO: 123 - DNA, 34 bp, synthetic construct
  misc_feature 1..34: note = Synthetic Polynucleotide
  source 1..34: mol_type = other DNA
ttcatcggatccggtggttcaggtggcagcggag

SEQ ID NO: 124 - DNA, 61 bp, synthetic construct
  misc_feature 1..61: note = Synthetic Polynucleotide
  source 1..61: mol_type = other DNA
ttcatcggatccggagggtccggaggtagtggcggcagcggtggttcaggtggcagcggag

SEQ ID NO: 125 - DNA, 100 bp, synthetic construct
  misc_feature 1..100: note = Synthetic Polynucleotide
  source 1..100: mol_type = other DNA
aataaccggttcagaccttccttttcttctttggggaacctcccttgtcgtcatcatccttataatcggagccaccgtcacccccaagctgtgacaaatc

SEQ ID NO: 126 - DNA, 38 bp, synthetic construct
  misc_feature 1..38: note = Synthetic Polynucleotide
  source 1..38: mol_type = other DNA
tgataaggatccaccctttggtggtcttccaaaccgcc

SEQ ID NO: 127 - DNA, 38 bp, synthetic construct
  misc_feature 1..38: note = Synthetic Polynucleotide
  source 1..38: mol_type = other DNA
tgataaggatccaccgctaccaccctttggtggtcttc

SEQ ID NO: 128 - DNA, 20 bp, synthetic construct
  misc_feature 1..20: note = Synthetic Polynucleotide
  source 1..20: mol_type = other DNA
agatccgcggccgctaatac

SEQ ID NO: 129 - DNA, 54 bp, synthetic construct
  misc_feature 1..54: note = Synthetic Polynucleotide
  source 1..54: mol_type = other DNA
ttgagtcgtctctatactcttcctttttcaatattattgaagcatttatcaggg

SEQ ID NO: 130 - DNA, 50 bp, synthetic construct
  misc_feature 1..50: note = Synthetic Polynucleotide
  source 1..50: mol_type = other DNA
ctggaacgtctcactgtcagaccaagtttactcatatatactttagattg

SEQ ID NO: 131 - DNA, 46 bp, synthetic construct
  misc_feature 1..46: note = Synthetic Polynucleotide
  source 1..46: mol_type = other DNA
ggtgtgcgtctctacagttatttgccgactaccttggtgatctcgc

SEQ ID NO: 132 - DNA, 36 bp, synthetic construct
  misc_feature 1..36: note = Synthetic Polynucleotide
  source 1..36: mol_type = other DNA
acaccacgtctctgtatgagggaagcggtgatcgcc

SEQ ID NO: 133 - DNA, 42 bp, synthetic construct
  misc_feature 1..42: note = Synthetic Polynucleotide
  source 1..42: mol_type = other DNA
catactcttcctttttcaatattattgaagcatttatcaggg

SEQ ID NO: 134 - DNA, 37 bp, synthetic construct
  misc_feature 1..37: note = Synthetic Polynucleotide
  source 1..37: mol_type = other DNA
ctgtcagaccaagtttactcatatatactttagattg

SEQ ID NO: 135 - DNA, 62 bp, synthetic construct
  misc_feature 1..62: note = Synthetic Polynucleotide
  source 1..62: mol_type = other DNA
caatctaaagtatatatgagtaaacttggtctgacagtttgccgactaccttggtgatctcg

SEQ ID NO: 136 - DNA, 65 bp, synthetic construct
  misc_feature 1..65: note = Synthetic Polynucleotide
  source 1..65: mol_type = other DNA
caatctaaagtatatatgagtaaacttggtctgacagttatttgccgactaccttggtgatctcg

SEQ ID NO: 137 - DNA, 42 bp, synthetic construct
  misc_feature 1..42: note = Synthetic Polynucleotide
  source 1..42: mol_type = other DNA
ccctgataaatgcttcaataatattgaaaaaggaagagtatg

SEQ ID NO: 138 - DNA, 21 bp, synthetic construct
  misc_feature 1..21: note = Synthetic Polynucleotide
  source 1..21: mol_type = other DNA
cgcaaatgggcggtaggcgtg

SEQ ID NO: 139 - DNA, 20 bp, synthetic construct
  misc_feature 1..20: note = Synthetic Polynucleotide
  source 1..20: mol_type = other DNA
ccgtgatggattggtgaatc

SEQ ID NO: 140 - DNA, 20 bp, synthetic construct
  misc_feature 1..20: note = Synthetic Polynucleotide
  source 1..20: mol_type = other DNA
cccatacgatttcacctgtc

SEQ ID NO: 141 - DNA, 20 bp, synthetic construct
  misc_feature 1..20: note = Synthetic Polynucleotide
  source 1..20: mol_type = other DNA
gggtattttccacaggatgc

SEQ ID NO: 142 - DNA, 20 bp, synthetic construct
  misc_feature 1..20: note = Synthetic Polynucleotide
  source 1..20: mol_type = other DNA
cttagaaaggcgggtttacg

SEQ ID NO: 143 - DNA, 20 bp, synthetic construct
  misc_feature 1..20: note = Synthetic Polynucleotide
  source 1..20: mol_type = other DNA
cttactaagctgcaatttgg

SEQ ID NO: 144 - DNA, 21 bp, synthetic construct
  misc_feature 1..21: note = Synthetic Polynucleotide
  source 1..21: mol_type = other DNA
tgtattcatcggttatgacag

SEQ ID NO: 145 - DNA, 19 bp, synthetic construct
  misc_feature 1..19: note = Synthetic Polynucleotide
  source 1..19: mol_type = other DNA
cagggtcaaggaaggcacg

SEQ ID NO: 146 - DNA, 17 bp, synthetic construct
  misc_feature 1..17: note = Synthetic Polynucleotide
  source 1..17: mol_type = other DNA
gttccgcgcacatttcc

SEQ ID NO: 147 - DNA, 19 bp, synthetic construct
  misc_feature 1..19: note = Synthetic Polynucleotide
  source 1..19: mol_type = other DNA
gcggagcctatggaaaaac

SEQ ID NO: 148 - DNA, 22 bp, synthetic construct
  misc_feature 1..22: note = Synthetic Polynucleotide
  source 1..22: mol_type = other DNA
gccttcttctttttcctacagc

SEQ ID NO: 149 - DNA, 16 bp, synthetic construct
  misc_feature 1..16: note = Synthetic Polynucleotide
  source 1..16: mol_type = other DNA
cgcatcgagcgagcac

SEQ ID NO: 150 - DNA, 27 bp, synthetic construct
  misc_feature 1..27: note = Synthetic Polynucleotide
  source 1..27: mol_type = other DNA
tcaagtagcaaaagaagtaggagtcag

SEQ ID NO: 151 - DNA, 22 bp, synthetic construct
  misc_feature 1..22: note = Synthetic Polynucleotide
  source 1..22: mol_type = other DNA
ttagatgcattcgtgcttgaag

SEQ ID NO: 152 - DNA, 29 bp, synthetic construct
  misc_feature 1..29: note = Synthetic Polynucleotide
  source 1..29: mol_type = other DNA
ttaatttctgctgctagaactaaatctgg

SEQ ID NO: 153 - DNA, 24 bp, synthetic construct
  misc_feature 1..24: note = Synthetic Polynucleotide
  source 1..24: mol_type = other DNA
gggaagaaaactggatggagaatg

SEQ ID NO: 154 - DNA, 22 bp, synthetic construct
  misc_feature 1..22: note = Synthetic Polynucleotide
  source 1..22: mol_type = other DNA
cataaatgacctagtggagctg

SEQ ID NO: 155 - DNA, 26 bp, synthetic construct
  misc_feature 1..26: note = Synthetic Polynucleotide
  source 1..26: mol_type = other DNA
tggttattttgcccattagttgatgc

SEQ ID NO: 156 - DNA, 1318 bp, synthetic construct
  misc_feature 1..1318: note = Synthetic Polynucleotide
  source 1..1318: mol_type = other DNA
acgcgtccccaattttcccaaacagaggtctgtaaaccgaggttttggaacctctgtttgggaaaattggggagtcgagtcggatttgatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagtcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccatcgataacttgtttattgcagcttataatggttacaaataaagcaatagcatcacaaatttcacaaataaagcatttttttcactgcattctagttgtggtttgtccaaactcatcaatgtatcttatcatgtctggatcaaatccgaacgcacccccaattttcccaaacagaggtctgtaaaccgaggttttggaacctctgtttgggaaaattggggctcgag

SEQ ID NO: 157 - DNA, 67 bp, synthetic construct
  misc_feature 1..67: note = Synthetic Polynucleotide
  source 1..67: mol_type = other DNA
ccccaattttcccaaacagaggttctgtaaaccgaggttttggaacctctgtttgggaaaattgggg

SEQ ID NO: 158 - DNA, 68 bp, synthetic construct
  misc_feature 1..68: note = Synthetic Polynucleotide
  source 1..68: mol_type = other DNA
ccccaattttcccaaacagaggttctgtaaaccgaggttttggcaacctctgtttgggaaaattgggg

SEQ ID NO: 159 - DNA, 70 bp, synthetic construct
  misc_feature 1..70: note = Synthetic Polynucleotide
  source 1..70: mol_type = other DNA
ccccaattttcccaaacagaggtatctgtaaaccgaggttttggctaacctctgtttgggaaaattgggg

SEQ ID NO: 160 - DNA, 72 bp, synthetic construct
  misc_feature 1..72: note = Synthetic Polynucleotide
  source 1..72: mol_type = other DNA
ccccaattttcccaaacagaggtaatctgtaaaccgaggttttggcttaacctctgtttgggaaaattgggg

SEQ ID NO: 161 - DNA, 74 bp, synthetic construct
  misc_feature 1..74: note = Synthetic Polynucleotide
  source 1..74: mol_type = other DNA
ccccaattttcccaaacagaggtaaatctgtaaaccgaggttttggcttaaacctctgtttgggaaaattgggg

SEQ ID NO: 162 - DNA, 76 bp, synthetic construct
  misc_feature 1..76: note = Synthetic Polynucleotide
  source 1..76: mol_type = other DNA
ccccaattttcccaaacagaggtgaaatctgtaaaccgaggttttggcttagaacctctgtttgggaaaattgggg

SEQ ID NO: 163 - DNA, 78 bp, synthetic construct
  misc_feature 1..78: note = Synthetic Polynucleotide
  source 1..78: mol_type = other DNA
ccccaattttcccaaacagaggtcgaaatctgtaaaccgaggttttggcttagcaacctctgtttgggaaaattgggg

SEQ ID NO: 164 - DNA, 80 bp, synthetic construct
  misc_feature 1..80: note = Synthetic Polynucleotide
  source 1..80: mol_type = other DNA
ccccaattttcccaaacagaggttcgaaatctgtaaaccgaggttttggcttagctaacctctgtttgggaaaattgggg

SEQ ID NO: 165 - DNA, 73 bp, synthetic construct
  misc_feature 1..73: note = Synthetic Polynucleotide
  source 1..73: mol_type = other DNA
ccccaattttcccaaacagaggttcgaaatctgtaaaccgaggttttggaacctctgtttgggaaaattgggg

SEQ ID NO: 166 - DNA, 74 bp, synthetic construct
  misc_feature 1..74: note = Synthetic Polynucleotide
  source 1..74: mol_type = other DNA
ccccaattttcccaaacagaggttcgaaatctgtaaaccgaggttttggcaacctctgtttgggaaaattgggg

SEQ ID NO: 167 - DNA, 74 bp, synthetic construct
  misc_feature 1..74: note = Synthetic Polynucleotide
  source 1..74: mol_type = other DNA
ccccaattttcccaaacagaggtcgaaatctgtaaaccgaggttttggctaacctctgtttgggaaaattgggg

SEQ ID NO: 168 - DNA, 76 bp, synthetic construct
  misc_feature 1..76: note = Synthetic Polynucleotide
  source 1..76: mol_type = other DNA
ccccaattttcccaaacagaggtcgaaatctgtaaaccgaggttttggcttaaacctctgtttgggaaaattgggg

SEQ ID NO: 169 - DNA, 77 bp, synthetic construct
  misc_feature 1..77: note = Synthetic Polynucleotide
  source 1..77: mol_type = other DNA
ccccaattttcccaaacagaggtcgaaatctgtaaaccgaggttttggcttagaacctctgtttgggaaaattgggg

SEQ ID NO: 170 - DNA, 72 bp, synthetic construct
  misc_feature 1..72: note = Synthetic Polynucleotide
  source 1..72: mol_type = other DNA
ccccaattttcccaaacagaggtctgtaaaccgaggttttggcttagcaacctctgtttgggaaaattgggg

SEQ ID NO: 171 - DNA, 73 bp, synthetic construct
  misc_feature 1..73: note = Synthetic Polynucleotide
  source 1..73: mol_type = other DNA
ccccaattttcccaaacagaggttctgtaaaccgaggttttggcttagcaacctctgtttgggaaaattgggg

SEQ ID NO: 172 - DNA, 74 bp, synthetic construct
  misc_feature 1..74: note = Synthetic Polynucleotide
  source 1..74: mol_type = other DNA
ccccaattttcccaaacagaggtatctgtaaaccgaggttttggcttagcaacctctgtttgggaaaattgggg

SEQ ID NO: 173 - DNA, 75 bp, synthetic construct
  misc_feature 1..75: note = Synthetic Polynucleotide
  source 1..75: mol_type = other DNA
ccccaattttcccaaacagaggtaatctgtaaaccgaggttttggcttagcaacctctgtttgggaaaattgggg

SEQ ID NO: 174 - DNA, 76 bp, synthetic construct
  misc_feature 1..76: note = Synthetic Polynucleotide
  source 1..76: mol_type = other DNA
ccccaattttcccaaacagaggtaaatctgtaaaccgaggttttggcttagcaacctctgtttgggaaaattgggg

SEQ ID NO: 175 - DNA, 77 bp, synthetic construct
  misc_feature 1..77: note = Synthetic Polynucleotide
  source 1..77: mol_type = other DNA
ccccaattttcccaaacagaggtgaaatctgtaaaccgaggttttggcttagcaacctctgtttgggaaaattgggg

SEQ ID NO: 176 - DNA, 78 bp, synthetic construct
  misc_feature 1..78: note = Synthetic Polynucleotide
  source 1..78: mol_type = other DNA
cccctcccatcacaggccctgaggtttaagagaaaaccatggttttgtgggccaggcccatgacccttctcctctggg

SEQ ID NO: 177 - DNA, 77 bp, synthetic construct
  misc_feature 1..77: note = Synthetic Polynucleotide
  source 1..77: mol_type = other DNA
ccttgtgttgtgtgtcttcaactcacagagttaaacgatgctttacacagagtagacttgaaacactctttttctgg

SEQ ID NO: 178 - DNA, 77 bp, synthetic construct
  misc_feature 1..77: note = Synthetic Polynucleotide
  source 1..77: mol_type = other DNA
ccaccggctcatgagaggtagagctaaggtccaaacctaggtttatctgagaccggaactcatgtgattaactgtgg

SEQ ID NO: 179 - DNA, 77 bp, synthetic construct
  misc_feature 1..77: note = Synthetic Polynucleotide
  source 1..77: mol_type = other DNA
ccttaagaacataaatccccaggaattcacagaaaccttggtttgagctttggatttcccgcaggatgtgggatagg

SEQ ID NO: 180 - DNA, 76 bp, synthetic construct
  misc_feature 1..76: note = Synthetic Polynucleotide
  source 1..76: mol_type = other DNA
ccactccctctcccccaaaaagtaaaggtagaaaaccaaggtttacaggcaacaaatagcacaatgaatggaatgg

SEQ ID NO: 181 - DNA, 77 bp, synthetic construct
  misc_feature 1..77: note = Synthetic Polynucleotide
  source 1..77: mol_type = other DNA
cctagggaagtgatcatagctgagtttctggaaaaacctaggttttaaagttgaggagacttaagtccaaaacctgg

SEQ ID NO: 182 - protein, 6 aa, synthetic construct
  REGION 1..6: note = Synthetic Polypeptide
GGSGGS

SEQ ID NO: 183 - protein, 24 aa, synthetic construct
  REGION 1..24: note = Synthetic Polypeptide
GGSGGSGGSGGSGGSGGSGGSGGS

SEQ ID NO: 184 - DNA, 4665 bp, synthetic construct
  misc_feature 1..4665: note = Synthetic Polynucleotide
  source 1..4665: mol_type = other DNA
atgctcattggctacgtgcgcgtctcaactaacgaccagaataccgatcttcagaggaacgcactggtttgtgcaggctgcgaacagattttcgaggacaaactcagcgggacacggacggacagacctggcctcaagcgagcactcaagaggctgcagaaaggagacactctggtggtctggaaattggaccgcctgggtcgaagcatgaagcatctcatttctctggttggcgaactgcgagaaagggggatcaactttcgaagtctgacggattccatagatacaagcagccccatgggccggttcttcttctacgtgatgggtgcactggctgaaatggaaagagaactcattatagagcgaaccatggcagggcttgcggctgccaggaataaaggcaggcggtttggaagaccaccaaagggtggatccggagggtccggaggtagtggcggcagcggtggttcaggtggcagcggagggtcaggaggctctgataaaaagtattctattggtttagctatcggcactaattccgttggatgggctgtcataaccgatgaatacaaagtaccttcaaagaaatttaaggtgttggggaacacagaccgtcattcgattaaaaagaatcttatcggtgccctcctattcgatagtggcgaaacggcagaggcgactcgcctgaaacgaaccgctcggagaaggtatacacgtcgcaagaaccgaatatgttacttacaagaaatttttagcaatgagatggccaaagttgacgattctttctttcaccgtttggaagagtccttccttgtcgaagaggacaagaaacatgaacggcaccccatctttggaaacatagtagatgaggtggcatatcatgaaaagtacccaacgatttatcacctcagaaaaaagctagttgactcaactgataaagcggacctgaggttaatctacttggctcttgcccatatgataaagttccgtgggcactttctcattgagggtgatctaaatccggacaactcggatgtcgacaaactgttcatccagttagtacaaacctataatcagttgtttgaagagaaccctataaatgcaagtggcgtggatgcgaaggctattcttagcgcccgcctctctaaatcccgacggctagaaaacctgatcgcacaattacccggagagaagaaaaatgggttgttcggtaaccttatagcgctctcactaggcctgacaccaaattttaagtcgaacttcgacttagctgaagatgccaaattgcagcttagtaaggacacgtacgatgacgatctcgacaatctactggcacaaattggagatcagtatgcggacttatttttggctgccaaaaaccttagcgatgcaatcctcctatctgacatactgagagttaatactgagattaccaaggcgccgttatccgcttcaatgatcaaaaggtacgatgaacatcaccaagacttgacacttctcaaggccctagtccgtcagcaactgcctgagaaatataaggaaatattctttgatcagtcgaaaaacgggtacgcaggttatattgacggcggagcgagtcaagaggaattctacaagtttatcaaacccatattagagaagatggatgggacggaagagttgcttgtaaaactcaatcgcgaagatctactgcgaaagcagcggactttcgacaacggtagcattccacatcaaatccacttaggcgaattgcatgctatacttagaaggcaggaggatttttatccgttcctcaaagacaatcgtgaaaagattgagaaaatcctaacctttcgcataccttactatgtgggacccctggcccgagggaactctcggttcgcatggatgacaagaaagtccgaagaaacgattactccatggaattttgaggaagttgtcgataaaggtgcgtcagctcaatcgttcatcgagaggatgaccaactttgacaagaatttaccgaacgaaaaagtattgcctaagcacagtttactttacgagtatttcacagtgtacaatgaactcacgaaagttaagtatgtcactgagggcatgcgtaaacccgcctttctaagcggagaacagaagaaagcaatagtagatctgttattcaagaccaaccgcaaagtgacagttaagcaattgaaagaggactactttaagaaaattgaatgcttcgattctgtcgagatctccggggtagaagatcgatttaatgcgtcacttggtacgtatcatgacctcctaaagataattaaagataaggacttcctggataacgaagagaatgaagatatcttagaagatatagtgttgactcttaccctctttgaagatcgggaaatgattgaggaaagactaaaaacatacgctcacctgttcgacgataaggttatgaaacagttaaagaggcgtcgctatacgggctggggacgattgtcgcggaaacttatcaacgggataagagacaagcaaagtggtaaaactattctcgattttctaaagagcgacggcttcgccaataggaactttatgcagctgatccatgatgactctttaaccttcaaagaggatatacaaaaggcacaggtttccggacaaggggactcattgcacgaacatattgcgaatcttgctggttcgccagccatcaaaaagggcatactccagacagtcaaagtagtggatgagctagttaaggtcatgggacgtcacaaaccggaaaacattgtaatcgagatggcacgcgaaaatcaaacgactcagaaggggcaaaaaaacagtcgagagcggatgaagagaatagaagagggtattaaagaactgggcagccagatcttaaaggagcatcctgtggaaaatacccaattgcagaacgagaaactttacctctattacctacaaaatggaagggacatgtatgttgatcaggaactggacataaaccgtttatctgattacgacgtcgatgccattgtaccccaatcctttttgaaggacgattcaatcgacaataaagtgcttacacgctcggataagaaccgagggaaaagtgacaatgttccaagcgaggaagtcgtaaagaaaatgaagaactattggcggcagctcctaaatgcgaaactgataacgcaaagaaagttcgataacttaactaaagctgagaggggtggcttgtctgaacttgacaaggccggatttattaaacgtcagctcgtggaaacccgccaaatcacaaagcatgttgcacagatactagattcccgaatgaatacgaaatacgacgagaacgataagctgattcgggaagtcaaagtaatcactttaaagtcaaaattggtgtcggacttcagaaaggattttcaattctataaagttagggagataaataactaccaccatgcgcacgacgcttatcttaatgccgtcgtagggaccgcactcattaagaaatacccgaagctagaaagtgagtttgtgtatggtgattacaaagtttatgacgtccgtaagatgatcgcgaaaagcgaacaggagataggcaaggctacagccaaatacttcttttattctaacattatgaatttctttaagacggaaatcactctggcaaacggagagatacgcaaacgacctttaattgaaaccaatggggagacaggtgaaatcgtatgggataagggccgggacttcgcgacggtgagaaaagttttgtccatgccccaagtcaacatagtaaagaaaactgaggtgcagaccggagggttttcaaaggaatcgattcttccaaaaaggaatagtgataagctcatcgctcgtaaaaaggactgggacccgaaaaagtacggtggcttcgatagccctacagttgcctattctgtcctagtagtggcaaaagttgagaagggaaaatccaagaaactgaagtcagtcaaagaattattggggataacgattatggagcgctcgtcttttgaaaagaaccccatcgacttccttgaggcgaaaggttacaaggaagtaaaaaaggatctcataattaaactaccaaagtatagtctgtttgagttagaaaatggccgaaaacggatgttggctagcgccggagagcttcaaaaggggaacgaactcgcactaccgtctaaatacgtgaatttcctgtatttagcgtcccattacgagaagttgaaaggttcacctgaagataacgaacagaagcaactttttgttgagcagcacaaacattatctcgacgaaatcatagagcaaatttcggaattcagtaagagagtcatcctagctgatgccaatctggacaaagtattaagcgcatacaacaagcacagggataaacccatacgtgagcaggcggaaaatattatccatttgtttactcttaccaacctcggcgctccagccgcattcaagtattttgacacaacgatagatcgcaaacgatacacttctaccaaggaggtgctagacgcgacactgattcaccaatccatcacgggattatatgaaactcggatagatttgtcacagcttgggggtgacggtggctccgattataaggatgatgacgacaagggaggttccccaaagaagaaaaggaaggtctga

SEQ ID NO: 185 - protein, 1554 aa, synthetic construct
  REGION 1..1554: note = Synthetic Polypeptide
MLIGYVRVSTNDQNTDLQRNALVCAGCEQIFEDKLSGTRTDRPGLKRALKRLQKGDTLVVWKLDRLGRSMKHLISLVGELRERGINFRSLTDSIDTSSPMGRFFFYVMGALAEMERELIIERTMAGLAAARNKGRRFGRPPKGGSGGSGGSGGSGGSGGSGGSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGGSDYKDDDDKGGSPKKKRKV

SEQ ID NO: 186 - DNA, 18 bp, synthetic construct
  misc_feature 1..18: note = Synthetic Polynucleotide
  source 1..18: mol_type = other DNA
ggtggtagcggtggatcc

SEQ ID NO: 187 - DNA, 45 bp, synthetic construct
  misc_feature 1..45: note = Synthetic Polynucleotide
  source 1..45: mol_type = other DNA
ggtggatccggtggttcaggtggcagcggagggtcaggaggctct

SEQ ID NO: 188 - DNA, 72 bp, synthetic construct
  misc_feature 1..72: note = Synthetic Polynucleotide
  source 1..72: mol_type = other DNA
ggtggatccggagggtccggaggtagtggcggcagcggtggttcaggtggcagcggagggtcaggaggctct

SEQ ID NO: 189 - DNA, 20 bp, synthetic construct
  misc_feature 1..20: note = Synthetic Polynucleotide
  source 1..20: mol_type = other DNA
acctctgtttgggaaaattg

SEQ ID NO: 190 - DNA, 21 bp, synthetic construct
  misc_feature 1..21: note = Synthetic Polynucleotide
  source 1..21: mol_type = other DNA
gcacactagttagggataaca

SEQ ID NO: 191 - DNA, 21 bp, synthetic construct
  misc_feature 1..21: note = Synthetic Polynucleotide
  source 1..21: mol_type = other DNA
gcctcagggcctgtgatggga

SEQ ID NO: 192 - DNA, 21 bp, synthetic construct
  misc_feature 1..21: note = Synthetic Polynucleotide
  source 1..21: mol_type = other DNA
gctcagggcctgtgatgggag

SEQ ID NO: 193 - DNA, 20 bp, synthetic construct
  misc_feature 1..20: note = Synthetic Polynucleotide
  source 1..20: mol_type = other DNA
ggcccatgacccttctcctc

SEQ ID NO: 194 - DNA, 20 bp, synthetic construct
  misc_feature 1..20: note = Synthetic Polynucleotide
  source 1..20: mol_type = other DNA
gcccatgacccttctcctct

SEQ ID NO: 195 - DNA, 20 bp, synthetic construct
  misc_feature 1..20: note = Synthetic Polynucleotide
  source 1..20: mol_type = other DNA
gacttgaaacactctttttc

SEQ ID NO: 196 - DNA, 21 bp, synthetic construct
  misc_feature 1..21: note = Synthetic Polynucleotide
  source 1..21: mol_type = other DNA
gagttgaagacacacaacaca

SEQ ID NO: 197 - DNA, 20 bp, synthetic construct
  misc_feature 1..20: note = Synthetic Polynucleotide
  source 1..20: mol_type = other DNA
ggaactcatgtgattaactg

SEQ ID NO: 198 - DNA, 21 bp, synthetic construct
  misc_feature 1..21: note = Synthetic Polynucleotide
  source 1..21: mol_type = other DNA
gtctacctctcatgagccggt

SEQ ID NO: 199 - DNA, 21 bp, synthetic construct
  misc_feature 1..21: note = Synthetic Polynucleotide
  source 1..21: mol_type = other DNA
gtttcccgcaggatgtgggat

SEQ ID NO: 200 - DNA, 21 bp, synthetic construct
  misc_feature 1..21: note = Synthetic Polynucleotide
  source 1..21: mol_type = other DNA
gcctggggatttatgttctta

SEQ ID NO: 201 - DNA, 21 bp, synthetic construct
  misc_feature 1..21: note = Synthetic Polynucleotide
  source 1..21: mol_type = other DNA
gaaatagcacaatgaatggaa

SEQ ID NO: 202 - DNA, 21 bp, synthetic construct
  misc_feature 1..21: note = Synthetic Polynucleotide
  source 1..21: mol_type = other DNA
gactttttgggggagagggag

SEQ ID NO: 203 - DNA, 20 bp, synthetic construct
  misc_feature 1..20: note = Synthetic Polynucleotide
  source 1..20: mol_type = other DNA
ggagacttaagtccaaaacc

SEQ ID NO: 204 - DNA, 21 bp, synthetic construct
  misc_feature 1..21: note = Synthetic Polynucleotide
  source 1..21: mol_type = other DNA
gtcagctatgatcacttccct

SEQ ID NO: 205 - DNA, 20 bp, synthetic construct
  misc_feature 1..20: note = Synthetic Polynucleotide
  source 1..20: mol_type = other DNA
gcagatgtagtgtttccaca

SEQ ID NO: 206 - DNA, 20 bp, synthetic construct
  misc_feature 1..20: note = Synthetic Polynucleotide
  source 1..20: mol_type = other DNA
gggtggggggagtttgctcc

SEQ ID NO: 207 - DNA, 21 bp, synthetic construct
  misc_feature 1..21: note = Synthetic Polynucleotide
  source 1..21: mol_type = other DNA
gatatccgtttatcagtgtca

SEQ ID NO: 208 - DNA, 21 bp, synthetic construct
  misc_feature 1..21: note = Synthetic Polynucleotide
  source 1..21: mol_type = other DNA
gttcctaagcttgggctgcag

SEQ ID NO: 209 - DNA, 21 bp, synthetic construct
  misc_feature 1..21: note = Synthetic Polynucleotide
  source 1..21: mol_type = other DNA
gcctaaaagtgactgggagaa

SEQ ID NO: 210 - DNA, 21 bp, synthetic construct
  misc_feature 1..21: note = Synthetic Polynucleotide
  source 1..21: mol_type = other DNA
gcacagtcccatatttcttgg

SEQ ID NO: 211 - DNA, 77 bp, synthetic construct
  misc_feature 1..77: note = Synthetic Polynucleotide
  source 1..77: mol_type = other DNA
cctttagtgaaaagtagacagctctgaatatgaaaggtaggttttcatttctgggaaagagacgccaagtgatgtgg

SEQ ID NO: 212 - DNA, 78 bp, synthetic construct
  misc_feature 1..78: note = Synthetic Polynucleotide
  source 1..78: mol_type = other DNA
cctccaataaatatgggactatgtggaaagaccaaacctacgtttgattggtgtacctgaaagtgacgggaagaatgg

SEQ ID NO: 213 - DNA, 78 bp, synthetic construct
  misc_feature 1..78: note = Synthetic Polynucleotide
  source 1..78: mol_type = other DNA
ccattctgcccgtcactttcaggtacaccaatcaaacgtaggtttagtcttttcacatagtcccatatttcttggagg

SEQ ID NO: 214 - DNA, 78 bp, synthetic construct
  misc_feature 1..78: note = Synthetic Polynucleotide
  source 1..78: mol_type = other DNA
ccattctccccgtcactttcaggtacaacaatcaaacgtaggtttggtcttttcacatagtcccatatttcttggagg

SEQ ID NO: 215 - DNA, 77 bp, synthetic construct
  misc_feature 1..77: note = Synthetic Polynucleotide
  source 1..77: mol_type = other DNA
ccttgtagtgtgtgtattcaactcacagagttaaacgatcctttacacagagcagacttgaaacactcttgttgtgg

SEQ ID NO: 216 - DNA, 77 bp, synthetic construct
  misc_feature 1..77: note = Synthetic Polynucleotide
  source 1..77: mol_type = other DNA
ccttgtagtgtgtgtattcaactcacagagttaaacgatcctttacacagagcatacttgaaacactctttttgtgg

SEQ ID NO: 217 - DNA, 77 bp, synthetic construct
  misc_feature 1..77: note = Synthetic Polynucleotide
  source 1..77: mol_type = other DNA
ccttgtgttgtgtgtattcaactcacagagttaaacgatcctttacacagagcagacttgaaacactctttttgtgg

SEQ ID NO: 218 - DNA, 77 bp, synthetic construct
  misc_feature 1..77: note = Synthetic Polynucleotide
  source 1..77: mol_type = other DNA
ccttgtgttgtgtttattcaactcacagagttaaacgatcctttacacagagcagacttgaaatactctttttgtgg

SEQ ID NO: 219 - DNA, 77 bp, synthetic construct; identical to SEQ ID NO: 216
  misc_feature 1..77: note = Synthetic Polynucleotide
  source 1..77: mol_type = other DNA

SEQ ID NO: 220 - DNA, 77 bp, synthetic construct
  misc_feature 1..77: note = Synthetic Polynucleotide
  source 1..77: mol_type = other DNA
ccttgtattgtgagtattcaactcacagagttaaacgatcctttacacagagcagacttgaaacactctttttgtgg

SEQ ID NO: 221 - DNA, 77 bp, synthetic construct; identical to SEQ ID NO: 177
  misc_feature 1..77: note = Synthetic Polynucleotide
  source 1..77: mol_type = other DNA

SEQ ID NO: 222 - DNA, 77 bp, synthetic construct
  misc_feature 1..77: note = Synthetic Polynucleotide
  source 1..77: mol_type = other DNA
ccttgtgttgtgtgtattcaactcacagagttaaacgatcctttacacagagcagacttgtaacactctttttgtgg

SEQ ID NO: 223 - DNA, 77 bp, synthetic construct
  misc_feature 1..77: note = Synthetic Polynucleotide
  source 1..77: mol_type = other DNA
ccttgtgttgtgtgtattcaactcacagagttaaacgatcctttacacagagcagacgtgaaacactctttttgtgg

SEQ ID NO: 224 - DNA, 77 bp, synthetic construct; identical to SEQ ID NO: 217
  misc_feature 1..77: note = Synthetic Polynucleotide
  source 1..77: mol_type = other DNA

SEQ ID NO: 225 - DNA, 77 bp, synthetic construct
  misc_feature 1..77: note = Synthetic Polynucleotide
  source 1..77: mol_type = other DNA
ccttgtgttgtgtgtattcaactcacagagttaaacgatcctttacacagaggagacttgtaacactctttttgtgg

SEQ ID NO: 226 - DNA, 77 bp, synthetic construct
  misc_feature 1..77: note = Synthetic Polynucleotide
  source 1..77: mol_type = other DNA
cctgaggttttccaggttttaaaaggaaacctaaaggtaggtttagcattaagtgtcttgaagtttattttaaaagg

SEQ ID NO: 227 - DNA, 76 bp, synthetic construct
  misc_feature 1..76: note = Synthetic Polynucleotide
  source 1..76: mol_type = other DNA
ccaaaattcccacaaaaccgaatgcatcagtcaaagcaaggtttgaagaaaagatttaccacttcagggagcttgg

SEQ ID NO: 228 - DNA, 77 bp, synthetic construct
  misc_feature 1..77: note = Synthetic Polynucleotide
  source 1..77: mol_type = other DNA
ccttttctggatatcgttgatgctctgtatgcaaaaggtaggtttttgggttatgttgttaaacagtgattgaatgg

SEQ ID NO: 229 - DNA, 78 bp, synthetic construct
  misc_feature 1..78: note = Synthetic Polynucleotide
  source 1..78: mol_type = other DNA
cctccaagaaatatggaactatgtgaaaagaccaaacctacgtttgattggtgtacctgaaagtgacagagagaatgg

SEQ ID NO: 230 - DNA, 78 bp, synthetic construct
  misc_feature 1..78: note = Synthetic Polynucleotide
  source 1..78: mol_type = other DNA
cctccaagaaatatgggactatgtgagaagaccaaacctacgtttgattggtgtacctgaaagtgatggggagaatgg

SEQ ID NO: 231 - DNA, 78 bp, synthetic construct
  misc_feature 1..78: note = Synthetic Polynucleotide
  source 1..78: mol_type = other DNA
ccattctccccatcgctttcaggtacaccaatcaaacgtaggtttggtcttttcacatagttccatattctttggagg

SEQ ID NO: 232 - DNA, 78 bp, synthetic construct
  misc_feature 1..78: note = Synthetic Polynucleotide
  source 1..78: mol_type = other DNA
ccattctccccatcactttcaggtgtaccgatcaaacgtaggtttggtcttttcacatagtcccatatttcttggagg

SEQ ID NO: 233 - DNA, 78 bp, synthetic construct
  misc_feature 1..78: note = Synthetic Polynucleotide
  source 1..78: mol_type = other DNA
cctccaagaaatatgggactatgtgaaaagaccaaacctacgtttgattggtgtacctgaaagtgacggggagaatgg

SEQ ID NO: 234 - DNA, 76 bp, synthetic construct
  misc_feature 1..76: note = Synthetic Polynucleotide
  source 1..76: mol_type = other DNA
ccttcagggcagaaacagctctactagcagagaaagcaagctttcaatattgtgcaatacaaaaacgagagcaggg

SEQ ID NO: 235 - DNA, 78 bp, synthetic construct
  misc_feature 1..78: note = Synthetic Polynucleotide
  source 1..78: mol_type = other DNA
ccattctcctcatctccttctggtactccaatcaaacgtaggtttggtcttttctcatagtctcatatttcttggagg

SEQ ID NO: 236 - DNA, 78 bp, synthetic construct
  misc_feature 1..78: note = Synthetic Polynucleotide
  source 1..78: mol_type = other DNA
cctccaagacatataggactatgtgaaaataccaaacctacgtttgattggtgtacctgaaagtgacagggagtatgg

SEQ ID NO: 237 - DNA, 76 bp, synthetic construct
  misc_feature 1..76: note = Synthetic Polynucleotide
  source 1..76: mol_type = other DNA
cctgccagataccagtagtcactgtgaattacaaagctacgtttcttccatagggaaagtttggagtccagccagg

SEQ ID NO: 238 - DNA, 78 bp, synthetic construct
  misc_feature 1..78: note = Synthetic Polynucleotide
  source 1..78: mol_type = other DNA
ccattctccctgtcactttcaggtacaccaatcaaacgtaggtttggtcttttcacatagtcccatatttcttggagg

SEQ ID NO: 239 - DNA, 78 bp, synthetic construct
  misc_feature 1..78: note = Synthetic Polynucleotide
  source 1..78: mol_type = other DNA
ccattctccccaccactttcaggtacaccaatcaaacgtaggtttggtcttttcacatagtcccatatttcttgtagg

SEQ ID NO: 240 - DNA, 78 bp, synthetic construct
  misc_feature 1..78: note = Synthetic Polynucleotide
  source 1..78: mol_type = other DNA
cctaaccagaaactaactaatagatatgggcagaaagcatcctttcacttttgttctgggagagggaagaagcaaagg

SEQ ID NO: 241 - DNA, 77 bp, synthetic construct
  misc_feature 1..77: note = Synthetic Polynucleotide
  source 1..77: mol_type = other DNA
ccattttggggaggccttgatgggaagctggaaaaggaagctttcctcccagtcctgctgaaggccttgccagctgg

SEQ ID NO: 242 - DNA, 78 bp, synthetic construct
  misc_feature 1..78: note = Synthetic Polynucleotide
  source 1..78: mol_type = other DNA
cctccaagaaacacaggactatgtgaaaagatcaaacctacgtttgattggtgttcctgaaagtgatggggagaatgg

SEQ ID NO: 243 - DNA, 78 bp, synthetic construct
  misc_feature 1..78: note = Synthetic Polynucleotide
  source 1..78: mol_type = other DNA
ccattctcttcatgactttcaggtacaccattgaaacgtaggtttggtcttttcacattgtcccatatttcttggagg

SEQ ID NO: 244 - DNA, 78 bp, synthetic construct
  misc_feature 1..78: note = Synthetic Polynucleotide
  source 1..78: mol_type = other DNA
ccattctccccatcactttcaggtacaccaatcaaacgtaggtttggtcttttcacatagtcccgtatttcttggtgg

SEQ ID NO: 245 - DNA, 78 bp, synthetic construct
  misc_feature 1..78: note = Synthetic Polynucleotide
  source 1..78: mol_type = other DNA
ccattctccctgtcactttcaggtacaccaatcaaacgtaggtttggtcttttcacatagtcccatatttcttggggg

SEQ ID NO: 246 - DNA, 77 bp, synthetic construct
  misc_feature 1..77: note = Synthetic Polynucleotide
  source 1..77: mol_type = other DNA
ccattctccctgtcactttcaggtacaccaatcaaacgtaggtttggtcttttcacatagtcccatatttcttgggg

SEQ ID NO: 247 - DNA, 78 bp, synthetic construct
  misc_feature 1..78: note = Synthetic Polynucleotide
  source 1..78: mol_type = other DNA
cctccaagaaatatgagattatatgaaaagaccaaacctacgtttgattggtgtactttaaagtgacggggagaatgg

SEQ ID NO: 248 - DNA, 78 bp, synthetic construct
  misc_feature 1..78: note = Synthetic Polynucleotide
  source 1..78: mol_type = other DNA
ccattctccccgtcattttcaggtacaccaatcaaacgtaggtttggtcttttcacatagtcccaaatttcttggagg

SEQ ID NO: 249 - DNA, 78 bp, synthetic construct
  misc_feature 1..78: note = Synthetic Polynucleotide
  source 1..78: mol_type = other DNA
cccccaagaaatgtgggactatatgaaaagaccaaacctacgtttgactggtgtacctaaaagtgatggggagaatgg

SEQ ID NO: 250 - DNA, 77 bp, synthetic construct
  misc_feature 1..77: note = Synthetic Polynucleotide
  source 1..77: mol_type = other DNA
ccccaagaaatgtgggactatatgaaaagaccaaacctacgtttgactggtgtacctaaaagtgatggggagaatgg

SEQ ID NO: 251 - DNA, 77 bp, synthetic construct
  misc_feature 1..77: note = Synthetic Polynucleotide
  source 1..77: mol_type = other DNA
cccattggtgctgaccagatggtgaaggaggcaaaggttgctttgaatgactgtgctctggggtgagccaggcctgg

SEQ ID NO: 252 - DNA, 76 bp, synthetic construct
  misc_feature 1..76: note = Synthetic Polynucleotide
  source 1..76: mol_type = other DNA
ccctttacagaggtgagctttgttattagtaaaaaggtaggtttccctgtttttctgaagaaaagctgtgagtggg

SEQ ID NO: 253 - DNA, 78 bp, synthetic construct
  misc_feature 1..78: note = Synthetic Polynucleotide
  source 1..78: mol_type = other DNA
ccactgcccattgacagagtggcgaggtgggtgaaaccttgctttcctcctggcccatgggcagggtggggctgtggg

SEQ ID NO: 254 - DNA, 77 bp, synthetic construct
  misc_feature 1..77: note = Synthetic Polynucleotide
  source 1..77: mol_type = other DNA
ccactgcccattgacagagtggcgaggtgggtgaaaccttgctttcctcctggcccatgggcagggtggggctgtgg

SEQ ID NO: 255 - DNA, 78 bp, synthetic construct
  misc_feature 1..78: note = Synthetic Polynucleotide
  source 1..78: mol_type = other DNA
ccattctccctgtcacttttagatacaccaatcaaacgtaggtttggtcttttcacatagtcccatgtttcttggagg

SEQ ID NO: 256 - DNA, 77 bp, synthetic construct
  misc_feature 1..77: note = Synthetic Polynucleotide
  source 1..77: mol_type = other DNA
cctccaagaaatatcaactgtgtgaaaagacgaaacctacgtttgattaatgtacctgaaagtgacagggagaatgg

SEQ ID NO: 257 - DNA, 78 bp, synthetic construct
  misc_feature 1..78: note = Synthetic Polynucleotide
  source 1..78: mol_type = other DNA
ccattctcccattaactttcaagtacaccaatcaaaggtaggtttggtgttttcccatagtcccgtatttcttggagg

SEQ ID NO: 258 - DNA, 78 bp, synthetic construct
  misc_feature 1..78: note = Synthetic Polynucleotide
  source 1..78: mol_type = other DNA
ccttttcatcatgcccctttcactttaaggtgaaaaccttgctttacatgtcagagaaaagaagagccctcagctggg

SEQ ID NO: 259 - DNA, 77 bp, synthetic construct
  misc_feature 1..77: note = Synthetic Polynucleotide
  source 1..77: mol_type = other DNA
ccttttcatcatgcccctttcactttaaggtgaaaaccttgctttacatgtcagagaaaagaagagccctcagctgg

SEQ ID NO: 260 - DNA, 78 bp, synthetic construct
  misc_feature 1..78: note = Synthetic Polynucleotide
  source 1..78: mol_type = other DNA
ccattcaccccgtcactttcaggtacaccaatcaaacgtaggtttggtcttttcacatagtcccatatttcttggagg

SEQ ID NO: 261 - DNA, 77 bp, synthetic construct
  misc_feature 1..77: note = Synthetic Polynucleotide
  source 1..77: mol_type = other DNA
cctccaagaaatatgggactatgtgaaaagaccaaacctacgtttgatggtgtacccgaaagtgacagggagaatgg

SEQ ID NO: 262 - DNA, 78 bp, synthetic construct
  misc_feature 1..78: note = Synthetic Polynucleotide
  source 1..78: mol_type = other DNA
ccaccaagaaatatgggactatgtgaaaagaccaaacctacgtttgataggtatacctgaaagtgacagggagaatgg

SEQ ID NO: 263 - DNA, 78 bp, synthetic construct
  misc_feature 1..78: note = Synthetic Polynucleotide
  source 1..78: mol_type = other DNA
ccattctccccatcactttcaggtgcaccaatcaaacgtaggtttggtcttttcacatagtcccatatttcttggagg

SEQ ID NO: 264 - DNA, 78 bp, synthetic construct
  misc_feature 1..78: note = Synthetic Polynucleotide
  source 1..78: mol_type = other DNA
ccctcaagaaatatgagactatgtgaaaagaccaaacctacgtttgactggtatacctgaaagtgacagggagaatgg

SEQ ID NO: 265 - DNA, 77 bp, synthetic construct
  misc_feature 1..77: note = Synthetic Polynucleotide
  source 1..77: mol_type = other DNA
cctcaagaaatatgagactatgtgaaaagaccaaacctacgtttgactggtatacctgaaagtgacagggagaatgg

SEQ ID NO: 266 - DNA, 78 bp, synthetic construct
  misc_feature 1..78: note = Synthetic Polynucleotide
  source 1..78: mol_type = other DNA
cctccaacaaatatgggactatgtgaaaagaccaaacctacgtttgattggtgtacctgaaagtgacggggataatgg

SEQ ID NO: 267 - DNA, 78 bp, synthetic construct
  misc_feature 1..78: note = Synthetic Polynucleotide
  source 1..78: mol_type = other DNA
ccattctctccctcactttcaagtacaccaatcaaacgtaggtttggtcttttcacatagtcttatatttcttggcgg

SEQ ID NO: 268 - DNA, 77 bp, synthetic construct
  misc_feature 1..77: note = Synthetic Polynucleotide
  source 1..77: mol_type = other DNA
ccattctccctgtcactgtcagtacaccaatcaaacgtaggtttggtctcttcacatagtcccatatttcttggagg

SEQ ID NO: 269 - DNA, 77 bp, synthetic construct
  misc_feature 1..77: note = Synthetic Polynucleotide
  source 1..77: mol_type = other DNA
cctccaagaaatatgggactatgtgaacagaccaaacctacgtttgattggtgtacctgaaagtgatggcagaatgg

SEQ ID NO: 270 - DNA, 77 bp, synthetic construct
  misc_feature 1..77: note = Synthetic Polynucleotide
  source 1..77: mol_type = other DNA
ccaccatgcctggccaccacacatttttttctaaagcttggttttggccacagtgagagtttcttgggctgtcaggg

SEQ ID NO: 271 - DNA, 76 bp, synthetic construct
  misc_feature 1..76: note = Synthetic Polynucleotide
  source 1..76: mol_type = other DNA
ccaccatgcctggccaccacacatttttttctaaagcttggttttggccacagtgagagtttcttgggctgtcagg

SEQ ID NO: 272 - DNA, 77 bp, synthetic construct
  misc_feature 1..77: note = Synthetic Polynucleotide
  source 1..77: mol_type = other DNA
cccactaggtggcgatatctgagggtccaatgaaaccatgctttttactcagatcttccactaaccacctcccccgg

SEQ ID NO: 273 - DNA, 78 bp, synthetic construct
  misc_feature 1..78: note = Synthetic Polynucleotide
  source 1..78: mol_type = other DNA
cctctaagaaatatgggactatgtgaaaagaccaaacctacgtttgactggtgtacctgaaagtgacggggagaatgg

SEQ ID NO: 274 - DNA, 78 bp, synthetic construct
  misc_feature 1..78: note = Synthetic Polynucleotide
  source 1..78: mol_type = other DNA
cctccaagaaatatgggactatgtgaaaagaccaaacctacgtttgattagtgtacctgaaagtgacggggagaatgg

SEQ ID NO: 275 - DNA, 78 bp, synthetic construct
  misc_feature 1..78: note = Synthetic Polynucleotide
  source 1..78: mol_type = other DNA
ccattctccctgtcactttcaggtacatcaatcaaacgtaggtttggtcttttcacatagtcccatatttcttggagg

SEQ ID NO: 276 - DNA, 78 bp, synthetic construct
  misc_feature 1..78: note = Synthetic Polynucleotide
  source 1..78: mol_type = other DNA
cctccaagaaatatgggactatgtgaaaagaccaaacctacgtttgactggtgtacctgaaagggatggggagaatgg

SEQ ID NO: 277 - DNA, 78 bp, synthetic construct
  misc_feature 1..78: note = Synthetic Polynucleotide
  source 1..78: mol_type = other DNA
cccccaagaaatatgagactatgtgaaaagaccaaacctacgtttgattggtgtacctgaaagtgacagggagaatgg

SEQ ID NO: 278 - DNA, 77 bp, synthetic construct
  misc_feature 1..77: note = Synthetic Polynucleotide
  source 1..77: mol_type = other DNA
ccccaagaaatatgagactatgtgaaaagaccaaacctacgtttgattggtgtacctgaaagtgacagggagaatgg

SEQ ID NO: 279 - DNA, 78 bp, synthetic construct
  misc_feature 1..78: note = Synthetic Polynucleotide
  source 1..78: mol_type = other DNA
cctctaagaaatatgggactatgtgaaaagaccaaacctacgtttgattggtgtaactgaaagtgacagggagaatgg

SEQ ID NO: 280 - DNA, 78 bp, synthetic construct
  misc_feature 1..78: note = Synthetic Polynucleotide
  source 1..78: mol_type = other DNA
cctccaagaaatatgcgcctatgtgaaaagaccaaacctacgtttgattggtatacctgaaagtgatggagagaatgg

SEQ ID NO: 281 - DNA, 78 bp, synthetic construct
  misc_feature 1..78: note = Synthetic Polynucleotide
  source 1..78: mol_type = other DNA
ccattctccctgtcactttgaggtacaccaatcaaacgtaggtttggtcttttcacatattcgcatatttcttggagg

SEQ ID NO: 282 - DNA, 78 bp, synthetic construct
  misc_feature 1..78: note = Synthetic Polynucleotide
  source 1..78: mol_type = other DNA
ccattctccccgtcactttcaggtacaccaaccaaacgttggtttggtcttttcacatagtcccatatttcttggagg

SEQ ID NO: 283 - DNA, 78 bp, synthetic construct
  misc_feature 1..78: note = Synthetic Polynucleotide
  source 1..78: mol_type = other DNA
ccattctccctgtcactttccagtacaccagtcaaacgtaggtttggtcttttcacatactcccatatttcttggagg

SEQ ID NO: 284 - DNA, 77 bp, synthetic construct
  misc_feature 1..77: note = Synthetic Polynucleotide
  source 1..77: mol_type = other DNA
cctggcctaatttttaattcttagtttgacttaaaccttgcttttagtgtgatggcgacaaaagctgagctgaaagg

SEQ ID NO: 285 - DNA, 76 bp, synthetic construct
  misc_feature 1..76: note = Synthetic Polynucleotide
  source 1..76: mol_type = other DNA
ccagtgctttttggttttaaaggcaagcctccaaaccttcctttctcctggatgctgtggtggttgccatgcatgg

SEQ ID NO: 286 - DNA, 76 bp, synthetic construct
  misc_feature 1..76: note = Synthetic Polynucleotide
  source 1..76: mol_type = other DNA
cccaactcctgcgagaagtagctcaccatgacaaagctacctttgcttttatcgttttgcaaaacaaaaaaggggg

SEQ ID NO: 287 - DNA, 78 bp, synthetic construct
  misc_feature 1..78: note = Synthetic Polynucleotide
  source 1..78: mol_type = other DNA
ccattctccccgtcactttgaggtgtgccaatcaaacgtaggtttggtcttttcacatagtcctatatttcttggagg

SEQ ID NO: 288 - DNA, 78 bp, synthetic construct
  misc_feature 1..78: note = Synthetic Polynucleotide
  source 1..78: mol_type = other DNA
cctccaaaaaatatgggactacgtaaaaagaccaaacctacgtttgattggtgtacctgaaactgacagggagaatgg

SEQ ID NO: 289 - DNA, 78 bp, synthetic construct
  misc_feature 1..78: note = Synthetic Polynucleotide
  source 1..78: mol_type = other DNA
ccattctccccgtcactttcaggtacaccaatcaaacgtaggtttggtcttttcacatagttccatatttcttggagg

SEQ ID NO: 290 - DNA, 78 bp, synthetic construct
  misc_feature 1..78: note = Synthetic Polynucleotide
  source 1..78: mol_type = other DNA
cctacaagatatatgggactatgtgaaaagaccaaacctacgttttactggtgtgcctgaaactgacggggagaatgg

SEQ ID NO: 291 - DNA, 78 bp, synthetic construct
  misc_feature 1..78: note = Synthetic Polynucleotide
  source 1..78: mol_type = other DNA
ccattctctctgtcactttcaggtacaccaatcaaacgtaggtttggtcttttcacatagtcccatatttcttggagg

SEQ ID NO: 292 - DNA, 78 bp, synthetic construct
  misc_feature 1..78: note = Synthetic Polynucleotide
  source 1..78: mol_type = other DNA
cctccaagaaatatgggactatgtgaaaagaccaaacctacgtttcattggtgtacctgaaagtgatagggagaatgg

SEQ ID NO: 293 - DNA, 78 bp, synthetic construct
  misc_feature 1..78: note = Synthetic Polynucleotide
  source 1..78: mol_type = other DNA
cctccaaaaaatatgggatgatgtgaaaagaccaaacctaggtttgactggtgtacctgaaaatgatggggagaatgg

SEQ ID NO: 294 - DNA, 78 bp, synthetic construct
  misc_feature 1..78: note = Synthetic Polynucleotide
  source 1..78: mol_type = other DNA
cctccaagaaatatgagactatgtgaaaagaccaaacctacgtttgattggtgtacctgaaagtgacagggagaatgg

SEQ ID NO: 295 - DNA, 78 bp, synthetic construct
  misc_feature 1..78: note = Synthetic Polynucleotide
  source 1..78: mol_type = other DNA
cctccaagaaatatgggactacgtgaaaagatcaaacctacgtttgattgttgtacctgaaagtgatggggagaatgg

SEQ ID NO: 296 - DNA, 78 bp, synthetic construct
  misc_feature 1..78: note = Synthetic Polynucleotide
  source 1..78: mol_type = other DNA
cctccaagaaatatgggactatgtgaaaagaccaaacctacgtttgattgttgtacctgaaagtgatggggagaatgg

SEQ ID NO: 297 - DNA, 78 bp, synthetic construct
  misc_feature 1..78: note = Synthetic Polynucleotide
  source 1..78: mol_type = other DNA
cctcaaaagtgttctggttttgttttgttttttaaaccatggttttacctctggcttagtgggactaaaaataggagg

SEQ ID NO: 298 - DNA, 78 bp, synthetic construct
  misc_feature 1..78: note = Synthetic Polynucleotide
  source 1..78: mol_type = other DNA
cctccaagaaatatgggactatgtgaaaagaccaaacctacgtttgactggtgtacctgaaagtgatggggaaaatgg

SEQ ID NO: 299 - DNA, 78 bp, synthetic construct
  misc_feature 1..78: note = Synthetic Polynucleotide
  source 1..78: mol_type = other DNA
cctccaagaaatatgggactgtgtgtaaagaccaaacctacgtttgattggtgtacctcaaagtgatggggagaatgg

SEQ ID NO: 300 - DNA, 78 bp, synthetic construct
  misc_feature 1..78: note = Synthetic Polynucleotide
  source 1..78: mol_type = other DNA
ccattctccccatcacattcaggtacaccaatcaaacgtaggtttggtcttttcacatagtcccatatttcttggagg

SEQ ID NO: 301 - DNA, 77 bp, synthetic construct
  misc_feature 1..77: note = Synthetic Polynucleotide
  source 1..77: mol_type = other DNA
cccctggaaaagttggagcatcacaggaaaagcaaaccaaccttttttctcccctaggtaaactggggagccagggg

SEQ ID NO: 302 - DNA, 76 bp, synthetic construct
  misc_feature 1..76: note = Synthetic Polynucleotide
  source 1..76: mol_type = other DNA
ccctggaaaagttggagcatcacaggaaaagcaaaccaaccttttttctcccctaggtaaactggggagccagggg

SEQ ID NO: 303 - DNA, 77 bp, synthetic construct
  misc_feature 1..77: note = Synthetic Polynucleotide
  source 1..77: mol_type = other DNA
ccttccccagttgcagcagacaagagtctcgaaaagcttgctttggttgctgcagtggatgggttggtaggcacagg

SEQ ID NO: 304 - DNA, 76 bp, synthetic construct
  misc_feature 1..76: note = Synthetic Polynucleotide
  source 1..76: mol_type = other DNA
cccccacctcccaagctgctggcttctcgaataaagctacctttccttttaccaaaacttgtctctcgaatgtcgg

SEQ ID NO: 305 - DNA, 77 bp, synthetic construct
  misc_feature 1..77: note = Synthetic Polynucleotide
  source 1..77: mol_type = other DNA
ccttggccctggacagctgcttttccttccctaaaccttggtttccccctttgtgcaggtgggtgggtttgggctgg

SEQ ID NO: 306 - DNA, 78 bp, synthetic construct
  misc_feature 1..78: note = Synthetic Polynucleotide
  source 1..78: mol_type = other DNA
cctcttctagtgaacccatggggttaccaagggaaagcaaccttttgataaatattcccatctttttatgttgtctgg

SEQ ID NO: 307 - DNA, 78 bp, synthetic construct
  misc_feature 1..78: note = Synthetic Polynucleotide
  source 1..78: mol_type = other DNA
ccacttgaaagggttaccaaggataagatttttaaagcttgctttcacaaacaactcatgctccaggcttgtcagtgg

SEQ ID NO: 308 - DNA, 78 bp, synthetic construct
  misc_feature 1..78: note = Synthetic Polynucleotide
  source 1..78: mol_type = other DNA
cctttctccccatcactttcaggtacaccaatcaaacgtaggtttgatcttttcacatagtcccatatttcttggagg

SEQ ID NO: 309 - DNA, 78 bp, synthetic construct
  misc_feature 1..78: note = Synthetic Polynucleotide
  source 1..78: mol_type = other DNA
ccattctccccatcaatttcagttacaccaatgaaacgtaggtttggccttttcacatagtcccatatttcttagagg

SEQ ID NO: 310 - DNA, 78 bp, synthetic construct
  misc_feature 1..78: note = Synthetic Polynucleotide
  source 1..78: mol_type = other DNA
ccattctccctgtcactctcaggtacaccaatcaaacgtaggtttggtcttttcatatagtcccatatttcttggagg

SEQ ID NO: 311 - DNA, 77 bp, synthetic construct
  misc_feature 1..77: note = Synthetic Polynucleotide
  source 1..77: mol_type = other DNA
cctccaagaaaattgggactatgtgaaaaaaccaaacctacgtttgattgatgtacctgaaagtgacaggagaatgg

SEQ ID NO: 312 - DNA, 78 bp, synthetic construct
  misc_feature 1..78: note = Synthetic Polynucleotide
  source 1..78: mol_type = other DNA
ccttcaagaaatatgggactatgtgaaaggacaaaacctacgttttattggtgtacctgaaagtgacagggagaatgg

SEQ ID NO: 313 - DNA, 78 bp, synthetic construct
  misc_feature 1..78: note = Synthetic Polynucleotide
  source 1..78: mol_type = other DNA
ccattctccccatcactttcaggtacgctaatcaaacgtaggtttgatcttttcacatagtcttatatttcttggagg

SEQ ID NO: 314 - DNA, 78 bp, synthetic construct
  misc_feature 1..78: note = Synthetic Polynucleotide
  source 1..78: mol_type = other DNA
cctccaagaaatatgggactatgtgaaaagaccaaacctacgtttgactggtgtacctcaatgtgacagggagaatgg

SEQ ID NO: 315 - DNA, 78 bp, synthetic construct
  misc_feature 1..78: note = Synthetic Polynucleotide
  source 1..78: mol_type = other DNA
ccattctccctgtcacttttaggtacaccaatcaaacgtacgtttggtcttttcacatagacccatatttcttggagg

SEQ ID NO: 316 - DNA, 78 bp, synthetic construct
  misc_feature 1..78: note = Synthetic Polynucleotide
  source 1..78: mol_type = other DNA
ccttcaagaaatatgggactgtgtgaaaagaccaaagctaggtttgattggtgtacctgaaagtgatggggagaatgg

SEQ ID NO: 317 - DNA, 76 bp, synthetic construct
  misc_feature 1..76: note = Synthetic Polynucleotide
  source 1..76: mol_type = other DNA
cctactattcacagagtaatgcagtttgctgaaaaggttggtttttgctgacctctgagagctcacattacagtgg

SEQ ID NO: 318 - DNA, 78 bp, synthetic construct
  misc_feature 1..78: note = Synthetic Polynucleotide
  source 1..78: mol_type = other DNA
ccattctctctgtcactttctggtacaccaatcaaacgtaggtttgctcttttcacataatcccatatttattgaagg

SEQ ID NO: 319 - DNA, 77 bp, synthetic construct
  misc_feature 1..77: note = Synthetic Polynucleotide
  source 1..77: mol_type = other DNA
ccataacatgtatttgctggtgctagactctccaaagctaggtttctttctacaacaatggctggaagtcttcttgg

SEQ ID NO: 320 - DNA, 78 bp, synthetic construct
  misc_feature 1..78: note = Synthetic Polynucleotide
  source 1..78: mol_type = other DNA
ccattctccccatcactttcaggtacaccaatcaaacgtaggtttggtcttctcacacagtcccatatttcttggagg

SEQ ID NO: 321 - DNA, 78 bp, synthetic construct
  misc_feature 1..78: note = Synthetic Polynucleotide
  source 1..78: mol_type = other DNA
ccattcttcccattactttcaggtacaccaatcaaacgtaggtttggtcttttcacatagtcccacatttcttggagg

SEQ ID NO: 322 - DNA, 78 bp, synthetic construct
  misc_feature 1..78: note = Synthetic Polynucleotide
  source 1..78: mol_type = other DNA
ccattctccccctcactttcaggtacaccaatcaaacgtaggtttggtcttttcacattgtcccatatttcttggagg

SEQ ID NO: 323 - DNA, 78 bp, synthetic construct
  misc_feature 1..78: note = Synthetic Polynucleotide
  source 1..78: mol_type = other DNA
ccattctccccagcacttacaggtacaccaatcaaacgtaggtttggtcatttcacatagtcccatatttcttggagg

SEQ ID NO: 324 - DNA, 78 bp, synthetic construct
  misc_feature 1..78: note = Synthetic Polynucleotide
  source 1..78: mol_type = other DNA
ccattctccctgtcactttcaggtacagcaatcaaacgtaggtttggtcttttcacatggtcccatatttcttggagg

SEQ ID NO: 325 - DNA, 78 bp, synthetic construct
  misc_feature 1..78: note = Synthetic Polynucleotide
  source 1..78: mol_type = other DNA
cctccaagaaatatgagactatgtgaaaagaccaaacctacgtttgattggtgtacctgaaagtgacggggaagatgg

SEQ ID NO: 326 - DNA, 78 bp, synthetic construct; identical to SEQ ID NO: 294
  misc_feature 1..78: note = Synthetic Polynucleotide
  source 1..78: mol_type = other DNA

SEQ ID NO: 327 - DNA, 78 bp, synthetic construct
  misc_feature 1..78: note = Synthetic Polynucleotide
  source 1..78: mol_type = other DNA
cctccaagagatatgagactatgtaaatagaccaaacctacctttgattggtgtacgtgaaagtgacaggaagaatgg

SEQ ID NO: 328 - DNA, 78 bp, synthetic construct
  misc_feature 1..78: note = Synthetic Polynucleotide
  source 1..78: mol_type = other DNA
ccattctccccatcactttcaggtacaccaaccaaacgtaggtttggtcttttcacatagtctcatatttcttggagg

SEQ ID NO: 329 - DNA, 76 bp, synthetic construct
  misc_feature 1..76: note = Synthetic Polynucleotide
  source 1..76: mol_type = other DNA
cctccattgactactccttatcattggctagaaaacctacctttcaaccagtttctaaggccaagaaacttggagg

SEQ ID NO: 330 - DNA, 78 bp, synthetic construct
  misc_feature 1..78: note = Synthetic Polynucleotide
  source 1..78: mol_type = other DNA
ccaccaagaaatatgggactacgtgaaaagaccaaacctacgtttgatgggtgtgcctgaaagtgacgggaagaatgg

SEQ ID NO: 331 - DNA, 78 bp, synthetic construct
  misc_feature 1..78: note = Synthetic Polynucleotide
  source 1..78: mol_type = other DNA
cctccaagaaataagggactatgtgaaaagaccaaacctacgtttgattggtgtacctgaaggtgacagggagaatgg

SEQ ID NO: 332 - DNA, 77 bp, synthetic construct
  misc_feature 1..77: note = Synthetic Polynucleotide
  source 1..77: mol_type = other DNA
ccaaagggcctttgtgattctactttgtaatataaaggatggtttcttactacggttggtgtccttgcaggagtggg

SEQ ID NO: 333 - DNA, 78 bp, synthetic construct
  misc_feature 1..78: note = Synthetic Polynucleotide
  source 1..78: mol_type = other DNA
cctccaagaaatatgggactatgtgaaaagaccaaacctacgtttgattggtgtacctgaaagtgatggggagaatgg

SEQ ID NO: 334 - DNA, 78 bp, synthetic construct
  misc_feature 1..78: note = Synthetic Polynucleotide
  source 1..78: mol_type = other DNA
ccattctccccgttactttcaggtacaccaataaaacctaggtttggtcttttcacatagtcccatatttcttggagg

SEQ ID NO: 335 - DNA, 76 bp, synthetic construct
  misc_feature 1..76: note = Synthetic Polynucleotide
  source 1..76: mol_type = other DNA
cccatatctctggcaagggcagctctctggctaaaccaagctttcctgtagagcttgagttccaaggcagcgttgg

SEQ ID NO: 336 - DNA, 77 bp, synthetic construct; identical to SEQ ID NO: 215
  misc_feature 1..77: note = Synthetic Polynucleotide
  source 1..77: mol_type = other DNA

SEQ ID NO: 337 - DNA, 77 bp, synthetic construct; identical to SEQ ID NO: 216
  misc_feature 1..77: note = Synthetic Polynucleotide
  source 1..77: mol_type = other DNA

SEQ ID NO: 338 - DNA, 77 bp, synthetic construct; identical to SEQ ID NO: 217
  misc_feature 1..77: note = Synthetic Polynucleotide
  source 1..77: mol_type = other DNA

SEQ ID NO: 339 - DNA, 77 bp, synthetic construct; identical to SEQ ID NO: 218
  misc_feature 1..77: note = Synthetic Polynucleotide
  source 1..77: mol_type = other DNA

SEQ ID NO: 340 - DNA, 77 bp, synthetic construct; identical to SEQ ID NO: 216
  misc_feature 1..77: note = Synthetic Polynucleotide
  source 1..77: mol_type = other DNA

SEQ ID NO: 341 - DNA, 77 bp, synthetic construct; identical to SEQ ID NO: 220
  misc_feature 1..77: note = Synthetic Polynucleotide
  source 1..77: mol_type = other DNA

SEQ ID NO: 342 - DNA, 77 bp, synthetic construct; identical to SEQ ID NO: 177
  misc_feature 1..77: note = Synthetic Polynucleotide
  source 1..77: mol_type = other DNA

SEQ ID NO: 343 - DNA, 77 bp, synthetic construct; identical to SEQ ID NO: 222
  misc_feature 1..77: note = Synthetic Polynucleotide
  source 1..77: mol_type = other DNA

SEQ ID NO: 344 - DNA, 77 bp, synthetic construct; identical to SEQ ID NO: 223
  misc_feature 1..77: note = Synthetic Polynucleotide
  source 1..77: mol_type = other DNA

SEQ ID NO: 345 - DNA, 77 bp, synthetic construct; identical to SEQ ID NO: 217
  misc_feature 1..77: note = Synthetic Polynucleotide
  source 1..77: mol_type = other DNA

SEQ ID NO: 346 - DNA, 77 bp, synthetic construct; identical to SEQ ID NO: 225
  misc_feature 1..77: note = Synthetic Polynucleotide
  source 1..77: mol_type = other DNA

SEQ ID NO: 347 - DNA, 77 bp, synthetic construct
  misc_feature 1..77: note = Synthetic Polynucleotide
  source 1..77: mol_type = other DNA
ccttttcataagaagaaaatcgactcatcattgaaaccaagctttggtacaatttcattgatgtttccagaagcagg

SEQ ID NO: 348 - DNA, 76 bp, synthetic construct
  misc_feature 1..76: note = Synthetic Polynucleotide
  source 1..76: mol_type = other DNA
cccatagactatgatagaaacaaaataacccaaaagctagctttctgattgagtttccataaatgcaatgtgaagg

SEQ ID NO: 349 - DNA, 77 bp, synthetic construct
  misc_feature 1..77: note = Synthetic Polynucleotide
  source 1..77: mol_type = other DNA
ccattcacttgtcactttctggtacaccaatcaaacgtaggtttggtcttttcacatagtctcatatttcttggagg

SEQ ID NO: 350 - DNA, 78 bp, synthetic construct
  misc_feature 1..78: note = Synthetic Polynucleotide
  source 1..78: mol_type = other DNA
cctccaagaaatatgggactctgtaaagagaccaaacctacgtttgattggtgtacctgaaagtgaaggggagaatgg

SEQ ID NO: 351 - DNA, 78 bp, synthetic construct
  misc_feature 1..78: note = Synthetic Polynucleotide
  source 1..78: mol_type = other DNA
ccattctccccgtcattttcaggtacaccaatcaaacctaggtttggtctttttacatagtcccatatttcttggagg

SEQ ID NO: 352 - DNA, 78 bp, synthetic construct
  misc_feature 1..78: note = Synthetic Polynucleotide
  source 1..78: mol_type = other DNA
cctccacgaaacatgggactatgtgaaaagaccaaacctacgtttgattggtgtacctgaaagtgacagggagaatgg

SEQ ID NO: 353 - DNA, 78 bp, synthetic construct
  misc_feature 1..78: note = Synthetic Polynucleotide
  source 1..78: mol_type = other DNA
ccaatttccccctcactttcagatacaccaatcaaacgtaggtttggtcttttcacatagttccatatttcctggagg

SEQ ID NO: 354 - DNA, 78 bp, synthetic construct
  misc_feature 1..78: note = Synthetic Polynucleotide
  source 1..78: mol_type = other DNA
ccattctccccatcactttcaggtacaccaatcaaacgtaggtttggtcttttcacatattcccatatgtcttggagg

SEQ ID NO: 355 - DNA, 78 bp, synthetic construct
  misc_feature 1..78: note = Synthetic Polynucleotide
  source 1..78: mol_type = other DNA
cccaccggctcatgagaggtagagctaaggtccaaacctaggtttatctgagaccggaactcatgtgattaactgtgg

SEQ ID NO: 356 - DNA, 77 bp, synthetic construct; identical to SEQ ID NO: 178
  misc_feature 1..77: note = Synthetic Polynucleotide
  source 1..77: mol_type = other DNA

SEQ ID NO: 357 - DNA, 78 bp, synthetic construct
  misc_feature 1..78: note = Synthetic Polynucleotide
  source 1..78: mol_type = other DNA
ccttcaagaaatatgggactatgtgaagagaccaaacctacgtttgattggtgtagccaaaagtgatggggaaaatgg

SEQ ID NO: 358 - DNA, 78 bp, synthetic construct
  misc_feature 1..78: note = Synthetic Polynucleotide
  source 1..78: mol_type = other DNA
cctcagattagatttacttgcaaagagacatttaaaggatcgttttgatactattttgaaagtactatacaaagatgg

SEQ ID NO: 359 - DNA, 77 bp, synthetic construct; identical to SEQ ID NO: 179
  misc_feature 1..77: note = Synthetic Polynucleotide
  source 1..77: mol_type = other DNA

SEQ ID NO: 360 - DNA, 78 bp, synthetic construct
  misc_feature 1..78: note = Synthetic Polynucleotide
  source 1..78: mol_type = other DNA
ccattctctctgtcactttcaggtacaccaatcaaacgtaggtttggtcttttctcatagtcccatatttcttggagg

SEQ ID NO: 361 - DNA, 76 bp, synthetic construct
  misc_feature 1..76: note = Synthetic Polynucleotide
  source 1..76: mol_type = other DNA
ccatttaccatcattctctgtcatggcaggtgaaagcaagcttttatatagacaatgttctacttagtttacaggg

SEQ ID NO: 362 - DNA, 77 bp, synthetic construct
  misc_feature 1..77: note = Synthetic Polynucleotide
  source 1..77: mol_type = other DNA
cccaaagttaattttactctttttctgaatcaaaaggaacctttcctccatgagaagaatcctgccatatttctagg

SEQ ID NO: 363 - DNA, 78 bp, synthetic construct
  misc_feature 1..78: note = Synthetic Polynucleotide
  source 1..78: mol_type = other DNA
cctccaagaaatatgggactatgtgaaaagaccaaacctacgtttgattgctatacatgaaagtgacggggagaatgg

SEQ ID NO: 364 - DNA, 78 bp, synthetic construct
  misc_feature 1..78: note = Synthetic Polynucleotide
  source 1..78: mol_type = other DNA
ccttcaagaaatatgggactatgtgaaaagaccaaacctacctttgattggtgtacctgaaagtgatgggaagaatgg

SEQ ID NO: 365 - DNA, 78 bp, synthetic construct
  misc_feature 1..78: note = Synthetic Polynucleotide
  source 1..78: mol_type = other DNA
ccattctccccatcactttcaggtacaccaatcaaacgtaggtttggtcttttcacatagtcccatagttcttggagg

SEQ ID NO: 366 - DNA, 78 bp, synthetic construct
  misc_feature 1..78: note = Synthetic Polynucleotide
  source 1..78: mol_type = other DNA
ccattctccccgtcactttcagggacaacaatcaaacgtaggtttggcctttgcacatagtcttatatttcttggagg

SEQ ID NO: 367 - DNA, 78 bp, synthetic construct
  misc_feature 1..78: note = Synthetic Polynucleotide
  source 1..78: mol_type = other DNA
ccattctccccatcactttcaggtacaccaatcaaacgtaggtttggtcttttcacatagtcccatatttcttggagg

SEQ ID NO: 368 - DNA, 78 bp, synthetic construct
  misc_feature 1..78: note = Synthetic Polynucleotide
  source 1..78: mol_type = other DNA
cctccaaaaaatatgggactatgtgagaagaccaaacctacgttttattagtgtacctcaaagtgacagggaggatgg

SEQ ID NO: 369 - DNA, 78 bp, synthetic construct
  misc_feature 1..78: note = Synthetic Polynucleotide
  source 1..78: mol_type = other DNA
ccattctccccatcactttcaggtacaccaatgaaacgtaggtttggccttttcacatagtttcatatttcttggagg

SEQ ID NO: 370 - DNA, 78 bp, synthetic construct; identical to SEQ ID NO: 333
  misc_feature 1..78: note = Synthetic Polynucleotide
  source 1..78: mol_type = other DNA

SEQ ID NO: 371 - DNA, 77 bp, synthetic construct
  misc_feature 1..77: note = Synthetic Polynucleotide
  source 1..77: mol_type = other DNA
cctacaagaaatatggaacttgtaaaaagaccaaacctacgtttgattggtgtacctgaaagtgacggggagaatgg

SEQ ID NO: 372 - DNA, 78 bp, synthetic construct
  misc_feature 1..78: note = Synthetic Polynucleotide
  source 1..78: mol_type = other DNA
cctccaagaaatatgggacaatgtgaaaaggccaaagctacgtttgattggtgtacctgaaagtgacagggagaatgg

SEQ ID NO: 373 - DNA, 78 bp, synthetic construct
  misc_feature 1..78: note = Synthetic Polynucleotide
  source 1..78: mol_type = other DNA
cctttcaaacttagaggtaaacaaaagtcctgaaaacctaggtttgaccataagttgggaccatacgagcatagaagg

SEQ ID NO: 374 - DNA, 78 bp, synthetic construct
  misc_feature 1..78: note = Synthetic Polynucleotide
  source 1..78: mol_type = other DNA
ccaaaaataaaaaaaaattgacttataagtaagaaaggttcgttttctcacattcagaaagagaacccacatgttggg

SEQ ID NO: 375 - DNA, 77 bp, synthetic construct
  misc_feature 1..77: note = Synthetic Polynucleotide
  source 1..77: mol_type = other DNA
ccaaaaataaaaaaaaattgacttataagtaagaaaggttcgttttctcacattcagaaagagaacccacatgttgg

SEQ ID NO: 376 - DNA, 78 bp, synthetic construct; identical to SEQ ID NO: 367
  misc_feature 1..78: note = Synthetic Polynucleotide
  source 1..78: mol_type = other DNA

SEQ ID NO: 377 - DNA, 78 bp, synthetic construct
  misc_feature 1..78: note = Synthetic Polynucleotide
  source 1..78: mol_type = other DNA
ccattctccccgtcactttcaggtacaccaatcaaacgttggtttagtctattcacatagtcccatatttcttggagg

SEQ ID NO: 378 - DNA, 78 bp, synthetic construct
  misc_feature 1..78: note = Synthetic Polynucleotide
  source 1..78: mol_type = other DNA
ccgaaaagaataagactatcagctgaagtcttaaaacgatcctttggcccccagtactctatatgcaggatagaaagg

SEQ ID NO: 379 - DNA, 77 bp, synthetic construct
  misc_feature 1..77: note = Synthetic Polynucleotide
  source 1..77: mol_type = other DNA
cctacaaaaataggggactatgtgataagaccaaacctacgtttgattggtgtacctgaaagtgatggggagaatgg

SEQ ID NO: 380 - DNA, 78 bp, synthetic construct
  misc_feature 1..78: note = Synthetic Polynucleotide
  source 1..78: mol_type = other DNA
ccattctacccatcactttcaggtacaccaatcaaacgtaggtttggccttttcatatagtctcatatttcttggagg

SEQ ID NO: 381 - DNA, 78 bp, synthetic construct
  misc_feature 1..78: note = Synthetic Polynucleotide
  source 1..78: mol_type = other DNA
ccattctccccatcactttctggtataccaatcaaacgtaggtttggtcttttcacatagtcccatatttcttagagg

SEQ ID NO: 382 - DNA, 78 bp, synthetic construct; identical to SEQ ID NO: 367
  misc_feature 1..78: note = Synthetic Polynucleotide
  source 1..78: mol_type = other DNA

SEQ ID NO: 383 - DNA, 78 bp, synthetic construct; identical to SEQ ID NO: 367
  misc_feature 1..78: note = Synthetic Polynucleotide
  source 1..78: mol_type = other DNA

SEQ ID NO: 384 - DNA, 78 bp, synthetic construct; identical to SEQ ID NO: 367
  misc_feature 1..78: note = Synthetic Polynucleotide
  source 1..78: mol_type = other DNA

SEQ ID NO: 385 - DNA, 78 bp, synthetic construct; identical to SEQ ID NO: 367
  misc_feature 1..78: note = Synthetic Polynucleotide
  source 1..78: mol_type = other DNA

SEQ ID NO: 386 - DNA, 78 bp, synthetic construct; identical to SEQ ID NO: 367
  misc_feature 1..78: note = Synthetic Polynucleotide
  source 1..78: mol_type = other DNA

SEQ ID NO: 387 - DNA, 78 bp, synthetic construct; identical to SEQ ID NO: 367
  misc_feature 1..78: note = Synthetic Polynucleotide
  source 1..78: mol_type = other DNA

SEQ ID NO: 388 - DNA, 77 bp, synthetic construct
  misc_feature 1..77: note = Synthetic Polynucleotide
  source 1..77: mol_type = other DNA
ccaccacacccagccttatgggatggttttcaaaagcatccttttttagaagtggattctgatatataatcggatgg

SEQ ID NO: 389 - DNA, 78 bp, synthetic construct
  misc_feature 1..78: note = Synthetic Polynucleotide
  source 1..78: mol_type = other DNA
ccattctcaatgtcactttcaggtacaccaatcaaacgtaggtttggtcttttcacatagtcccatatttcttggagg

SEQ ID NO: 390 - DNA, 78 bp, synthetic construct
  misc_feature 1..78: note = Synthetic Polynucleotide
  source 1..78: mol_type = other DNA
ccattctctctgtcactttcaggtacaccagtcaaaggtaggtttgttttattcacacgttcacatatttcttggagg

SEQ ID NO: 391 - DNA, 78 bp, synthetic construct
  misc_feature 1..78: note = Synthetic Polynucleotide
  source 1..78: mol_type = other DNA
ccattcgccccatcactttcaggtacactagtaaaacgtaggtttggtcttttcacatagttccatatttcttggagg

SEQ ID NO: 392 - DNA, 78 bp, synthetic construct
  misc_feature 1..78: note = Synthetic Polynucleotide
  source 1..78: mol_type = other DNA
cctccaagaaatatgggactatgtgaagagatcaaacctaggtttgattgttgtacctgaaagtgataagaagaatgg

SEQ ID NO: 393 - DNA, 78 bp, synthetic construct
  misc_feature 1..78: note = Synthetic Polynucleotide
  source 1..78: mol_type = other DNA
cctccaataaatatggggctatgtgaaaagaccaaacctacgtttgattggtgtacctgaaagtgacagggagaatgg

SEQ ID NO: 394 - DNA, 78 bp, synthetic construct
  misc_feature 1..78: note = Synthetic Polynucleotide
  source 1..78: mol_type = other DNA
cccttttccctgtcactttcaggtacaccagtcaaacgtaggtttggtcttttcacatagtcgaatatttcttcaagg

SEQ ID NO: 395 - DNA, 77 bp, synthetic construct
  misc_feature 1..77: note = Synthetic Polynucleotide
  source 1..77: mol_type = other DNA
ccttttccctgtcactttcaggtacaccagtcaaacgtaggtttggtcttttcacatagtcgaatatttcttcaagg

SEQ ID NO: 396 - DNA, 78 bp, synthetic construct
  misc_feature 1..78: note = Synthetic Polynucleotide
  source 1..78: mol_type = other DNA
ccattctccctgtcactttcaggtacactaatcaaacgtaggtttggtgtattcacacagtcccatatttcttggagg

SEQ ID NO: 397 - DNA, 78 bp, synthetic construct
  misc_feature 1..78: note = Synthetic Polynucleotide
  source 1..78: mol_type = other DNA
ccattcttcctgtcactttcaggtataccaatcaaacgtaggtttggtcttttcacatagtcccatgtttcttggagg

SEQ ID NO: 398 - DNA, 78 bp, synthetic construct
  misc_feature 1..78: note = Synthetic Polynucleotide
  source 1..78: mol_type = other DNA
cctccaagaaatatgagactatatgaaaataccaaacctacgtttgattggtgtacctgaaagagacagggagaatgg

SEQ ID NO: 399 - DNA, 78 bp, synthetic construct
  misc_feature 1..78: note = Synthetic Polynucleotide
  source 1..78: mol_type = other DNA
ccattctccctatcactttcaggtacaccaatcaaacgtaggtttggtcttttcatgtagtcccatatttcttggagg

SEQ ID NO: 400 - DNA, 78 bp, synthetic construct
  misc_feature 1..78: note = Synthetic Polynucleotide
  source 1..78: mol_type = other DNA
ccattctgcccgtcactttcaggtacaccaatcaaacgtaggtttggtcttttcacatagtcccatatttcttggagg

SEQ ID NO: 401 - DNA, 77 bp, synthetic construct
  misc_feature 1..77: note = Synthetic Polynucleotide
  source 1..77: mol_type = other DNA
ccgtccgattatatatcagaatctacttctaaaaaaggatgcttttgaaaaccatcccataaggctgggtgtggtgg

SEQ ID NO: 402 - DNA, 78 bp, synthetic construct
  misc_feature 1..78: note = Synthetic Polynucleotide
  source 1..78: mol_type = other DNA
cctacaaggaatataggactatgtgaaaataccaaacctacgtttcactgctgtacctgaaggtgacagggagaatgg

SEQ ID NO: 403 - DNA, 78 bp, synthetic construct
  misc_feature 1..78: note = Synthetic Polynucleotide
  source 1..78: mol_type = other DNA
ccattctccccatcatttccaggtaaaccaatcaaaggtaggtttggtcatttcacatagtcccatatttcttggagg

SEQ ID NO: 404 - DNA, 78 bp, synthetic construct
  misc_feature 1..78: note = Synthetic Polynucleotide
  source 1..78: mol_type = other DNA
ccattctccccgtcactttcaggtacaccagtcaaacgtaggtttggtcttttcacacagtcccatatttcctggagg

SEQ ID NO: 405 - DNA, 78 bp, synthetic construct
  misc_feature 1..78: note = Synthetic Polynucleotide
  source 1..78: mol_type = other DNA
ccattctccccatcactttcaggtacagcaatcaaacgtaggtttggtcttttcacatagtcccatatttcttggagg

SEQ ID NO: 406 - DNA, 76 bp, synthetic construct
  misc_feature 1..76: note = Synthetic Polynucleotide
  source 1..76: mol_type = other DNA
ccactacagattcttgggtcaagatgtgtgcaaaaggatgctttagggtgatggatatgagtgggatgaaatgagg

SEQ ID NO: 407 - DNA, 77 bp, synthetic construct
  misc_feature 1..77: note = Synthetic Polynucleotide
  source 1..77: mol_type = other DNA
cctgaaaaaaaaccctgccagccagcaactctgaaaggatgctttgtgtgagtgagcagtgtctgagatggacaggg

SEQ ID NO: 408 - DNA, 78 bp, synthetic construct
  misc_feature 1..78: note = Synthetic Polynucleotide
  source 1..78: mol_type = other DNA
ccattctccccatcactttcaggtacgccaatcaaacgtaggtttggtcttttgacatagtcccatatttcttggagg

SEQ ID NO: 409 - DNA, 78 bp, synthetic construct
  misc_feature 1..78: note = Synthetic Polynucleotide
  source 1..78: mol_type = other DNA
ccgttctccccatcacttttaggtacaccaatcaaacgtaggtttggtcttttcacatagtctcatatttcttggagg

SEQ ID NO: 410 - DNA, 78 bp, synthetic construct
  misc_feature 1..78: note = Synthetic Polynucleotide
  source 1..78: mol_type = other DNA
ccattctcctggtcactttcaggtataccaatcaaacgtaggtttggtcttttcatgtagtcccatatttcttggagg

SEQ ID NO: 411 - DNA, 78 bp, synthetic construct
  misc_feature 1..78: note = Synthetic Polynucleotide
  source 1..78: mol_type = other DNA
cctccaagaaatatgggactacatgaaaagaccaaacctacgtttgattggtatacctgaaagtgaccaggagaatgg

SEQ ID NO: 412 - DNA, 78 bp, synthetic construct
  misc_feature 1..78: note = Synthetic Polynucleotide
  source 1..78: mol_type = other DNA
cctccaagaactatgggactatgtgaaaagaccaaacctacgtttgattggtgtacctgaaagtgacggggagaatgg

SEQ ID NO: 413 - DNA, 78 bp, synthetic construct
  misc_feature 1..78: note = Synthetic Polynucleotide
  source 1..78: mol_type = other DNA
ccattctccccgtcactttcaggtacaccaatcaaacgtaggtttggtcttttcacatagtcccatagttcttggagg

SEQ ID NO: 414 - DNA, 78 bp, synthetic construct
  misc_feature 1..78: note = Synthetic Polynucleotide
  source 1..78: mol_type = other DNA
ccattctccccgtcactttcaggtacaccaatcaaacgtaggtttggtcttttcacagagtcccatatttcttggagg

SEQ ID NO: 415 - DNA, 78 bp, synthetic construct
  misc_feature 1..78: note = Synthetic Polynucleotide
  source 1..78: mol_type = other DNA
ccattctccccgtcactttcatgtacaccaagcaaacgtaggtttgatctttccacatagtcccgtgtttcttggagg

SEQ ID NO: 416 - DNA, 78 bp, synthetic construct
  misc_feature 1..78: note = Synthetic Polynucleotide
  source 1..78: mol_type = other DNA
cctccaagaaatatgggactatgtgaaaagaccaaacctacgtttgattggtgtacttgaaagtgacagggagaatgg

SEQ ID NO: 417 - DNA, 78 bp, synthetic construct
  misc_feature 1..78: note = Synthetic Polynucleotide
  source 1..78: mol_type = other DNA
cctccaagaaatatgggactatgtgaaaagacaaaacctacgtttcactggtgtacctgaaagtgacagggaggatgg

SEQ ID NO: 418 - DNA, 77 bp, synthetic construct
  misc_feature 1..77: note = Synthetic Polynucleotide
  source 1..77: mol_type = other DNA
cccccaccttttaaaaacatgcatacatacggaaacgttgctttctgcacgatttcattttaatggaacagaacagg

SEQ ID NO: 419 - DNA, 78 bp, synthetic construct
  misc_feature 1..78: note = Synthetic Polynucleotide
  source 1..78: mol_type = other DNA
ccatttcccctgtcactttcaggtacaccaatcaaacgtaggtttggtcttttcacatagtatcatatttcttggagg

SEQ ID NO: 420 - DNA, 77 bp, synthetic construct
  misc_feature 1..77: note = Synthetic Polynucleotide
  source 1..77: mol_type = other DNA
ccattctccccgtcactttcaggtacaccaatcaaacgtaggtttggtcttttcacatagtcccatatttctggagg

SEQ ID NO: 421 - DNA, 77 bp, synthetic construct
  misc_feature 1..77: note = Synthetic Polynucleotide
  source 1..77: mol_type = other DNA
ccttttgttaaagtaatagaattctgcttcttaaaggaacctttcaggcaagatggtggttagagcacctaaatggg

SEQ ID NO: 422 - DNA, 76 bp, synthetic construct
  misc_feature 1..76: note = Synthetic Polynucleotide
  source 1..76: mol_type = other DNA
ccttttgttaaagtaatagaattctgcttcttaaaggaacctttcaggcaagatggtggttagagcacctaaatgg

SEQ ID NO: 423 - DNA, 78 bp, synthetic construct; identical to SEQ ID NO: 412
  misc_feature 1..78: note = Synthetic Polynucleotide
  source 1..78: mol_type = other DNA

SEQ ID NO: 424 - DNA, 78 bp, synthetic construct
  misc_feature 1..78: note = Synthetic Polynucleotide
  source 1..78: mol_type = other DNA
ccattctccccgtcactttcaggtacaccaatcaaacgtaggtttggccttttcacatagtcccatagttcttggagg

SEQ ID NO: 425 - DNA, 77 bp, synthetic construct
  misc_feature 1..77: note = Synthetic Polynucleotide
  source 1..77: mol_type = other DNA
cctccaagaaatatgggactggtgaaaagaccaaacctacgtttgactggtgtacctgaaagtgacggggagactgg

SEQ ID NO: 426 - DNA, 78 bp, synthetic construct
  misc_feature 1..78: note = Synthetic Polynucleotide
  source 1..78: mol_type = other DNA
cctccaagaaacatgggaatgtgtgaaaagaccaaacctacgtttgattggcgtacctgaaagtgacggggagtatgg

SEQ ID NO: 427 - DNA, 78 bp, synthetic construct
  misc_feature 1..78: note = Synthetic Polynucleotide
  source 1..78: mol_type = other DNA
cctccaagaaatatgggactgtgtgaaaagaccaaacctacgtttgattggtatacctgaaagtgacagagagaatgg

SEQ ID NO: 428 - DNA, 78 bp, synthetic construct
  misc_feature 1..78: note = Synthetic Polynucleotide
  source 1..78: mol_type = other DNA
ccattctccccttcactatcaggtacaccaatcaaacgtaggtttagtcttttcacatagtcccatatttcttggagg

SEQ ID NO: 429 - DNA, 78 bp, synthetic construct
  misc_feature 1..78: note = Synthetic Polynucleotide
  source 1..78: mol_type = other DNA
ccattctccccgtcactttcagatacaccagtcaaacgtaggtttggtcttttcacatagtcccatatttcttggagg

SEQ ID NO: 430 - DNA, 78 bp, synthetic construct
  misc_feature 1..78: note = Synthetic Polynucleotide
  source 1..78: mol_type = other DNA
ccatcttactttgtactacactgttctttagagaaagcttccttttggagaccaaccaggactccttagaagcagagg

SEQ ID NO: 431 - DNA, 78 bp, synthetic construct; identical to SEQ ID NO: 430
  misc_feature 1..78: note = Synthetic Polynucleotide
  source 1..78: mol_type = other DNA

SEQ ID NO: 432 - DNA, 78 bp, synthetic construct
  misc_feature 1..78: note = Synthetic Polynucleotide
  source 1..78: mol_type = other DNA
cctctgcttctaaggagtcctggttggtctccaaaaggaagctttctctaaagaacagtgtagtacaaagtaagatgg

SEQ ID NO: 433 - DNA, 78 bp, synthetic construct; identical to SEQ ID NO: 432
  misc_feature 1..78: note = Synthetic Polynucleotide
  source 1..78: mol_type = other DNA

SEQ ID NO: 434 - DNA, 78 bp, synthetic construct; identical to SEQ ID NO: 432
  misc_feature 1..78: note = Synthetic Polynucleotide
  source 1..78: mol_type = other DNA

SEQ ID NO: 435 - DNA, 77 bp, synthetic construct
  misc_feature 1..77: note = Synthetic Polynucleotide
  source 1..77: mol_type = other DNA
ccaccactgtgcctggccattttcactattcttaaaggaagctttggtttacaaaggtttgctactgtacttccagg

SEQ ID NO: 436 - DNA, 78 bp, synthetic construct
  misc_feature 1..78: note = Synthetic Polynucleotide
  source 1..78: mol_type = other DNA
ccattctccctgtcactttcaggtacaccattcaaacgtaggtttggtcttttctcatagtcccatatttcttggagg

SEQ ID NO: 437 - DNA, 78 bp, synthetic construct
  misc_feature 1..78: note = Synthetic Polynucleotide
  source 1..78: mol_type = other DNA
cctccaagaaattcgggactatgtgaaaagacaaaacctacgtttaattggtgtgtggtgtacctgaaagtgacaagg

SEQ ID NO: 438 - DNA, 78 bp, synthetic construct
  misc_feature 1..78: note = Synthetic Polynucleotide
  source 1..78: mol_type = other DNA
cctccaagaaatatgggactatgtgaaaagaccaaacctacgtttgattggtgtacctgaaagtgaccagaagaatgg

SEQ ID NO: 439 - DNA, 77 bp, synthetic construct
  misc_feature 1..77: note = Synthetic Polynucleotide
  source 1..77: mol_type = other DNA
cctccaagaaatatgggactatgtgaaaagcccaaacctacgtttgactgatgtacctaaagtgacggggagaatgg

SEQ ID NO: 440 - DNA, 76 bp, synthetic construct
  misc_feature 1..76: note = Synthetic Polynucleotide
  source 1..76: mol_type = other DNA
cccgcactgtgagcttggccgagtgctgtctgaaagcatcctttcccttcacctggagactggagcgccatagagg

SEQ ID NO: 441 - DNA, 78 bp, synthetic construct
  misc_feature 1..78: note = Synthetic Polynucleotide
  source 1..78: mol_type = other DNA
cctgtctcccccattccatgcaaaataaaacacaaaccaagctttgctttaagtgctccctgatgcagttcagcgtgg

SEQ ID NO: 442 - DNA, 78 bp, synthetic construct
  misc_feature 1..78: note = Synthetic Polynucleotide
  source 1..78: mol_type = other DNA
ccattcttcccgtcacattcaggtacaccaatcaaacgtaggtttggtcttttcccatagtcccatatttcttagagg

SEQ ID NO: 443 - DNA, 77 bp, synthetic construct
  misc_feature 1..77: note = Synthetic Polynucleotide
  source 1..77: mol_type = other DNA
ccccctgctcagcttggggaagaaaaatacaaaaacgatgcttttaggcattttaaacaacttcactacattgaggg

SEQ ID NO: 444 - DNA, 76 bp, synthetic construct
  misc_feature 1..76: note = Synthetic Polynucleotide
  source 1..76: mol_type = other DNA
ccccctgctcagcttggggaagaaaaatacaaaaacgatgcttttaggcattttaaacaacttcactacattgagg

SEQ ID NO: 445 - DNA, 78 bp, synthetic construct
  misc_feature 1..78: note = Synthetic Polynucleotide
  source 1..78: mol_type = other DNA
cctttgtgttgtgtgtattcaactcacagagtgaaaccttcctttattcagagcagttttgaaacactctttttgtgg

SEQ ID NO: 446 - DNA, 78 bp, synthetic construct
  misc_feature 1..78: note = Synthetic Polynucleotide
  source 1..78: mol_type = other DNA
cctttgtgttgtgtgtattcaactcacagagtgaaaccttcctttattcagagcagttttgaaaaacactttttgtgg

SEQ ID NO: 447 - DNA, 78 bp, synthetic construct
  misc_feature 1..78: note = Synthetic Polynucleotide
  source 1..78: mol_type = other DNA
cctttgtgttgtgtgtattcaactcacagagtgaaaccttcctttattcagagcagttttgaaaaactctttttgtgg

SEQ ID NO: 448 - DNA, 78 bp, synthetic construct; identical to SEQ ID NO: 445
  misc_feature 1..78: note = Synthetic Polynucleotide
  source 1..78: mol_type = other DNA

SEQ ID NO: 449 - DNA, 78 bp, synthetic construct
  misc_feature 1..78: note = Synthetic Polynucleotide
  source 1..78: mol_type = other DNA
cctttgtgttgtgtgtattcaactcacagagtgaaaccttcctttattcagagcagttttgaaatactctttttgtgg

SEQ ID NO: 450 - DNA, 78 bp, synthetic construct; identical to SEQ ID NO: 445
  misc_feature 1..78: note = Synthetic Polynucleotide
  source 1..78: mol_type = other DNA

SEQ ID NO: 451 - DNA, 78 bp, synthetic construct
  misc_feature 1..78: note = Synthetic Polynucleotide
  source 1..78: mol_type = other DNA
ccattctccctgtcactttcaagtacaccaatcaaacctaggtttggtcttttcacatagttccatatttcttggagg

SEQ ID NO: 452 - DNA, 78 bp, synthetic construct; identical to SEQ ID NO: 176
  misc_feature 1..78: note = Synthetic Polynucleotide
  source 1..78: mol_type = other DNA

SEQ ID NO: 453 - DNA, 77 bp, synthetic construct
  misc_feature 1..77: note = Synthetic Polynucleotide
  source 1..77: mol_type = other DNA
cccctcccatcacaggccctgaggtttaagagaaaaccatggttttgtgggccaggcccatgacccttctcctctgg

SEQ ID NO: 454 - DNA, 77 bp, synthetic construct
  misc_feature 1..77: note = Synthetic Polynucleotide
  source 1..77: mol_type = other DNA
ccctcccatcacaggccctgaggtttaagagaaaaccatggttttgtgggccaggcccatgacccttctcctctggg

SEQ ID NO: 455 - DNA, 76 bp, synthetic construct
  misc_feature 1..76: note = Synthetic Polynucleotide
  source 1..76: mol_type = other DNA
ccctcccatcacaggccctgaggtttaagagaaaaccatggttttgtgggccaggcccatgacccttctcctctgg

SEQ ID NO: 456 - DNA, 78 bp, synthetic construct
  misc_feature 1..78: note = Synthetic Polynucleotide
  source 1..78: mol_type = other DNA
ccattctccccatcactttcaggtacaccaatcaaacgtaggtttcatcttttcacatagtcccacggtttttggagg

SEQ ID NO: 457 - DNA, 78 bp, synthetic construct
  misc_feature 1..78: note = Synthetic Polynucleotide
  source 1..78: mol_type = other DNA
cctccaagatatatgggactatgtgaaaagaccaaacctacgtttgattggtgtacctgaaattgatggggagaatgg

SEQ ID NO: 458 - DNA, 78 bp, synthetic construct
  misc_feature 1..78: note = Synthetic Polynucleotide
  source 1..78: mol_type = other DNA
cctccaagaaatatgggactgtgtgaaaagaacaaacctacgtttgattggtgtacgtgaaagtgatggggagaatgg

SEQ ID NO: 459 - DNA, 78 bp, synthetic construct
  misc_feature 1..78: note = Synthetic Polynucleotide
  source 1..78: mol_type = other DNA
ccattcctcccgtcactttcagatacaccaaaaaaacgtaggtttggtctcttcacatagtcccacatttcttggagg

SEQ ID NO: 460 - DNA, 78 bp, synthetic construct
  misc_feature 1..78: note = Synthetic Polynucleotide
  source 1..78: mol_type = other DNA
cctccaagaaatgtgggactatgtgaagagaccaaacctacgtttttttggtgtatctgaaagtgacgggaggaatgg

SEQ ID NO: 461 - DNA, 77 bp, synthetic construct
  misc_feature 1..77: note = Synthetic Polynucleotide
  source 1..77: mol_type = other DNA
cctccaaggggaatctgagttctctgaagacaaaaagcatggtttcttttcttctgtatttcttattgtttcctagg

SEQ ID NO: 462 - DNA, 78 bp, synthetic construct
  misc_feature 1..78: note = Synthetic Polynucleotide
  source 1..78: mol_type = other DNA
ccattctccctatcactttccagtacaccaatcaaacgtaggtttggtcttttcacatagtcccatatttcttggagg

SEQ ID NO: 463 - DNA, 78 bp, synthetic construct
  misc_feature 1..78: note = Synthetic Polynucleotide
  source 1..78: mol_type = other DNA
cctccaagaaatatgggactatgtgaaaagaccaaacctacgtttgattggtatacttgaaattgacaaggagaatgg

SEQ ID NO: 464 - DNA, 78 bp, synthetic construct
  misc_feature 1..78: note = Synthetic Polynucleotide
  source 1..78: mol_type = other DNA
cctccaagaaatatgggactatgtggaaagaccaaacctacgtttgactggtgtacctgaaagtgatggggagaatgg

SEQ ID NO: 465 - DNA, 78 bp, synthetic construct
  misc_feature 1..78: note = Synthetic Polynucleotide
  source 1..78: mol_type = other DNA
cctctaagaaatatgggactatgtgaagagatgaaacctacgtttgattggtgtacctgaaagtgacgaggagaatgg

SEQ ID NO: 466 - DNA, 77 bp, synthetic construct
  misc_feature 1..77: note = Synthetic Polynucleotide
  source 1..77: mol_type = other DNA
ccctcgtatactacatgctatagtcaaagcagtaaaccttcctttccttaagcagaccacactctttcatgcctggg

SEQ ID NO: 467 - DNA, 76 bp, synthetic construct
  misc_feature 1..76: note = Synthetic Polynucleotide
  source 1..76: mol_type = other DNA
cctcgtatactacatgctatagtcaaagcagtaaaccttcctttccttaagcagaccacactctttcatgcctggg

SEQ ID NO: 468 - DNA, 78 bp, synthetic construct
  misc_feature 1..78: note = Synthetic Polynucleotide
  source 1..78: mol_type = other DNA
ccattctccccatcactttcaggtatactaatcaaaggtaggtttggtcttttcacatagtcccatatttcatggagg

SEQ ID NO: 469 - DNA, 77 bp, synthetic construct
  misc_feature 1..77: note = Synthetic Polynucleotide
  source 1..77: mol_type = other DNA
ccattcccccgtcactttcaggtacaccaatcaaacgtaggtttggtcttttcacatagtcccatatttcttggagg

SEQ ID NO: 470 - DNA, 78 bp, synthetic construct
  misc_feature 1..78: note = Synthetic Polynucleotide
  source 1..78: mol_type = other DNA
ccattctccccgtcactttcaggtacaccaatcaaacgtaggttttgtcttttcttatagtcccatatttcttggagg

SEQ ID NO: 471 - DNA, 78 bp, synthetic construct
  misc_feature 1..78: note = Synthetic Polynucleotide
  source 1..78: mol_type = other DNA
ccactgcacctgaccaagatccttaatttttctaaacctacgtttatcatctataaaatgagccatcttttcacatgg

SEQ ID NO: 472 - DNA, 78 bp, synthetic construct
  misc_feature 1..78: note = Synthetic Polynucleotide
  source 1..78: mol_type = other DNA
cctccgagaaatatgggactatgtgaaaagaccaaacctacgtttgattgttgtacctgaaagtgacagggagaatgg

SEQ ID NO: 473 - DNA, 78 bp, synthetic construct
  misc_feature 1..78: note = Synthetic Polynucleotide
  source 1..78: mol_type = other DNA
ccattctccccatcacttttaggtacaccaatcaaacgtaggtttggtccttttgcatagacccatatttcttggagg

SEQ ID NO: 474 - DNA, 78 bp, synthetic construct
  misc_feature 1..78: note = Synthetic Polynucleotide
  source 1..78: mol_type = other DNA
ccattttccccgtcagtttcatatacacctatcaaacgtaggtttactgttttcacatagtcccttatttcttggagg

SEQ ID NO: 475 - DNA, 78 bp, synthetic construct
  misc_feature 1..78: note = Synthetic Polynucleotide
  source 1..78: mol_type = other DNA
cctccaagaaatatgggactatgtgaaaagaccaaacctacctttgattggtgtacctgaaagtgacgggcaggatgg

SEQ ID NO: 476 - DNA, 78 bp, synthetic construct
  misc_feature 1..78: note = Synthetic Polynucleotide
  source 1..78: mol_type = other DNA
ccattcttctcgtcattttcaagtacaccaatcaaacgtaggtttggtcttttcgcatagtcccatatttcttggagg

SEQ ID NO: 477 - DNA, 78 bp, synthetic construct
  misc_feature 1..78: note = Synthetic Polynucleotide
  source 1..78: mol_type = other DNA
ccattcttctcgtcactttcaagtacaccaatcaaacgtaggtttggtcttttcacatagtcccatatttcttggagg

SEQ ID NO: 478 - DNA, 78 bp, synthetic construct
  misc_feature 1..78: note = Synthetic Polynucleotide
  source 1..78: mol_type = other DNA
cctccaagaaatataggactatgtgaaaagaccaaacctacgtttgattggtgtacttgaaagtgacagggagaatgg

SEQ ID NO: 479 - DNA, 78 bp, synthetic construct
  misc_feature 1..78: note = Synthetic Polynucleotide
  source 1..78: mol_type = other DNA
cctccaagaaatgtggaactatgtgaaaagaccaaacctacgtttgattggtgtacctgaaagtgacagggagaatgg

SEQ ID NO: 480 - DNA, 76 bp, synthetic construct
  misc_feature 1..76: note = Synthetic Polynucleotide
  source 1..76: mol_type = other DNA
ccctgacactgataaacggatatgaagagaaaaaagctaggttttcgctggaattcctaagcttgggctgcagtgg

SEQ ID NO: 481 - DNA, 78 bp, synthetic construct
  misc_feature 1..78: note = Synthetic Polynucleotide
  source 1..78: mol_type = other DNA
cccttctcccagtcacttttaggtacaccaatgaaacgtaggtttggtcttttcacacagtcccatatttcttggagg

SEQ ID NO: 482 - DNA, 77 bp, synthetic construct
  misc_feature 1..77: note = Synthetic Polynucleotide
  source 1..77: mol_type = other DNA
ccttctcccagtcacttttaggtacaccaatgaaacgtaggtttggtcttttcacacagtcccatatttcttggagg

SEQ ID NO: 483 - DNA, 76 bp, synthetic construct; identical to SEQ ID NO: 180
  misc_feature 1..76: note = Synthetic Polynucleotide
  source 1..76: mol_type = other DNA

SEQ ID NO: 484 - DNA, 78 bp, synthetic construct
  misc_feature 1..78: note = Synthetic Polynucleotide
  source 1..78: mol_type = other DNA
ccaaacccgcatcgcacaccctgtgagggggacaaaggaacctttccgttccaacatcaaggttgttttgacccaagg

SEQ ID NO: 485 - DNA, 78 bp, synthetic construct
  misc_feature 1..78: note = Synthetic Polynucleotide
  source 1..78: mol_type = other DNA
ccattctttctgtcactttcaggtataccagtcaaacctaggtttggtcttttcacatagtcccatatttcttggagg

SEQ ID NO: 486 - DNA, 78 bp, synthetic construct; identical to SEQ ID NO: 367
  misc_feature 1..78: note = Synthetic Polynucleotide
  source 1..78: mol_type = other DNA

SEQ ID NO: 487 - DNA, 77 bp, synthetic construct
  misc_feature 1..77: note = Synthetic Polynucleotide
  source 1..77: mol_type = other DNA
ccacacggtagaggataaactaggtggattctcaaagcaacctttgaaataatctatgcagtttttctgggtactgg

SEQ ID NO: 488 - DNA, 78 bp, synthetic construct
  misc_feature 1..78: note = Synthetic Polynucleotide
  source 1..78: mol_type = other DNA
ccaccaagaaacatgggactatgtgaaaagaccaaacctacgtttggttggtgtacctggaagtgacggggagagtgg

SEQ ID NO: 489 - DNA, 78 bp, synthetic construct
  misc_feature 1..78: note = Synthetic Polynucleotide
  source 1..78: mol_type = other DNA
cctccaagaaatatgggaccatgtgaaaagaccaaacctacgtttgattggtgtacctgaaagtgacagggagaatgg

SEQ ID NO: 490 - DNA, 77 bp, synthetic construct
  misc_feature 1..77: note = Synthetic Polynucleotide
  source 1..77: mol_type = other DNA
cctgtaaaaaggtcacatggtcaggtgtgcctaaacgatccttttatttatttatttatttatttttaagaaacagg

SEQ ID NO: 491 - DNA, 77 bp, synthetic construct
  misc_feature 1..77: note = Synthetic Polynucleotide
  source 1..77: mol_type = other DNA
ccagccccaaaatgtcaggggcttagaacaacaaaggttccttttcatgtttatactacatgtttgtcatgggctgg

SEQ ID NO: 492 - DNA, 78 bp, synthetic construct
  misc_feature 1..78: note = Synthetic Polynucleotide
  source 1..78: mol_type = other DNA
ccgttttccccatcactttcaggtacaccagtcaaacgtaggtttggtcttttcacatggtcccacatttcttggagg

SEQ ID NO: 493 - DNA, 78 bp, synthetic construct
  misc_feature 1..78: note = Synthetic Polynucleotide
  source 1..78: mol_type = other DNA
cctggaatagctttcctgactgtctgacttcaaaaaccttggtttgaccacttcgtctatatcatgaggaaggactgg

SEQ ID NO: 494 - DNA, 77 bp, synthetic construct
  misc_feature 1..77: note = Synthetic Polynucleotide
  source 1..77: mol_type = other DNA
ccctactctgaacctaccttgataaagcctagaaaaccaagctttgacaagatttgacaagagatggaatttggagg

SEQ ID NO: 495 - DNA, 76 bp, synthetic construct
  misc_feature 1..76: note = Synthetic Polynucleotide
  source 1..76: mol_type = other DNA
cctactctgaacctaccttgataaagcctagaaaaccaagctttgacaagatttgacaagagatggaatttggagg

SEQ ID NO: 496 - DNA, 77 bp, synthetic construct
  misc_feature 1..77: note = Synthetic Polynucleotide
  source 1..77: mol_type = other DNA
cccttataaaactgaaaactttaaccttttttaaagcatgcttttgaataaattcttttattacaaaaaagaccagg

SEQ ID NO: 497 - DNA, 78 bp, synthetic construct
  misc_feature 1..78: note = Synthetic Polynucleotide
  source 1..78: mol_type = other DNA
ccattctccctgtcactttcaggtacaccaatcaaacgtaggtttggtcttttcacgtagtcccatatttcttggagg

SEQ ID NO: 498 - DNA, 77 bp, synthetic construct
  misc_feature 1..77: note = Synthetic Polynucleotide
  source 1..77: mol_type = other DNA
ccctttattatccaagtggtttcctgctcttcaaaccttcctttcaaaattttgtctcctacttaaaacaagttagg

SEQ ID NO: 499 - DNA, 77 bp, synthetic construct
  misc_feature 1..77: note = Synthetic Polynucleotide
  source 1..77: mol_type = other DNA
ccttctgttgagacctactgctaagaaaacaaaaaaggttcctttcaaatattattgtgaatcaataatgtacctgg

SEQ ID NO: 500 - DNA, 78 bp, synthetic construct
  misc_feature 1..78: note = Synthetic Polynucleotide
  source 1..78: mol_type = other DNA
cctccaagaaatatgggactatgtgaaaagaccaaacctacgtttcattgatggacctgaaagtgatggggagaatgg

SEQ ID NO: 501 - DNA, 77 bp, synthetic construct
  misc_feature 1..77: note = Synthetic Polynucleotide
  source 1..77: mol_type = other DNA
ccattctcccttcactttcagttacaccaatcaaacgtaggtttggtcttttcacatagtcccatatttcttggagg

SEQ ID NO: 502 - DNA, 77 bp, synthetic construct; identical to SEQ ID NO: 181
  misc_feature 1..77: note = Synthetic Polynucleotide
  source 1..77: mol_type = other DNA

SEQ ID NO: 503 - DNA, 77 bp, synthetic construct; identical to SEQ ID NO: 501
  misc_feature 1..77: note = Synthetic Polynucleotide
  source 1..77: mol_type = other DNA

SEQ ID NO: 504 - DNA, 78 bp, synthetic construct
  misc_feature 1..78: note = Synthetic Polynucleotide
  source 1..78: mol_type = other DNA
cctccaagaaatatgggactatgtgaaaagactaaacctacgtttgattggtgtacctgaaagtgacagggagaatgg

SEQ ID NO: 505 - DNA, 78 bp, synthetic construct
  misc_feature 1..78: note = Synthetic Polynucleotide
  source 1..78: mol_type = other DNA
ccattctccctgtcactttcaggtatgccagtcaaacgtaggtttggtcttttcacatagtcccatattccttggagg

SEQ ID NO: 506 - DNA, 78 bp, synthetic construct
  misc_feature 1..78: note = Synthetic Polynucleotide
  source 1..78: mol_type = other DNA
cctccaagaaatatgggactatgtaaaaagacgaaacctacgtttgattggtgtacttaaaagtgacgaggagaatgg

SEQ ID NO: 507 - DNA, 78 bp, synthetic construct
  misc_feature 1..78: note = Synthetic Polynucleotide
  source 1..78: mol_type = other DNA
cctccaagaaatatgggactatgtgaaaagaccaaacctacgtttgatttgtgtacctgaaagtgatggggagaatgg

SEQ ID NO: 508 - DNA, 78 bp, synthetic construct
  misc_feature 1..78: note = Synthetic Polynucleotide
  source 1..78: mol_type = other DNA
ccattctccccgtcactttcaggcacaccaatcaaacgtaggtttagtcttttcacatagtcccatatttcttagagg

SEQ ID NO: 509 - DNA, 77 bp, synthetic construct
  misc_feature 1..77: note = Synthetic Polynucleotide
  source 1..77: mol_type = other DNA
ccttaatgcattcatatttcatattttaaataaaaccatggtttcccacagagtgacttctactctaagaaatgggg

SEQ ID NO: 510 - DNA, 76 bp, synthetic construct
  misc_feature 1..76: note = Synthetic Polynucleotide
  source 1..76: mol_type = other DNA
ccttaatgcattcatatttcatattttaaataaaaccatggtttcccacagagtgacttctactctaagaaatggg

SEQ ID NO: 511 - DNA, 78 bp, synthetic construct
  misc_feature 1..78: note = Synthetic Polynucleotide
  source 1..78: mol_type = other DNA
ccgttctttccgtcactttcaggtacaccagtcaaacgtaggtttggtcttttcacatagtcccatatttcttggagg

SEQ ID NO: 512 - DNA, 77 bp, synthetic construct
  misc_feature 1..77: note = Synthetic Polynucleotide
  source 1..77: mol_type = other DNA
ccattctccccatcactttcatgtacaccaatcaaacgtaggtttggtctttgttaacatagtcccatatttcttgg

SEQ ID NO: 513 - DNA, 77 bp, synthetic construct
  misc_feature 1..77: note = Synthetic Polynucleotide
  source 1..77: mol_type = other DNA
ccctataaagcttagagaaacacagggctctttaaacgatcctttttctcttttctgttttaaatttcatcacttgg

SEQ ID NO: 514 - DNA, 76 bp, synthetic construct
  misc_feature 1..76: note = Synthetic Polynucleotide
  source 1..76: mol_type = other DNA
cctataaagcttagagaaacacagggctctttaaacgatcctttttctcttttctgttttaaatttcatcacttgg

SEQ ID NO: 515 - DNA, 78 bp, synthetic construct
  misc_feature 1..78: note = Synthetic Polynucleotide
  source 1..78: mol_type = other DNA
ccattctccccatcactttcaggtacactaatcaaaggtaggtttggtcttttcacatggtcctatatttcttggagg

SEQ ID NO: 516 - DNA, 78 bp, synthetic construct
  misc_feature 1..78: note = Synthetic Polynucleotide
  source 1..78: mol_type = other DNA
ccccatagcacgatcacatgggacattcaggggaaagcaaccttttccaggaaggaaaacccaatgctgggacccagg

SEQ ID NO: 517 - DNA, 77 bp, synthetic construct
  misc_feature 1..77: note = Synthetic Polynucleotide
  source 1..77: mol_type = other DNA
cccatagcacgatcacatgggacattcaggggaaagcaaccttttccaggaaggaaaacccaatgctgggacccagg

SEQ ID NO: 518 - DNA, 77 bp, synthetic construct
  misc_feature 1..77: note = Synthetic Polynucleotide
  source 1..77: mol_type = other DNA
ccctttcagcgctcacaggctatggttttataaaaggaacctttgattttgttcatgtgaaactacaaaatgccagg

SEQ ID NO: 519 - DNA, 78 bp, synthetic construct; identical to SEQ ID NO: 516
  misc_feature 1..78: note = Synthetic Polynucleotide
  source 1..78: mol_type = other DNA

SEQ ID NO: 520 - DNA, 77 bp, synthetic construct; identical to SEQ ID NO: 517
  misc_feature 1..77: note = Synthetic Polynucleotide
  source 1..77: mol_type = other DNA

SEQ ID NO: 521 - DNA, 78 bp, synthetic construct
  misc_feature 1..78: note = Synthetic Polynucleotide
  source 1..78: mol_type = other DNA
cctccaagaaatattggagtatgtgataagaccaaaccttcgtttgactggtgtacctgaaagtgatggggagaatgg

SEQ ID NO: 522 - DNA, 78 bp, synthetic construct
  misc_feature 1..78: note = Synthetic Polynucleotide
  source 1..78: mol_type = other DNA
ccattctccccgtcactttcaggtacaccaatcaaacgtaggtttggtcttttcacatagtcccatatttcttggagg

SEQ ID NO: 523 - DNA, 78 bp, synthetic construct; identical to SEQ ID NO: 522
  misc_feature 1..78: note = Synthetic Polynucleotide
  source 1..78: mol_type = other DNA

SEQ ID NO: 524 - DNA, 78 bp, synthetic construct
  misc_feature 1..78: note = Synthetic Polynucleotide
  source 1..78: mol_type = other DNA
ccattctccccatcactttcaggtacaccagtcaaacgaaggtttggtcttatcacatactccaatatttcttggagg

SEQ ID NO: 525 - DNA, 78 bp, synthetic construct
  misc_feature 1..78: note = Synthetic Polynucleotide
  source 1..78: mol_type = other DNA
cctccaagatatatgggactatgtgaaaaggccaaacctacctttgattgatacacctgaaaatgacagggagaatgg

SEQ ID NO: 526 - DNA, 78 bp, synthetic construct
  misc_feature 1..78: note = Synthetic Polynucleotide
  source 1..78: mol_type = other DNA
cctccaagaaatatgcgactatgtgaaaagaccaaacctacgtttcattggtgtacctgaaagtgatggggagaatgg

SEQ ID NO: 527 - DNA, 77 bp, synthetic construct
  misc_feature 1..77: note = Synthetic Polynucleotide
  source 1..77: mol_type = other DNA
cctccaagaaatatgggactatgtggaaagaccaaacctacgtttgtttggtgtacctgaaagtgaggggagaatgg

SEQ ID NO: 528 - DNA, 78 bp, synthetic construct
  misc_feature 1..78: note = Synthetic Polynucleotide
  source 1..78: mol_type = other DNA
ccattctcctcatcactttcaagtacaccaatcaaacgtaggtttggtcttttcacatagtcttatatttcttggagg

SEQ ID NO: 529 - DNA, 78 bp, synthetic construct; identical to SEQ ID NO: 522
  misc_feature 1..78: note = Synthetic Polynucleotide
  source 1..78: mol_type = other DNA

SEQ ID NO: 530 - DNA, 78 bp, synthetic construct; identical to SEQ ID NO: 524
  misc_feature 1..78: note = Synthetic Polynucleotide
  source 1..78: mol_type = other DNA

SEQ ID NO: 531 - DNA, 78 bp, synthetic construct; identical to SEQ ID NO: 522
  misc_feature 1..78: note = Synthetic Polynucleotide
  source 1..78: mol_type = other DNA

SEQ ID NO: 532 - DNA, 78 bp, synthetic construct
  misc_feature 1..78: note = Synthetic Polynucleotide
  source 1..78: mol_type = other DNA
ccagcagaagaatctggggcacagtctgtgaaaaaaggtacctttcttaagcagggttcttatccttcatgggtctgg

SEQ ID NO: 533 - DNA, 78 bp, synthetic construct
  misc_feature 1..78: note = Synthetic Polynucleotide
  source 1..78: mol_type = other DNA
cctccaagaaatatgggactatgtgaaaagaccaaacctacgtttgattgttgtacctgaaagtgagggggagaatgg

SEQ ID NO: 534 - DNA, 77 bp, synthetic construct
  misc_feature 1..77: note = Synthetic Polynucleotide
  source 1..77: mol_type = other DNA
ccttgtgttgtgtgtattcaactcacagagttaaacgatcctttacacagagcagatttgaaacactgtttttctgg

SEQ ID NO: 535 - DNA, 77 bp, synthetic construct; identical to SEQ ID NO: 534
  misc_feature 1..77: note = Synthetic Polynucleotide
  source 1..77: mol_type = other DNA

SEQ ID NO: 536 - DNA, 77 bp, synthetic construct; identical to SEQ ID NO: 534
  misc_feature 1..77: note = Synthetic Polynucleotide
  source 1..77: mol_type = other DNA

SEQ ID NO: 537 - DNA, 77 bp, synthetic construct
  misc_feature 1..77: note = Synthetic Polynucleotide
  source 1..77: mol_type = other DNA
ccttgtgttgtgtgtattcaactcacagagttaaacgatcctttacacacagcagatttgaaacactgtttttctgg

SEQ ID NO: 538 - DNA, 77 bp, synthetic construct; identical to SEQ ID NO: 534
  misc_feature 1..77: note = Synthetic Polynucleotide
  source 1..77: mol_type = other DNA

SEQ ID NO: 539 - DNA, 77 bp, synthetic construct; identical to SEQ ID NO: 534
  misc_feature 1..77: note = Synthetic Polynucleotide
  source 1..77: mol_type = other DNA

SEQ ID NO: 540 - DNA, 77 bp, synthetic construct; identical to SEQ ID NO: 534
  misc_feature 1..77: note = Synthetic Polynucleotide
  source 1..77: mol_type = other DNA

SEQ ID NO: 541 - DNA, 77 bp, synthetic construct; identical to SEQ ID NO: 534
  misc_feature 1..77: note = Synthetic Polynucleotide
  source 1..77: mol_type = other DNA

SEQ ID NO: 542 - DNA, 77 bp, synthetic construct; identical to SEQ ID NO: 534
  misc_feature 1..77: note = Synthetic Polynucleotide
  source 1..77: mol_type = other DNA

SEQ ID NO: 543 - DNA, 77 bp, synthetic construct; identical to SEQ ID NO: 534
  misc_feature 1..77: note = Synthetic Polynucleotide
  source 1..77: mol_type = other DNA

SEQ ID NO: 544 - DNA, 77 bp, synthetic construct; identical to SEQ ID NO: 534
  misc_feature 1..77: note = Synthetic Polynucleotide
  source 1..77: mol_type = other DNA

SEQ ID NO: 545 - DNA, 77 bp, synthetic construct; identical to SEQ ID NO: 534
  misc_feature 1..77: note = Synthetic Polynucleotide
  source 1..77: mol_type = other DNA

SEQ ID NO: 546 - DNA, 77 bp, synthetic construct; identical to SEQ ID NO: 534
  misc_feature 1..77: note = Synthetic Polynucleotide
  source 1..77: mol_type = other DNA

SEQ ID NO: 547 - DNA, 77 bp, synthetic construct; identical to SEQ ID NO: 537
  misc_feature 1..77: note = Synthetic Polynucleotide
  source 1..77: mol_type = other DNA

SEQ ID NO: 548 - DNA, 77 bp, synthetic construct; identical to SEQ ID NO: 534
  misc_feature 1..77: note = Synthetic Polynucleotide
  source 1..77: mol_type = other DNA

SEQ ID NO: 549 - DNA, 77 bp, synthetic construct; identical to SEQ ID NO: 534
  misc_feature 1..77: note = Synthetic Polynucleotide
  source 1..77: mol_type = other DNA

SEQ ID NO: 550 - DNA, 77 bp, synthetic construct; identical to SEQ ID NO: 534
  misc_feature 1..77: note = Synthetic Polynucleotide
  source 1..77: mol_type = other DNA

SEQ ID NO: 551 - DNA, 77 bp, synthetic construct
  misc_feature 1..77: note = Synthetic Polynucleotide
  source 1..77: mol_type = other DNA
ccttgtgttgtgtgtattcaactcaccgagttaaacgatcctttacacagagcagatttgaaacactgtttttctgg

SEQ ID NO: 552 - DNA, 77 bp, synthetic construct; identical to SEQ ID NO: 534
  misc_feature 1..77: note = Synthetic Polynucleotide
  source 1..77: mol_type = other DNA

SEQ ID NO: 553 - DNA, 77 bp, synthetic construct; identical to SEQ ID NO: 534
  misc_feature 1..77: note = Synthetic Polynucleotide
  source 1..77: mol_type = other DNA

SEQ ID NO: 554 - DNA, 77 bp, synthetic construct; identical to SEQ ID NO: 534
  misc_feature 1..77: note = Synthetic Polynucleotide
  source 1..77: mol_type = other DNA

SEQ ID NO: 555 - DNA, 77 bp, synthetic construct; identical to SEQ ID NO: 534
  misc_feature 1..77: note = Synthetic Polynucleotide
  source 1..77: mol_type = other DNA

SEQ ID NO: 556 - DNA, 77 bp, synthetic construct; identical to SEQ ID NO: 534
  misc_feature 1..77: note = Synthetic Polynucleotide
  source 1..77: mol_type = other DNA

SEQ ID NO: 557 - DNA, 77 bp, synthetic construct; identical to SEQ ID NO: 534
  misc_feature 1..77: note = Synthetic Polynucleotide
  source 1..77: mol_type = other DNA

SEQ ID NO: 558 - DNA, 77 bp, synthetic construct; identical to SEQ ID NO: 534
  misc_feature 1..77: note = Synthetic Polynucleotide
  source 1..77: mol_type = other DNA

SEQ ID NO: 559 - DNA, 77 bp, synthetic construct; identical to SEQ ID NO: 537
  misc_feature 1..77: note = Synthetic Polynucleotide
  source 1..77: mol_type = other DNA

SEQ ID NO: 560 - DNA, 77 bp, synthetic construct; identical to SEQ ID NO: 534
  misc_feature 1..77: note = Synthetic Polynucleotide
  source 1..77: mol_type = other DNA

SEQ ID NO: 561 - DNA, 77 bp, synthetic construct; identical to SEQ ID NO: 534
  misc_feature 1..77: note = Synthetic Polynucleotide
  source 1..77: mol_type = other DNA

SEQ ID NO: 562 - DNA, 77 bp, synthetic construct; identical to SEQ ID NO: 534
  misc_feature 1..77: note = Synthetic Polynucleotide
  source 1..77: mol_type = other DNA

SEQ ID NO: 563 - DNA, 77 bp, synthetic construct; identical to SEQ ID NO: 534
  misc_feature 1..77: note = Synthetic Polynucleotide
  source 1..77: mol_type = other DNA

SEQ ID NO: 564 - DNA, 77 bp, synthetic construct; identical to SEQ ID NO: 534
  misc_feature 1..77: note = Synthetic Polynucleotide
  source 1..77: mol_type = other DNA

SEQ ID NO: 565 - DNA, 77 bp, synthetic construct; identical to SEQ ID NO: 534
  misc_feature 1..77: note = Synthetic Polynucleotide
  source 1..77: mol_type = other DNA

SEQ ID NO: 566 - DNA, 77 bp, synthetic construct
  misc_feature 1..77: note = Synthetic Polynucleotide
  source 1..77: mol_type = other DNA
ccttgtgttgtgtgtattcaactcacagagttaaacgatcctttacacagagcagatttgtaacactgtttttctgg

SEQ ID NO: 567 - DNA, 77 bp, synthetic construct; identical to SEQ ID NO: 534
  misc_feature 1..77: note = Synthetic Polynucleotide
  source 1..77: mol_type = other DNA

SEQ ID NO: 568 - DNA, 77 bp, synthetic construct; identical to SEQ ID NO: 534
  misc_feature 1..77: note = Synthetic Polynucleotide
  source 1..77: mol_type = other DNA

SEQ ID NO: 569 - DNA, 77 bp, synthetic construct; identical to SEQ ID NO: 534
  misc_feature 1..77: note = Synthetic Polynucleotide
  source 1..77: mol_type = other DNA

SEQ ID NO: 570 - DNA, 77 bp, synthetic construct; identical to SEQ ID NO: 534
  misc_feature 1..77: note = Synthetic Polynucleotide
  source 1..77: mol_type = other DNA

SEQ ID NO: 571 - DNA, 77 bp, synthetic construct; identical to SEQ ID NO: 534
  misc_feature 1..77: note = Synthetic Polynucleotide
  source 1..77: mol_type = other DNA

SEQ ID NO: 572 - DNA, 77 bp, synthetic construct; identical to SEQ ID NO: 534
  misc_feature 1..77: note = Synthetic Polynucleotide
  source 1..77: mol_type = other DNA

SEQ ID NO: 573 - DNA, 77 bp, synthetic construct; identical to SEQ ID NO: 534
  misc_feature 1..77: note = Synthetic Polynucleotide
  source 1..77: mol_type = other DNA

SEQ ID NO: 574 - DNA, 77 bp, synthetic construct; identical to SEQ ID NO: 534
  misc_feature 1..77: note = Synthetic Polynucleotide
  source 1..77: mol_type = other DNA

SEQ ID NO: 575 - DNA, 77 bp, synthetic construct
  misc_feature 1..77: note = Synthetic Polynucleotide
  source 1..77: mol_type = other DNA
ccttgtgttgtgtgtattcaactcacagagttaaacgatcctttacacagagcagatttgaaacactgtttttgtgg

SEQ ID NO: 576 - DNA, 77 bp, synthetic construct; identical to SEQ ID NO: 534
  misc_feature 1..77: note = Synthetic Polynucleotide
  source 1..77: mol_type = other DNA

SEQ ID NO: 577 - DNA, 77 bp, synthetic construct; identical to SEQ ID NO: 534
  misc_feature 1..77: note = Synthetic Polynucleotide
  source 1..77: mol_type = other DNA

SEQ ID NO: 578 - DNA, 77 bp, synthetic construct; identical to SEQ ID NO: 534
  misc_feature 1..77: note = Synthetic Polynucleotide
  source 1..77: mol_type = other DNA

SEQ ID NO: 579 - DNA, 77 bp, synthetic construct; identical to SEQ ID NO: 534
  misc_feature 1..77: note = Synthetic Polynucleotide
  source 1..77: mol_type = other DNA

SEQ ID NO: 580 - DNA, 77 bp, synthetic construct; identical to SEQ ID NO: 534
  misc_feature 1..77: note = Synthetic Polynucleotide
  source 1..77: mol_type = other DNA

SEQ ID NO: 581 - DNA, 77 bp, synthetic construct
  misc_feature 1..77: note = Synthetic Polynucleotide
  source 1..77: mol_type = other DNA
ccttgtgttgtgtgtattcaactcacagagttaaacgatcctttccacagagcagatttgaaacactgtttttctgg

SEQ ID NO: 582 - DNA, 77 bp, synthetic construct; identical to SEQ ID NO: 534
  misc_feature 1..77: note = Synthetic Polynucleotide
  source 1..77: mol_type = other DNA

SEQ ID NO: 583 - DNA, 77 bp, synthetic construct; identical to SEQ ID NO: 534
  misc_feature 1..77: note = Synthetic Polynucleotide
  source 1..77: mol_type = other DNA

SEQ ID NO: 584 - DNA, 77 bp, synthetic construct; identical to SEQ ID NO: 534
  misc_feature 1..77: note = Synthetic Polynucleotide
  source 1..77: mol_type = other DNA

SEQ ID NO: 585 - DNA, 77 bp, synthetic construct; identical to SEQ ID NO: 534
  misc_feature 1..77: note = Synthetic Polynucleotide
  source 1..77: mol_type = other DNA

SEQ ID NO: 586 - DNA, 77 bp, synthetic construct; identical to SEQ ID NO: 534
  misc_feature 1..77: note = Synthetic Polynucleotide
  source 1..77: mol_type = other DNA

SEQ ID NO: 587 - DNA, 77 bp, synthetic construct; identical to SEQ ID NO: 534
  misc_feature 1..77: note = Synthetic Polynucleotide
  source 1..77: mol_type = other DNA

SEQ ID NO: 588 - DNA, 77 bp, synthetic construct; identical to SEQ ID NO: 534
  misc_feature 1..77: note = Synthetic Polynucleotide
  source 1..77: mol_type = other DNA

SEQ ID NO: 589 - DNA, 77 bp, synthetic construct; identical to SEQ ID NO: 534
  misc_feature 1..77: note = Synthetic Polynucleotide
  source 1..77: mol_type = other DNA

SEQ ID NO: 590 - DNA, 77 bp, synthetic construct; identical to SEQ ID NO: 534
  misc_feature 1..77: note = Synthetic Polynucleotide
  source 1..77: mol_type = other DNA

SEQ ID NO: 591 - DNA, 77 bp, synthetic construct; identical to SEQ ID NO: 534
  misc_feature 1..77: note = Synthetic Polynucleotide
  source 1..77: mol_type = other DNA

SEQ ID NO: 592 - DNA, 77 bp, synthetic construct; identical to SEQ ID NO: 551
  misc_feature 1..77: note = Synthetic Polynucleotide
  source 1..77: mol_type = other DNA

SEQ ID NO: 593 - DNA, 77 bp, synthetic construct; identical to SEQ ID NO: 534
  misc_feature 1..77: note = Synthetic Polynucleotide
  source 1..77: mol_type = other DNA

SEQ ID NO: 594 - DNA, 77 bp, synthetic construct; identical to SEQ ID NO: 534
  misc_feature 1..77: note = Synthetic Polynucleotide
  source 1..77: mol_type = other DNA

SEQ ID NO: 595 - DNA, 77 bp, synthetic construct; identical to SEQ ID NO: 534
  misc_feature 1..77: note = Synthetic Polynucleotide
  source 1..77: mol_type = other DNA

SEQ ID NO: 596 - DNA, 77 bp, synthetic construct; identical to SEQ ID NO: 534
  misc_feature 1..77: note = Synthetic Polynucleotide
  source 1..77: mol_type = other DNA

SEQ ID NO: 597 - DNA, 77 bp, synthetic construct; identical to SEQ ID NO: 534
  misc_feature 1..77: note = Synthetic Polynucleotide
  source 1..77: mol_type = other DNA

SEQ ID NO: 598 - DNA, 77 bp, synthetic construct
  misc_feature 1..77: note = Synthetic Polynucleotide
  source 1..77: mol_type = other DNA
ccttgtgttgtgtgtattcaactcacagagttaaacgatcctttacacagagcagatttgaaaaactgtttttctgg

SEQ ID NO: 599 - DNA, 77 bp, synthetic construct
  misc_feature 1..77: note = Synthetic Polynucleotide
  source 1..77: mol_type = other DNA
ccttgtgttgtgtgtattcaactcacagagtaaaacgatcctttacacagagcagatttgaaacactgtttttctgg

SEQ ID NO: 600 - DNA, 77 bp, synthetic construct; identical to SEQ ID NO: 575
  misc_feature 1..77: note = Synthetic Polynucleotide
  source 1..77: mol_type = other DNA

SEQ ID NO: 601 - DNA, 77 bp, synthetic construct; identical to SEQ ID NO: 534
  misc_feature 1..77: note = Synthetic Polynucleotide
  source 1..77: mol_type = other DNA

SEQ ID NO: 602 - DNA, 77 bp, synthetic construct
  misc_feature 1..77: note = Synthetic Polynucleotide
  source 1..77: mol_type = other DNA
ccttgtgttgtgtgtatttaactcacagagttaaacgatcctttacacagagcagatttgaaacactgtttttctgg

SEQ ID NO: 603 - DNA, 77 bp, synthetic construct; identical to SEQ ID NO: 534
  misc_feature 1..77: note = Synthetic Polynucleotide
  source 1..77: mol_type = other DNA

SEQ ID NO: 604 - DNA, 77 bp, synthetic construct; identical to SEQ ID NO: 534
  misc_feature 1..77: note = Synthetic Polynucleotide
  source 1..77: mol_type = other DNA

SEQ ID NO: 605 - DNA, 77 bp, synthetic construct; identical to SEQ ID NO: 534
  misc_feature 1..77: note = Synthetic Polynucleotide
  source 1..77: mol_type = other DNA

SEQ ID NO: 606 - DNA, 77 bp, synthetic construct; identical to SEQ ID NO: 534
  misc_feature 1..77: note = Synthetic Polynucleotide
  source 1..77: mol_type = other DNA

SEQ ID NO: 607 - DNA, 77 bp, synthetic construct; identical to SEQ ID NO: 534
  misc_feature 1..77: note = Synthetic Polynucleotide
  source 1..77: mol_type = other DNA

SEQ ID NO: 608 - DNA, 77 bp, synthetic construct; identical to SEQ ID NO: 534
  misc_feature 1..77: note = Synthetic Polynucleotide
  source 1..77: mol_type = other DNA

SEQ ID NO: 609 - DNA, 77 bp, synthetic construct; identical to SEQ ID NO: 534
  misc_feature 1..77: note = Synthetic Polynucleotide
  source 1..77: mol_type = other DNA

SEQ ID NO: 610 - DNA, 77 bp, synthetic construct; identical to SEQ ID NO: 534
  misc_feature 1..77: note = Synthetic Polynucleotide
  source 1..77: mol_type = other DNA

SEQ ID NO: 611 - DNA, 77 bp, synthetic construct; identical to SEQ ID NO: 534
  misc_feature 1..77: note = Synthetic Polynucleotide
  source 1..77: mol_type = other DNA

SEQ ID NO: 612 - DNA, 77 bp, synthetic construct; identical to SEQ ID NO: 534
  misc_feature 1..77: note = Synthetic Polynucleotide
  source 1..77: mol_type = other DNA

SEQ ID NO: 613 - DNA, 77 bp, synthetic construct; identical to SEQ ID NO: 534
  misc_feature 1..77: note = Synthetic Polynucleotide
  source 1..77: mol_type = other DNA

SEQ ID NO: 614 - DNA, 78 bp, synthetic construct
  misc_feature 1..78: note = Synthetic Polynucleotide
  source 1..78: mol_type = other DNA
ccattctccctatcactttcaggtacaccaatcaaacgtaggtttggtcttttcacatagtcccatatttcttggagg

SEQ ID NO: 615 - DNA, 77 bp, synthetic construct
  misc_feature 1..77: note = Synthetic Polynucleotide
  source 1..77: mol_type = other DNA
cctcgtcactgccagattttgtggctaccagcaaaggatcgttttaagctgcaactcaggaaattgagaaaatatgg

SEQ ID NO: 616 - DNA, 78 bp, synthetic construct
  misc_feature 1..78: note = Synthetic Polynucleotide
  source 1..78: mol_type = other DNA
cctccaagaaatatgggactatgtgaaaaaaccaaacctacgtttgattggtgtacctgaaagtgacagggagaatgg

SEQ ID NO: 617 - DNA, 77 bp, synthetic construct
  misc_feature 1..77: note = Synthetic Polynucleotide
  source 1..77: mol_type = other DNA
ccctgtgttcttttatactaaaacaagccagcaaaccaacctttgagatgtgttgccttaaacattactgaatgggg

SEQ ID NO: 618 - DNA, 76 bp, synthetic construct
  misc_feature 1..76: note = Synthetic Polynucleotide
  source 1..76: mol_type = other DNA
ccctgtgttcttttatactaaaacaagccagcaaaccaacctttgagatgtgttgccttaaacattactgaatggg

SEQ ID NO: 619 - DNA, 77 bp, synthetic construct
  misc_feature 1..77: note = Synthetic Polynucleotide
  source 1..77: mol_type = other DNA
ccgagaaacggctttagcaacaaataaatatcaaaaggatgctttctcttcagaataatctaaagtaagttgggagg

SEQ ID NO: 620 - DNA, 78 bp, synthetic construct
  misc_feature 1..78: note = Synthetic Polynucleotide
  source 1..78: mol_type = other DNA
ccatgttactccggataaggacagcaaaggaggaaaggaaccttttctgggccaccagaaggatgagcttgggcttgg

SEQ ID NO: 621 - DNA, 78 bp, synthetic construct
  misc_feature 1..78: note = Synthetic Polynucleotide
  source 1..78: mol_type = other DNA
cccagggatatgctggccacggggaggagccggaaaccaacctttgtgtcactgtgtagtgacaagtgcctttggagg

SEQ ID NO: 622 - DNA, 77 bp, synthetic construct
  misc_feature 1..77: note = Synthetic Polynucleotide
  source 1..77: mol_type = other DNA
ccagggatatgctggccacggggaggagccggaaaccaacctttgtgtcactgtgtagtgacaagtgcctttggagg

SEQ ID NO: 623 - DNA, 78 bp, synthetic construct
  misc_feature 1..78: note = Synthetic Polynucleotide
  source 1..78: mol_type = other DNA
ccttagggacccataatggccacaaccaggagaaaagcaagctttgatgcttaaacactacttacagacatgtacagg

SEQ ID NO: 624 - DNA, 78 bp, synthetic construct
  misc_feature 1..78: note = Synthetic Polynucleotide
  source 1..78: mol_type = other DNA
cctgcctctgttcctccttcctgatggtggcggaaaggatgcttttgccagatcaacagtcacacacaacacaccagg

SEQ ID NO: 625 - DNA, 78 bp, synthetic construct
  misc_feature 1..78: note = Synthetic Polynucleotide
  source 1..78: mol_type = other DNA
cctgactccagccctccttgacaaggtctccgtaaagcatgctttctcttagggaccctcagagggaggcttggtggg

SEQ ID NO: 626 - DNA, 77 bp, synthetic construct
  misc_feature 1..77: note = Synthetic Polynucleotide
  source 1..77: mol_type = other DNA
cctgactccagccctccttgacaaggtctccgtaaagcatgctttctcttagggaccctcagagggaggcttggtgg

SEQ ID NO: 627 - DNA, 77 bp, synthetic construct
  misc_feature 1..77: note = Synthetic Polynucleotide
  source 1..77: mol_type = other DNA
ccttatttggaatgtgacaagacccatttgtttaaaccttggtttttatgcagaaagaaaaggaaggctgcagtggg

SEQ ID NO: 628 - DNA, 78 bp, synthetic construct
  misc_feature 1..78: note = Synthetic Polynucleotide
  source 1..78: mol_type = other DNA
ccattctccctgtcactttcaggtacactaatcaaacgtaggtttgctgtttttacataggctcatatttcttggagg

SEQ ID NO: 629 - DNA, 78 bp, synthetic construct
  misc_feature 1..78: note = Synthetic Polynucleotide
  source 1..78: mol_type = other DNA
ccattctccccatcactttcaggtacaccagtcaaacgtaggtttggtcttttcacatagtcccatatttcttggagg

SEQ ID NO: 630 - DNA, 76 bp, synthetic construct
  misc_feature 1..76: note = Synthetic Polynucleotide
  source 1..76: mol_type = other DNA
cctgtttgttattttagctaatgtcaaaaagaaaaccttgctttttctgaaccctttcagaggcagaaagtggggg

SEQ ID NO: 631 - DNA, 77 bp, synthetic construct
  misc_feature 1..77: note = Synthetic Polynucleotide
  source 1..77: mol_type = other DNA
ccattttccccaccactttcacgtacagcaatcaaacgtaggtttggtcttttcactagtcccatatttcttggagg

SEQ ID NO: 632 - DNA, 77 bp, synthetic construct; identical to SEQ ID NO: 215
  misc_feature 1..77: note = Synthetic Polynucleotide
  source 1..77: mol_type = other DNA

SEQ ID NO: 633 - DNA, 77 bp, synthetic construct; identical to SEQ ID NO: 216
  misc_feature 1..77: note = Synthetic Polynucleotide
  source 1..77: mol_type = other DNA

SEQ ID NO: 634 - DNA, 77 bp, synthetic construct; identical to SEQ ID NO: 217
  misc_feature 1..77: note = Synthetic Polynucleotide
  source 1..77: mol_type = other DNA

SEQ ID NO: 635 - DNA, 77 bp, synthetic construct; identical to SEQ ID NO: 218
  misc_feature 1..77: note = Synthetic Polynucleotide
  source 1..77: mol_type = other DNA

SEQ ID NO: 636 - DNA, 77 bp, synthetic construct; identical to SEQ ID NO: 216
  misc_feature 1..77: note = Synthetic Polynucleotide
  source 1..77: mol_type = other DNA

SEQ ID NO: 637 - DNA, 77 bp, synthetic construct; identical to SEQ ID NO: 220
  misc_feature 1..77: note = Synthetic Polynucleotide
  source 1..77: mol_type = other DNA

SEQ ID NO: 638 - DNA, 77 bp, synthetic construct; identical to SEQ ID NO: 177
  misc_feature 1..77: note = Synthetic Polynucleotide
  source 1..77: mol_type = other DNA

SEQ ID NO: 639 - DNA, 77 bp, synthetic construct; identical to SEQ ID NO: 222
  misc_feature 1..77: note = Synthetic Polynucleotide
  source 1..77: mol_type = other DNA

SEQ ID NO: 640 - DNA, 77 bp, synthetic construct; identical to SEQ ID NO: 223
  misc_feature 1..77: note = Synthetic Polynucleotide
  source 1..77: mol_type = other DNA

SEQ ID NO: 641 - DNA, 77 bp, synthetic construct; identical to SEQ ID NO: 217
  misc_feature 1..77: note = Synthetic Polynucleotide
  source 1..77: mol_type = other DNA

SEQ ID NO: 642 - DNA, 77 bp, synthetic construct; identical to SEQ ID NO: 225
  misc_feature 1..77: note = Synthetic Polynucleotide
  source 1..77: mol_type = other DNA

SEQ ID NO: 643 - DNA, 78 bp, synthetic construct
  misc_feature 1..78: note = Synthetic Polynucleotide
  source 1..78: mol_type = other DNA
ccaggaaaaaatttaaactttcttaacttgataaaaggtagctttcaaaacctacaataaataacatacttagagtgg

SEQ ID NO: 644 - DNA, 78 bp, synthetic construct
  misc_feature 1..78: note = Synthetic Polynucleotide
  source 1..78: mol_type = other DNA
ccattctcctcgtcactttcaggtacaccaaacaaacgtaggtttggtctttttacgtagtcccatatttcttggagg

SEQ ID NO: 645 - DNA, 78 bp, synthetic construct
  misc_feature 1..78: note = Synthetic Polynucleotide
  source 1..78: mol_type = other DNA
ccctcttgaagttagggaagtagcatttaagggaaacgtagctttactattaagaatttcaaacagcacttgtcaggg

SEQ ID NO: 646 - DNA, 77 bp, synthetic construct
  misc_feature 1..77: note = Synthetic Polynucleotide
  source 1..77: mol_type = other DNA
ccctcttgaagttagggaagtagcatttaagggaaacgtagctttactattaagaatttcaaacagcacttgtcagg

SEQ ID NO: 647 - DNA, 77 bp, synthetic construct
  misc_feature 1..77: note = Synthetic Polynucleotide
  source 1..77: mol_type = other DNA
cctcttgaagttagggaagtagcatttaagggaaacgtagctttactattaagaatttcaaacagcacttgtcaggg

SEQ ID NO: 648 - DNA, 76 bp, synthetic construct
  misc_feature 1..76: note = Synthetic Polynucleotide
  source 1..76: mol_type = other DNA
cctcttgaagttagggaagtagcatttaagggaaacgtagctttactattaagaatttcaaacagcacttgtcagg

SEQ ID NO: 649 - DNA, 78 bp, synthetic construct
  misc_feature 1..78: note = Synthetic Polynucleotide
  source 1..78: mol_type = other DNA
ccattctccccgtcactttcaggtacaccaatcaaacgtaggtttggtcttttcacatattcccatatttcttggagg

SEQ ID NO: 650 - DNA, 77 bp, synthetic construct
  misc_feature 1..77: note = Synthetic Polynucleotide
  source 1..77: mol_type = other DNA
ccattctcccttcactttcaggtacaccaatcaaacgtaggtttggtcttttcacatagtcccatattttttggagg

SEQ ID NO: 651 - DNA, 78 bp, synthetic construct
  misc_feature 1..78: note = Synthetic Polynucleotide
  source 1..78: mol_type = other DNA
cctatagtctcagttacttgggaggctgaggtaaaaggatcgtttgagcccaggaggtggaggttgcagtgagccggg

SEQ ID NO: 652 - DNA, 77 bp, synthetic construct
  misc_feature 1..77: note = Synthetic Polynucleotide
  source 1..77: mol_type = other DNA
cctatagtctcagttacttgggaggctgaggtaaaaggatcgtttgagcccaggaggtggaggttgcagtgagccgg

SEQ ID NO: 653 - DNA, 77 bp, synthetic construct
  misc_feature 1..77: note = Synthetic Polynucleotide
  source 1..77: mol_type = other DNA
cctttcccaactctgctattgcccccacatcctaaaggaacctttctttttttatatattttattttaagttccagg

SEQ ID NO: 654 - DNA, 78 bp, synthetic construct
  misc_feature 1..78: note = Synthetic Polynucleotide
  source 1..78: mol_type = other DNA
cctccaagaaatatggaactatgtgaaaagaccaaacctacgtttgattgacgtacctgaaagtgacagggagaatgg

SEQ ID NO: 655 - DNA, 76 bp, synthetic construct
  misc_feature 1..76: note = Synthetic Polynucleotide
  source 1..76: mol_type = other DNA
cctcttctgaaagcattgataatcaacattttaaacgtagcttttccccatattgctaggaaggctcattcccggg

SEQ ID NO: 656 - DNA, 78 bp, synthetic construct
  misc_feature 1..78: note = Synthetic Polynucleotide
  source 1..78: mol_type = other DNA
cctccaagaaatatgggactatgtgaaaaggccaaacctacgtttgattgctgtacccgagagtgacggggagaatgg

SEQ ID NO: 657 - DNA, 78 bp, synthetic construct; identical to SEQ ID NO: 333
  misc_feature 1..78: note = Synthetic Polynucleotide
  source 1..78: mol_type = other DNA

SEQ ID NO: 658 - DNA, 77 bp, synthetic construct
  misc_feature 1..77: note = Synthetic Polynucleotide
  source 1..77: mol_type = other DNA
cccggggcctgggtgcccagtgccagtggtcagaaaggttgctttggtgtttttcattgttagtgagacagagatgg

SEQ ID NO: 659 - DNA, 76 bp, synthetic construct
  misc_feature 1..76: note = Synthetic Polynucleotide
  source 1..76: mol_type = other DNA
ccggggcctgggtgcccagtgccagtggtcagaaaggttgctttggtgtttttcattgttagtgagacagagatgg

SEQ ID NO: 660 - DNA, 78 bp, synthetic construct
  misc_feature 1..78: note = Synthetic Polynucleotide
  source 1..78: mol_type = other DNA
ccattctccccatcattttcaggtacaccaatcaaacgtaggtttgatcttttcacatagccccatatttcttggagg

SEQ ID NO: 661 - DNA, 77 bp, synthetic construct
  misc_feature 1..77: note = Synthetic Polynucleotide
  source 1..77: mol_type = other DNA
ccaccagcacttctgttagaagttgcagcagagaaaggatcctttaggcacatctcccagatccttgcgaagagggg

SEQ ID NO: 662 - DNA, 78 bp, synthetic construct
  misc_feature 1..78: note = Synthetic Polynucleotide
  source 1..78: mol_type = other DNA
cctgtgccagggtccttccactgggactggcagaaacgtaggtttgcatggagtgagaagcaggggagaggttgaggg

SEQ ID NO: 663 - DNA, 77 bp, synthetic construct
  misc_feature 1..77: note = Synthetic Polynucleotide
  source 1..77: mol_type = other DNA
cctgtgccagggtccttccactgggactggcagaaacgtaggtttgcatggagtgagaagcaggggagaggttgagg

SEQ ID NO: 664 - DNA, 78 bp, synthetic construct
  misc_feature 1..78: note = Synthetic Polynucleotide
  source 1..78: mol_type = other DNA
ccctcagcctctcccctgcttctcactccatgcaaacctacgtttctgccagtcccagcagaaggaccctggcacggg

SEQ ID NO: 665 - DNA, 77 bp, synthetic construct
  misc_feature 1..77: note = Synthetic Polynucleotide
  source 1..77: mol_type = other DNA
ccctcagcctctcccctgcttctcactccatgcaaacctacgtttctgccagtcccagcagaaggaccctggcacgg

SEQ ID NO: 666 - DNA, 77 bp, synthetic construct
  misc_feature 1..77: note = Synthetic Polynucleotide
  source 1..77: mol_type = other DNA
cctcagcctctcccctgcttctcactccatgcaaacctacgtttctgccagtcccagcagaaggaccctggcacggg

SEQ ID NO: 667 - DNA, 76 bp, synthetic construct
  misc_feature 1..76: note = Synthetic Polynucleotide
  source 1..76: mol_type = other DNA
cctcagcctctcccctgcttctcactccatgcaaacctacgtttctgccagtcccagcagaaggaccctggcacgg

SEQ ID NO: 668 - DNA, 78 bp, synthetic construct
  misc_feature 1..78: note = Synthetic Polynucleotide
  source 1..78: mol_type = other DNA
cctccaagaaatatggggctatgtgaaaagaccaaacctacctttgattggtgtatctgaaagtgacggggagaatgg

SEQ ID NO: 669 - DNA, 78 bp, synthetic construct; identical to SEQ ID NO: 507
  misc_feature 1..78: note = Synthetic Polynucleotide
  source 1..78: mol_type = other DNA

SEQ ID NO: 670 - DNA, 78 bp, synthetic construct
  misc_feature 1..78: note = Synthetic Polynucleotide
  source 1..78: mol_type = other DNA
ccattctccccgtcactttcaggtacaccaatcaaacgtaggtttggtcttttctcattgtcccatatttcttggagg

SEQ ID NO: 671 - DNA, 76 bp, synthetic construct
  misc_feature 1..76: note = Synthetic Polynucleotide
  source 1..76: mol_type = other DNA
cccatcaagagcggttgtgcatggcaacagtaaaaggatggtttgttacactagtacaaaaagaggtggccagagg

SEQ ID NO: 672 - DNA, 78 bp, synthetic construct; identical to SEQ ID NO: 291
  misc_feature 1..78: note = Synthetic Polynucleotide
  source 1..78: mol_type = other DNA

SEQ ID NO: 673 - DNA, 78 bp, synthetic construct
  misc_feature 1..78: note = Synthetic Polynucleotide
  source 1..78: mol_type = other DNA
cctccaagaaatacgggactatgtgaaaagaccaaacgtacgtttgattggtgtacctgaaagtgatagggagaatgg

SEQ ID NO: 674 - DNA, 78 bp, synthetic construct
  misc_feature 1..78: note = Synthetic Polynucleotide
  source 1..78: mol_type = other DNA
cctccaagaaatatgggactatgtgaaaagaccaaacctacgtttgattggtgtacctgaaagtgactgggagaatgg

SEQ ID NO: 675 - DNA, 78 bp, synthetic construct
  misc_feature 1..78: note = Synthetic Polynucleotide
  source 1..78: mol_type = other DNA
ccattctccctgtcactttcaggtacacgaatcaaacgtaggtttcatcttttcacatagtcccatatttcttagagg

SEQ ID NO: 676 - DNA, 77 bp, synthetic construct
  misc_feature 1..77: note = Synthetic Polynucleotide
  source 1..77: mol_type = other DNA
ccattctctctgtcactttctggtacaccaatcaaacgtaggtttggtcttttcacatagtttcacatatttcttgg

SEQ ID NO: 677 - DNA, 78 bp, synthetic construct
  misc_feature 1..78: note = Synthetic Polynucleotide
  source 1..78: mol_type = other DNA
cctccaagaaatatgggactatgtgaaaagaccaaacctacgtttgattggtgtacctgaaagtgacaaggaaaatgg

SEQ ID NO: 678 - DNA, 78 bp, synthetic construct
  misc_feature 1..78: note = Synthetic Polynucleotide
  source 1..78: mol_type = other DNA
cctgaaaaacattgtttccaacctggtaaatcaaaaggaaggtttaactttgttagataagtccacatatcaccaagg

SEQ ID NO: 679 - DNA, 78 bp, synthetic construct
  misc_feature 1..78: note = Synthetic Polynucleotide
  source 1..78: mol_type = other DNA
cctccaagaaatgtgggactatgggaaaagaccaaacctacctttgtttggtgtacctgaaagtgacggggagaaagg

SEQ ID NO: 680 - DNA, 78 bp, synthetic construct
  misc_feature 1..78: note = Synthetic Polynucleotide
  source 1..78: mol_type = other DNA
cctccaagaaatatgggactatgtgaaaagaccaaacctacgtttcattggtgtacctgaaagtgatgggtagaatgg

SEQ ID NO: 681 - DNA, 78 bp, synthetic construct
  misc_feature 1..78: note = Synthetic Polynucleotide
  source 1..78: mol_type = other DNA
cctacaagaaatatgggactatgggaaaagaccaaacctacgtttgattggtacactggaaagtgacagggataatgg

SEQ ID NO: 682 - DNA, 78 bp, synthetic construct
  misc_feature 1..78: note = Synthetic Polynucleotide
  source 1..78: mol_type = other DNA
ccattctccctgtcactttctggtacaccaatcaaaggtaggtttggtcttttcacatagtcccatatttcttggagg

SEQ ID NO: 683 - DNA, 78 bp, synthetic construct; identical to SEQ ID NO: 333
  misc_feature 1..78: note = Synthetic Polynucleotide
  source 1..78: mol_type = other DNA

SEQ ID NO: 684 - DNA, 78 bp, synthetic construct
  misc_feature 1..78: note = Synthetic Polynucleotide
  source 1..78: mol_type = other DNA
ccattctctttgtcactttcaggtataccaatcaaacgttggtttggtctttttgcatagtcccatattttgtggagg

SEQ ID NO: 685 - DNA, 78 bp, synthetic construct
  misc_feature 1..78: note = Synthetic Polynucleotide
  source 1..78: mol_type = other DNA
cctccaagaaatatgagactatgtgaaaagaccaaacctacgtttgattagtgtacctgaaaatgatggggagaatgg

SEQ ID NO: 686 - DNA, 78 bp, synthetic construct
  misc_feature 1..78: note = Synthetic Polynucleotide
  source 1..78: mol_type = other DNA
ccattctttctgtcactttcaggtacaccaatcaaacgtaggtttggtcttttcacatagtcccatatttcttggagg

SEQ ID NO: 687 - DNA, 78 bp, synthetic construct
  misc_feature 1..78: note = Synthetic Polynucleotide
  source 1..78: mol_type = other DNA
ccattctccctgtcactttcaggtacaccaatcaaacgtaggtttgttcttttcacatagtcccatatttcttggagg

SEQ ID NO: 688 - DNA, 78 bp, synthetic construct
  misc_feature 1..78: note = Synthetic Polynucleotide
  source 1..78: mol_type = other DNA
ccattatccccatcactttcaggtacaccaatcaaacgtaggtttggttttttcacatagttcaatatttctttgagg

SEQ ID NO: 689 - DNA, 78 bp, synthetic construct
  misc_feature 1..78: note = Synthetic Polynucleotide
  source 1..78: mol_type = other DNA
cctccaagaaatatgggactatctgaaaagatcaaacctacgtttgattggtgtacctgaaagtgacagggagaatgg

SEQ ID NO: 690 - DNA, 78 bp, synthetic construct
  misc_feature 1..78: note = Synthetic Polynucleotide
  source 1..78: mol_type = other DNA
cctttctccccatcactttcaggtacaccaatcaaacgtaggtttggtcttttcatatagtcccatatttcttggagg

SEQ ID NO: 691 - DNA, 78 bp, synthetic construct
  misc_feature 1..78: note = Synthetic Polynucleotide
  source 1..78: mol_type = other DNA
cctccaagaaatatgggactatgtgcaaagatcaaacctacgtttgattgctgtacctgaaagtgatggggagaatgg

SEQ ID NO: 692 - DNA, 78 bp, synthetic construct
  misc_feature 1..78: note = Synthetic Polynucleotide
  source 1..78: mol_type = other DNA
ccattctccccatcactttcaggtacaccagtcaaacgtaggtttggtcttttcacataatcccatatttcttggagg

SEQ ID NO: 693 - DNA, 77 bp, synthetic construct
  misc_feature 1..77: note = Synthetic Polynucleotide
  source 1..77: mol_type = other DNA
cctccaagaagtatgggaccatggaaaagatcaaacctacgtttgactggtgtacctgaaagtgactgggagaatgg

SEQ ID NO: 694 - DNA, 78 bp, synthetic construct
  misc_feature 1..78: note = Synthetic Polynucleotide
  source 1..78: mol_type = other DNA
cctccaagaaatatgggactatgtgaaaagaccaaacctacgtttgattggagtacttgaaaatgacagggataatgg

SEQ ID NO: 695 - DNA, 77 bp, synthetic construct
  misc_feature 1..77: note = Synthetic Polynucleotide
  source 1..77: mol_type = other DNA
cctttaaagacatgctctttgtgccagaaattcaaaggttgcttttatgtccagtggggtggagggaggaagctcgg

SEQ ID NO: 696 - DNA, 78 bp, synthetic construct
  misc_feature 1..78: note = Synthetic Polynucleotide
  source 1..78: mol_type = other DNA
ccattctccccgtcactttcagggacctcaatcaaacgtaggttttgtcttttcacatagtcccatatttcttggagg

SEQ ID NO: 697 - DNA, 78 bp, synthetic construct
  misc_feature 1..78: note = Synthetic Polynucleotide
  source 1..78: mol_type = other DNA
cctccaagaaatataggactatgtgaaaagaccaaacctacgtttgactggtgtacctgaaagtgacagggagaatgg

SEQ ID NO: 698 - DNA, 78 bp, synthetic construct
  misc_feature 1..78: note = Synthetic Polynucleotide
  source 1..78: mol_type = other DNA
ccattctccccatcactttcaggtacaccaatcaaaggtaggtttggtcttttcacatagtccgatatttcctgcagg

SEQ ID NO: 699 - DNA, 12 bp, synthetic construct
  misc_feature 1..12: note = Synthetic Polynucleotide
  misc_feature 4..5: note = s is g or c
  misc_feature 6..7: note = w is a, t or u
  misc_feature 8..9: note = s is g or c
  source 1..12: mol_type = other DNA
aaasswwssttt

SEQ ID NO: 700 - DNA, 20 bp, synthetic construct
  misc_feature 1..20: note = Synthetic Polynucleotide
  source 1..20: mol_type = other DNA
ctgtaaaccgaggttttgga

SEQ ID NO: 701 - protein, 15 aa, synthetic construct
  REGION 1..15: note = Synthetic Polypeptide
GGSGGSGGSGGSGGS

SEQ ID NO: 702 - protein, 7 aa, synthetic construct
  REGION 1..7: note = Synthetic Polypeptide
PKKKRKV

SEQ ID NO: 703 - DNA, 20 bp, synthetic construct
  misc_feature 1..20: note = Synthetic Polynucleotide
  source 1..20: mol_type = other DNA
tccaaaacctcggtttacag

SEQ ID NO: 704 - DNA, 78 bp, synthetic construct; identical to SEQ ID NO: 176
  misc_feature 1..78: note = Synthetic Polynucleotide
  source 1..78: mol_type = other DNA

SEQ ID NO: 705 - DNA, 78 bp, synthetic construct
  misc_feature 1..78: note = Synthetic Polynucleotide
  source 1..78: mol_type = other DNA
cccagaggagaagggtcatgggcctggcccacaaaaccatggttttctcttaaacctcagggcctgtgatgggagggg

SEQ ID NO: 706 - DNA, 76 bp, synthetic construct; identical to SEQ ID NO: 480
  misc_feature 1..76: note = Synthetic Polynucleotide
  source 1..76: mol_type = other DNA

SEQ ID NO: 707 - DNA, 76 bp, synthetic construct
  misc_feature 1..76: note = Synthetic Polynucleotide
  source 1..76: mol_type = other DNA
ccactgcagcccaagcttaggaattccagcgaaaacctagcttttttctcttcatatccgtttatcagagtcaggg

SEQ ID NO: 708 - DNA, 78 bp, synthetic construct; identical to SEQ ID NO: 481
  misc_feature 1..78: note = Synthetic Polunucleotide
  source 1..78: mol_type = other DNA

SEQ ID NO: 709 - DNA, 78 bp, synthetic construct
  misc_feature 1..78: note = Synthetic Polynucleotide
  source 1..78: mol_type = other DNA
cctccaagaaatatgggactgtgtgaaaagaccaaacctacgtttcattggtgtacctaaaagtgactgggagaaggg

SEQ ID NO: 710 - DNA, 77 bp, synthetic construct
  misc_feature 1..77: note = Synthetic Polynucleotide
  source 1..77: mol_type = other DNA
ccctgacactgataaacggatatgaagagaaaaaagctaggtttggtcttttcacacagtcccatatttcttggagg

SEQ ID NO: 711 - DNA, 77 bp, synthetic construct
  misc_feature 1..77: note = Synthetic Polynucleotide
  source 1..77: mol_type = other DNA
cctccaagaaatatgggactgtgtgaaaagaccaaacctagcttttttctcttcatatccgtttatcagagtcaggg

SEQ ID NO: 712 - protein, 1367 aa, synthetic construct
  REGION 1..1367: note = Synthetic Polypeptide
DKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 713 - protein, 142 aa, synthetic construct
  REGION 1..142: note = Synthetic Polypeptide
MLIGYVRVSTNDQNTDLQRNALVCAGCEQIFEDKLSGTRTDRPGLKRALKRLQKGDTLVVWKLDRLGRSMKHLISLVGELRERGINFRSLTDSIDTSSPMGRFFFYVMGALAEMERELIIERTMAGLAAARNKGRRFGRPPK

SEQ ID NO: 714 - protein, 1300 aa, unidentified
  source 1..1300: note = Francisella novicida
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 715 - protein, 1300 aa, unidentified
  source 1..1300: note = Francisella novicida
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIARGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 716 - protein, 1300 aa, unidentified
  source 1..1300: note = Francisella novicida
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFADLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 717 - protein, 1300 aa, unidentified
  source 1..1300: note = Francisella novicida
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDAAANGAYHIGLKGLMLLGRIKNNQEGKKLNLVIKNEEYFEFVQNRNN

SEQ ID NO: 718 - protein, 887 aa, Natronobacterium gregoryi
MTVIDLDSTTTADELTSGHTYDISVTLTGVYDNTDEQHPRMSLAFEQDNGERRYITLWKNTTPKDVFTYDYATGSTYIFTNIDYEVKDGYENLTATYQTTVENATAQEVGTTDEDETFAGGEPLDHHLDDALNETPDDAETESDSGHVMTSFASRDQLPEWTLHTYTLTATDGAKTDTEYARRTLAYTVRQELYTDHDAAPVATDGLMLLTPEPLGETPLDLDCGVRVEADETRTLDYTTAKDRLLARELVEEGLKRSLWDDYLVRGIDEVLSKEPVLTCDEFDLHERYDLSVEVGHSGRAYLHINFRHRFVPKLTLADIDDDNIYPGLRVKTTYRPRRGHIVWGLRDECATDSLNTLGNQSVVAYHRNNQTPINTDLLDAIEAADRRVVETRRQGHGDDAVSFPQELLAVEPNTHQIKQFASDGFHQQARSKTRLSASRCSEKAQAFAERLDPVRLNGSTVEFSSEFFTGNNEQQLRLLYENGESVLTFRDGARGAHPDETFSKGIVNPPESFEVAVVLPEQQADTCKAQWDTMADLLNQAGAPPTRSETVQYDAFSSPESISLNVAGAIDPSEVDAAFVVLPPDQEGFADLASPTETYDELKKALANMGIYSQMAYFDRFRDAKIFYTRNVALGLLAAAGGVAFTTEHAMPGDADMFIGIDVSRSYPEDGASGQINIAATATAVYKDGTILGHSSTRPQLGEKLQSTDVRDIMKNAILGYQQVTGESPTHIVIHRDGFMNEDLDPATEFLNEQGVEYDIVEIRKQPQTRLLAVSDVQYDTPVKSIAAINQNEPRATVATFGAPEYLATRDGGGLPRPIQIERVAGETDIETLTRQVYLLSQSHIQVHNSTARLPITTAYADQASTHATKGYLVQTGAFESNVGFL

SEQ ID NO: 719 - protein, 1544 aa, synthetic construct
  REGION 1..1544: note = Synthetic Polypeptide
MLIGYVRVSTNDQNTDLQRNALVCAGCEQIFEDKLSGTRTDRPGLKRALKRLQKGDTLVVWKLDRLGRSMKHLISLVGELRERGINFRSLTDSIDTSSPMGRFFFYVMGALAEMERELIIERTMAGLAAARNKGRRFGRPPKGGSGGSGGSGGSGGSGGSGGSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGGSDYKDDDDK

SEQ ID NO: 720 - protein, 1367 aa, synthetic construct
  REGION 1..1367: note = Synthetic polypeptide
DKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTAFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGALSRKLINGIRDKQSGKTILDFLKSDGFANRNFMALIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRAITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 721 - protein, 1612 aa, synthetic construct
  REGION 1..1612: note = Synthetic polypeptide
MSSETGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPHVTLFIYIARLYHHADPRNRQGLRDLISSGVTIQIMTEQESGYCWRNFVNYSPSNEAHWPRYPHLWVRLYVLELYCIILGLPPCLNILRRKQPQLTFFTIALQSCHYQRLPPHILWATGLKSGSETPGTSESATPESDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTAFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGALSRKLINGIRDKQSGKTILDFLKSDGFANRNFMALIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRAITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 722 - protein, 5 aa, synthetic construct
  REGION 1..5: note = Synthetic polypeptide
GGGGS

SEQ ID NO: 723 - protein, 5 aa, synthetic construct
  REGION 1..5: note = Synthetic polypeptide
EAAAK

SEQ ID NO: 724 - protein, 16 aa, synthetic construct
  REGION 1..16: note = Synthetic polypeptide
SGSETPGTSESATPES

SEQ ID NO: 725 - protein, 343 aa, synthetic construct
  REGION 1..343: note = Synthetic polypeptide
MSNLLTVHQNLPALPVDATSDEVRKNLMDMFRDRQAFSEHTWKMLLSVCRSWAAWCKLNNRKWFPAEPEDVRDYLLYLQARGLAVKTIQQHLGQLNMLHRRSGLPRPSDSNAVSLVMRRIRKENVDAGERAKQALAFERTDFDQVRSLMENSDRCQDIRNLAFLGIAYNTLLRIAEIARIRVKDISRTDGGRMLIHIGRTKTLVSTAGVEKALSLGVTKLVERWISVSGVADDPNNYLFCRVRKNGVAAPSATSQLSTRALEGIFEATHRLIYGAKDDSGQRYLAWSGHSARVGAARDMARAGVSIPEIMQAGGWTNVNIVMNYIRNLDSETGAMVRLLEDGD

SEQ ID NO: 726 - protein, 423 aa, synthetic construct
  REGION 1..423: note = Synthetic polypeptide
MPQFGILCKTPPKVLVRQFVERFERPSGEKIALCAAELTYLCWMITHNGTAIKRATFMSYNTIISNSLSFDIVNKSLQFKYKTQKATILEASLKKLIPAWEFTIIPYYGQKHQSDITDIVSSLQLQFESSEEADKGNSHSKKMLKALLSEGESIWEITEKILNSFEYTSRFTKTKTLYQFLFLATFINCGRFSDIKNVDPKSFKLVQNKYLGVIIQCLVTETKTSVSRHIYFFSARGRIDPLVYLDEFLRNSEPVLKRVNRTGNSSSNKQEYQLLKDNLVRSYNKALKKNAPYSIFAIKNGPKSHIGRHLMTSFLSMKGLTELTNVVGNWSDKRASAVARTTYTHQITAIPDHYFALVSRYYAYDPISKEMIALKDETNPIEEWQHIEQLKGSAEGSIRYPAWNGIISQEVLDYLSSYINRRI

SEQ ID NO: 727 - protein, 144 aa, synthetic construct
  REGION 1..144: note = Synthetic polypeptide
MRLFGYARVSTSQQSLDIQVRALKDAGVKANRIFTDKASGSSSDRKGLDLLRMKVEEGDVILVKKLDRLGRDTADMIQLIKEFDAQGVSIRFIDDGISTDGEMGKMVVTILSAVAQAERQRILERTNEGRQEAMAKGVVFGRKR

SEQ ID NO: 728 - protein, 144 aa, synthetic construct; identical to SEQ ID NO: 727
  REGION 1..144: note = Synthetic polypeptide

SEQ ID NO: 729 - protein, 144 aa, synthetic construct
  REGION 1..144: note = Synthetic polypeptide
MRLFGYARVSTSQQSLDIQVRALKDAGVKANRIFTDKASGSSSDRKGLDLLRMKVEEGDVILVKKLDRLGRDTADMIQLIKEFDAQGVSIRFIDDGISTDGYMGKMVVTILSAVAQAERQRILQRTNEGRQEAMAKGVVFGRKR

SEQ ID NO: 730 - protein, 147 aa, synthetic construct
  REGION 1..147: note = Synthetic polypeptide
MAKIGYARVSSKEQNLDRQLQALQGVSKVFSDKLSGQSVERPQLQAMLNYIREGDIVVVTELDRLGRNNKELTELMNAIQQKGATLEVLDLPSMNGIEDENLRRLINNLVIELYKYQAESERKRIKERQAQGIEIAKSKGKFKGRQH

SEQ ID NO: 731 - protein, 147 aa, synthetic construct
  REGION 1..147: note = Synthetic polypeptide
MAKIGYARVSSKEQNLDRQLQALQGVSKVFSDKLSGQSVERPQLQAMLNYIREGDIVVVTELDRLGRNNKELTELMNAIQQKGATLEVLDLPSMDGIEDENLRRLINNLVIELYKYQAESERKRIKERQAQGIEIAKSKGKFKGRQH

SEQ ID NO: 732 - protein, 150 aa, synthetic construct
  REGION 1..150: note = Synthetic polypeptide
MIIGYARVSSLDQNLERQLENLKTFGAEKIFTEKQSGKSIENRPILQKALNFVRMGDRFIVESIDRLGRNYNEVIHTVNYLKDKEVQLMITSLPMMNEVIGNPLLDKFMKDLIIQILAMVSEQERNESKRRQAQGIQVAKEKGVYKGRPL

SEQ ID NO: 733 - protein, 150 aa, synthetic construct
  REGION 1..150: note = Synthetic polypeptide
MIIGYARVSSLDQNLERQLENLKTFGAEKIFTEKQSGKSIENRPILQKALNFVRMGDRFIVESIDRLGRNYNEVIHTVNYLKDKEVRLMITSLPMMNEVIGNPLLDKFMKDLIIRILAMVSEQERNESKRRQAQGIQVAKEKGVYKGRPL

SEQ ID NO: 734 - protein, 144 aa, synthetic construct
  REGION 1..144: note = Synthetic polypeptide
MRLFGYARVSTSQQSLDLQVRALKDAGVKANRIFTDKASGSSTDREGLDLLRMKVKEGDVILVKKLDRLGRDTADMLQLIKEFDAQGVAVRFIDDGISTDGDMGQMVVTILSAVAQAERRRILERTNEGRQEAKLKGIKFGRRR

SEQ ID NO: 735 - protein, 144 aa, synthetic construct
  REGION 1..144: note = Sythetic polypeptide
MRLFGYARVSTSQQSLDLQVRALKDAGVKANRIFTDKASGSSTDREGLDLLRMKVKEGDVILVKKLDRLSRDTADMLQLIKEFDAQGVAVRFIDDGISTDGYMGQMVVTILSAVAQAERRRILQRTNEGRQEAKLKGIKFGRRR

SEQ ID NO: 736 - protein, 142 aa, synthetic construct
  REGION 1..142: note = Synthetic polypeptide
MATIGYIRVSTIDQNIDLQRNALTSANCDRIFEDRISGKIANRPGLKRALKYVNKGDTLVVWKLDRLGRSVKNLVALISELHERGAHFHSLTDSIDTSSAMGRFFFHVMSALAEMERELIVERTLAGLAAARAQGRLGGRPV

SEQ ID NO: 737 - protein, 142 aa, synthetic construct
  REGION 1..142: note = Synthetic polypeptide
MATIGYIRVSTIDQNIDLQRNALTSANCDRIFEDRISGKIANRPGLKRALKYVNKGDTLVVWKLDRLGRSVKNLVALISELHERGAHFHSLTDSIDTSSAMGRFFFYVMSALAEMERELIVERTLAGLAAARAQGRLGGRPV

SEQ ID NO: 738 - protein, 608 aa, synthetic construct
  REGION 1..608: note = Synthetic polypeptide
MDTYAGAYDRQSRERENSSAASPATQRSANEDKAADLQREVERDGGRFRFVGHFSEAPGTSAFGTAERPEFERILNECRAGRLNMIIVYDVSRFSRLKVMDAIPIVSELLALGVTIVSTQEGVFRQGNVMDLIHLIMRLDASHKESSLKSAKILDTKNLQRELGGYVGGKAPYGFELVSETKEITRNGRMVNVVINKLAHSTTPLTGPFEFEPDVIRWWWREIKTHKHLPFKPGSQAAIHPGSITGLCKRMDADAVPTRGETIGKKTASSAWDPATVMRILRDPRIAGFAAEVIYKKKPDGTPTTKIEGYRIQRDPITLRPVELDCGPIIEPAEWYELQAWLDGRGRGKGLSRGQAILSAMDKLYCECGAVMTSKRGEESIKDSYRCRRRKVVDPSAPGQHEGTCNVSMAALDKFVAERIFNKIRHAEGDEETLALLWEAARRFGKLTEAPEKSGERANLVAERADALNALEELYEDRAAGAYDGPVGRKHFRKQQAALTLRQQGAEERLAELEAAEAPKLPLDQWFPEDADADPTGPKSWWGRASVDDKRVFVGLFVDKIVVTKSTTGRGQGTPIEKRASITWAKPPTDDDEDDAQDGTEDVAATGA

SEQ ID NO: 739 - DNA, 34 bp, synthetic construct
  misc_feature 1..34: note = Synthetic polynucleotide
  source 1..34: mol_type = other DNA
ataacttcgtatagcatacattatacgaagttat

SEQ ID NO: 740 - DNA, 34 bp, synthetic construct
  misc_feature 1..34: note = Synthetic polynucleotide
  source 1..34: mol_type = other DNA
gaagttcctattctctagaaagtataggaacttc

SEQ ID NO: 741 - protein, 4 aa, synthetic construct
  REGION 1..4: note = Synthetic polypeptide
NGAN

SEQ ID NO: 742 - protein, 4 aa, synthetic construct
  REGION 1..4: note = Synthetic polypeptide
NGNG

SEQ ID NO: 743 - protein, 4 aa, synthetic construct
  REGION 1..4: note = Synthetic polypeptide
NGAG

SEQ ID NO: 744 - protein, 4 aa, synthetic construct
  REGION 1..4: note = Synthetic polypeptide
NGCG

SEQ ID NO: 745 - protein, 6 aa, synthetic construct
  REGION 1..6: note = Synthetic polypeptide
NNGRRT

SEQ ID NO: 746 - protein, 5 aa, synthetic construct
  REGION 1..5: note = Synthetic polypeptide
NGRRN

SEQ ID NO: 747 - protein, 6 aa, synthetic construct
  REGION 1..6: note = Synthetic polypeptide
NNNRRT

SEQ ID NO: 748 - protein, 7 aa, synthetic construct
  REGION 1..7: note = Synthetic polypeptide
NNNGATT

SEQ ID NO: 749 - protein, 7 aa, synthetic construct
  REGION 1..7: note = Synthetic polypeptide
NNAGAAW

SEQ ID NO: 750 - protein, 5 aa, synthetic construct
  REGION 1..5: note = Synthetic polypeptide
NAAAC

SEQ ID NO: 751 - protein, 4 aa, synthetic construct
  REGION 1..4: note = Synthetic polypeptide
TTTN

SEQ ID NO: 752 - protein, 1367 aa, synthetic construct; identical to SEQ ID NO: 712
  REGION 1..1367: note = Synthetic Polypeptide

SEQ ID NO: 753 - protein, 1367 aa, synthetic construct
  REGION 1..1367: note = Synthetic Polypeptide
DKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 754 - protein, 1367 aa, synthetic construct
  REGION 1..1367: note = Synthetic Polypeptide
DKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 755 - protein, 345 aa, unidentified
  source 1..345: note = Sulfolobus islandicus
MEVPLYNIFGDNYIIQVATEAENSTIYNNKVEIDDEELRNVLNLAYKIAKNNEDAAAERRGKAKKKKGEEGETTTSNIILPLSGNDKNPWTETLKCYNFPTTVALSEVFKNFSQVKECEEVSAPSFVKPEFYEFGRSPGMVERTRRVKLEVEPHYLIIAAAGWVLTRLGKAKVSEGDYVGVNVFTPTRGILYSLIQNVNGIVPGIKPETAFGLWIARKVVSSVTNPNVSVVRIYTISDAVGQNPTTINGGFSIDLTKLLEKRYLLSERLEAIARNALSISSNMRERYIVLANYIYEYLTGSKRLEDLLYFANRDLIMNLNSDDGKVRDLKLISAYVNGELIRGEG

SEQ ID NO: 756 - protein, 345 aa, unidentified
  source 1..345: note = Sulfolobus islandicus
MEVPLYNIFGDNYIIQVATEAENSTIYNNKVEIDDEELRNVLNLAYKIAKNNEDAAAERRGKAKKKKGEEGETTTSNIILPLSGNDKNPWTETLKCYNFPTTVALSEVFKNFSQVKECEEVSAPSFVKPEFYKFGRSPGMVERTRRVKLEVEPHYLIMAAAGWVLTRLGKAKVSEGDYVGVNVFTPTRGILYSLIQNVNGIVPGIKPETAFGLWIARKVVSSVTNPNVSVVSIYTISDAVGQNPTTINGGFSIDLTKLLEKRDLLSERLEAIARNALSISSNMRERYIVLANYIYEYLTGSKRLEDLLYFANRDLIMNLNSDDGKVRDLKLISAYVNGELIRGEG

SEQ ID NO: 757 - protein, 1210 aa, unidentified
  REGION 1..1210: note = Percubacteria
MSKRHPRISGVKGYRLHAQRLEYTGKSGAMRTIKYPLYSSPSGGRTVPREIVSAINDDYVGLYGLSNFDDLYNAEKRNEEKVYSVLDFWYDCVQYGAVFSYTAPGLLKNVAEVRGGSYELTKTLKGSHLYDELQIDKVIKFLNKKEISRANGSLDKLKKDIIDCFKAEYRERHKDQCNKLADDIKNAKKDAGASLGERQKKLFRDFFGISEQSENDKPSFTNPLNLTCCLLPFDTVNNNRNRGEVLFNKLKEYAQKLDKNEGSLEMWEYIGIGNSGTAFSNFLGEGFLGRLRENKITELKKAMMDITDAWRGQEQEEELEKRLRILAALTIKLREPKFDNHWGGYRSDINGKLSSWLQNYINQTVKIKEDLKGHKKDLKKAKEMINRFGESDTKEEAVVSSLLESIEKIVPDDSADDEKPDIPAIAIYRRFLSDGRLTLNRFVQREDVQEALIKERLEAEKKKKPKKRKKKSDAEDEKETIDFKELFPHLAKPLKLVPNFYGDSKRELYKKYKNAAIYTDALWKAVEKIYKSAFSSSLKNSFFDTDFDKDFFIKRLQKIFSVYRRFNTDKWKPIVKNSFAPYCDIVSLAENEVLYKPKQSRSRKSAAIDKNRVRLPSTENIAKAGIALARELSVAGFDWKDLLKKEEHEEYIDLIELHKTALALLLAVTETQLDISALDFVENGTVKDFMKTRDGNLVLEGRFLEMFSQSIVFSELRGLAGLMSRKEFITRSAIQTMNGKQAELLYIPHEFQSAKITTPKEMSRAFLDLAPAEFATSLEPESLSEKSLLKLKQMRYYPHYFGYELTRTGQGIDGGVAENALRLEKSPVKKREIKCKQYKTLGRGQNKIVLYVRSSYYQTQFLEWFLHRPKNVQTDVAVSGSFLIDEKKVKTRWNYDALTVALEPVSGSERVFVSQPFTIFPEKSAEEEGQRYLGIDIGEYGIAYTALEITGDSAKILDQNFISDPQLKTLREEVKGLKLDQRRGTFAMPSTKIARIRESLVHSLRNRIHHLALKHKAKIVYELEVSRFEEGKQKIKKVYATLKKADVYSEIDADKNLQTTVWGKLAVASEISASYTSQFCGACKKLWRAEMQVDETITTQELIGTVRVIKGGTLIDAIKDFMRPPIFDENDTPFPKYRDFCDKHHISKKMRGNSCLFICPFCRANADADIQASQTIALLRYVKEEKKVEDYFERFRKLKNIKVLGQMKKI

SEQ ID NO: 758 - protein, 4 aa, synthetic construct
  REGION 1..4: note = Synthetic Polypeptide
SGGS

SEQ ID NO: 759 - protein, 4 aa, synthetic construct
  REGION 1..4: note = Synthetic Polypeptide
GGGS

SEQ ID NO: 760 - DNA, 91 bp, synthetic construct
  misc_feature 1..91: note = Synthetic Polynucleotide
  source 1..91: mol_type = other DNA
cctagggaagtgatcatagctgagtttctatctcatggtttatgctaaactatatgttgacatgttgaggagacttaagtccaaaacctgg

SEQ ID NO: 761 - protein, 30 aa, synthetic construct
  REGION 1..30: note = Synthetic Polypeptide
MDSLLMNRRKFLYQFKNVRWAKGRRETYLC

SEQ ID NO: 762 - protein, 1129 aa, Alicyclobacillus acidoterrestris
MAVKSIKVKLRLDDMPEIRAGLWKLHKEVNAGVRYYTEWLSLLRQENLYRRSPNGDGEQECDKTAEECKAELLERLRARQVENGHRGPAGSDDELLQLARQLYELLVPQAIGAKGDAQQIARKFLSPLADKDAVGGLGIAKAGNKPRWVRMREAGEPGWEEEKEKAETRKSADRTADVLRALADFGLKPLMRVYTDSEMSSVEWKPLRKGQAVRTWDRDMFQQAIERMMSWESWNQRVGQEYAKLVEQKNRFEQKNFVGQEHLVHLVNQLQQDMKEASPGLESKEQTAHYVTGRALRGSDKVFEKWGKLAPDAPFDLYDAEIKNVQRRNTRRFGSHDLFAKLAEPEYQALWREDASFLTRYAVYNSILRKLNHAKMFATFTLPDATAHPIWTRFDKLGGNLHQYTFLFNEFGERRHAIRFHKLLKVENGVAREVDDVTVPISMSEQLDNLLPRDPNEPIALYFRDYGAEQHFTGEFGGAKIQCRRDQLAHMHRRRGARDVYLNVSVRVQSQSEARGERRPPYAAVFRLVGDNHRAFVHFDKLSDYLAEHPDDGKLGSEGLLSGLRVMSVDLGLRTSASISVFRVARKDELKPNSKGRVPFFFPIKGNDNLVAVHERSQLLKLPGETESKDLRAIREERQRTLRQLRTQLAYLRLLVRCGSEDVGRRERSWAKLIEQPVDAANHMTPDWREAFENELQKLKSLHGICSDKEWMDAVYESVRRVWRHMGKQVRDWRKDVRSGERPKIRGYAKDVVGGNSIEQIEYLERQYKFLKSWSFFGKVSGQVIRAEKGSRFAITLREHIDHAKEDRLKKLADRIIMEALGYVYALDERGKGKWVAKYPPCQLILLEELSEYQFNNDRPPSENNQLMQWSHRGVFQELINQAQVHDLLVGTMYAAFSSRFDARTGAPGIRCRRVPARCTQEHNPEPFPWWLNKFVVEHTLDACPLRADDLIPTGEGEIFVSPFSAEEGDFHQIHADLNAAQNLQQRLWSDFDISQIRLRCDWGEVDGELVLIPRLTGKRTADSYSNKVFYTNTGVTYYERERGKKRRKVFAQEKLSEEEAELLVEADEAREKSVVLMRDPSGIINRGNWTRQKEFWSMVNQRIEGYLVKQIRSRVPLQDSACENTGDI

SEQ ID NO: 763 - protein, 5 aa, synthetic construct
  REGION 1..5: note = Synthetic Polypeptide
NGGNG

SEQ ID NO: 764 - protein, 1389 aa, Leptotrichia shahii
MGNLFGHKRWYEVRDKKDFKIKRKVKVKRNYDGNKYILNINENNNKEKIDNNKFIRKYINYKKNDNILKEFTRKFHAGNILFKLKGKEGIIRIENNDDFLETEEVVLYIEAYGKSEKLKALGITKKKIIDEAIRQGITKDDKKIEIKRQENEEEIEIDIRDEYTNKTLNDCSIILRIIENDELETKKSIYEIFKNINMSLYKIIEKIIENETEKVFENRYYEEHLREKLLKDDKIDVILTNFMEIREKIKSNLEILGFVKFYLNVGGDKKKSKNKKMLVEKILNINVDLTVEDIADFVIKELEFWNITKRIEKVKKVNNEFLEKRRNRTYIKSYVLLDKHEKFKIERENKKDKIVKFFVENIKNNSIKEKIEKILAEFKIDELIKKLEKELKKGNCDTEIFGIFKKHYKVNFDSKKFSKKSDEEKELYKIIYRYLKGRIEKILVNEQKVRLKKMEKIEIEKILNESILSEKILKRVKQYTLEHIMYLGKLRHNDIDMTTVNTDDFSRLHAKEELDLELITFFASTNMELNKIFSRENINNDENIDFFGGDREKNYVLDKKILNSKIKIIRDLDFIDNKNNITNNFIRKFTKIGTNERNRILHAISKERDLQGTQDDYNKVINIIQNLKISDEEVSKALNLDVVFKDKKNIITKINDIKISEENNNDIKYLPSFSKVLPEILNLYRNNPKNEPFDTIETEKIVLNALIYVNKELYKKLILEDDLEENESKNIFLQELKKTLGNIDEIDENIIENYYKNAQISASKGNNKAIKKYQKKVIECYIGYLRKNYEELFDFSDFKMNIQEIKKQIKDINDNKTYERITVKTSDKTIVINDDFEYIISIFALLNSNAVINKIRNRFFATSVWLNTSEYQNIIDILDEIMQLNTLRNECITENWNLNLEEFIQKMKEIEKDFDDFKIQTKKEIFNNYYEDIKNNILTEFKDDINGCDVLEKKLEKIVIFDDETKFEIDKKSNILQDEQRKLSNINKKDLKKKVDQYIKDKDQEIKSKILCRIIFNSDFLKKYKKEIDNLIEDMESENENKFQEIYYPKERKNELYIYKKNLFLNIGNPNFDKIYGLISNDIKMADAKFLFNIDGKNIRKNKISEIDAILKNLNDKLNGYSKEYKEKYIKKLKENDDFFAKNIQNKNYKSFEKDYNRVSEYKKIRDLVEFNYLNKIESYLIDINWKLAIQMARFERDMHYIVNGLRELGIIKLSGYNTGISRAYPKRNGSDGFYTTTAYYKFFDEESYKKFEKICYGFGIDLSENSEINKPENESIRNYISHFYIVRNPFADYSIAEQIDRVSNLLSYSTRYNNSTYASVFEVFKKDVNLDYDELKKKFKLIGNNDILERLMKPKKVSVLELESYNSDYIKNLIIELLTKIENTNDTL

SEQ ID NO: 765 - DNA, 5268 bp, synthetic construct
  misc_feature 1..5268: note = Synthetic Polynucleotide
  source 1..5268: mol_type = other DNA
atgtccaacctccttaccgtccaccagaatctccctgcccttccggtggatgccacctctgatgaagtgcgaaaaaacctgatggatatgtttcgcgataggcaagctttttctgaacacacgtggaagatgctcctgtcagtgtgtagaagctgggcagcttggtgcaagttgaacaaccgaaaatggtttcctgccgaacccgaagatgtgagagactacctcctctacctgcaggctcgagggctcgccgtgaaaacaatccaacaacacttgggtcagctcaacatgctgcacaggagatctgggctgccccggccgagtgactctaatgccgttagtctcgtaatgcggcgcattcgcaaagagaatgtggatgctggagaacgggcgaaacaggcactggcttttgaacggaccgacttcgatcaggtgcggagtcttatggagaatagtgacagatgccaggacattcggaaccttgcattcctgggtatcgcgtataataccctgctgagaatcgctgagatcgccagaatcagggtaaaggatatttctcgaacggacgggggacggatgttgattcatatcggtcgcactaaaacacttgtgagtaccgccggggtagagaaagccctgagccttggagttactaaactggtggagcggtggattagcgtgtccggcgtggcggatgacccaaacaattacttgttttgtagggtgcggaaaaatggtgtagccgctccatccgctacctcacagttgagtacacgcgcgttggaggggattttcgaagccacacatcgcttgatctacggcgccaaggacgattcaggccagcgatatcttgcctggagcgggcatagtgcccgggtgggtgccgcccgagacatggcaagggctggcgtgtcaattcctgaaatcatgcaggccggcgggtggaccaacgtgaacattgtgatgaactatatccggaacctggatagcgagaccggagcaatggtcagactgcttgaggatggcgacggtggatccggagggtccggaggtagtggcggcagcggtggttcaggtggcagcggagggtcaggaggctctgataaaaagtattctattggtttagctatcggcactaattccgttggatgggctgtcataaccgatgaatacaaagtaccttcaaagaaatttaaggtgttggggaacacagaccgtcattcgattaaaaagaatcttatcggtgccctcctattcgatagtggcgaaacggcagaggcgactcgcctgaaacgaaccgctcggagaaggtatacacgtcgcaagaaccgaatatgttacttacaagaaatttttagcaatgagatggccaaagttgacgattctttctttcaccgtttggaagagtccttccttgtcgaagaggacaagaaacatgaacggcaccccatctttggaaacatagtagatgaggtggcatatcatgaaaagtacccaacgatttatcacctcagaaaaaagctagttgactcaactgataaagcggacctgaggttaatctacttggctcttgcccatatgataaagttccgtgggcactttctcattgagggtgatctaaatccggacaactcggatgtcgacaaactgttcatccagttagtacaaacctataatcagttgtttgaagagaaccctataaatgcaagtggcgtggatgcgaaggctattcttagcgcccgcctctctaaatcccgacggctagaaaacctgatcgcacaattacccggagagaagaaaaatgggttgttcggtaaccttatagcgctctcactaggcctgacaccaaattttaagtcgaacttcgacttagctgaagatgccaaattgcagcttagtaaggacacgtacgatgacgatctcgacaatctactggcacaaattggagatcagtatgcggacttatttttggctgccaaaaaccttagcgatgcaatcctcctatctgacatactgagagttaatactgagattaccaaggcgccgttatccgcttcaatgatcaaaaggtacgatgaacatcaccaagacttgacacttctcaaggccctagtccgtcagcaactgcctgagaaatataaggaaatattctttgatcagtcgaaaaacgggtacgcaggttatattgacggcggagcgagtcaagaggaattctacaagtttatcaaacccatattagagaagatggatgggacggaagagttgcttgtaaaactcaatcgcgaagatctactgcgaaagcagcggactttcgacaacggtagcattccacatcaaatccacttaggcgaattgcatgctatacttagaaggcaggaggatttttatccgttcctcaaagacaatcgtgaaaagattgagaaaatcctaacctttcgcataccttactatgtgggacccctggcccgagggaactctcggttcgcatggatgacaagaaagtccgaagaaacgattactccatggaattttgaggaagttgtcgataaaggtgcgtcagctcaatcgttcatcgagaggatgaccaactttgacaagaatttaccgaacgaaaaagtattgcctaagcacagtttactttacgagtatttcacagtgtacaatgaactcacgaaagttaagtatgtcactgagggcatgcgtaaacccgcctttctaagcggagaacagaagaaagcaatagtagatctgttattcaagaccaaccgcaaagtgacagttaagcaattgaaagaggactactttaagaaaattgaatgcttcgattctgtcgagatctccggggtagaagatcgatttaatgcgtcacttggtacgtatcatgacctcctaaagataattaaagataaggacttcctggataacgaagagaatgaagatatcttagaagatatagtgttgactcttaccctctttgaagatcgggaaatgattgaggaaagactaaaaacatacgctcacctgttcgacgataaggttatgaaacagttaaagaggcgtcgctatacgggctggggacgattgtcgcggaaacttatcaacgggataagagacaagcaaagtggtaaaactattctcgattttctaaagagcgacggcttcgccaataggaactttatgcagctgatccatgatgactctttaaccttcaaagaggatatacaaaaggcacaggtttccggacaaggggactcattgcacgaacatattgcgaatcttgctggttcgccagccatcaaaaagggcatactccagacagtcaaagtagtggatgagctagttaaggtcatgggacgtcacaaaccggaaaacattgtaatcgagatggcacgcgaaaatcaaacgactcagaaggggcaaaaaaacagtcgagagcggatgaagagaatagaagagggtattaaagaactgggcagccagatcttaaaggagcatcctgtggaaaatacccaattgcagaacgagaaactttacctctattacctacaaaatggaagggacatgtatgttgatcaggaactggacataaaccgtttatctgattacgacgtcgatgccattgtaccccaatcctttttgaaggacgattcaatcgacaataaagtgcttacacgctcggataagaaccgagggaaaagtgacaatgttccaagcgaggaagtcgtaaagaaaatgaagaactattggcggcagctcctaaatgcgaaactgataacgcaaagaaagttcgataacttaactaaagctgagaggggtggcttgtctgaacttgacaaggccggatttattaaacgtcagctcgtggaaacccgccaaatcacaaagcatgttgcacagatactagattcccgaatgaatacgaaatacgacgagaacgataagctgattcgggaagtcaaagtaatcactttaaagtcaaaattggtgtcggacttcagaaaggattttcaattctataaagttagggagataaataactaccaccatgcgcacgacgcttatcttaatgccgtcgtagggaccgcactcattaagaaatacccgaagctagaaagtgagtttgtgtatggtgattacaaagtttatgacgtccgtaagatgatcgcgaaaagcgaacaggagataggcaaggctacagccaaatacttcttttattctaacattatgaatttctttaagacggaaatcactctggcaaacggagagatacgcaaacgacctttaattgaaaccaatggggagacaggtgaaatcgtatgggataagggccgggacttcgcgacggtgagaaaagttttgtccatgccccaagtcaacatagtaaagaaaactgaggtgcagaccggagggttttcaaaggaatcgattcttccaaaaaggaatagtgataagctcatcgctcgtaaaaaggactgggacccgaaaaagtacggtggcttcgatagccctacagttgcctattctgtcctagtagtggcaaaagttgagaagggaaaatccaagaaactgaagtcagtcaaagaattattggggataacgattatggagcgctcgtcttttgaaaagaaccccatcgacttccttgaggcgaaaggttacaaggaagtaaaaaaggatctcataattaaactaccaaagtatagtctgtttgagttagaaaatggccgaaaacggatgttggctagcgccggagagcttcaaaaggggaacgaactcgcactaccgtctaaatacgtgaatttcctgtatttagcgtcccattacgagaagttgaaaggttcacctgaagataacgaacagaagcaactttttgttgagcagcacaaacattatctcgacgaaatcatagagcaaatttcggaattcagtaagagagtcatcctagctgatgccaatctggacaaagtattaagcgcatacaacaagcacagggataaacccatacgtgagcaggcggaaaatattatccatttgtttactcttaccaacctcggcgctccagccgcattcaagtattttgacacaacgatagatcgcaaacgatacacttctaccaaggaggtgctagacgcgacactgattcaccaatccatcacgggattatatgaaactcggatagatttgtcacagcttgggggtgacggtggctccgattataaggatgatgacgacaagggaggttccccaaagaagaaaaggaaggtctga

SEQ ID NO: 766 - protein, 1755 aa, synthetic construct
  REGION 1..1755: note = Synthetic Polypeptide
MSNLLTVHQNLPALPVDATSDEVRKNLMDMFRDRQAFSEHTWKMLLSVCRSWAAWCKLNNRKWFPAEPEDVRDYLLYLQARGLAVKTIQQHLGQLNMLHRRSGLPRPSDSNAVSLVMRRIRKENVDAGERAKQALAFERTDFDQVRSLMENSDRCQDIRNLAFLGIAYNTLLRIAEIARIRVKDISRTDGGRMLIHIGRTKTLVSTAGVEKALSLGVTKLVERWISVSGVADDPNNYLFCRVRKNGVAAPSATSQLSTRALEGIFEATHRLIYGAKDDSGQRYLAWSGHSARVGAARDMARAGVSIPEIMQAGGWTNVNIVMNYIRNLDSETGAMVRLLEDGDGGSGGSGGSGGSGGSGGSGGSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGGSDYKDDDDKGGSPKKKRKV

SEQ ID NO: 767 - DNA, 95 bp, synthetic construct
  misc_feature 1..95: note = Synthetic Polynucleotide
  source 1..95: mol_type = other DNA
cctgaaataatgcaagtgtagaataactttttaaaatctcatggtttatgctaaactatatgttgacataagagtggtgataaggcaacagtagg

SEQ ID NO: 768 - DNA, 91 bp, synthetic construct; identical to SEQ ID NO: 760
  misc_feature 1..91: note = Synthetic Polynucleotide
  source 1..91: mol_type = other DNA

SEQ ID NO: 769 - DNA, 105 bp, synthetic construct
  misc_feature 1..105: note = Synthetic Polynucleotide
  source 1..105: mol_type = other DNA
atgtcctgaaataatgcaagtgtagaataactttttaaaatctcatggtttatgctaaactatatgttgacataagagtggtgataaggcaacagtaggtaaaag

SEQ ID NO: 770 - protein, 6 aa, synthetic construct
  REGION 1..6: note = Synthetic Polypeptide
NNASVE

SEQ ID NO: 771 - protein, 4 aa, synthetic construct
  REGION 1..4: note = Synthetic Polypeptide
NAAR

SEQ ID NO: 772 - protein, 4 aa, synthetic construct
  REGION 1..4: note = Synthetic Polypeptide
AGVF

SEQ ID NO: 773 - protein, 4 aa, synthetic construct
  REGION 1..4: note = Synthetic Polypeptide
GFLG

SEQ ID NO: 774 - protein, 4 aa, synthetic construct
  REGION 1..4: note = Synthetic Polypeptide
ALAL

SEQ ID NO: 775 - protein, 5 aa, synthetic construct
  REGION 1..5: note = Synthetic Polypeptide
ALALA

SEQ ID NO: 776 - protein, 14 aa, synthetic construct
  REGION 1..14: note = Synthetic polypeptide
LFKISWFMLNYMLT

SEQ ID NO: 777 - protein, 4 aa, synthetic construct
  REGION 1..4: note = Synthetic polypeptide
GNSR

(19) United States
(12) Patent Application Publication
(10) Pub. No.: US 2024/0209329 A1
(43) Pub. Date: Jun. 27, 2024

(54) PROGRAMMABLE CAS9-RECOMBINASE FUSION PROTEINS AND USES THEREOF

(71) Applicant: President and Fellows of Harvard College, Cambridge, MA (US)

(72) Inventors: David R. Liu, Cambridge, MA (US); Brian Chaikind, Cambridge, MA (US); Jeffrey L. Bessen, Cambridge, MA (US)

(73) Assignee: President and Fellows of Harvard College, Cambridge, MA (US)

(21) Appl. No.: 18/324,394

(22) Filed: May 26, 2023

Related U.S. Application Data
(62) Division of application No. 16/324,476, filed on Feb. 8, 2019, now Pat. No. 11,661,590.
(60) Provisional application No. 62/456,048, filed on Feb. 7, 2017.
(60) Provisional application No. 62/372,755, filed on Aug. 9, 2016.

(51) Int. Cl. C12N 9/12; C07K 4/00; C12N 9/22; C12N 15/113

(52) U.S. Cl. CPC C12N 9/1241; C07K 4/00; C12N 9/22; C12N 15/113; C07K 2319/09; C07K 2319/21; C07K 2319/43; C12N 2310/20; C12N 2320/11; C12Y 207/07

(57) ABSTRACT

Some aspects of this disclosure provide a fusion protein comprising a guide nucleotide sequence-programmable DNA binding protein domain (e.g., a nuclease-inactive variant of Cas9 such as dCas9), an optional linker, and a recombinase catalytic domain (e.g., a tyrosine recombinase catalytic domain or a serine recombinase catalytic domain such as a Gin recombinase catalytic domain). This fusion protein can recombine DNA sites containing a minimal recombinase core site flanked by guide RNA-specified sequences. The instant disclosure represents a step toward programmable, scarless genome editing in unmodified cells that is independent of endogenous cellular machinery or cell state.

### RELATED APPLICATIONS

This application is a national stage filing under 35 U.S.C. § 371 of international PCT application, PCT/US2017/046144, filed Aug. 9, 2017, which claims priority under 35 U.S.C. § 119(e) to U.S. provisional patent application, U.S. Ser. No. 62/372,755, filed Aug. 9, 2016, and U.S. provisional patent application, U.S. Ser. No. 62/456,048, filed Feb. 7, 2017, each of which is incorporated herein by reference.

### GOVERNMENT FUNDING

This invention was made with government support under EB022376 and GM118062 awarded by the National Institutes of Health (NIH). The government has certain rights in this invention.

### BACKGROUND OF THE INVENTION

Efficient, programmable, and site-specific homologous recombination remains a longstanding goal of genetics and genome editing. Early attempts at directing recombination to loci of interest relied on the transfection of donor DNA with long flanking sequences that are homologous to a target locus. This strategy was hampered by very low efficiency and thus the need for a stringent selection to identify integrants. More recent efforts have exploited the ability of double-stranded DNA breaks (DSBs) to induce homology-directed repair (HDR). Homing endonucleases and later programmable endonucleases such as zinc finger nucleases, TALE nucleases, Cas9, and fCas9 have been used to introduce targeted DSBs and induce HDR in the presence of donor DNA. In most post-mitotic cells, however, DSB-induced HDR is strongly down regulated and generally inefficient. Moreover, repair of DSBs by error-prone repair pathways such as non-homologous end-joining (NHEJ) or single-strand annealing (SSA) causes random insertions or deletions (indels) of nucleotides at the DSB site at a higher frequency than HDR. The efficiency of HDR can be increased if cells are subjected to conditions forcing cell-cycle synchronization or if the enzymes involved in NHEJ are inhibited. However, such conditions can cause many random and unpredictable events, limiting potential applications. The instant disclosure provides a fusion protein that can recombine DNA sites containing a minimal recombinase core site flanked by guide RNA-specified sequences and represents a step toward programmable, scarless genome editing in unmodified cells that is independent of endogenous cellular machinery or cell state.

### SUMMARY OF THE INVENTION

The instant disclosure describes the development of a fusion protein comprising a guide nucleotide sequence-programmable DNA binding protein domain, an optional linker, and a recombinase catalytic domain (e.g., a serine recombinase catalytic domain such as a Gin recombinase catalytic domain, a tyrosine recombinase catalytic domain, or any evolved recombinase catalytic domain). This fusion protein operates on a minimal gix core recombinase site (NNNNAAASSWWSSTTTNNNN, SEQ ID NO: 19) flanked by two guide RNA-specified DNA sequences. Recombination mediated by the described fusion protein is dependent on both guide RNAs, resulting in orthogonality among different guide nucleotide:fusion protein complexes, and functions efficiently in cultured human cells on DNA sequences matching those found in the human genome. The fusion protein of the disclosure can also operate directly on the genome of human cells (e.g., cultured human cells), catalyzing a deletion, insertion, inversion, translocation, or recombination between two recCas9 psuedosites located approximately 14 kilobases apart. This work provides engineered enzymes that can catalyze gene insertion, deletion, inversion, or chromosomal translocation with user-defined, single base-pair resolution in unmodified genomes.

In one aspect, the instant disclosure provides a fusion protein comprising: (i) a guide nucleotide sequence-programmable DNA binding protein domain; (ii) an optional linker; and (iii) a recombinase catalytic domain such as any serine recombinase catalytic domain (including but not limited to a Gin, Sin, Tn3, Hin, β, γδ, or PhiC31 recombinase catalytic domain), any tyrosine recombinase domain (including, but not limited to a Cre or FLP recombinase catalytic domain), or any evolved recombinase catalytic domain.

The guide nucleotide sequence-programmable DNA binding protein domain may be selected from the group consisting of nuclease inactive Cas9 (dCas9) domains, nuclease inactive Cpf1 domains, nuclease inactive Argonaute domains, and variants thereof. In certain embodiments, the guide nucleotide sequence-programmable DNA-binding protein domain is a nuclease inactive Cas9 (dCas9) domain. In certain embodiments, the amino acid sequence of the dCas9 domain comprises mutations corresponding to a D10A and/or H840A mutation in SEQ ID NO: 1. In another embodiment, the amino acid sequence of the dCas9 domain comprises a mutation corresponding to a D10A mutation in SEQ ID NO: 1 and a mutation corresponding to an H840A mutation in SEQ ID NO: 1. In another embodiment, the amino acid sequence of the dCas9 domain further does not include the N-terminal methionine shown in SEQ ID NO: 1. In a certain embodiment, the amino acid sequence of the dCas9 domain comprises SEQ ID NO: 712. In one embodiment, the amino acid sequence of the dCas9 domain has a greater than 95% sequence identity with SEQ ID NO: 712. In one embodiment, the amino acid sequence of the dCas9 domain has a greater than 96, 97, 98, 99% or greater sequence identity with SEQ ID NO: 712. In some embodiments, the recombinase catalytic domain is a serine recombinase catalytic domain or a tyrosine recombinase catalytic domain.

In one embodiment, the amino acid sequence of the recombinase catalytic domain is a Gin recombinase catalytic domain. In some embodiments, the Gin recombinase catalytic domain comprises a mutation corresponding to one or more of the mutations selected from: a H106Y, I127L, I136R and/or G137F mutation in SEQ ID NO: 713. In an embodiment, the amino acid sequence of the Gin recombinase catalytic domain comprises mutations corresponding to two or more of the mutations selected from: a I127L, I136R and/or G137F mutation in SEQ ID NO: 713. In an embodiment, the amino acid sequence of the Gin recombinase catalytic domain comprises mutations corresponding to a I127L, I136R and G137F mutation in SEQ ID NO: 713. In another embodiment, the amino acid sequence of the Gin recombinase has been further mutated. In a specific embodiment, the amino acid sequence of the Gin recombinase catalytic domain comprises SEQ ID NO: 713.

In another embodiment, the amino acid sequence of the recombinase catalytic domain is a Hin recombinase, β recombinase, Sin recombinase, Tn3 recombinase, γδ recombinase, Cre recombinase; FLP recombinase; or a phiC31 recombinase catalytic domain.

In one embodiment, the amino acid sequence of the Cre recombinase is truncated. In another embodiment, the tyrosine recombinase catalytic domain is the 25 kDa carboxy-terminal domain of the Cre recombinase. In another embodiment, the Cre recombinase begins with amino acid R118, A127, E138, or R154 (preceded in each case by methionine). In one embodiment, the amino acid sequence of the recombinase has been further mutated. In certain embodiments, the recombinase catalytic domain is an evolved recombinase catalytic domain. In some embodiments, the amino acid sequence of the recombinase has been further mutated.

In some embodiments, the linker (e.g., the first, second, or third linker) may have a length of about 0 angstroms to about 81 angstroms. The linker typically has a length of about 33 angstroms to about 81 angstroms. The linker may be peptidic, non-peptidic, or a combination of both types of linkers. In certain embodiments, the linker is a peptide linker. In certain embodiments, the peptide linker comprises an XTEN linker SGSETPGTSESATPES (SEQ ID NO: 7), SGSETPGTSESA (SEQ ID NO: 8), or SGSETPGTSESATPEGGSGGS (SEQ ID NO: 9), an amino acid sequence comprising one or more repeats of the tri-peptide GGS, or any of the following amino acid sequences: VPFLLEPDNINGKTC (SEQ ID NO: 10), GSAGSAAGSGEF (SEQ ID NO: 11), SIVAQLSRPDPA (SEQ ID NO: 12), MKIIEQLPSA (SEQ ID NO: 13), VRHKLKRVGS (SEQ ID NO: 14), GHGTGSTGSGSS (SEQ ID NO: 15), MSRPDPA (SEQ ID NO: 16), or GGSM (SEQ ID NO: 17). In another embodiment, the peptide linker comprises one or more repeats of the tri-peptide GGS. In one embodiment, the peptide linker comprises from one to five repeats of the tri-peptide GGS. In another embodiment, the peptide linker comprises from six to ten repeats of the tri-peptide GGS. In a specific embodiment, the peptide linker comprises eight repeats of the tri-peptide GGS. In another embodiment, the peptide linker is from 18 to 27 amino acids long. In certain embodiments, the peptide linker is 24 amino acids long. In certain embodiments, the peptide linker has the amino acid sequence

(SEQ ID NO: 183)
GGSGGSGGSGGSGGSGGSGGSGGS.

In certain embodiments, the linker is a non-peptide linker. In certain embodiments, the non-peptide linker comprises polyethylene glycol (PEG), polypropylene glycol (PPG), co-poly(ethylene/propylene) glycol, polyoxyethylene (POE), polyurethane, polyphosphazene, polysaccharides, dextran, polyvinyl alcohol, polyvinylpyrrolidones, polyvinyl ethyl ether, polyacryl amide, polyacrylate, polycyanoacrylates, lipid polymers, chitins, hyaluronic acid, heparin, or an alkyl linker. In certain embodiments, the alkyl linker has the formula: —NH—(CH₂)ₛ—C(O)—, wherein s is any integer between 1 and 100, inclusive. In certain embodiments, s is any integer from 1-20, inclusive.

In another embodiment, the fusion protein further comprises a nuclear localization signal (NLS) domain. In certain embodiments, the NLS domain is bound to the guide nucleotide sequence-programmable DNA binding protein domain or the recombinase catalytic domain via one or more second linkers.

In one embodiment, the fusion protein comprises the structure NH₂-[recombinase catalytic domain]-[optional linker sequence]-[guide nucleotide sequence-programmable DNA binding protein domain]-[optional, second linker sequence]-[NLS domain]-COOH. In certain embodiments, the fusion protein has greater than 85%, 90%, 95%, 98%, or 99% sequence identity with the amino acid sequence shown in SEQ ID NO: 719. In a specific embodiment, the fusion protein comprises the amino acid sequence shown in SEQ ID NO: 719. In one embodiment, the fusion protein consists of the amino acid sequence shown in SEQ ID NO: 719.

In another embodiment, the fusion protein further comprises one or more affinity tags. In one embodiment, the affinity tag is selected from the group consisting of a FLAG tag, a polyhistidine (poly-His) tag, a polyarginine (poly-Arg) tag, a Myc tag, and an HA tag. In an embodiment, the affinity tag is a FLAG tag. In a specific embodiment, the FLAG tag has the sequence PKKKRKV (SEQ ID NO: 702). In another embodiment, the one or more affinity tags are bound to the guide nucleotide sequence-programmable DNA binding protein domain, the recombinase catalytic domain, or the NLS domain via one or more third linkers. In certain embodiments, the third linker is a peptide linker.

The elements of the fusion protein described herein may be in any order, without limitation. In some embodiments, the fusion protein has the structure NH₂-[recombinase catalytic domain]-[linker sequence]-[guide nucleotide sequence-programmable DNA binding protein domain]-[optional linker sequence]-[NLS domain]-[optional linker sequence]-[optional affinity tag]-COOH, NH₂-[guide nucleotide sequence-programmable DNA binding protein domain]-[linker sequence]-[recombinase catalytic domain]-[optional linker sequence]-[NLS domain]-[optional linker sequence]-[optional affinity tag]-COOH, or NH₂-[N-terminal portion of a bifurcated or circularly permuted guide nucleotide sequence-programmable DNA binding protein domain]-[optional linker sequence]-[recombinase catalytic domain]-[optional linker sequence]-[C-terminal portion of a bifurcated or circularly permuted guide nucleotide sequence-programmable DNA binding protein domain]-[optional linker sequence]-[NLS domain]-[optional linker sequence]-[optional affinity tag]-COOH.

In some embodiments, the fusion protein has the structure NH₂-[optional affinity tag]-[optional linker sequence]-[recombinase catalytic domain]-[linker sequence]-[guide nucleotide sequence-programmable DNA binding protein domain]-[optional linker sequence]-[NLS domain]-COOH, NH₂-[optional affinity tag]-[optional linker sequence]-[guide nucleotide sequence-programmable DNA binding protein domain]-[linker sequence]-[recombinase catalytic domain]-[optional linker sequence]-[NLS domain]-COOH, or NH₂-[optional affinity tag]-[optional linker sequence]-[N-terminal portion of a bifurcated or circularly permuted guide nucleotide sequence-programmable DNA binding protein domain]-[optional linker sequence]-[recombinase catalytic domain]-[optional linker sequence]-[C-terminal portion of a bifurcated or circularly permuted guide nucleotide sequence-programmable DNA binding protein domain]-[optional linker sequence]-[NLS domain]-COOH.

In a certain embodiment, the fusion protein has greater than 85%, 90%, 95%, 98%, or 99% sequence identity with the amino acid sequence shown in SEQ ID NO: 185. In a specific embodiment, the fusion protein has the amino acid sequence shown in SEQ ID NO: 185. In certain embodiments, the recombinase catalytic domain of the fusion protein has greater than 85%, 90%, 95%, 98%, or 99% sequence identity with the amino acid sequence shown in amino acids 1-142 of SEQ ID NO: 185, which is identical to the sequence shown in SEQ ID NO: 713. In certain embodiments, the dCas9 domain has greater than 90%, 95%, or 99% sequence identity with the amino acid sequence shown in amino acids 167-1533 of SEQ ID NO: 185, which is identical to the sequence shown in SEQ ID NO: 712. In certain embodiments, the fusion protein of the instant disclosure has greater than 90%, 95%, or 99% sequence identity with the amino acid sequence shown in amino acids 1-1544 of SEQ ID NO: 185, which is identical to the sequence shown in SEQ ID NO: 719. In one embodiment, the fusion protein is bound to a guide RNA (gRNA).

In one aspect, the instant disclosure provides a dimer of the fusion protein described herein. In certain embodiments, the dimer is bound to a target DNA molecule. In certain embodiments, each fusion protein of the dimer is bound to the same strand of the target DNA molecule. In certain embodiments, each fusion protein of the dimer is bound to an opposite strand of the target DNA molecule. In certain embodiments, the gRNAs of the dimer hybridize to gRNA binding sites flanking a recombinase site of the target DNA molecule. In certain embodiments, the recombinase site comprises a res, gix, hix, six, resH, LoxP, FTR, or att core, or related core sequence. In certain embodiments, the recombinase site comprises a gix core or gix-related core sequence. In further embodiments, the distance between the gix core or gix-related core sequence and at least one gRNA binding site is from 3 to 7 base pairs. In certain embodiments, the distance between the gix core or gix-related core sequence and at least one gRNA binding site is from 5 to 6 base pairs.

In certain embodiments, a first dimer binds to a second dimer thereby forming a tetramer of the fusion protein. In one aspect, the instant disclosure provides a tetramer of the fusion protein described herein. In certain embodiments, the tetramer is bound to a target DNA molecule. In certain embodiments, each dimer is bound to an opposite strand of DNA. In other embodiments, each dimer is bound to the same strand of DNA.

In another aspect, the instant disclosure provides methods for site-specific recombination between two DNA molecules, comprising: (a) contacting a first DNA with a first fusion protein, wherein the guide nucleotide sequence-programmable DNA binding protein domain binds a first gRNA that hybridizes to a first region of the first DNA; (b) contacting the first DNA with a second fusion protein, wherein the guide nucleotide sequence-programmable DNA binding protein domain of the second fusion protein binds a second gRNA that hybridizes to a second region of the first DNA; (c) contacting a second DNA with a third fusion protein, wherein the guide nucleotide sequence-programmable DNA binding protein domain of the third fusion protein binds a third gRNA that hybridizes to a first region of the second DNA; and (d) contacting the second DNA with a fourth fusion protein, wherein the guide nucleotide sequence-programmable DNA binding protein domain of the fourth fusion protein binds a fourth gRNA that hybridizes to a second region of the second DNA; wherein the binding of the fusion proteins in steps (a)-(d) results in the tetramerization of the recombinase catalytic domains of the fusion proteins, under conditions such that the DNAs are recombined, and wherein the first, second, third, and/or fourth fusion protein is any of the fusion proteins described herein.

In one embodiment, the first and second DNA molecules have different sequences. In another embodiment, the gRNAs of steps (a) and (b) hybridize to opposing strands of the first DNA, and the gRNAs of steps (c) and (d) hybridize to opposing strands of the second DNA. In another embodiment, wherein the gRNAs of steps (a) and (b); and/or the gRNAs of steps (c) and (d) hybridize to regions of their respective DNAs that are no more than 10, no more than 15, no more than 20, no more than 25, no more than 30, no more than 40, no more than 50, no more than 60, no more than 70, no more than 80, no more than 90, or no more than 100 base pairs apart. In certain embodiments, the gRNAs of steps (a) and (b), and/or the gRNAs of steps (c) and (d) hybridize to regions of their respective DNAs at gRNA binding sites that flank a recombinase site (see, for example, FIG. 1D). In certain embodiments, the recombinase site comprises a res, gix, hix, six, resH, LoxP, FTR, or att core, or related core sequence. In certain embodiments, the recombinase site comprises a gix core or gix-related core sequence. In certain embodiments, the distance between the gix core or gix-related core sequence and at least one gRNA binding site is from 3 to 7 base pairs. In certain embodiments, the distance between the gix core or gix-related core sequence and at least one gRNA binding site is from 5 to 6 base pairs.

The method for site-specific recombination provided herein may also be used with a single DNA molecule. In one aspect, the instant disclosure provides a method for site-specific recombination between two regions of a single DNA molecule, comprising: (a) contacting the DNA with a first fusion protein, wherein the guide nucleotide sequence-programmable DNA binding protein domain binds a first gRNA that hybridizes to a first region of the DNA; (b) contacting the DNA with a second fusion protein, wherein the guide nucleotide sequence-programmable DNA binding protein domain of the second fusion protein binds a second gRNA that hybridizes to a second region of the DNA; (c) contacting the DNA with a third fusion protein, wherein the guide nucleotide sequence-programmable DNA binding protein domain of the third fusion protein binds a third gRNA that hybridizes to a third region of the DNA; and (d) contacting the DNA with a fourth fusion protein, wherein the guide nucleotide sequence-programmable DNA binding protein domain of the fourth fusion protein binds a fourth gRNA that hybridizes to a fourth region of the DNA; wherein the binding of the fusion proteins in steps (a)-(d) results in the tetramerization of the recombinase catalytic domains of the fusion proteins, under conditions such that the DNA is recombined, and wherein the first, second, third, and/or fourth fusion protein is any of the fusion proteins described.

In certain embodiments, the two regions of the single DNA molecule that are recombined have different sequences. In another embodiment, the recombination results in the deletion of a region of the DNA molecule. In a specific embodiment, the region of the DNA molecule that is deleted is prone to cross-over events in meiosis. In one embodiment, the first and second gRNAs of steps (a)-(d) hybridize to the same strand of the DNA, and the third and fourth gRNAs of steps (a)-(d) hybridize to the opposing strand of the DNA. In another embodiment, the gRNAs of steps (a) and (b) hybridize to regions of the DNA that are no more than 50, no more than 60, no more than 70, no more than 80, no more than 90, or no more than 100 base pairs apart, and the gRNAs of steps (c) and (d) hybridize to regions of the DNA that are no more than 10, no more than 15, no more than 20, no more than 25, no more than 30, no more than 40, no more than 50, no more than 60, no more than 70, no more than 80, no more than 90, or no more than 100 base pairs apart. In certain embodiments, the gRNAs of steps (a) and (b); and/or the gRNAs of steps (c) and (d) hybridize to gRNA binding sites flanking a recombinase site. In certain embodiments, the recombinase site comprises a res, gix, hix, six, resH, LoxP, FTR, or att core or related core sequence. In one embodiment, the recombinase site comprises a gix core or gix-related core sequence. In certain embodiments, the distance between the gix core or gix-related core sequence and at least one gRNA binding site is from 3 to 7 base pairs. In certain embodiments, the distance between the gix core or gix-related core sequence and at least one gRNA binding site is from 5 to 6 base pairs.

The DNA described herein may be in a cell. In certain embodiments, the cell is a eukaryotic cell. In certain embodiments, the cell is a plant cell. In certain embodiments, the cell is a prokaryotic cell. In some embodiments, the cell may be a mammalian cell. In some embodiments, the cell may be a human cell. In certain embodiments, the cell is in a subject. In some embodiments, the subject may be a mammal. In certain embodiments, the subject is a human. In certain embodiments, the cell may be a plant cell.

In one aspect, the instant disclosure provides a polynucleotide encoding any of the fusion proteins disclosed herein. In certain embodiments, the instant disclosure provides a vector comprising the polynucleotide encoding any of the fusion proteins disclosed herein.

In another aspect, the instant disclosure provides a cell comprising a genetic construct for expressing any fusion protein disclosed herein.

In one aspect, the instant disclosure provides a kit comprising any fusion protein disclosed herein. In another aspect, the instant disclosure provides a kit comprising a polynucleotide encoding any fusion protein disclosed herein. In another aspect, the instant disclosure provides a kit comprising a vector for recombinant protein expression, wherein the vector comprises a polynucleotide encoding any fusion protein disclosed herein. In another aspect, the instant disclosure provides a kit comprising a cell that comprises a genetic construct for expressing any fusion protein disclosed herein. In one embodiment, the kit further comprises one or more gRNAs and/or vectors for expressing one or more gRNAs.

The details of certain embodiments of the invention are set forth in the Detailed Description of Certain Embodiments, as described below. Other features, objects, and advantages of the invention will be apparent from the Definitions, Examples, Figures, and Claims.

### BRIEF DESCRIPTION OF THE DRAWINGS

FIGS. 1A-1D. Overview of the experimental setup. Cells are transfected with (FIG. 1A) guide RNA expression vector(s) under the control of an hU6 promoter, (FIG. 1B) a recCas9 expression vector under the control of a CMV promoter, and (FIG. 1C) a recCas9 reporter plasmid. Co-transfection of these components results in reassembly of guide RNA-programmed recCas9 at the target sites (FIG. 1D). This will mediate deletion of the polyA terminator, allowing transcription of GFP. Guide RNA expression vectors and guide RNA sequences are abbreviated as gRNA.

FIGS. 2A-2F. Optimization of fusion linker lengths and target site spacer variants. A single target guide RNA expression vector, pHU6-NT1, or non-target vector pHU6-BC74 was used in these experiments. The sequences can be found in Tables 6-9. (FIG. 2A) A portion of the target site is shown with guide RNA target sites in black with dashed underline and a gix core sequence site in black. The 5′ and 3′ sequences on either side of the pseudo-gix sites are identical, but inverted, and are recognized by pHU6-NT1. The number of base pairs spacers separating the gix pseudo-site from the 5′ and 3′ binding sites is represented by an X and Y, respectively. This figure depicts SEQ ID NOs: 700 and 703, respectively. (FIG. 2B) Z represents the number of GGS repeats connecting Ginβ to dCas9. recCas9 activity is assessed when X=Y for (FIG. 2C) (GGS)₂ (SEQ ID NO: 182), (FIG. 2D) (GGS)₅ (SEQ ID NO: 701), and (FIG. 2E) (GGS)₈ (SEQ ID NO: 183) linkers connecting the Gin catalytic domain to the dCas9 domain. (FIG. 2F) The activity of recCas9 on target sites composed of uneven base pair spacers (X≠Y) was determined; X=Y=6 is included for comparison. All experiments are performed in triplicate and background fluorescence is subtracted from these experiments. The percentage of eGFP-positive cells is of only those transfected (i.e., expressing a constitutively expressed iRFP gene) and at least 6,000 live events are recorded for each experiment. Guide RNA expression vectors and guide RNA sequences are abbreviated as “gRNA”. Values and error bars represent the mean and standard deviation, respectively, of three independent biological replicates.

FIGS. 3A-3B. The dependence of forward and reverse guide RNAs on recCas9 activity. (FIG. 3A) A sequence found within PCDH15 replaces the target site tested in FIGS. 1A-1D. Two offset sequences can be targeted by guide RNAs on both the 5′ and 3′ sides of a pseudo-gix core site. This figure depicts SEQ ID NOs: 704-705, respectively. (FIG. 3B) recCas9 activity was measured by co-transfecting a recCas9 expression vector and reporter plasmid with all four guide RNA expression vector pairs and individual guide RNA vectors with off target (O.T.) guide RNA vectors. The off-target forward and reverse contained guide RNA sequences targeting CLTA and VEGF, respectively. Control experiments transfected with the reporter plasmid but without a target guide RNA are also shown. The results of reporter plasmid cotransfected with different guide RNA expression vectors, but without recCas9 expression vectors, are also shown. All experiments were performed in quadruplicate, and background fluorescence is not subtracted from these experiments. The percentage of eGFP-positive cells is of only those transfected (i.e., expressing a constitutively expressed iRFP gene), and at least 6,000 live events are recorded for each experiment. Guide RNA expression vectors and guide RNA sequences are abbreviated as gRNA. Values and error bars represent the mean and standard deviation, respectively, of four independent biological replicates.

FIGS. 4A-4D. recCas9 can target multiple sequences identical to those in the human genome. (FIG. 4A) The target sites shown in FIGS. 1A-1D are replaced by sequences found within the human genome. See Table 6 for sequences. A recCas9 expression vector was cotransformed with all combinations of guide RNA vectors pairs and reporter plasmids. Off-target guide RNA vectors were also cotransformed with the recCas9 expression vector and reporter plasmids and contain guide RNA sequences targeting CLTA and VEGF (see, e.g., Guilinger et al., Fusion of catalytically inactive Cas9 to FokI nuclease improves the specificity of genome modification. Nature biotechnology, (2014), the entire contents of which is hereby incorporated by reference). The percentage of eGFP-positive cells reflects that of transfected (iRFP-positive) cells. At least 6,000 live events are recorded for each experiment. Values and error bars represent the mean and standard deviation, respectively, of at least three independent biological replicates. (FIG. 4B) Transfection experiments were performed again, replacing the resistance marker in the recCas9 expression vector and pUC with SpecR. After cotransfection and incubation, episomal DNA was extracted, transformed into E. coli and selected for carbenicillin resistance. Colonies were then sequenced to determine (FIG. 4C) the ratio of recombined to fully intact plasmids. (FIG. 4D) Sequencing data from episomal extractions isolated from transfected cells. Columns and rows represent the transfection conditions. Each cell shows the percent of recombined plasmid and the ratio. The values shown reflect the mean and standard deviation of two independent biological replicates. The average difference between the mean and each replicate is shown as the error. Guide RNA expression vectors and guide RNA sequences are abbreviated as gRNA.

FIGS. 5A-5D. recCas9 mediates guide RNA- and recCas9-dependent deletion of genomic DNA in cultured human cells. (FIG. 5A) Schematic showing predicted recCas9 target sites located within an intronic region of the FAM19A2 locus of chromosome 12 and the positions of primers used for nested PCR. This figure depicts SEQ ID NOs: 706-709 from top to bottom and left to right, respectively. (FIG. 5B) Representative results of nested genomic PCR of template from cells transfected with the indicated expression vectors (n=3 biological replicates; NTC=no template control). The asterisk indicates the position of the 1.3-kb predicted primary PCR product. Arrow indicates the predicted deletion product after the secondary PCR. Both panes are from the same gel but were cut to remove blank lanes. (FIG. 5C) Sanger sequencing of PCR products resulting from nested genomic PCR of cells transfected with all four gRNA expression vectors, and the recCas9 expression vector matches the predicted post-recombination product. This figure depicts SEQ ID NOs: 710 and 711 from top to bottom, respectively. (FIG. 5D) Estimated minimum deletion efficiency of FAM19A2 locus determined by limiting-dilution nested PCR. The values shown reflect the mean and standard deviation of three replicates.

FIG. 6. Reporter plasmid construction. Golden Gate assembly was used to construct the reporter plasmids described in this work. All assemblies started with a common plasmid, pCALNL-EGFP-Esp3I, that was derived from pCALNL-EGFP and contained to Esp3I restriction sites. The fragments shown are flanked by Esp3I sites. Esp3I digestion creates a series of compatible, unique 4-base pair 5′ overhangs so that assembly occurs in the order shown. To assemble the target sites, Esp3I (ThermoFisher Scientific, Waltham, MA) and five fragments were added to a single reaction tube to allow for iterative cycles of Esp3I digestion and T7 ligation. Reactions were then digested with Plasmid-Safe-ATP-dependent DNAse (Epicentre, Madison, WI) to reduce background. Colonies were analyzed by colony PCR to identify PCR products that matched the expected full length 5 part assembly product; plasmid from these colonies was then sent for sanger sequencing. For the genomic reporters shown in FIG. 4, fragments 1 and 2 as well as fragments 4 and 5 were combined into two gBlocks (IDT, Coralville, IA) fragments encoding the entire target site (not shown in the figure). Assembly was then completed as described above. Details for construction can be found in the methods for the supporting material. Oligonucleotides and gBLOCKS for creation of fragments can be found in Table 2.

FIGS. 7A and 7B. A Cre recombinase evolved to target a site in the Rosa locus of the human genome called “36C6” was fused to dCas9. This fusion was then used to recombine a plasmid-based reporter containing the Rosa target site in a guide-RNA dependent fashion. FIG. 7A demonstrates the results of linker optimization using wild-type Cre and 36C6. A GinB construct, targeting its cognate reporter, is shown for reference. The 1× 2×, 5×, and 8× linkers shown are the number of GGS repeats in the linker. FIG. 7B shows the results of a reversion analysis which demonstrated that making mutations to 36C6 fused to dCas9 could impact the relative guide dependence of the chimeric fusion. A GinB construct, targeting its cognate reporter, is shown for reference. GGS-36C6: 1× GGS linker; 2GGS-36C6 (using linker SEQ ID NO: 181): 2× GGS linker (using linker SEQ ID NO: 181).

FIG. 8. PAMs were identified flanking the Rosa26 site in the human genome that could support dCas9 binding (see at top). Guide RNAs and a plasmid reporter were designed to test whether the endogenous protospacers could support dCas9-36C6 activity. A GinB construct, targeting the gix reporter, is shown for reference. Mix: equal parts mixture of all 5 linker variants between Cas9 and 36C6. The sequences correspond to SEQ ID NO: 769 (the nucleotide sequence) and 770, 776, and 777 (the amino acid sequences from left to right).

FIGS. 9A-9B. Locations of various tested truncations of Cre recombinase are shown in FIG. 9A. Truncated variants of Cre recombinase fused to dCas9 show both appreciable recombinase activity as well as a strict reliance on the presence of guide RNA in a Lox plasmid reporter system (FIG. 9B). Wild type Cre fused to dCas9 is shown as a positive control.

### DEFINITIONS

As used herein, the singular forms “a,” “an,” and “the” include the singular and the plural reference unless the context clearly indicates otherwise. Thus, for example, a reference to “an agent” includes a single agent and a plurality of such agents.

Non-limiting, exemplary RNA-programmable DNA-binding proteins include Cas9 nucleases, Cas9 nickases, nuclease inactive Cas9 (dCas9), CasX, CasY, Cpf1, C2c1, C2c2, C2C3, and Argonaute. The term “Cas9” or “Cas9 nuclease” refers to an RNA-guided nuclease comprising a Cas9 protein, or a fragment thereof (e.g., a protein comprising an active or inactive DNA cleavage domain of Cas9, and/or the gRNA binding domain of Cas9). Cas9 has two cleavage domains, which cut specific DNA strands (e.g., sense and antisense strands). Cas9 nickases can be generated that cut either strand (including, but not limited to D10A and H840A of spCas9). A Cas9 domain (e.g., nuclease active Cas9, nuclease inactive Cas9, or Cas9 nickases) may be used without limitation in the fusion proteins and methods described herein. Further, any of the guide nucleotide sequence-programmable DNA binding proteins described herein may be useful as nickases.

A Cas9 nuclease is also referred to sometimes as a casn1 nuclease or a CRISPR (clustered regularly interspaced short palindromic repeat)-associated nuclease. CRISPR is an adaptive immune system that provides protection against mobile genetic elements (viruses, transposable elements, and conjugative plasmids). CRISPR clusters contain spacers, sequences complementary to antecedent mobile elements, and target invading nucleic acids. CRISPR clusters are transcribed and processed into CRISPR RNA (crRNA). In type II CRISPR systems correct processing of pre-crRNA requires a trans-encoded small RNA (tracrRNA), endogenous ribonuclease 3 (rnc), and a Cas9 protein. The tracrRNA serves as a guide for ribonuclease 3-aided processing of pre-crRNA. Subsequently, Cas9/crRNA/tracrRNA endonucleolytically cleaves a linear or circular dsDNA target complementary to the spacer. The target strand not complementary to crRNA is first cut endonucleolytically, then trimmed 3′-5′ exonucleolytically. In nature, DNA-binding and cleavage typically requires protein and both RNA sequences. However, single guide RNAs (“sgRNA”, or simply “gRNA”) can be engineered so as to incorporate aspects of both the crRNA and tracrRNA into a single RNA species. See, e.g., Jinek M., Chylinski K., Fonfara I., Hauer M., Doudna J. A., Charpentier E. Science 337:816-821(2012), the entire contents of which is hereby incorporated by reference. Cas9 recognizes a short motif in the CRISPR repeat sequences (the PAM or protospacer adjacent motif) to help distinguish self versus non-self. Cas9 nuclease sequences and structures are well known to those of skill in the art (see, e.g., “Complete genome sequence of an M1 strain of Streptococcus pyogenes.” Ferretti et al., J. J., McShan W. M., Ajdic D. J., Savic D. J., Savic G., Lyon K., Primeaux C., Sezate S., Suvorov A. N., Kenton S., Lai H. S., Lin S. P., Qian Y., Jia H. G., Najar F. Z., Ren Q., Zhu H., Song L., White J., Yuan X., Clifton S. W., Roe B. A., McLaughlin R. E., Proc. Natl. Acad. Sci. U.S.A. 98:4658-4663(2001); “CRISPR RNA maturation by trans-encoded small RNA and host factor RNase III.” Deltcheva E., Chylinski K., Sharma C. M., Gonzales K., Chao Y., Pirzada Z. A., Eckert M. R., Vogel J., Charpentier E., Nature 471:602-607(2011); and “A programmable dual-RNA-guided DNA endonuclease in adaptive bacterial immunity.” Jinek M., Chylinski K., Fonfara I., Hauer M., Doudna J. A., Charpentier E. Science 337:816-821(2012), the entire contents of each of which are incorporated herein by reference). Cas9 orthologs have been described in various species, including, but not limited to, S. pyogenes and S. thermophilus. Additional suitable Cas9 nucleases and sequences will be apparent to those of skill in the art based on this disclosure, and such Cas9 nucleases and sequences include Cas9 sequences from the organisms and loci disclosed in Chylinski, Rhun, and Charpentier, “The tracrRNA and Cas9 families of type II CRISPR-Cas immunity systems” (2013) RNA Biology 10:5, 726-737; the entire contents of which are incorporated herein by reference. In some embodiments, a Cas9 nuclease has an inactive (e.g., an inactivated) DNA cleavage domain, that is, the Cas9 is a nickase. As one example, the Cas9 nuclease (e.g., Cas9 nickase) may cleave the DNA strand that is bound to the gRNA. As another example, the Cas9 nuclease (e.g., Cas9 nickase) may cleave the DNA strand that is not bound to the gRNA. In another embodiment, any of the guide nucleotide sequence-programmable DNA binding proteins may have an inactive (e.g., an inactivated) DNA cleavage domain, that is, the guide nucleotide sequence-programmable DNA binding protein is a nickase. As one example, the guide nucleotide sequence-programmable DNA binding protein may cleave the DNA strand that is bound to the gRNA. As another example, the guide nucleotide sequence-programmable DNA binding protein may cleave the DNA strand that is not bound to the gRNA.

Additional exemplary Cas9 sequences may be found in International Publication No.: WO/2017/070633, published Apr. 27, 2017, and entitled “Evolved Cas9 Proteins for Gene Editing.”

A nuclease-inactivated Cas9 protein may interchangeably be referred to as a “dCas9” protein (for nuclease “dead” Cas9). In some embodiments, dCas9 corresponds to, or comprises in part or in whole, the amino acid set forth as SEQ ID NO: 1, below. In some embodiments, variants of dCas9 (e.g., variants of SEQ ID NO: 1) are provided. For example, in some embodiments, variants having mutations other than D10A and H840A are provided, which e.g., result in nuclease inactivated Cas9 (dCas9). Such mutations, by way of example, include other amino acid substitutions at D10 and H840, or other substitutions within the nuclease domains of Cas9 (e.g., substitutions in the HNH nuclease subdomain and/or the RuvC1 subdomain). In some embodiments, variants or homologues of dCas9 (e.g., variants of SEQ ID NO: 1) are provided which are at least about 70% identical, at least about 80% identical, at least about 90% identical, at least about 95% identical, at least about 98% identical, at least about 99% identical, at least about 99.5% identical, or at least about 99.9% to SEQ ID NO: 1. In some embodiments, variants of dCas9 (e.g., variants of SEQ ID NO: 1) are provided having amino acid sequences which are shorter, or longer than SEQ ID NO: 1, by about 5 amino acids, by about 10 amino acids, by about 15 amino acids, by about 20 amino acids, by about 25 amino acids, by about 30 amino acids, by about 40 amino acids, by about 50 amino acids, by about 75 amino acids, by about 100 amino acids, or more.

dCas9 (D10A and H840A):
(SEQ ID NO: 1)
MDKKYSIGLAIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDAIVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEV
QTGGFSKESILPKRNSDKLIARKKDWDPKKYGGFDSPTVAYSVLVVAKVE
KGKSKKLKSVKELLGITIMERSSFEKNPIDFLEAKGYKEVKKDLIIKLPK
YSLFELENGRKRMLASAGELQKGNELALPSKYVNFLYLASHYEKLKGSPE
DNEQKQLFVEQHKHYLDEIIEQISEFSKRVILADANLDKVLSAYNKHRDK
PIREQAENIIHLFTLTNLGAPAAFKYFDTTIDRKRYTSTKEVLDATLIHQ
SITGLYETRIDLSQLGGD

Methods for generating a Cas9 protein (or a fragment thereof) having an inactive DNA cleavage domain are known (See, e.g., Jinek et al., Science. 337:816-821(2012); Qi et al., “Repurposing CRISPR as an RNA-Guided Platform for Sequence-Specific Control of Gene Expression” (2013) Cell. 28; 152(5):1173-83, the entire contents of each of which are incorporated herein by reference). For example, the DNA cleavage domain of Cas9 is known to include two subdomains, the HNH nuclease subdomain and the RuvC1 subdomain. The HNH subdomain cleaves the strand complementary to the gRNA, whereas the RuvC1 subdomain cleaves the non-complementary strand. Mutations within these subdomains can silence the nuclease activity of Cas9. For example, the mutations D10A and H840A completely inactivate the nuclease activity of S. pyogenes Cas9 (See e.g., Jinek et al., Science. 337:816-821(2012); Qi et al., Cell. 28; 152(5):1173-83 (2013)). In some embodiments, proteins comprising fragments of Cas9 are provided. For example, in some embodiments, a protein comprises one of two Cas9 domains: (1) the gRNA binding domain of Cas9; or (2) the DNA cleavage domain of Cas9. In some embodiments, proteins comprising Cas9, or fragments thereof, are referred to as “Cas9 variants.” A Cas9 variant shares homology to Cas9, or a fragment thereof. For example, a Cas9 variant is at least about 70% identical, at least about 80% identical, at least about 85% identical, at least about 90% identical, at least about 95% identical, at least about 98% identical, at least about 99% identical, at least about 99.5% identical, or at least about 99.9% to wild type Cas9. In some embodiments, the Cas9 variant comprises a fragment of Cas9 (e.g., a gRNA binding domain or a DNA-cleavage domain), such that the fragment is at least about 70% identical, at least about 80% identical, at least about 85% identical, at least about 90% identical, at least about 95% identical, at least about 98% identical, at least about 99% identical, at least about 99.5% identical, or at least about 99.9% to the corresponding fragment of wild type Cas9. In some embodiments, wild type Cas9 corresponds to Cas9 from Streptococcus pyogenes (NCBI Reference Sequence: NC_017053.1, SEQ ID NO: 2 (nucleotide); SEQ ID NO: 3 (amino acid)). In some embodiments the Cas9 domain comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to wild type Cas9. In some embodiments, the Cas9 domain comprises an amino acid sequence that has 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50 or more or more mutations compared to wild type Cas9. In some embodiments, the Cas9 domain comprises an amino acid sequence that has at least 10, at least 15, at least 20, at least 30, at least 40, at least 50, at least 60, at least 70, at least 80, at least 90, at least 100, at least 150, at least 200, at least 250, at least 300, at least 350, at least 400, at least 500, at least 600, at least 700, at least 800, at least 900, at least 1000, at least 1100, or at least 1200 identical contiguous amino acid residues as compared to wild type Cas9. In some embodiments, the Cas9 variant comprises a fragment of Cas9 (e.g., a gRNA binding domain or a DNA-cleavage domain), such that the fragment is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to the corresponding fragment of wild type Cas9. In some embodiments, the fragment is at least 30%, at least 35%, at least 40%, at least 45%, at least 50%, at least 55%, at least 60%, at least 65%, at least 70%, at least 75%, at least 80%, at least 85%, at least 90%, at least 95% identical, at least 96%, at least 97%, at least 98%, at least 99%, or at least 99.5% of the amino acid length of a corresponding wild type Cas9.

In some embodiments, the fragment is at least 100 amino acids in length. In some embodiments, the fragment is at least 100, 150, 200, 250, 300, 350, 400, 450, 500, 550, 600, 650, 700, 750, 800, 850, 900, 950, 1000, 1050, 1100, 1150, 1200, 1250, or 1300 amino acids in length.

(SEQ ID NO: 2)
ATGGATAAGAAATACTCAATAGGCTTAGATATCGGCACAAATAGCGTCGG
ATGGGCGGTGATCACTGATGATTATAAGGTTCCGTCTAAAAAGTTCAAGG
TTCTGGGAAATACAGACCGCCACAGTATCAAAAAAAATCTTATAGGGGCT
CTTTTATTTGGCAGTGGAGAGACAGCGGAAGCGACTCGTCTCAAACGGAC
AGCTCGTAGAAGGTATACACGTCGGAAGAATCGTATTTGTTATCTACAGG
AGATTTTTTCAAATGAGATGGCGAAAGTAGATGATAGTTTCTTTCATCGA
CTTGAAGAGTCTTTTTTGGTGGAAGAAGACAAGAAGCATGAACGTCATCC
TATTTTTGGAAATATAGTAGATGAAGTTGCTTATCATGAGAAATATCCAA
CTATCTATCATCTGCGAAAAAAATTGGCAGATTCTACTGATAAAGCGGAT
TTGCGCTTAATCTATTTGGCCTTAGCGCATATGATTAAGTTTCGTGGTCA
TTTTTTGATTGAGGGAGATTTAAATCCTGATAATAGTGATGTGGACAAAC
TATTTATCCAGTTGGTACAAATCTACAATCAATTATTTGAAGAAAACCCT
ATTAACGCAAGTAGAGTAGATGCTAAAGCGATTCTTTCTGCACGATTGAG
TAAATCAAGACGATTAGAAAATCTCATTGCTCAGCTCCCCGGTGAGAAGA
GAAATGGCTTGTTTGGGAATCTCATTGCTTTGTCATTGGGATTGACCCCT
AATTTTAAATCAAATTTTGATTTGGCAGAAGATGCTAAATTACAGCTTTC
AAAAGATACTTACGATGATGATTTAGATAATTTATTGGCGCAAATTGGAG
ATCAATATGCTGATTTGTTTTTGGCAGCTAAGAATTTATCAGATGCTATT
TTACTTTCAGATATCCTAAGAGTAAATAGTGAAATAACTAAGGCTCCCCT
ATCAGCTTCAATGATTAAGCGCTACGATGAACATCATCAAGACTTGACTC
TTTTAAAAGCTTTAGTTCGACAACAACTTCCAGAAAAGTATAAAGAAATC
TTTTTTGATCAATCAAAAAACGGATATGCAGGTTATATTGATGGGGGAGC
TAGCCAAGAAGAATTTTATAAATTTATCAAACCAATTTTAGAAAAAATGG
ATGGTACTGAGGAATTATTGGTGAAACTAAATCGTGAAGATTTGCTGCGC
AAGCAACGGACCTTTGACAACGGCTCTATTCCCCATCAAATTCACTTGGG
TGAGCTGCATGCTATTTTGAGAAGACAAGAAGACTTTTATCCATTTTTAA
AAGACAATCGTGAGAAGATTGAAAAAATCTTGACTTTTCGAATTCCTTAT
TATGTTGGTCCATTGGCGCGTGGCAATAGTCGTTTTGCATGGATGACTCG
GAAGTCTGAAGAAACAATTACCCCATGGAATTTTGAAGAAGTTGTCGATA
AAGGTGCTTCAGCTCAATCATTTATTGAACGCATGACAAACTTTGATAAA
AATCTTCCAAATGAAAAAGTACTACCAAAACATAGTTTGCTTTATGAGTA
TTTTACGGTTTATAACGAATTGACAAAGGTCAAATATGTTACTGAGGGAA
TGCGAAAACCAGCATTTCTTTCAGGTGAACAGAAGAAAGCCATTGTTGAT
TTACTCTTCAAAACAAATCGAAAAGTAACCGTTAAGCAATTAAAAGAAGA
TTATTTCAAAAAAATAGAATGTTTTGATAGTGTTGAAATTTCAGGAGTTG
AAGATAGATTTAATGCTTCATTAGGCGCCTACCATGATTTGCTAAAAATT
ATTAAAGATAAAGATTTTTTGGATAATGAAGAAAATGAAGATATCTTAGA
GGATATTGTTTTAACATTGACCTTATTTGAAGATAGGGGGATGATTGAGG
AAAGACTTAAAACATATGCTCACCTCTTTGATGATAAGGTGATGAAACAG
CTTAAACGTCGCCGTTATACTGGTTGGGGACGTTTGTCTCGAAAATTGAT
TAATGGTATTAGGGATAAGCAATCTGGCAAAACAATATTAGATTTTTTGA
AATCAGATGGTTTTGCCAATCGCAATTTTATGCAGCTGATCCATGATGAT
AGTTTGACATTTAAAGAAGATATTCAAAAAGCACAGGTGTCTGGACAAGG
CCATAGTTTACATGAACAGATTGCTAACTTAGCTGGCAGTCCTGCTATTA
AAAAAGGTATTTTACAGACTGTAAAAATTGTTGATGAACTGGTCAAAGTA
ATGGGGCATAAGCCAGAAAATATCGTTATTGAAATGGCACGTGAAAATCA
GACAACTCAAAAGGGCCAGAAAAATTCGCGAGAGCGTATGAAACGAATCG
AAGAAGGTATCAAAGAATTAGGAAGTCAGATTCTTAAAGAGCATCCTGTT
GAAAATACTCAATTGCAAAATGAAAAGCTCTATCTCTATTATCTACAAAA
TGGAAGAGACATGTATGTGGACCAAGAATTAGATATTAATCGTTTAAGTG
ATTATGATGTCGATCACATTGTTCCACAAAGTTTCATTAAAGACGATTCA
ATAGACAATAAGGTACTAACGCGTTCTGATAAAAATCGTGGTAAATCGGA
TAACGTTCCAAGTGAAGAAGTAGTCAAAAAGATGAAAAACTATTGGAGAC
AACTTCTAAACGCCAAGTTAATCACTCAACGTAAGTTTGATAATTTAACG
AAAGCTGAACGTGGAGGTTTGAGTGAACTTGATAAAGCTGGTTTTATCAA
ACGCCAATTGGTTGAAACTCGCCAAATCACTAAGCATGTGGCACAAATTT
TGGATAGTCGCATGAATACTAAATACGATGAAAATGATAAACTTATTCGA
GAGGTTAAAGTGATTACCTTAAAATCTAAATTAGTTTCTGACTTCCGAAA
AGATTTCCAATTCTATAAAGTACGTGAGATTAACAATTACCATCATGCCC
ATGATGCGTATCTAAATGCCGTCGTTGGAACTGCTTTGATTAAGAAATAT
CCAAAACTTGAATCGGAGTTTGTCTATGGTGATTATAAAGTTTATGATGT
TCGTAAAATGATTGCTAAGTCTGAGCAAGAAATAGGCAAAGCAACCGCAA
AATATTTCTTTTACTCTAATATCATGAACTTCTTCAAAACAGAAATTACA
CTTGCAAATGGAGAGATTCGCAAACGCCCTCTAATCGAAACTAATGGGGA
AACTGGAGAAATTGTCTGGGATAAAGGGCGAGATTTTGCCACAGTGCGCA
AAGTATTGTCCATGCCCCAAGTCAATATTGTCAAGAAAACAGAAGTACAG
ACAGGCGGATTCTCCAAGGAGTCAATTTTACCAAAAAGAAATTCGGACAA
GCTTATTGCTCGTAAAAAAGACTGGGATCCAAAAAAATATGGTGGTTTTG
ATAGTCCAACGGTAGCTTATTCAGTCCTAGTGGTTGCTAAGGTGGAAAAA
GGGAAATCGAAGAAGTTAAAATCCGTTAAAGAGTTACTAGGGATCACAAT
TATGGAAAGAAGTTCCTTTGAAAAAAATCCGATTGACTTTTTAGAAGCTA
AAGGATATAAGGAAGTTAAAAAAGACTTAATCATTAAACTACCTAAATAT
AGTCTTTTTGAGTTAGAAAACGGTCGTAAACGGATGCTGGCTAGTGCCGG
AGAATTACAAAAAGGAAATGAGCTGGCTCTGCCAAGCAAATATGTGAATT
TTTTATATTTAGCTAGTCATTATGAAAAGTTGAAGGGTAGTCCAGAAGAT
AACGAACAAAAACAATTGTTTGTGGAGCAGCATAAGCATTATTTAGATGA
GATTATTGAGCAAATCAGTGAATTTTCTAAGCGTGTTATTTTAGCAGATG
CCAATTTAGATAAAGTTCTTAGTGCATATAACAAACATAGAGACAAACCA
ATACGTGAACAAGCAGAAAATATTATTCATTTATTTACGTTGACGAATCT
TGGAGCTCCCGCTGCTTTTAAATATTTTGATACAACAATTGATCGTAAAC
GATATACGTCTACAAAAGAAGTTTTAGATGCCACTCTTATCCATCAATCC
ATCACTGGTCTTTATGAAACACGCATTGATTTGAGTCAGCTAGGAGGTGA
CTGA
(SEQ ID NO: 3)
MDKKYSIGLDIGTNSVGWAVITDDYKVPSKKFKVLGNTDRHSIKKNLIGA
LLFGSGETAEATRLKRTARRRYTRRKNRICYLQEIFSNEMAKVDDSFFHR
LEESFLVEEDKKHERHPIFGNIVDEVAYHEKYPTIYHLRKKLADSTDKAD
LRLIYLALAHMIKFRGHFLIEGDLNPDNSDVDKLFIQLVQIYNQLFEENP
INASRVDAKAILSARLSKSRRLENLIAQLPGEKRNGLFGNLIALSLGLTP
NFKSNFDLAEDAKLQLSKDTYDDDLDNLLAQIGDQYADLFLAAKNLSDAI
LLSDILRVNSEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGAYHDLLKI
IKDKDFLDNEENEDILEDIVLTLTLFEDRGMIEERLKTYAHLFDDKVMKQ
LKRRRYTGWGRLSRKLINGIRDKQSGKTILDFLKSDGFANRNFMQLIHDD
SLTFKEDIQKAQVSGQGHSLHEQIANLAGSPAIKKGILQTVKIVDELVKV
MGHKPENIVIEMARENQTTQKGQKNSRERMKRIEEGIKELGSQILKEHPV
ENTQLQNEKLYLYYLQNGRDMYVDQELDINRLSDYDVDHIVPQSFIKDDS
IDNKVLTRSDKNRGKSDNVPSEEVVKKMKNYWRQLLNAKLITQRKFDNLT
KAERGGLSELDKAGFIKRQLVETRQITKHVAQILDSRMNTKYDENDKLIR
EVKVITLKSKLVSDFRKDFQFYKVREINNYHHAHDAYLNAVVGTALIKKY
PKLESEFVYGDYKVYDVRKMIAKSEQEIGKATAKYFFYSNIMNFFKTEIT
LANGEIRKRPLIETNGETGEIVWDKGRDFATVRKVLSMPQVNIVKKTEVQ
TGGFSKESILPKRNSDKLIARKKDWDPKKYGGFDSPTVAYSVLVVAKVEK
GKSKKLKSVKELLGITIMERSSFEKNPIDFLEAKGYKEVKKDLIIKLPKY
SLFELENGRKRMLASAGELQKGNELALPSKYVNFLYLASHYEKLKGSPED
NEQKQLFVEQHKHYLDEIIEQISEFSKRVILADANLDKVLSAYNKHRDKP
IREQAENIIHLFTLTNLGAPAAFKYFDTTIDRKRYTSTKEVLDATLIHQS
ITGLYETRIDLSQLGGD

In some embodiments, wild type Cas9 corresponds to, or comprises, SEQ ID NO: 4 (nucleotide) and/or SEQ ID NO: 5 (amino acid).

(SEQ ID NO: 4)
ATGGATAAAAAGTATTCTATTGGTTTAGACATCGGCAC
TAATTCCGTTGGATGGGCTGTCATAACCGATGAATAC
AAAGTACCTTCAAAGAAATTTAAGGTGTTGGGGAACAC
AGACCGTCATTCGATTAAAAAGAATCTTATCGGTGCC
CTCCTATTCGATAGTGGCGAAACGGCAGAGGCGACTCG
CCTGAAACGAACCGCTCGGAGAAGGTATACACGTCGC
AAGAACCGAATATGTTACTTACAAGAAATTTTTAGCAA
TGAGATGGCCAAAGTTGACGATTCTTTCTTTCACCGT
TTGGAAGAGTCCTTCCTTGTCGAAGAGGACAAGAAACA
TGAACGGCACCCCATCTTTGGAAACATAGTAGATGAG
GTGGCATATCATGAAAAGTACCCAACGATTTATCACCT
CAGAAAAAAGCTAGTTGACTCAACTGATAAAGCGGAC
CTGAGGTTAATCTACTTGGCTCTTGCCCATATGATAAA
GTTCCGTGGGCACTTTCTCATTGAGGGTGATCTAAAT
CCGGACAACTCGGATGTCGACAAACTGTTCATCCAGTT
AGTACAAACCTATAATCAGTTGTTTGAAGAGAACCCT
ATAAATGCAAGTGGCGTGGATGCGAAGGCTATTCTTAG
CGCCCGCCTCTCTAAATCCCGACGGCTAGAAAACCTG
ATCGCACAATTACCCGGAGAGAAGAAAAATGGGTTGTT
CGGTAACCTTATAGCGCTCTCACTAGGCCTGACACCA
AATTTTAAGTCGAACTTCGACTTAGCTGAAGATGCCAA
ATTGCAGCTTAGTAAGGACACGTACGATGACGATCTC
GACAATCTACTGGCACAAATTGGAGATCAGTATGCGGA
CTTATTTTTGGCTGCCAAAAACCTTAGCGATGCAATC
CTCCTATCTGACATACTGAGAGTTAATACTGAGATTAC
CAAGGCGCCGTTATCCGCTTCAATGATCAAAAGGTAC
GATGAACATCACCAAGACTTGACACTTCTCAAGGCCCT
AGTCCGTCAGCAACTGCCTGAGAAATATAAGGAAATA
TTCTTTGATCAGTCGAAAAACGGGTACGCAGGTTATAT
TGACGGCGGAGCGAGTCAAGAGGAATTCTACAAGTTT
ATCAAACCCATATTAGAGAAGATGGATGGGACGGAAGA
GTTGCTTGTAAAACTCAATCGCGAAGATCTACTGCGA
AAGCAGCGGACTTTCGACAACGGTAGCATTCCACATCA
AATCCACTTAGGCGAATTGCATGCTATACTTAGAAGG
CAGGAGGATTTTTATCCGTTCCTCAAAGACAATCGTGA
AAAGATTGAGAAAATCCTAACCTTTCGCATACCTTAC
TATGTGGGACCCCTGGCCCGAGGGAACTCTCGGTTCGC
ATGGATGACAAGAAAGTCCGAAGAAACGATTACTCCA
TGGAATTTTGAGGAAGTTGTCGATAAAGGTGCGTCAGC
TCAATCGTTCATCGAGAGGATGACCAACTTTGACAAG
AATTTACCGAACGAAAAAGTATTGCCTAAGCACAGTTT
ACTTTACGAGTATTTCACAGTGTACAATGAACTCACG
AAAGTTAAGTATGTCACTGAGGGCATGCGTAAACCCGC
CTTTCTAAGCGGAGAACAGAAGAAAGCAATAGTAGAT
CTGTTATTCAAGACCAACCGCAAAGTGACAGTTAAGCA
ATTGAAAGAGGACTACTTTAAGAAAATTGAATGCTTC
GATTCTGTCGAGATCTCCGGGGTAGAAGATCGATTTAA
TGCGTCACTTGGTACGTATCATGACCTCCTAAAGATA
ATTAAAGATAAGGACTTCCTGGATAACGAAGAGAATGA
AGATATCTTAGAAGATATAGTGTTGACTCTTACCCTC
TTTGAAGATCGGGAAATGATTGAGGAAAGACTAAAAAC
ATACGCTCACCTGTTCGACGATAAGGTTATGAAACAG
TTAAAGAGGCGTCGCTATACGGGCTGGGGACGATTGTC
GCGGAAACTTATCAACGGGATAAGAGACAAGCAAAGT
GGTAAAACTATTCTCGATTTTCTAAAGAGCGACGGCTT
CGCCAATAGGAACTTTATGCAGCTGATCCATGATGAC
TCTTTAACCTTCAAAGAGGATATACAAAAGGCACAGGT
TTCCGGACAAGGGGACTCATTGCACGAACATATTGCG
AATCTTGCTGGTTCGCCAGCCATCAAAAAGGGCATACT
CCAGACAGTCAAAGTAGTGGATGAGCTAGTTAAGGTC
ATGGGACGTCACAAACCGGAAAACATTGTAATCGAGAT
GGCACGCGAAAATCAAACGACTCAGAAGGGGCAAAAA
AACAGTCGAGAGCGGATGAAGAGAATAGAAGAGGGTAT
TAAAGAACTGGGCAGCCAGATCTTAAAGGAGCATCCT
GTGGAAAATACCCAATTGCAGAACGAGAAACTTTACCT
CTATTACCTACAAAATGGAAGGGACATGTATGTTGAT
CAGGAACTGGACATAAACCGTTTATCTGATTACGACGT
CGATCACATTGTACCCCAATCCTTTTTGAAGGACGAT
TCAATCGACAATAAAGTGCTTACACGCTCGGATAAGAA
CCGAGGGAAAAGTGACAATGTTCCAAGCGAGGAAGTC
GTAAAGAAAATGAAGAACTATTGGCGGCAGCTCCTAAA
TGCGAAACTGATAACGCAAAGAAAGTTCGATAACTTA
ACTAAAGCTGAGAGGGGTGGCTTGTCTGAACTTGACAA
GGCCGGATTTATTAAACGTCAGCTCGTGGAAACCCGC
CAAATCACAAAGCATGTTGCACAGATACTAGATTCCCG
AATGAATACGAAATACGACGAGAACGATAAGCTGATT
CGGGAAGTCAAAGTAATCACTTTAAAGTCAAAATTGGT
GTCGGACTTCAGAAAGGATTTTCAATTCTATAAAGTT
AGGGAGATAAATAACTACCACCATGCGCACGACGCTTA
TCTTAATGCCGTCGTAGGGACCGCACTCATTAAGAAA
TACCCGAAGCTAGAAAGTGAGTTTGTGTATGGTGATTA
CAAAGTTTATGACGTCCGTAAGATGATCGCGAAAAGC
GAACAGGAGATAGGCAAGGCTACAGCCAAATACTTCTT
TTATTCTAACATTATGAATTTCTTTAAGACGGAAATC
ACTCTGGCAAACGGAGAGATACGCAAACGACCTTTAAT
TGAAACCAATGGGGAGACAGGTGAAATCGTATGGGAT
AAGGGCCGGGACTTCGCGACGGTGAGAAAAGTTTTGTC
CATGCCCCAAGTCAACATAGTAAAGAAAACTGAGGTG
CAGACCGGAGGGTTTTCAAAGGAATCGATTCTTCCAAA
AAGGAATAGTGATAAGCTCATCGCTCGTAAAAAGGAC
TGGGACCCGAAAAAGTACGGTGGCTTCGATAGCCCTAC
AGTTGCCTATTCTGTCCTAGTAGTGGCAAAAGTTGAG
AAGGGAAAATCCAAGAAACTGAAGTCAGTCAAAGAATT
ATTGGGGATAACGATTATGGAGCGCTCGTCTTTTGAA
AAGAACCCCATCGACTTCCTTGAGGCGAAAGGTTACAA
GGAAGTAAAAAAGGATCTCATAATTAAACTACCAAAG
TATAGTCTGTTTGAGTTAGAAAATGGCCGAAAACGGAT
GTTGGCTAGCGCCGGAGAGCTTCAAAAGGGGAACGAA
CTCGCACTACCGTCTAAATACGTGAATTTCCTGTATTT
AGCGTCCCATTACGAGAAGTTGAAAGGTTCACCTGAA
GATAACGAACAGAAGCAACTTTTTGTTGAGCAGCACAA
ACATTATCTCGACGAAATCATAGAGCAAATTTCGGAA
TTCAGTAAGAGAGTCATCCTAGCTGATGCCAATCTGGA
CAAAGTATTAAGCGCATACAACAAGCACAGGGATAAA
CCCATACGTGAGCAGGCGGAAAATATTATCCATTTGTT
TACTCTTACCAACCTCGGCGCTCCAGCCGCATTCAAG
TATTTTGACACAACGATAGATCGCAAACGATACACTTC
TACCAAGGAGGTGCTAGACGCGACACTGATTCACCAA
TCCATCACGGGATTATATGAAACTCGGATAGATTTGTC
ACAGCTTGGGGGTGACGGATCCCCCAAGAAGAAGAGG
AAAGTCTCGAGCGACTACAAAGACCATGACGGTGATTA
TAAAGATCATGACATCGATTACAAGGATGACGATGAC
AAGGCTGCAGGA
(SEQ ID NO: 5)
MDKKYSIGLAIGTNSVGWAVITDEYKVPSKKFKVLGNT
DRHSIKKNLIGALLFDSGETAEATRLKRTARRRYTRR
KNRICYLQEIFSNEMAKVDDSFFHRLEESFLVEEDKKH
ERHPIFGNIVDEVAYHEKYPTIYHLRKKLVDSTDKAD
LRLIYLALAHMIKFRGHFLIEGDLNPDNSDVDKLFIQL
VQTYNQLFEENPINASGVDAKAILSARLSKSRRLENL
IAQLPGEKKNGLFGNLIALSLGLTPNFKSNFDLAEDAK
LQLSKDTYDDDLDNLLAQIGDQYADLFLAAKNLSDAI
LLSDILRVNTEITKAPLSASMIKRYDEHHQDLTLLKAL
VRQQLPEKYKEIFFDQSKNGYAGYIDGGASQEEFYKF
IKPILEKMDGTEELLVKLNREDLLRKQRTFDNGSIPHQ
IHLGELHAILRRQEDFYPFLKDNREKIEKILTFRIPY
YVGPLARGNSRFAWMTRKSEETITPWNFEEVVDKGASA
QSFIERMTNFDKNLPNEKVLPKHSLLYEYFTVYNELT
KVKYVTEGMRKPAFLSGEQKKAIVDLLFKTNRKVTVKQ
LKEDYFKKIECFDSVEISGVEDRFNASLGTYHDLLKI
IKDKDFLDNEENEDILEDIVLTLTLFEDREMIEERLKT
YAHLFDDKVMKQLKRRRYTGWGRLSRKLINGIRDKQS
GKTILDFLKSDGFANRNFMQLIHDDSLTFKEDIQKAQV
SGQGDSLHEHIANLAGSPAIKKGILQTVKVVDELVKV
MGRHKPENIVIEMARENQTTQKGQKNSRERMKRIEEGI
KELGSQILKEHPVENTQLQNEKLYLYYLQNGRDMYVD
QELDINRLSDYDVDHIVPQSFLKDDSIDNKVLTRSDKN
RGKSDNVPSEEVVKKMKNYWRQLLNAKLITQRKFDNL
TKAERGGLSELDKAGFIKRQLVETRQITKHVAQILDSR
MNTKYDENDKLIREVKVITLKSKLVSDFRKDFQFYKV
REINNYHHAHDAYLNAVVGTALIKKYPKLESEFVYGDY
KVYDVRKMIAKSEQEIGKATAKYFFYSNIMNFFKTEI
TLANGEIRKRPLIETNGETGEIVWDKGRDFATVRKVLS
MPQVNIVKKTEVQTGGFSKESILPKRNSDKLIARKKD
WDPKKYGGFDSPTVAYSVLVVAKVEKGKSKKLKSVKEL
LGITIMERSSFEKNPIDFLEAKGYKEVKKDLIIKLPK
YSLFELENGRKRMLASAGELQKGNELALPSKYVNFLYL
ASHYEKLKGSPEDNEQKQLFVEQHKHYLDEIIEQISE
FSKRVILADANLDKVLSAYNKHRDKPIREQAENIIHLF
TLTNLGAPAAFKYFDTTIDRKRYTSTKEVLDATLIHQ
SITGLYETRIDLSQLGGD

In some embodiments, Cas9 refers to Cas9 from: Corynebacterium ulcerans (NCBI Refs: NC_015683.1, NC_017317.1); Corynebacterium diphtheria (NCBI Refs: NC_016782.1, NC_016786.1); Spiroplasma syrphidicola (NCBI Ref: NC_021284.1); Prevotella intermedia (NCBI Ref: NC_017861.1); Spiroplasma taiwanense (NCBI Ref: NC_021846.1); Streptococcus iniae (NCBI Ref: NC_021314.1); Belliella baltica (NCBI Ref: NC_018010.1); Psychroflexus torquisI (NCBI Ref: NC_018721.1); Streptococcus thermophilus (NCBI Ref: YP_820832.1), Listeria innocua (NCBI Ref: NP_472073.1); Campylobacter jejuni (NCBI Ref: YP_002344900.1); or Neisseria meningitidis (NCBI Ref: YP_002342100.1) or to a Cas9 from any other organism.

Cas9 recognizes a short motif (PAM motif) in the CRISPR repeat sequences in the target DNA sequence. A “PAM motif,” or “protospacer adjacent motif,” as used herein, refers a DNA sequence immediately following the DNA sequence targeted by the Cas9 nuclease in the CRISPR bacterial adaptive immune system. PAM is a component of the invading virus or plasmid, but is not a component of the bacterial CRISPR locus. Naturally, Cas9 will not successfully bind to or cleave the target DNA sequence if it is not followed by the PAM sequence. PAM is a targeting component (not found in the bacterial genome) which distinguishes bacterial self from non-self DNA, thereby preventing the CRISPR locus from being targeted and destroyed by the Cas9 nuclease activity.

Wild-type Streptococcus pyogenes Cas9 recognizes a canonical PAM sequence (e.g., Cas9 from Streptococcus thermophiles, Staphylococcus aureus, Neisseria meningitidis, or Treponema denticolaor) and Cas9 variants thereof have been described in the art to have different, or more relaxed PAM requirements. Typically, Cas9 proteins, such as Cas9 from S. pyogenes (spCas9), require a canonical NGG PAM sequence to bind a particular nucleic acid region, where the “N” in “NGG” is adenine (A), thymine (T), guanine (G), or cytosine (C), and the G is guanine. This may limit the ability to edit desired bases within a genome. In some embodiments, the base editing fusion proteins provided herein need to be positioned at a precise location, for example, where a target base is within a 4 base region (e.g., a “deamination window”), which is approximately 15 bases upstream of the PAM. See Komor, A. C., et al., “Programmable editing of a target base in genomic DNA without double-stranded DNA cleavage” Nature 533, 420-424 (2016), the entire contents of which are hereby incorporated by reference. In some embodiments, the deamination window is within a 2, 3, 4, 5, 6, 7, 8, 9, or 10 base region. In some embodiments, the deamination window is 5, 6, 7, 8, 9, 10, 11, 12, 13, 14, 15, 16, 17, 18, 19, 20, 21, 22, 23, 24, or 25 bases upstream of the PAM. Accordingly, in some embodiments, any of the fusion proteins provided herein may contain a Cas9 domain that is capable of binding a nucleotide sequence that does not contain a canonical (e.g., NGG) PAM sequence. Cas9 domains that bind to non-canonical PAM sequences have been described in the art and would be apparent to the skilled artisan. For example, Cas9 domains that bind non-canonical PAM sequences have been described in Kleinstiver, B. P., et al., “Engineered CRISPR-Cas9 nucleases with altered PAM specificities” Nature 523, 481-485 (2015); and Kleinstiver, B. P., et al., “Broadening the targeting range of Staphylococcus aureus CRISPR-Cas9 by modifying PAM recognition” Nature Biotechnology 33, 1293-1298 (2015); the entire contents of each are hereby incorporated by reference. See also: Klenstiver et al., Nature 529, 490-495, 2016; Ran et al., Nature, April 9; 520(7546): 186-191, 2015; Hou et al., Proc Natl Acad Sci USA, 110(39):15644-9, 2014; Prykhozhij et al., PLoS One, 10(3): e0119372, 2015; Zetsche et al., Cell 163, 759-771, 2015; Gao et al., Nature Biotechnology, doi:10.1038/nbt.3547, 2016; Want et al., Nature 461, 754-761, 2009; Chavez et al., doi: dx dot doi dot org/10.1101/058974; Fagerlund et al., Genome Biol. 2015; 16: 25, 2015; Zetsche et al., Cell, 163, 759-771, 2015; and Swarts et al., Nat Struct Mol Biol, 21(9):743-53, 2014, the entire contents of each of which is incorporated herein by reference.

Thus, the guide nucleotide sequence-programmable DNA-binding protein of the present disclosure may recognize a variety of PAM sequences including, without limitation: NGG, NGAN (SEQ ID NO: 741), NGNG (SEQ ID NO: 742), NGAG (SEQ ID NO: 743), NGCG (SEQ ID NO: 744), NNGRRT (SEQ ID NO: 745), NGRRN (SEQ ID NO: 746), NNNRRT (SEQ ID NO: 747), NNNGATT (SEQ ID NO: 748), NNAGAAW (SEQ ID NO: 749), NAAAC (SEQ ID NO: 750), TTN, TTTN (SEQ ID NO: 751), and YTN, wherein Y is a pyrimidine, and N is any nucleobase.

One example of an RNA-programmable DNA-binding protein that has different PAM specificity is Clustered Regularly Interspaced Short Palindromic Repeats from Prevotella and Francisella 1 (Cpf1). Similar to Cas9, Cpf1 is also a class 2 CRISPR effector. It has been shown that Cpf1 mediates robust DNA interference with features distinct from Cas9. Cpf1 is a single RNA-guided endonuclease lacking tracrRNA, and it utilizes a T-rich protospacer-adjacent motif (TTN, TTTN (SEQ ID NO: 751), or YTN). Moreover, Cpf1 cleaves DNA via a staggered DNA double-stranded break. Out of 16 Cpf1-family proteins, two enzymes from Acidaminococcus and Lachnospiraceae are shown to have efficient genome-editing activity in human cells.

Also provided herein are nuclease-inactive Cpf1 (dCpf1) variants that may be used as a RNA-programmable DNA-binding protein domain. The Cpf1 protein has a RuvC-like endonuclease domain that is similar to the RuvC domain of Cas9 but does not have a HNH endonuclease domain, and the N-terminal of Cpf1 does not have the alpha-helical recognition lobe of Cas9. It was shown in Zetsche et al., Cell, 163, 759-771, 2015 (the entire contents of which is incorporated herein by reference) that the RuvC-like domain of Cpf1 is responsible for cleaving both DNA strands and inactivation of the RuvC-like domain inactivates Cpf1 nuclease activity. For example, mutations corresponding to D917A, E1006A, or D1255A in Francisella novicida Cpf1 (SEQ ID NO: 714) inactivates Cpf1 nuclease activity. In some embodiments, the dCpf1 of the present disclosure comprises mutations corresponding to D917A, E1006A, D1255A, D917A/E1006A, D917A/D1255A, E1006A/D1255A, or D917A/E1006A/D1255A in SEQ ID NO: 714. It is to be understood that any mutations, e.g., substitution mutations, deletions, or insertions that inactivates the RuvC domain of Cpf1 may be used in accordance with the present disclosure.

In some embodiments, the guide nucleotide sequence-programmable DNA-binding protein domain of the present disclosure has no requirements for a PAM sequence. One example of such a guide nucleotide sequence-programmable DNA-binding protein may be an Argonaute protein from Natronobacterium gregoryi (NgAgo). NgAgo is a ssDNA-guided endonuclease. NgAgo binds 5′ phosphorylated ssDNA of ˜-24 nucleotides (gDNA) to guide it to its target site and will make DNA double-strand breaks at gDNA site. In contrast to Cas9, the NgAgo-gDNA system does not require a protospacer-adjacent motif (PAM). Using a nuclease inactive NgAgo (dNgAgo) can greatly expand the codons that may be targeted. The characterization and use of NgAgo have been described in Gao et al., Nat Biotechnol. Epub 2016 May 2. PubMed PMID: 27136078; Swarts et al., Nature. 507(7491) (2014):258-61; and Swarts et al., Nucleic Acids Res. 43(10) (2015):5120-9, the entire contents of each of which are incorporated herein by reference. The sequence of Natronobacterium gregoryi Argonaute is provided in SEQ ID NO: 718.

Also provided herein are Cas9 variants that have relaxed PAM requirements (PAMless Cas9). PAMless Cas9 exhibits an increased activity on a target sequence that does not comprise a canonical PAM (NGG) at its 3′-end as compared to Streptococcus pyogenes Cas9 as provided by SEQ ID NO: 1, e.g., increased activity by at least 5-fold, at least 10-fold, at least 50-fold, at least 100-fold, at least 500-fold, at least 1,000-fold, at least 5,000-fold, at least 10,000-fold, at least 50,000-fold, at least 100,000-fold, at least 500,000-fold, or at least 1,000,000-fold. Thus, the dCas9 or Cas9 nickase of the present disclosure may further comprise mutations that relax the PAM requirements, e.g., mutations that correspond to A262T, K294R, S409I, E480K, E543D, M694I, or E1219V in SEQ ID NO: 1.

It should be appreciated that additional Cas9 proteins (e.g., a nuclease dead Cas9 (dCas9), a Cas9 nickase (nCas9), or a nuclease active Cas9), including variants and homologs thereof, are within the scope of this disclosure. Exemplary Cas9 proteins include, without limitation, those provided below. In some embodiments, the Cas9 protein is a nuclease dead Cas9 (dCas9). In some embodiments, the dCas9 comprises the amino acid sequence shown below. In some embodiments, the Cas9 protein is a Cas9 nickase (nCas9). In some embodiments, the nCas9 comprises the amino acid sequence shown below. In some embodiments, the Cas9 protein is a nuclease active Cas9. In some embodiments, the nuclease active Cas9 comprises the amino acid sequence shown below.

Exemplary catalytically inactive Cas9 (dCas9):
(SEQ ID NO: 752)
DKKYSIGLAIGTNSVGWAVITDEYKVPSKKFKVLGNTDRHSIKKNLIGALLFDSGETA
EATRLKRTARRRYTRRKNRICYLQEIFSNEMAKVDDSFFHRLEESFLVEEDKKHERH
PIFGNIVDEVAYHEKYPTIYHLRKKLVDSTDKADLRLIYLALAHMIKFRGHFLIEGDL
NPDNSDVDKLFIQLVQTYNQLFEENPINASGVDAKAILSARLSKSRRLENLIAQLPGE
KKNGLFGNLIALSLGLTPNFKSNFDLAEDAKLQLSKDTYDDDLDNLLAQIGDQYADL
FLAAKNLSDAILLSDILRVNTEITKAPLSASMIKRYDEHHQDLTLLKALVRQQLPEKY
KEIFFDQSKNGYAGYIDGGASQEEFYKFIKPILEKMDGTEELLVKLNREDLLRKQRTF
DNGSIPHQIHLGELHAILRRQEDFYPFLKDNREKIEKILTFRIPYYVGPLARGNSRFAW
MTRKSEETITPWNFEEVVDKGASAQSFIERMTNFDKNLPNEKVLPKHSLLYEYFTVY
NELTKVKYVTEGMRKPAFLSGEQKKAIVDLLFKTNRKVTVKQLKEDYFKKIECFDS
VEISGVEDRFNASLGTYHDLLKIIKDKDFLDNEENEDILEDIVLTLTLFEDREMIEERL
KTYAHLFDDKVMKQLKRRRYTGWGRLSRKLINGIRDKQSGKTILDFLKSDGFANRN
FMQLIHDDSLTFKEDIQKAQVSGQGDSLHEHIANLAGSPAIKKGILQTVKVVDELVK
VMGRHKPENIVIEMARENQTTQKGQKNSRERMKRIEEGIKELGSQILKEHPVENTQL
QNEKLYLYYLQNGRDMYVDQELDINRLSDYDVDAIVPQSFLKDDSIDNKVLTRSDK
NRGKSDNVPSEEVVKKMKNYWRQLLNAKLITQRKFDNLTKAERGGLSELDKAGFIK
RQLVETRQITKHVAQILDSRMNTKYDENDKLIREVKVITLKSKLVSDFRKDFQFYKV
REINNYHHAHDAYLNAVVGTALIKKYPKLESEFVYGDYKVYDVRKMIAKSEQEIGK
ATAKYFFYSNIMNFFKTEITLANGEIRKRPLIETNGETGEIVWDKGRDFATVRKVLSM
PQVNIVKKTEVQTGGFSKESILPKRNSDKLIARKKDWDPKKYGGFDSPTVAYSVLVV
AKVEKGKSKKLKSVKELLGITIMERSSFEKNPIDFLEAKGYKEVKKDLIIKLPKYSLFE
LENGRKRMLASAGELQKGNELALPSKYVNFLYLASHYEKLKGSPEDNEQKQLFVEQ
HKHYLDEIIEQISEFSKRVILADANLDKVLSAYNKHRDKPIREQAENIIHLFTLTNLGA
PAAFKYFDTTIDRKRYTSTKEVLDATLIHQSITGLYETRIDLSQLGGD
Exemplary Cas9 nickase (nCas9):
(SEQ ID NO: 753)
DKKYSIGLAIGTNSVGWAVITDEYKVPSKKFKVLGNTDRHSIKKNLIGALLFDSGETA
EATRLKRTARRRYTRRKNRICYLQEIFSNEMAKVDDSFFHRLEESFLVEEDKKHERH
PIFGNIVDEVAYHEKYPTIYHLRKKLVDSTDKADLRLIYLALAHMIKFRGHFLIEGDL
NPDNSDVDKLFIQLVQTYNQLFEENPINASGVDAKAILSARLSKSRRLENLIAQLPGE
KKNGLFGNLIALSLGLTPNFKSNFDLAEDAKLQLSKDTYDDDLDNLLAQIGDQYADL
FLAAKNLSDAILLSDILRVNTEITKAPLSASMIKRYDEHHQDLTLLKALVRQQLPEKY
KEIFFDQSKNGYAGYIDGGASQEEFYKFIKPILEKMDGTEELLVKLNREDLLRKQRTF
DNGSIPHQIHLGELHAILRRQEDFYPFLKDNREKIEKILTFRIPYYVGPLARGNSRFAW
MTRKSEETITPWNFEEVVDKGASAQSFIERMTNFDKNLPNEKVLPKHSLLYEYFTVY
NELTKVKYVTEGMRKPAFLSGEQKKAIVDLLFKTNRKVTVKQLKEDYFKKIECFDS
VEISGVEDRFNASLGTYHDLLKIIKDKDFLDNEENEDILEDIVLTLTLFEDREMIEERL
KTYAHLFDDKVMKQLKRRRYTGWGRLSRKLINGIRDKQSGKTILDFLKSDGFANRN
FMQLIHDDSLTFKEDIQKAQVSGQGDSLHEHIANLAGSPAIKKGILQTVKVVDELVK
VMGRHKPENIVIEMARENQTTQKGQKNSRERMKRIEEGIKELGSQILKEHPVENTQL
QNEKLYLYYLQNGRDMYVDQELDINRLSDYDVDHIVPQSFLKDDSIDNKVLTRSDK
NRGKSDNVPSEEVVKKMKNYWRQLLNAKLITQRKFDNLTKAERGGLSELDKAGFIK
RQLVETRQITKHVAQILDSRMNTKYDENDKLIREVKVITLKSKLVSDFRKDFQFYKV
REINNYHHAHDAYLNAVVGTALIKKYPKLESEFVYGDYKVYDVRKMIAKSEQEIGK
ATAKYFFYSNIMNFFKTEITLANGEIRKRPLIETNGETGEIVWDKGRDFATVRKVLSM
PQVNIVKKTEVQTGGFSKESILPKRNSDKLIARKKDWDPKKYGGFDSPTVAYSVLVV
AKVEKGKSKKLKSVKELLGITIMERSSFEKNPIDFLEAKGYKEVKKDLIIKLPKYSLFE
LENGRKRMLASAGELQKGNELALPSKYVNFLYLASHYEKLKGSPEDNEQKQLFVEQ
HKHYLDEIIEQISEFSKRVILADANLDKVLSAYNKHRDKPIREQAENIIHLFTLTNLGA
PAAFKYFDTTIDRKRYTSTKEVLDATLIHQSITGLYETRIDLSQLGGD
Exemplary catalytically active Cas9:
(SEQ ID NO: 754)
DKKYSIGLDIGTNSVGWAVITDEYKVPSKKFKVLGNTDRHSIKKNLIGALLFDSGETA
EATRLKRTARRRYTRRKNRICYLQEIFSNEMAKVDDSFFHRLEESFLVEEDKKHERH
PIFGNIVDEVAYHEKYPTIYHLRKKLVDSTDKADLRLIYLALAHMIKFRGHFLIEGDL
NPDNSDVDKLFIQLVQTYNQLFEENPINASGVDAKAILSARLSKSRRLENLIAQLPGE
KKNGLFGNLIALSLGLTPNFKSNFDLAEDAKLQLSKDTYDDDLDNLLAQIGDQYADL
FLAAKNLSDAILLSDILRVNTEITKAPLSASMIKRYDEHHQDLTLLKALVRQQLPEKY
KEIFFDQSKNGYAGYIDGGASQEEFYKFIKPILEKMDGTEELLVKLNREDLLRKQRTF
DNGSIPHQIHLGELHAILRRQEDFYPFLKDNREKIEKILTFRIPYYVGPLARGNSRFAW
MTRKSEETITPWNFEEVVDKGASAQSFIERMTNFDKNLPNEKVLPKHSLLYEYFTVY
NELTKVKYVTEGMRKPAFLSGEQKKAIVDLLFKTNRKVTVKQLKEDYFKKIECFDS
VEISGVEDRFNASLGTYHDLLKIIKDKDFLDNEENEDILEDIVLTLTLFEDREMIEERL
KTYAHLFDDKVMKQLKRRRYTGWGRLSRKLINGIRDKQSGKTILDFLKSDGFANRN
FMQLIHDDSLTFKEDIQKAQVSGQGDSLHEHIANLAGSPAIKKGILQTVKVVDELVK
VMGRHKPENIVIEMARENQTTQKGQKNSRERMKRIEEGIKELGSQILKEHPVENTQL
QNEKLYLYYLQNGRDMYVDQELDINRLSDYDVDHIVPQSFLKDDSIDNKVLTRSDK
NRGKSDNVPSEEVVKKMKNYWRQLLNAKLITQRKFDNLTKAERGGLSELDKAGFIK
RQLVETRQITKHVAQILDSRMNTKYDENDKLIREVKVITLKSKLVSDFRKDFQFYKV
REINNYHHAHDAYLNAVVGTALIKKYPKLESEFVYGDYKVYDVRKMIAKSEQEIGK
ATAKYFFYSNIMNFFKTEITLANGEIRKRPLIETNGETGEIVWDKGRDFATVRKVLSM
PQVNIVKKTEVQTGGFSKESILPKRNSDKLIARKKDWDPKKYGGFDSPTVAYSVLVV
AKVEKGKSKKLKSVKELLGITIMERSSFEKNPIDFLEAKGYKEVKKDLIIKLPKYSLFE
LENGRKRMLASAGELQKGNELALPSKYVNFLYLASHYEKLKGSPEDNEQKQLFVEQ
HKHYLDEIIEQISEFSKRVILADANLDKVLSAYNKHRDKPIREQAENIIHLFTLTNLGA
PAAFKYFDTTIDRKRYTSTKEVLDATLIHQSITGLYETRIDLSQLGGD

In some embodiments, Cas9 refers to a Cas9 from arehaea (e.g. nanoarchaea), which constitute a domain and kingdom of single-celled prokaryotic microbes. In some embodiments, Cas9 refers to CasX or CasY, which have been described in, for example, Burstein et al., “New CRISPR-Cas systems from uncultivated microbes.” Cell Res. 2017 Feb. 21. doi: 10.1038/cr.2017.21, the entire contents of which is hereby incorporated by reference. Using genome-resolved metagenomics, a number of CRISPR-Cas systems were identified, including the first reported Cas9 in the archaeal domain of life. This divergent Cas9 protein was found in little-studied nanoarchaea as part of an active CRISPR-Cas system. In bacteria, two previously unknown systems were discovered, CRISPR-CasX and CRISPR-CasY, which are among the most compact systems yet discovered. In some embodiments, Cas9 refers to CasX, or a variant of CasX. In some embodiments, Cas9 refers to a CasY, or a variant of CasY. It should be appreciated that other RNA-guided DNA binding proteins may be used as a guide nucleotide sequence-programmable DNA-binding protein, and are within the scope of this disclosure.

In some embodiments, the guide nucleotide sequence-programmable DNA-binding protein domain of any of the fusion proteins provided herein may be a CasX or CasY protein. In some embodiments, guide nucleotide sequence-programmable DNA-binding protein domain is a CasX protein. In some embodiments, the guide nucleotide sequence-programmable DNA-binding protein domain is a CasY protein. In some embodiments, the guide nucleotide sequence-programmable DNA-binding protein domain comprises an amino acid sequence that is at least 85%, at least 90%, at least 91%, at least 92%, at least 93%, at least 94%, at least 95%, at least 96%, at least 97%, at least 98%, at least 99%, or at ease 99.5% identical to a naturally-occurring CasX or CasY protein. In some embodiments, the guide nucleotide sequence-programmable DNA-binding protein domain is a naturally-occurring CasX or CasY protein. In some embodiments, the guide nucleotide sequence-programmable DNA-binding protein domain comprises an amino acid sequence that is at least 85%, at least 90%, at least 91%, at least 92%, at least 93%, at least 94%, at least 95%, at least 96%, at least 97%, at least 98%, at least 99%, or at least 99.5% identical to any one of of the exemplary CasX or CasY proteins described herein. In some embodiments, the guide nucleotide sequence-programmable DNA-binding protein domain comprises an amino acid sequence of any one of of the exemplary CasX or CasY proteins described herein. It should be appreciated that CasX and CasY from other bacterial species may also be used in accordance with the present disclosure.

CasX (uniprot.org/uniprot/F0NN87; uniprot.org/
uniprot/F0NH53)
>tr|F0NN87|F0NN87_SULIH CRISPR-associated
Casx protein OS = Sulfolobus islandicus
(strain HVE10/4)
GN = SiH_0402 PE = 4 SV = 1
(SEQ ID NO: 755)
MEVPLYNIFGDNYIIQVATEAENSTIYNNKVEIDDEELRNVLNLAYKIAK
NNEDAAAERRGKAKKKKGEEGETTTSNIILPLSGNDKNPWTETLKCYNFP
TTVALSEVFKNFSQVKECEEVSAPSFVKPEFYEFGRSPGMVERTRRVKLE
VEPHYLIIAAAGWVLTRLGKAKVSEGDYVGVNVFTPTRGILYSLIQNVNG
IVPGIKPETAFGLWIARKVVSSVTNPNVSVVRIYTISDAVGQNPTTINGG
FSIDLTKLLEKRYLLSERLEAIARNALSISSNMRERYIVLANYIYEYLTG
SKRLEDLLYFANRDLIMNLNSDDGKVRDLKLISAYVNGELIRGEG
>tr|F0NH53|F0NH53_SULIR CRISPR associated protein,
Casx OS = Sulfolobus islandicus (strain REY15A)
GN = SiRe_0771 PE = 4 SV = 1
(SEQ ID NO: 756)
MEVPLYNIFGDNYIIQVATEAENSTIYNNKVEIDDEELRNVLNLAYKIAK
NNEDAAAERRGKAKKKKGEEGETTTSNIILPLSGNDKNPWTETLKCYNFP
TTVALSEVFKNFSQVKECEEVSAPSFVKPEFYKFGRSPGMVERTRRVKLE
VEPHYLIMAAAGWVLTRLGKAKVSEGDYVGVNVFTPTRGILYSLIQNVNG
IVPGIKPETAFGLWIARKVVSSVTNPNVSVVSIYTISDAVGQNPTTINGG
FSIDLTKLLEKRDLLSERLEAIARNALSISSNMRERYIVLANYIYEYLTG
SKRLEDLLYFANRDLIMNLNSDDGKVRDLKLISAYVNGELIRGEG
CasY (ncbi.nlm.nih.gov/protein/APG80656.1)
>APG80656.1 CRISPR-associated protein CasY
[uncultured Parcubacteria group bacterium]
(SEQ ID NO: 757)
MSKRHPRISGVKGYRLHAQRLEYTGKSGAMRTIKYPLYSSPSGGRTVPRE
IVSAINDDYVGLYGLSNFDDLYNAEKRNEEKVYSVLDFWYDCVQYGAVFS
YTAPGLLKNVAEVRGGSYELTKTLKGSHLYDELQIDKVIKFLNKKEISRA
NGSLDKLKKDIIDCFKAEYRERHKDQCNKLADDIKNAKKDAGASLGERQK
KLFRDFFGISEQSENDKPSFTNPLNLTCCLLPFDTVNNNRNRGEVLFNKL
KEYAQKLDKNEGSLEMWEYIGIGNSGTAFSNFLGEGFLGRLRENKITELK
KAMMDITDAWRGQEQEEELEKRLRILAALTIKLREPKFDNHWGGYRSDIN
GKLSSWLQNYINQTVKIKEDLKGHKKDLKKAKEMINRFGESDTKEEAVVS
SLLESIEKIVPDDSADDEKPDIPAIAIYRRFLSDGRLTLNRFVQREDVQE
ALIKERLEAEKKKKPKKRKKKSDAEDEKETIDFKELFPHLAKPLKLVPNF
YGDSKRELYKKYKNAAIYTDALWKAVEKIYKSAFSSSLKNSFFDTDFDKD
FFIKRLQKIFSVYRRFNTDKWKPIVKNSFAPYCDIVSLAENEVLYKPKQS
RSRKSAAIDKNRVRLPSTENIAKAGIALARELSVAGFDWKDLLKKEEHEE
YIDLIELHKTALALLLAVTETQLDISALDFVENGTVKDFMKTRDGNLVLE
GRFLEMFSQSIVFSELRGLAGLMSRKEFITRSAIQTMNGKQAELLYIPHE
FQSAKITTPKEMSRAFLDLAPAEFATSLEPESLSEKSLLKLKQMRYYPHY
FGYELTRTGQGIDGGVAENALRLEKSPVKKREIKCKQYKTLGRGQNKIVL
YVRSSYYQTQFLEWFLHRPKNVQTDVAVSGSFLIDEKKVKTRWNYDALTV
ALEPVSGSERVFVSQPFTIFPEKSAEEEGQRYLGIDIGEYGIAYTALEIT
GDSAKILDQNFISDPQLKTLREEVKGLKLDQRRGTFAMPSTKIARIRESL
VHSLRNRIHHLALKHKAKIVYELEVSRFEEGKQKIKKVYATLKKADVYSE
IDADKNLQTTVWGKLAVASEISASYTSQFCGACKKLWRAEMQVDETITTQ
ELIGTVRVIKGGTLIDAIKDFMRPPIFDENDTPFPKYRDFCDKHHISKKM
RGNSCLFICPFCRANADADIQASQTIALLRYVKEEKKVEDYFERFRKLKN
IKVLGQMKKI

The terms “conjugating,” “conjugated,” and “conjugation” refer to an association of two entities, for example, of two molecules such as two proteins, two domains (e.g., a binding domain and a cleavage domain), or a protein and an agent, e.g., a protein binding domain and a small molecule. In some aspects, the association is between a protein (e.g., RNA-programmable nuclease) and a nucleic acid (e.g., a guide RNA). The association can be, for example, via a direct or indirect (e.g., via a linker) covalent linkage. In some embodiments, the association is covalent. In some embodiments, two molecules are conjugated via a linker connecting both molecules. For example, in some embodiments where two proteins are conjugated to each other, e.g., a binding domain and a cleavage domain of an engineered nuclease, to form a protein fusion, the two proteins may be conjugated via a polypeptide linker, e.g., an amino acid sequence connecting the C-terminus of one protein to the N-terminus of the other protein.

The term “consensus sequence,” as used herein in the context of nucleic acid sequences, refers to a calculated sequence representing the most frequent nucleotide residues found at each position in a plurality of similar sequences. Typically, a consensus sequence is determined by sequence alignment in which similar sequences are compared to each other and similar sequence motifs are calculated. In the context of recombinase target site sequences, a consensus sequence of a recombinase target site may, in some embodiments, be the sequence most frequently bound, or bound with the highest affinity, by a given recombinase.

The term “engineered,” as used herein refers to a protein molecule, a nucleic acid, complex, substance, or entity that has been designed, produced, prepared, synthesized, and/or manufactured by a human. Accordingly, an engineered product is a product that does not occur in nature.

The term “effective amount,” as used herein, refers to an amount of a biologically active agent that is sufficient to elicit a desired biological response. In some embodiments, an effective amount of a recombinase may refer to the amount of the recombinase that is sufficient to induce recombination at a target site specifically bound and recombined by the recombinase. As will be appreciated by the skilled artisan, the effective amount of an agent, e.g., a nuclease, a recombinase, a hybrid protein, a fusion protein, a protein dimer, a complex of a protein (or protein dimer) and a polynucleotide, or a polynucleotide, may vary depending on various factors as, for example, on the desired biological response, the specific allele, genome, target site, cell, or tissue being targeted, and the agent being used.

A “guide nucleotide sequence-programmable DNA-binding protein,” as used herein, refers to a protein, a polypeptide, or a domain that is able to bind DNA, and the binding to its target DNA sequence is mediated by a guide nucleotide sequence. The “guide nucleotide” may be an RNA or DNA molecule (e.g., a single-stranded DNA or ssDNA molecule) that is complementary to the target sequence and can guide the DNA binding protein to the target sequence. As such, a guide nucleotide sequence-programmable DNA-binding protein may be a RNA-programmable DNA-binding protein, or an ssDNA-programmable DNA-binding protein. “Programmable” means the DNA-binding protein may be programmed to bind any DNA sequence that the guide nucleotide targets. The guide nucleotide sequence-programmable DNA-binding protein referred to herein may be any guide nucleotide sequence-programmable DNA-binding protein known in the art without limitation including, but not limited to, a bifurcated or circularly permuted guide nucleotide sequence-programmable DNA-binding protein. The term “circularly permuted” refers to proteins in which the order of the amino acids in a protein has been altered, resulting in a protein structure with altered connectivity but a similar (overall) three-dimensional shape. Circular permutations are formed when the original n and c terminal amino acids are connected via a peptide bond; the peptide sequence is then broken in another location within the peptide sequence, causing a new n and c-terminus. Circular permutations may occur through a number of processes including evolutionary events, post-translational modifications, or artificially engineered mutations. For example, circular permutations may be used to improve the catalytic activity or thermostability of proteins. A circularly permuted guide nucleotide sequence-programmable DNA-binding protein may be used with any of the embodiments described herein. The term “bifurcated” typically refers to a monomeric protein that is split into two parts. Typically both parts are required for the function of the monomeric protein. Bifurcated proteins may or may not dimerize on their own to reconstitute a functional protein. Bifurcations may occur through a number of processes including evolutionary events, post-translational modifications, or artificially engineered mutations. Other protein domains, when fused to bifurcated domains, can be used to force the reassembly of the bifurcated protein. In some cases, protein domains, whose interaction depends on a small molecule, can be fused to each bifurcated domain, resulting in the small-molecule regulated dimerization of the bifurcated protein.

The term “homologous,” as used herein, is an art-understood term that refers to nucleic acids or polypeptides that are highly related at the level of nucleotide and/or amino acid sequence. Nucleic acids or polypeptides that are homologous to each other are termed “homologues.” Homology between two sequences can be determined by sequence alignment methods known to those of skill in the art. In accordance with the invention, two sequences are considered to be homologous if they are at least about 50-60% identical, e.g., share identical residues (e.g., amino acid residues) in at least about 50-60% of all residues comprised in one or the other sequence, at least about 70% identical, at least about 80% identical, at least about 85% identical, at least about 90% identical, at least about 95% identical, at least about 98% identical, at least about 99% identical, at least about 99.5% identical, or at least about 99.9% identical, for at least one stretch of at least 20, at least 30, at least 40, at least 50, at least 60, at least 70, at least 80, at least 90, at least 100, at least 120, at least 150, or at least 200 amino acids.

The term “sequence identity” or “percent sequence identity” as used herein, may refer to the percentage of nucleic acid or amino acid residues within a given DNA or protein, respectively, that are identical to the reference sequence. See, for example: Christopher M. Holman, Protein Similarity Score: A Simplified Version of the BLAST Score as a Superior Alternative to Percent Identity for Claiming Genuses of Related Protein Sequences, 21 SANTA CLARA COMPUTER & HIGH TECH. L. J. 55, 60 (2004), which is herein incorporated by reference in its entirety.

The term “linker,” as used herein, refers to a bond (e.g., covalent bond), chemical group, or a molecule linking two molecules or moieties, e.g., two domains of a fusion protein, such as, for example, a nuclease-inactive Cas9 domain and a nucleic acid-editing domain (e.g., an adenosine deaminase). In some embodiments, a linker joins a gRNA binding domain of an RNA-programmable nuclease, including a Cas9 nuclease domain, and the catalytic domain of a nucleic-acid editing protein. In some embodiments, a linker joins a dCas9 and a nucleic-acid editing protein. Typically, the linker is positioned between, or flanked by, two groups, molecules, or other moieties and connected to each one via a covalent bond, thus connecting the two. In some embodiments, the linker is an amino acid or a plurality of amino acids (e.g., a peptide or protein). In some embodiments, the linker is an organic molecule, group, polymer, or chemical moiety. In some embodiments, the linker is 5-100 amino acids in length, for example, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, 90-100, 100-150, or 150-200 amino acids in length. Longer or shorter linkers are also contemplated. In some embodiments, a linker comprises the amino acid sequence SGSETPGTSESATPES (SEQ ID NO: 7), which may also be referred to as the XTEN linker. In some embodiments, a linker comprises the amino acid sequence SGGS (SEQ ID NO: 758). In some embodiments, a linker comprises (SGGS)ₙ(SEQ ID NO: 758), (GGGS)ₙ(SEQ ID NO: 759), (GGGGS)ₙ (SEQ ID NO: 722), (G)ₙ, (EAAAK)ₙ (SEQ ID NO: 723), (GGS)ₙ, or (XP)ₙ motif, or a combination of any of these, wherein n is independently an integer between 1 and 30, and wherein X is any amino acid. In some embodiments, n is 1, 2, 3, 4, 5, 6, 7, 8, 9, 10, 11, 12, 13, 14, or 15.

The term “mutation,” as used herein, refers to a substitution of a residue within a sequence, e.g., a nucleic acid or amino acid sequence, with another residue, or a deletion or insertion of one or more residues within a sequence. Mutations are typically described herein by identifying the original residue followed by the position of the residue within the sequence and by the identity of the newly substituted residue. Various methods for making the amino acid substitutions (mutations) provided herein are well known in the art, and are provided by, for example, Green and Sambrook, Molecular Cloning: A Laboratory Manual (4^{th} ed., Cold Spring Harbor Laboratory Press, Cold Spring Harbor, N.Y. (2012)).

The term “nuclear localization sequence” or “NLS” refers to an amino acid sequence that promotes import of a protein into the cell nucleus, for example, by nuclear transport. Nuclear localization sequences are known in the art and would be apparent to the skilled artisan. For example, NLS sequences are described in Plank et al., international PCT application, PCT/EP2000/011690, filed Nov. 23, 2000, published as WO/2001/038547 on May 31, 2001, the contents of which are incorporated herein by reference for their disclosure of exemplary nuclear localization sequences. In some embodiments, a NLS comprises the amino acid sequence PKKKRKV (SEQ ID NO: 702) or MDSLLMNRRKFLYQFKNVRWAKGRRETYLC (SEQ ID NO: 761).

The term “nuclease,” as used herein, refers to an agent, for example, a protein, capable of cleaving a phosphodiester bond connecting two nucleotide residues in a nucleic acid molecule. In some embodiments, “nuclease” refers to a protein having an inactive DNA cleavage domain, such that the nuclease is incapable of cleaving a phosphodiester bond. In some embodiments, a nuclease is a protein, e.g., an enzyme that can bind a nucleic acid molecule and cleave a phosphodiester bond connecting nucleotide residues within the nucleic acid molecule. A nuclease may be an endonuclease, cleaving a phosphodiester bonds within a polynucleotide chain, or an exonuclease, cleaving a phosphodiester bond at the end of the polynucleotide chain. In some embodiments, a nuclease is a site-specific nuclease, binding and/or cleaving a specific phosphodiester bond within a specific nucleotide sequence, which is also referred to herein as the “recognition sequence,” the “nuclease target site,” or the “target site.” In some embodiments, a nuclease is a RNA-guided (i.e., RNA-programmable) nuclease, which is associated with (e.g., binds to) an RNA (e.g., a guide RNA, “gRNA”) having a sequence that complements a target site, thereby providing the sequence specificity of the nuclease. In some embodiments, a nuclease recognizes a single stranded target site, while in other embodiments, a nuclease recognizes a double-stranded target site, for example, a double-stranded DNA target site. The target sites of many naturally occurring nucleases, for example, many naturally occurring DNA restriction nucleases, are well known to those of skill in the art. A nuclease protein typically comprises a “binding domain” that mediates the interaction of the protein with the nucleic acid substrate, and also, in some cases, specifically binds to a target site, and a “cleavage domain” that catalyzes the cleavage of the phosphodiester bond within the nucleic acid backbone. In some embodiments a nuclease protein can bind and cleave a nucleic acid molecule in a monomeric form, while, in other embodiments, a nuclease protein has to dimerize or multimerize in order to cleave a target nucleic acid molecule. Binding domains and cleavage domains of naturally occurring nucleases, as well as modular binding domains and cleavage domains that can be fused to create nucleases binding specific target sites, are well known to those of skill in the art. For example, the binding domain of a guide nucleotide sequence-programmable DNA binding protein such as an RNA-programmable nucleases (e.g., Cas9), or a Cas9 protein having an inactive DNA cleavage domain, can be used as a binding domain (e.g., that binds a gRNA to direct binding to a target site) to specifically bind a desired target site, and fused or conjugated to a cleavage domain.

The terms “nucleic acid” and “nucleic acid molecule,” as used herein, refer to a compound comprising a nucleobase and an acidic moiety, e.g., a nucleoside, a nucleotide, or a polymer of nucleotides. Typically, polymeric nucleic acids, e.g., nucleic acid molecules comprising three or more nucleotides are linear molecules, in which adjacent nucleotides are linked to each other via a phosphodiester linkage. In some embodiments, “nucleic acid” refers to individual nucleic acid residues (e.g., nucleotides and/or nucleosides). In some embodiments, “nucleic acid” refers to an oligonucleotide chain comprising three or more individual nucleotide residues. As used herein, the terms “oligonucleotide” and “polynucleotide” can be used interchangeably to refer to a polymer of nucleotides (e.g., a string of at least three nucleotides). In some embodiments, “nucleic acid” encompasses RNA as well as single and/or double-stranded DNA. Nucleic acids may be naturally occurring, for example, in the context of a genome, a transcript, an mRNA, tRNA, rRNA, siRNA, snRNA, gRNA, plasmid, cosmid, chromosome, chromatid, or other naturally occurring nucleic acid molecule. On the other hand, a nucleic acid molecule may be a non-naturally occurring molecule, e.g., a recombinant DNA or RNA, an artificial chromosome, an engineered genome, or fragment thereof, or a synthetic DNA, RNA, DNA/RNA hybrid, or including non-naturally occurring nucleotides or nucleosides. Furthermore, the terms “nucleic acid,” “DNA,” “RNA,” and/or similar terms include nucleic acid analogs, i.e., analogs having other than a phosphodiester backbone. Nucleic acids can be purified from natural sources, produced using recombinant expression systems and optionally purified, chemically synthesized, etc. Where appropriate, e.g., in the case of chemically synthesized molecules, nucleic acids can comprise nucleoside analogs such as analogs having chemically modified bases or sugars, and backbone modifications. A nucleic acid sequence is presented in the 5′ to 3′ direction unless otherwise indicated. In some embodiments, a nucleic acid is or comprises natural nucleosides (e.g., adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

The term “orthogonal” refers to biological components that interact minimally, if at all. Recombinase target sites containing different gRNA binding sites are orthogonal if the gRNA-directed recCas9 proteins do not interact, or interact minimally, with other potential recombinase sites. The term “orthogonality” refers to the idea that system components can be varied independently without affecting the performance of the other components. The gRNA directed nature of the complex makes the set of gRNA molecules complexed to recCas9 proteins capable of directing recombinase activity at only the gRNA-directed site. Orthogonality of the system is demonstrated by the complete or near complete dependence of the set of gRNA molecules on the enzymatic activity on a targeted recombinase site.

The term “pharmaceutical composition,” as used herein, refers to a composition that can be administrated to a subject in the context of treatment and/or prevention of a disease or disorder. In some embodiments, a pharmaceutical composition comprises an active ingredient, e.g., a recombinase fused to a Cas9 protein, or fragment thereof (or a nucleic acid encoding a such a fusion), and optionally a pharmaceutically acceptable excipient. In some embodiments, a pharmaceutical composition comprises inventive Cas9 variant/fusion (e.g., fCas9) protein(s) and gRNA(s) suitable for targeting the Cas9 variant/fusion protein(s) to a target nucleic acid. In some embodiments, the target nucleic acid is a gene. In some embodiments, the target nucleic acid is an allele associated with a disease, wherein the allele is cleaved by the action of the Cas9 variant/fusion protein(s). In some embodiments, the allele is an allele of the CLTA gene, the VEGF gene, the PCDH15, gene or the FAM19A2 gene. See, e.g., the Examples.

The term “proliferative disease,” as used herein, refers to any disease in which cell or tissue homeostasis is disturbed in that a cell or cell population exhibits an abnormally elevated proliferation rate. Proliferative diseases include hyperproliferative diseases, such as pre-neoplastic hyperplastic conditions and neoplastic diseases. Neoplastic diseases are characterized by an abnormal proliferation of cells and include both benign and malignant neoplasms. Malignant neoplasia is also referred to as cancer. In some embodiments, the compositions and methods provided herein are useful for treating a proliferative disease. For example, in some embodiments, pharmaceutical compositions comprising Cas9 (e.g., fCas9) protein(s) and gRNA(s) suitable for targeting the Cas9 protein(s) to an VEGF allele, wherein the allele is inactivated by the action of the Cas9 protein(s). See, e.g., the Examples.

The terms “protein,” “peptide,” and “polypeptide” are used interchangeably herein, and refer to a polymer of amino acid residues linked together by peptide (amide) bonds. The terms refer to a protein, peptide, or polypeptide of any size, structure, or function. Typically, a protein, peptide, or polypeptide will be at least three amino acids long. A protein, peptide, or polypeptide may refer to an individual protein or a collection of proteins. One or more of the amino acids in a protein, peptide, or polypeptide may be modified, for example, by the addition of a chemical entity such as a carbohydrate group, a hydroxyl group, a phosphate group, a farnesyl group, an isofarnesyl group, a fatty acid group, a linker for conjugation, functionalization, or other modification, etc. A protein, peptide, or polypeptide may also be a single molecule or may be a multi-molecular complex. A protein, peptide, or polypeptide may be just a fragment of a naturally occurring protein or peptide. A protein, peptide, or polypeptide may be naturally occurring, recombinant, or synthetic, or any combination thereof. The term “fusion protein” as used herein refers to a hybrid polypeptide that comprises protein domains from at least two different proteins. One protein may be located at the amino-terminal (N-terminal) portion of the fusion protein or at the carboxy-terminal (C-terminal) protein thus forming an “amino-terminal fusion protein” or a “carboxy-terminal fusion protein,” respectively. Any of the proteins provided herein may be produced by any method known in the art. For example, the proteins provided herein may be produced via recombinant protein expression and purification, which is especially suited for fusion proteins comprising a peptide linker. Methods for recombinant protein expression and purification are well known, and include those described by Green and Sambrook, Molecular Cloning: A Laboratory Manual (4^{th} ed., Cold Spring Harbor Laboratory Press, Cold Spring Harbor, N.Y. (2012)), the entire contents of which are incorporated herein by reference. A specific fusion protein referred to herein is recCas9, an RNA programmed small serine recombinase capable of functioning in mammalian cells created by fusion a catalytically inactive dCas9 to the catalytic domain of recombinase.

A “pseudo-gix” site or a “gix pseudo-site” as discussed herein is a specific pseudo-palindromic core DNA sequence that resembles the Gix recombinases' natural DNA recognition sequence. See, for example, N. D. F. Grindley, K. L. Whiteson, P. A. Rice, Mechanisms of site-specific recombination. Annu Rev Biochem 75, 567-605 (2006), which is incorporated by reference herein in its entirety. Similarly, a “pseudo-hix” or “hix-pseudo-site;” a “pseudo-six” or “six-pseudo site;” a “pseudo-resH” or “resH-pseudo-site;” “pseudo-res”or “res-pseudo-site;” “pseudo-LoxP” or “LoxP-pseudo-site;” “pseudo-att” or “att-pseudo-site;” “pseudo-FTR” or “FTR-pseudo-site” is a specific pseudo-palindromic core DNA sequence that resembles the Hin recombinase's, β recombinase's, Sin recombinase's, Tn3 or γδ recombinase's, Cre recombinase's, λ phage integrase's, or FLP recombinase's natural DNA recognition sequence.

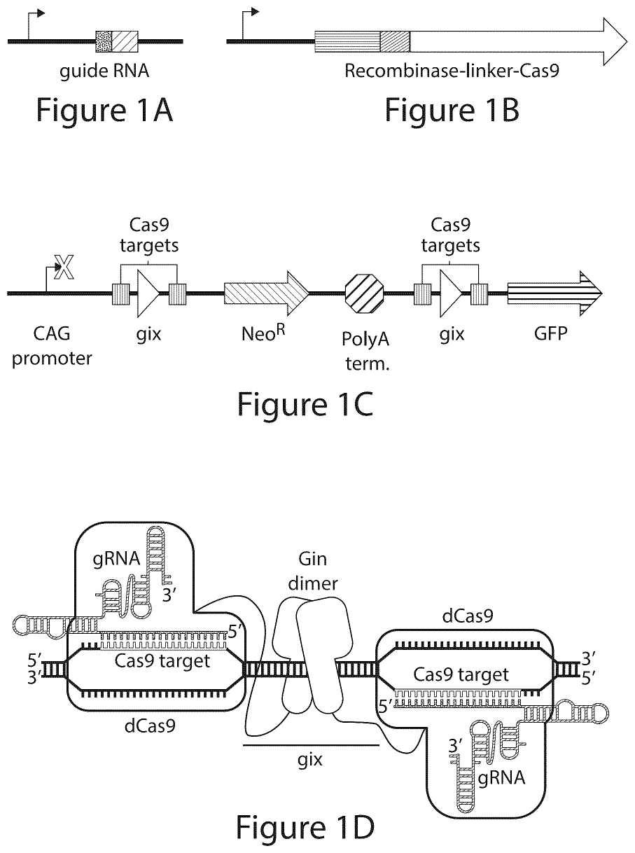

The terms “RNA-programmable nuclease” and “RNA-guided nuclease” are used interchangeably herein and refer to a nuclease that forms a complex with (e.g., binds or associates with) one or more RNA that is not a target for cleavage. In some embodiments, an RNA-programmable nuclease, when in a complex with an RNA, may be referred to as a nuclease:RNA complex. Typically, the bound RNA(s) is referred to as a guide RNA (gRNA). gRNAs can exist as a complex of two or more RNAs, or as a single RNA molecule. gRNAs that exist as a single RNA molecule may be referred to as single-guide RNAs (sgRNAs), though “gRNA” is used interchangeabley to refer to guide RNAs that exist as either single molecules or as a complex of two or more molecules. Typically, gRNAs that exist as single RNA species comprise two domains: (1) a domain that shares homology to a target nucleic acid (e.g., and directs binding of a Cas9 complex to the target); and (2) a domain that binds a Cas9 protein. In some embodiments, domain (2) corresponds to a sequence known as a tracrRNA, and comprises a stem-loop structure. For example, in some embodiments, domain (2) is homologous to a tracrRNA as depicted in FIG. 1E of Jinek et al., Science 337:816-821(2012), the entire contents of which is incorporated herein by reference. Other examples of gRNAs (e.g., those including domain 2) can be found in U.S. Provisional Patent Application, U.S. Ser. No. 61/874,682, filed Sep. 6, 2013, entitled “Switchable Cas9 Nucleases And Uses Thereof;” U.S. Provisional Patent Application, U.S. Ser. No. 61/874,746, filed Sep. 6, 2013, entitled “Delivery System For Functional Nucleases;” PCT Application WO 2013/176722, filed Mar. 15, 2013, entitled “Methods and Compositions for RNA-Directed Target DNA Modification and for RNA-Directed Modulation of Transcription;” and PCT Application WO 2013/142578, filed Mar. 20, 2013, entitled “RNA-Directed DNA Cleavage by the Cas9-crRNA Complex;” the entire contents of each are hereby incorporated by reference in their entirety. Still other examples of gRNAs are provided herein. See e.g., the Examples. In some embodiments, a gRNA comprises two or more of domains (1) and (2), and may be referred to as an “extended gRNA.” For example, an extended gRNA will e.g., bind two or more Cas9 proteins and bind a target nucleic acid at two or more distinct regions, as described herein. The gRNA comprises a nucleotide sequence that complements a target site, which mediates binding of the nuclease/RNA complex to said target site, providing the sequence specificity of the nuclease:RNA complex. In some embodiments, the guide nucleotide sequence-programmable DNA binding protein is an RNA-programmable nuclease such as the (CRISPR-associated system) Cas9 endonuclease, for example, Cas9 (Csnl) from Streptococcus pyogenes (see, e.g., “Complete genome sequence of an M1 strain of Streptococcus pyogenes.” Ferretti J. J., McShan W. M., Ajdic D. J., Savic D. J., Savic G., Lyon K., Primeaux C., Sezate S., Suvorov A. N., Kenton S., Lai H. S., Lin S. P., Qian Y., Jia H. G., Najar F. Z., Ren Q., Zhu H., Song L., White J., Yuan X., Clifton S. W., Roe B. A., McLaughlin R. E., Proc. Natl. Acad. Sci. U.S.A. 98:4658-4663(2001); “CRISPR RNA maturation by trans-encoded small RNA and host factor RNase III.” Deltcheva E., Chylinski K., Sharma C. M., Gonzales K., Chao Y., Pirzada Z. A., Eckert M. R., Vogel J., Charpentier E., Nature 471:602-607(2011); and “A programmable dual-RNA-guided DNA endonuclease in adaptive bacterial immunity.” Jinek M., Chylinski K., Fonfara I., Hauer M., Doudna J. A., Charpentier E. Science 337:816-821(2012), the entire contents of each of which are incorporated herein by reference.

Because RNA-programmable nucleases (e.g., Cas9) use RNA:DNA hybridization to determine target DNA cleavage sites, these proteins are able to cleave, in principle, any sequence specified by the guide RNA. Methods of using RNA-programmable nucleases, such as Cas9, for site-specific cleavage (e.g., to modify a genome) are known in the art (see e.g., Cong, L. et al. Multiplex genome engineering using CRISPR/Cas systems. Science 339, 819-823 (2013); Mali, P. et al. RNA-guided human genome engineering via Cas9. Science 339, 823-826 (2013); Hwang, W. Y. et al. Efficient genome editing in zebrafish using a CRISPR-Cas system. Nature biotechnology 31, 227-229 (2013); Jinek, M. et al. RNA-programmed genome editing in human cells. eLife 2, e00471 (2013); Dicarlo, J. E. et al. Genome engineering in Saccharomyces cerevisiae using CRISPR-Cas systems. Nucleic acids research (2013); Jiang, W. et al. RNA-guided editing of bacterial genomes using CRISPR-Cas systems. Nature biotechnology 31, 233-239 (2013); the entire contents of each of which are incorporated herein by reference).

The term “recombinase,” as used herein, refers to a site-specific enzyme that mediates the recombination of DNA between recombinase recognition sequences, which results in the excision, integration, inversion, or exchange (e.g., translocation) of DNA fragments between the recombinase recognition sequences. Recombinases can be classified into two distinct families: serine recombinases (e.g., resolvases and invertases) and tyrosine recombinases (e.g., integrases). Examples of serine recombinases include, without limitation, Hin, Gin, Tn3, β-six, CinH, ParA, γδ, Bxb1, ϕC31, TP901, TG1, φBT1, R4, φRVl, φFC1, MR11, A118, U153, and gp29. Examples of tyrosine recombinases include, without limitation, Cre, FLP, R, Lambda, HK101, HK022, and pSAM2. The Gin recombinase referred to herein may be any Gin recombinase known in the art including, but not limited to, the Gin recombinases presented in T. Gaj et al., A comprehensive approach to zinc-finger recombinase customization enables genomic targeting in human cells. Nucleic Acids Research 41, 3937-3946 (2013), incorporated herein by reference in its entirety. In certain embodiments, the Gin recombinase catalytic domain has greater than 85%, 90%, 95%, 98%, or 99% sequence identity with the amino acid sequence shown in SEQ ID NO: 713. In another embodiment, the amino acid sequence of the Gin recombinase catalytic domain comprises a mutation corresponding to H106Y, and/or I127L, and/or I136R and/or G137F. In yet another embodiment, the amino acid sequence of the Gin recombinase catalytic domain comprises a mutation corresponding to H106Y, I127L, I136R, and G137F. In a further embodiment, the amino acid sequence of the Gin recombinase has been further mutated. In a specific embodiment, the amino acid sequence of the Gin recombinase catalytic domain comprises SEQ ID NO: 713. Gin recombinases bind to gix target sites (also referred to herein as “gix core,” “minimal gix core,” or “gix-related core” sequences). The minimal gix core recombinase site is NNNNAAASSWWSSTTTNNNN (SEQ ID NO: 19), wherein N is defined as any amino acid, W is an A or a T, and S is a G or a C. The gix target site may include any other mutations known in the art. In certain embodiments, the gix target site has greater than 90%, 95%, or 99% sequence identity with the amino acid sequence shown in SEQ ID NO: 19. The distance between the gix core or gix-related core sequence and at least one gRNA binding site may be from 1 to 10 base pairs, from 3 to 7 base pairs, from 5 to 7 base pairs, or from 5 to 6 base pairs. The distance between the gix core or gix-related core sequence and at least one gRNA binding site may be 1, 2, 3, 4, 5, 6, 7, 8, 9, or 10 base pairs.

The serine and tyrosine recombinase names stem from the conserved nucleophilic amino acid residue that the recombinase uses to attack the DNA and which becomes covalently linked to the DNA during strand exchange. Recombinases have numerous applications, including the creation of gene knockouts/knock-ins and gene therapy applications. See, e.g., Brown et al., “Serine recombinases as tools for genome engineering.” Methods. 2011; 53(4):372-9; Hirano et al., “Site-specific recombinases as tools for heterologous gene integration.” Appl. Microbiol. Biotechnol. 2011; 92(2):227-39; Chavez and Calos, “Therapeutic applications of the ΦC31 integrase system.” Curr. Gene Ther. 2011; 11(5):375-81; Turan and Bode, “Site-specific recombinases: from tag-and-target- to tag-and-exchange-based genomic modifications.” FASEB J. 2011; 25(12):4088-107; Venken and Bellen, “Genome-wide manipulations of Drosophila melanogaster with transposons, Flp recombinase, and <DC31 integrase.” Methods Mol. Biol. 2012; 859:203-28; Murphy, “Phage recombinases and their applications.” Adv. Virus Res. 2012; 83:367-414; Zhang et al., “Conditional gene manipulation: Creating a new biological era.” J. Zhejiang Univ. Sci. B. 2012; 13(7):511-24; Karpenshif and Bernstein, “From yeast to mammals: recent advances in genetic control of homologous recombination.” DNA Repair (Amst). 2012; 1; 11(10):781-8; the entire contents of each are hereby incorporated by reference in their entirety. The recombinases provided herein are not meant to be exclusive examples of recombinases that can be used in embodiments of the invention. The methods and compositions of the invention can be expanded by mining databases for new orthogonal recombinases or designing synthetic recombinases with defined DNA specificities (See, e.g., Groth et al., “Phage integrases: biology and applications.” J. Mol. Biol. 2004; 335, 667-678; Gordley et al., “Synthesis of programmable integrases.” Proc. Natl. Acad. Sci. USA. 2009; 106, 5053-5058; the entire contents of each are hereby incorporated by reference in their entirety).

Other examples of recombinases that are useful in the methods and compositions described herein are known to those of skill in the art, and any new recombinase that is discovered or generated is expected to be able to be used in the different embodiments of the invention. In some embodiments, the catalytic domains of a recombinase are fused to a nuclease-inactivated RNA-programmable nuclease (e.g., dCas9, or a fragment thereof), such that the recombinase domain does not comprise a nucleic acid binding domain or is unable to bind to a target nucleic acid that subsequently results in enzymatic catalysis (e.g., the recombinase domain is engineered such that it does not have specific DNA binding activity). Recombinases lacking part of their DNA binding activity and those that act independently of accessory proteins and methods for engineering such are known, and include those described by Klippel et al., “Isolation and characterisation of unusual gin mutants.” EMBO J. 1988; 7: 3983-3989: Burke et al., “Activating mutations of Tn3 resolvase marking interfaces important in recombination catalysis and its regulation. Mol Microbiol. 2004; 51: 937-948; Olorunniji et al., “Synapsis and catalysis by activated Tn3 resolvase mutants.” Nucleic Acids Res. 2008; 36: 7181-7191; Rowland et al., “Regulatory mutations in Sin recombinase support a structure-based model of the synaptosome.” Mol Microbiol. 2009; 74: 282-298; Akopian et al., “Chimeric recombinases with designed DNA sequence recognition.” Proc Natl Acad Sci USA. 2003; 100: 8688-8691; Gordley et al., “Evolution of programmable zinc finger-recombinases with activity in human cells. J Mol Biol. 2007; 367: 802-813; Gordley et al., “Synthesis of programmable integrases.” Proc Natl Acad Sci USA. 2009; 106: 5053-5058; Arnold et al., “Mutants of Tn3 resolvase which do not require accessory binding sites for recombination activity.” EMBO J. 1999; 18: 1407-1414; Gaj et al., “Structure-guided reprogramming of serine recombinase DNA sequence specificity.” Proc Natl Acad Sci USA. 2011; 108(2):498-503; and Proudfoot et al., “Zinc finger recombinases with adaptable DNA sequence specificity.” PLoS One. 2011; 6(4):e19537; the entire contents of each are hereby incorporated by reference. For example, serine recombinases of the resolvase-invertase group, e.g., Tn3 and γδ resolvases and the Hin and Gin invertases, have modular structures with partly autonomous catalytic and DNA-binding domains (See, e.g., Grindley et al., “Mechanism of site-specific recombination.” Ann Rev Biochem. 2006; 75: 567-605, the entire contents of which are incorporated by reference). The catalytic domains of these recombinases are therefore amenable to being recombined with nuclease-inactivated RNA-programmable nucleases (e.g., dCas9, or a fragment thereof) as described herein, e.g., following the isolation of ‘activated’ recombinase mutants which do not require any accessory factors (e.g., DNA binding activities) (See, e.g., Klippel et al., “Isolation and characterisation of unusual gin mutants.” EMBO J. 1988; 7: 3983-3989: Burke et al., “Activating mutations of Tn3 resolvase marking interfaces important in recombination catalysis and its regulation. Mol Microbiol. 2004; 51: 937-948; Olorunniji et al., “Synapsis and catalysis by activated Tn3 resolvase mutants.” Nucleic Acids Res. 2008; 36: 7181-7191; Rowland et al., “Regulatory mutations in Sin recombinase support a structure-based model of the synaptosome.” Mol Microbiol. 2009; 74: 282-298; Akopian et al., “Chimeric recombinases with designed DNA sequence recognition.” Proc Natl Acad Sci USA. 2003; 100: 8688-8691).

Additionally, many other natural serine recombinases having an N-terminal catalytic domain and a C-terminal DNA binding domain are known (e.g., phiC31 integrase, TnpX transposase, IS607 transposase), and their catalytic domains can be co-opted to engineer programmable site-specific recombinases as described herein (See, e.g., Smith et al., “Diversity in the serine recombinases.” Mol Microbiol. 2002; 44: 299-307, the entire contents of which are incorporated by reference). Similarly, the core catalytic domains of tyrosine recombinases (e.g., Cre, λ integrase) are known, and can be similarly co-opted to engineer programmable site-specific recombinases as described herein (See, e.g., Guo et al., “Structure of Cre recombinase complexed with DNA in a site-specific recombination synapse.” Nature. 1997; 389:40-46; Hartung et al., “Cre mutants with altered DNA binding properties.” J Biol Chem 1998; 273:22884-22891; Shaikh et al., “Chimeras of the Flp and Cre recombinases: Tests of the mode of cleavage by Flp and Cre. J Mol Biol. 2000; 302:27-48; Rongrong et al., “Effect of deletion mutation on the recombination activity of Cre recombinase.” Acta Biochim Pol. 2005; 52:541-544; Kilbride et al., “Determinants of product topology in a hybrid Cre-Tn3 resolvase site-specific recombination system.” J Mol Biol. 2006; 355:185-195; Warren et al., “A chimeric cre recombinase with regulated directionality.” Proc Natl Acad Sci USA. 2008 105:18278-18283; Van Duyne, “Teaching Cre to follow directions.” Proc Natl Acad Sci USA. 2009 Jan. 6; 106(1):4-5; Numrych et al., “A comparison of the effects of single-base and triple-base changes in the integrase arm-type binding sites on the site-specific recombination of bacteriophage λ.” Nucleic Acids Res. 1990; 18:3953-3959; Tirumalai et al., “The recognition of core-type DNA sites by λ integrase.” J Mol Biol. 1998; 279:513-527; Aihara et al., “A conformational switch controls the DNA cleavage activity of λ integrase.” Mol Cell. 2003; 12:187-198; Biswas et al., “A structural basis for allosteric control of DNA recombination by λ integrase.” Nature. 2005; 435:1059-1066; and Warren et al., “Mutations in the amino-terminal domain of λ-integrase have differential effects on integrative and excisive recombination.” Mol Microbiol. 2005; 55:1104-1112; the entire contents of each are incorporated by reference).

The term “recombine” or “recombination,” in the context of a nucleic acid modification (e.g., a genomic modification), is used to refer to the process by which two or more nucleic acid molecules, or two or more regions of a single nucleic acid molecule, are modified by the action of a recombinase protein (e.g., an inventive recombinase fusion protein provided herein). Recombination can result in, inter alia, the insertion, inversion, excision, or translocation of nucleic acids, e.g., in or between one or more nucleic acid molecules.

The term “recombinant” as used herein in the context of proteins or nucleic acids refers to proteins or nucleic acids that do not occur in nature, but are the product of human engineering. For example, in some embodiments, a recombinant protein or nucleic acid molecule comprises an amino acid or nucleotide sequence that comprises at least one, at least two, at least three, at least four, at least five, at least six, or at least seven mutations as compared to any naturally occurring sequence.

The term “subject,” as used herein, refers to an individual organism, for example, an individual mammal. In some embodiments, the subject is a human. In some embodiments, the subject is a non-human mammal. In some embodiments, the subject is a non-human primate. In some embodiments, the subject is a rodent. In some embodiments, the subject is a sheep, a goat, a cattle, a cat, or a dog. In some embodiments, the subject is a vertebrate, an amphibian, a reptile, a fish, an insect, a fly, or a nematode. In some embodiments, the subject is a research animal. In some embodiments, the subject is genetically engineered, e.g., a genetically engineered non-human subject. The subject may be of either sex and at any stage of development. In some embodiments, the subject is genetically engineered, e.g., a genetically engineered non-human subject. The subject may be of either sex and at any stage of development.

The terms “target nucleic acid,” and “target genome,” as used herein in the context of nucleases, refer to a nucleic acid molecule or a genome, respectively, that comprises at least one target site of a given nuclease. In the context of fusions comprising a (nuclease-inactivated) RNA-programmable nuclease and a recombinase domain, a “target nucleic acid” and a “target genome” refers to one or more nucleic acid molecule(s), or a genome, respectively, that comprises at least one target site. In some embodiments, the target nucleic acid(s) comprises at least two, at least three, at least four, at least five, at least six, at least seven, or at least eight target sites. In some embodiments, the target nucleic acid(s) comprise four target sites.

The term “target site” refers to a sequence within a nucleic acid molecule that is bound and recombined (e.g., at or nearby the target site) by a recombinase (e.g., a dCas9-recombinase fusion protein provided herein). A target site may be single-stranded or double-stranded. For example, in some embodiments, four recombinase monomers are coordinated to recombine a target nucleic acid(s), each monomer being fused to a (nuclease-inactivated) Cas9 protein guided by a gRNA. In such an example, each Cas9 domain is guided by a distinct gRNA to bind a target nucleic acid(s), thus the target nucleic acid comprises four target sites, each site targeted by a separate dCas9-recombinase fusion (thereby coordinating four recombinase monomers which recombine the target nucleic acid(s)). For the RNA-guided nuclease-inactivated Cas9 (or gRNA-binding domain thereof) and inventive fusions of Cas9, the target site may be, in some embodiments, 17-20 base pairs plus a 3 base pair PAM (e.g., NNN, wherein N independently represents any nucleotide). Typically, the first nucleotide of a PAM can be any nucleotide, while the two downstream nucleotides are specified depending on the specific RNA-guided nuclease. Exemplary target sites (e.g., comprising a PAM) for RNA-guided nucleases, such as Cas9, are known to those of skill in the art and include, without limitation, NNG, NGN, NAG, and NGG, wherein each N is independently any nucleotide. In addition, Cas9 nucleases from different species (e.g., S. thermophilus instead of S. pyogenes) recognize a PAM that comprises the sequence NGGNG (SEQ ID NO: 763). Additional PAM sequences are known, including, but not limited to, NNAGAAW (SEQ ID NO: 749) and NAAR (SEQ ID NO: 771) (see, e.g., Esvelt and Wang, Molecular Systems Biology, 9:641 (2013), the entire contents of which are incorporated herein by reference). In some aspects, the target site of an RNA-guided nuclease, such as, e.g., Cas9, may comprise the structure [Nz]-[PAM], where each N is independently any nucleotide, and z is an integer between 1 and 50, inclusive. In some embodiments, z is at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, or at least 50. In some embodiments, z is 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50. In some embodiments, z is 20. In certain embodiments, a “PAMless” RNA-guided nuclease (e.g., a Pamless Cas9) or an RNA-guided nuclease with relaxed PAM requirements as further described herein may be used. In some embodiments, “target site” may also refer to a sequence within a nucleic acid molecule that is bound but not cleaved by a nuclease. For example, certain embodiments described herein provide proteins comprising an inactive (or inactivated) Cas9 DNA cleavage domain. Such proteins (e.g., when also including a Cas9 RNA binding domain) are able to bind the target site specified by the gRNA; however, because the DNA cleavage site is inactivated, the target site is not cleaved by the particular protein. In some embodiments, such proteins are conjugated, fused, or bound to a recombinase (or a catalytic domain of a recombinase), which mediates recombination of the target nucleic acid. In some embodiments, the sequence actually cleaved or recombined will depend on the protein (e.g., recombinase) or molecule that mediates cleavage or recombination of the nucleic acid molecule, and in some cases, for example, will relate to the proximity or distance from which the inactivated Cas9 protein(s) is/are bound.

The term “Transcriptional Activator-Like Effector,” (TALE) as used herein, refers to bacterial proteins comprising a DNA binding domain, which contains a highly conserved 33-34 amino acid sequence comprising a highly variable two-amino acid motif (Repeat Variable Diresidue, RVD). The RVD motif determines binding specificity to a nucleic acid sequence and can be engineered according to methods known to those of skill in the art to specifically bind a desired DNA sequence (see, e.g., Miller, Jeffrey; et.al. (February 2011). “A TALE nuclease architecture for efficient genome editing”. Nature Biotechnology 29 (2): 143-8; Zhang, Feng; et.al. (February 2011). “Efficient construction of sequence-specific TAL effectors for modulating mammalian transcription” Nature Biotechnology 29 (2): 149-53; Geipler, R.; Scholze, H.; Hahn, S.; Streubel, J.; Bonas, U.; Behrens, S. E.; Boch, J. (2011), Shiu, Shin-Han. ed. “Transcriptional Activators of Human Genes with Programmable DNA-Specificity”. PLoS ONE 6 (5): e19509; Boch, Jens (February 2011). “TALEs of genome targeting”. Nature Biotechnology 29 (2): 135-6; Boch, Jens; et. al. (December 2009). “Breaking the Code of DNA Binding Specificity of TAL-Type III Effectors”. Science 326 (5959): 1509-12; and Moscou, Matthew J.; Adam J. Bogdanove (December 2009). “A Simple Cipher Governs DNA Recognition by TAL Effectors” Science 326 (5959): 1501; the entire contents of each of which are incorporated herein by reference). The simple relationship between amino acid sequence and DNA recognition has allowed for the engineering of specific DNA binding domains by selecting a combination of repeat segments containing the appropriate RVDs.

The term “Transcriptional Activator-Like Element Nuclease,” (TALEN) as used herein, refers to an artificial nuclease comprising a transcriptional activator-like effector DNA binding domain to a DNA cleavage domain, for example, a FokI domain. A number of modular assembly schemes for generating engineered TALE constructs have been reported (see e.g., Zhang, Feng; et. al. (February 2011). “Efficient construction of sequence-specific TAL effectors for modulating mammalian transcription”. Nature Biotechnology 29 (2): 149-53; Geipler, R.; Scholze, H.; Hahn, S.; Streubel, J.; Bonas, U.; Behrens, S. E.; Boch, J. (2011), Shiu, Shin-Han. ed. “Transcriptional Activators of Human Genes with Programmable DNA-Specificity”. PLoS ONE 6 (5): e19509; Cermak, T.; Doyle, E. L.; Christian, M.; Wang, L.; Zhang, Y.; Schmidt, C.; Baller, J. A.; Somia, N. V. et al. (2011). “Efficient design and assembly of custom TALEN and other TAL effector-based constructs for DNA targeting”. Nucleic Acids Research; Morbitzer, R.; Elsaesser, J.; Hausner, J.; Lahaye, T. (2011). “Assembly of custom TALE-type DNA binding domains by modular cloning”. Nucleic Acids Research; Li, T.; Huang, S.; Zhao, X.; Wright, D. A.; Carpenter, S.; Spalding, M. H.; Weeks, D. P.; Yang, B. (2011). “Modularly assembled designer TAL effector nucleases for targeted gene knockout and gene replacement in eukaryotes”. Nucleic Acids Research.; Weber, E.; Gruetzner, R.; Werner, S.; Engler, C.; Marillonnet, S. (2011). Bendahmane, Mohammed. ed. “Assembly of Designer TAL Effectors by Golden Gate Cloning”. PLoS ONE 6 (5): e19722; the entire contents of each of which are incorporated herein by reference).

The terms “treatment,” “treat,” and “treating,” refer to a clinical intervention aimed to reverse, alleviate, delay the onset of, or inhibit the progress of a disease or disorder, or one or more symptoms thereof, as described herein. As used herein, the terms “treatment,” “treat,” and “treating” refer to a clinical intervention aimed to reverse, alleviate, delay the onset of, or inhibit the progress of a disease or disorder, or one or more symptoms thereof, as described herein. In some embodiments, treatment may be administered after one or more symptoms have developed and/or after a disease has been diagnosed. In other embodiments, treatment may be administered in the absence of symptoms, e.g., to prevent or delay onset of a symptom or inhibit onset or progression of a disease. For example, treatment may be administered to a susceptible individual prior to the onset of symptoms (e.g., in light of a history of symptoms and/or in light of genetic or other susceptibility factors). Treatment may also be continued after symptoms have resolved, for example, to prevent or delay their recurrence.

The term “vector” refers to a polynucleotide comprising one or more recombinant polynucleotides of the present invention, e.g., those encoding a Cas9 protein (or fusion thereof) and/or gRNA provided herein. Vectors include, but are not limited to, plasmids, viral vectors, cosmids, artificial chromosomes, and phagemids. The vector may be able to replicate in a host cell and may further be characterized by one or more endonuclease restriction sites at which the vector may be cut and into which a desired nucleic acid sequence may be inserted. Vectors may contain one or more marker sequences suitable for use in the identification and/or selection of cells which have or have not been transformed or genomically modified with the vector. Markers include, for example, genes encoding proteins which increase or decrease either resistance or sensitivity to antibiotics (e.g., kanamycin, ampicillin) or other compounds, genes which encode enzymes whose activities are detectable by standard assays known in the art (e.g., β-galactosidase, alkaline phosphatase, or luciferase), and genes which visibly affect the phenotype of transformed or transfected cells, hosts, colonies, or plaques. Any vector suitable for the transformation of a host cell (e.g., E. coli, mammalian cells such as CHO cell, insect cells, etc.) as embraced by the present invention, for example, vectors belonging to the pUC series, pGEM series, pET series, pBAD series, pTET series, or pGEX series. In some embodiments, the vector is suitable for transforming a host cell for recombinant protein production. Methods for selecting and engineering vectors and host cells for expressing proteins (e.g., those provided herein), transforming cells, and expressing/purifying recombinant proteins are well known in the art, and are provided by, for example, Green and Sambrook, Molecular Cloning: A Laboratory Manual (4^{th} ed., Cold Spring Harbor Laboratory Press, Cold Spring Harbor, N.Y. (2012)).

The term “zinc finger,” as used herein, refers to a small nucleic acid-binding protein structural motif characterized by a fold and the coordination of one or more zinc ions that stabilize the fold. Zinc fingers encompass a wide variety of differing protein structures (see, e.g., Klug A, Rhodes D (1987). “Zinc fingers: a novel protein fold for nucleic acid recognition”. Cold Spring Harb. Symp. Quant. Biol. 52: 473-82, the entire contents of which are incorporated herein by reference). Zinc fingers can be designed to bind a specific sequence of nucleotides, and zinc finger arrays comprising fusions of a series of zinc fingers, can be designed to bind virtually any desired target sequence. Such zinc finger arrays can form a binding domain of a protein, for example, of a nuclease, e.g., if conjugated to a nucleic acid cleavage domain. Different types of zinc finger motifs are known to those of skill in the art, including, but not limited to, Cys₂His₂, Gag knuckle, Treble clef, Zinc ribbon, Zn₂/Cys₆, and TAZ2 domain-like motifs (see, e.g., Krishna S S, Majumdar I, Grishin N V (January 2003). “Structural classification of zinc fingers: survey and summary”. Nucleic Acids Res. 31 (2): 532-50). Typically, a single zinc finger motif binds 3 or 4 nucleotides of a nucleic acid molecule. Accordingly, a zinc finger domain comprising 2 zinc finger motifs may bind 6-8 nucleotides, a zinc finger domain comprising 3 zinc finger motifs may bind 9-12 nucleotides, a zinc finger domain comprising 4 zinc finger motifs may bind 12-16 nucleotides, and so forth. Any suitable protein engineering technique can be employed to alter the DNA-binding specificity of zinc fingers and/or design novel zinc finger fusions to bind virtually any desired target sequence from 3-30 nucleotides in length (see, e.g., Pabo C O, Peisach E, Grant R A (2001). “Design and selection of novel cys2His2 Zinc finger proteins”. Annual Review of Biochemistry 70: 313-340; Jamieson A C, Miller J C, Pabo C O (2003). “Drug discovery with engineered zinc-finger proteins”. Nature Reviews Drug Discovery 2 (5): 361-368; and Liu Q, Segal D J, Ghiara J B, Barbas C F (May 1997). “Design of polydactyl zinc-finger proteins for unique addressing within complex genomes”. Proc. Natl. Acad. Sci. U.S.A. 94 (11); the entire contents of each of which are incorporated herein by reference). Fusions between engineered zinc finger arrays and protein domains that cleave a nucleic acid can be used to generate a “zinc finger nuclease.” A zinc finger nuclease typically comprises a zinc finger domain that binds a specific target site within a nucleic acid molecule, and a nucleic acid cleavage domain that cuts the nucleic acid molecule within or in proximity to the target site bound by the binding domain. Typical engineered zinc finger nucleases comprise a binding domain having between 3 and 6 individual zinc finger motifs and binding target sites ranging from 9 base pairs to 18 base pairs in length. Longer target sites are particularly attractive in situations where it is desired to bind and cleave a target site that is unique in a given genome.

The term “zinc finger nuclease,” as used herein, refers to a nuclease comprising a nucleic acid cleavage domain conjugated to a binding domain that comprises a zinc finger array. In some embodiments, the cleavage domain is the cleavage domain of the type II restriction endonuclease FokI. Zinc finger nucleases can be designed to target virtually any desired sequence in a given nucleic acid molecule for cleavage, and the possibility to design zinc finger binding domains to bind unique sites in the context of complex genomes allows for targeted cleavage of a single genomic site in living cells, for example, to achieve a targeted genomic alteration of therapeutic value. Targeting a double-strand break to a desired genomic locus can be used to introduce frame-shift mutations into the coding sequence of a gene due to the error-prone nature of the non-homologous DNA repair pathway. Zinc finger nucleases can be generated to target a site of interest by methods well known to those of skill in the art. For example, zinc finger binding domains with a desired specificity can be designed by combining individual zinc finger motifs of known specificity. The structure of the zinc finger protein Zif268 bound to DNA has informed much of the work in this field and the concept of obtaining zinc fingers for each of the 64 possible base pair triplets and then mixing and matching these modular zinc fingers to design proteins with any desired sequence specificity has been described (Pavletich N P, Pabo C O (May 1991). “Zinc finger-DNA recognition: crystal structure of a Zif268-DNA complex at 2.1 A”. Science 252 (5007): 809-17, the entire contents of which are incorporated herein). In some embodiments, separate zinc fingers that each recognizes a 3 base pair DNA sequence are combined to generate 3-, 4-, 5-, or 6-finger arrays that recognize target sites ranging from 9 base pairs to 18 base pairs in length. In some embodiments, longer arrays are contemplated. In other embodiments, 2-finger modules recognizing 6-8 nucleotides are combined to generate 4-, 6-, or 8-zinc finger arrays. In some embodiments, bacterial or phage display is employed to develop a zinc finger domain that recognizes a desired nucleic acid sequence, for example, a desired nuclease target site of 3-30 bp in length. Zinc finger nucleases, in some embodiments, comprise a zinc finger binding domain and a cleavage domain fused or otherwise conjugated to each other via a linker, for example, a polypeptide linker. The length of the linker determines the distance of the cut from the nucleic acid sequence bound by the zinc finger domain. If a shorter linker is used, the cleavage domain will cut the nucleic acid closer to the bound nucleic acid sequence, while a longer linker will result in a greater distance between the cut and the bound nucleic acid sequence. In some embodiments, the cleavage domain of a zinc finger nuclease has to dimerize in order to cut a bound nucleic acid. In some such embodiments, the dimer is a heterodimer of two monomers, each of which comprise a different zinc finger binding domain. For example, in some embodiments, the dimer may comprise one monomer comprising zinc finger domain A conjugated to a FokI cleavage domain, and one monomer comprising zinc finger domain B conjugated to a FokI cleavage domain. In this non-limiting example, zinc finger domain A binds a nucleic acid sequence on one side of the target site, zinc finger domain B binds a nucleic acid sequence on the other side of the target site, and the dimerize FokI domain cuts the nucleic acid in between the zinc finger domain binding sites.

### DETAILED DESCRIPTION OF CERTAIN EMBODIMENTS OF THE INVENTION

The function and advantage of these and other embodiments of the present invention will be more fully understood from the Examples below. The following Examples are intended to illustrate the benefits of the present invention and to describe particular embodiments, but are not intended to exemplify the full scope of the invention. Accordingly, it will be understood that the Examples are not meant to limit the scope of the invention.

### Guide Nucleotide Sequence-Programmable DNA Binding Protein

The fusion proteins and methods described herein may use any programmable DNA binding domain.

In some embodiments, the programmable DNA binding protein domain comprises the DNA binding domain of a zinc finger nuclease (ZFN) or a transcription activator-like effector domain (TALE). In some embodiments, the programmable DNA binding protein domain may be programmed by a guide nucleotide sequence and is thus referred as a “guide nucleotide sequence-programmable DNA binding-protein domain.” In some embodiments, the guide nucleotide sequence-programmable DNA binding protein is a nuclease inactive Cas9, or dCas9. A dCas9, as used herein, encompasses a Cas9 that is completely inactive in its nuclease activity, or partially inactive in its nuclease activity (e.g., a Cas9 nickase). Thus, in some embodiments, the guide nucleotide sequence-programmable DNA binding protein is a Cas9 nickase. In some embodiments, the guide nucleotide sequence-programmable DNA binding protein is a nuclease inactive Cpf1. In some embodiments, the guide nucleotide sequence-programmable DNA binding protein is a nuclease inactive Argonaute.

In some embodiments, the guide nucleotide sequence-programmable DNA binding protein is a dCas9 domain. In some embodiments, the guide nucleotide sequence-programmable DNA binding protein is a Cas9 nickase. In some embodiments, the dCas9 domain comprises an amino acid sequence of SEQ ID NO: 2 or SEQ ID NO: 3. In some embodiments, the dCas9 domain comprises an amino acid sequence that is at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or at least 99.5% identical to any one of the Cas9 domains provided herein, and comprises mutations corresponding to D10X (X is any amino acid except for D) and/or H840X (X is any amino acid except for H) in SEQ ID NO: 1. In some embodiments, the dCas9 domain comprises an amino acid sequence that is at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or at least 99.5% identical to any one of the Cas9 domains provided herein, and comprises mutations corresponding to D10A and/or H840A in SEQ ID NO: 1. In some embodiments, the Cas9 nickase comprises an amino acid sequence that is at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or at least 99.5% identical to any one of the Cas9 domains provided herein, and comprises mutations corresponding to D10X (X is any amino acid except for D) in SEQ ID NO: 1 and a histidine at a position correspond to position 840 in SEQ ID NO: 1. In some embodiments, the Cas9 nickase comprises an amino acid sequence that is at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or at least 99.5% identical to any one of the Cas9 domains provided herein, and comprises mutations corresponding to D10A in SEQ ID NO: 1 and a histidine at a position correspond to position 840 in SEQ ID NO: 1. In some embodiments, variants or homologues of dCas9 or Cas9 nickase (e.g., variants of SEQ ID NO: 2 or SEQ ID NO: 3, respectively) are provided which are at least about 70% identical, at least about 80% identical, at least about 90% identical, at least about 95% identical, at least about 98% identical, at least about 99% identical, at least about 99.5% identical, or at least about 99.9% identical to SEQ ID NO: 2 or SEQ ID NO: 3, respectively, and comprises mutations corresponding to D10A and/or H840A in SEQ ID NO: 1. In some embodiments, variants of Cas9 (e.g., variants of SEQ ID NO: 2) are provided having amino acid sequences which are shorter, or longer than SEQ ID NO: 2, by about 5 amino acids, by about 10 amino acids, by about 15 amino acids, by about 20 amino acids, by about 25 amino acids, by about 30 amino acids, by about 40 amino acids, by about 50 amino acids, by about 75 amino acids, by about 100 amino acids, or more, provided that the dCas9 variants comprise mutations corresponding to D10A and/or H840A in SEQ ID NO: 1. In some embodiments, variants of Cas9 nickase (e.g., variants of SEQ ID NO: 3) are provided having amino acid sequences which are shorter, or longer than SEQ ID NO: 3, by about 5 amino acids, by about 10 amino acids, by about 15 amino acids, by about 20 amino acids, by about 25 amino acids, by about 30 amino acids, by about 40 amino acids, by about 50 amino acids, by about 75 amino acids, by about 100 amino acids, or more, provided that the dCas9 variants comprise mutations corresponding to D10A and comprises a histidine at a position corresponding to position 840 in SEQ ID NO: 1.

Additional suitable nuclease-inactive dCas9 domains will be apparent to those of skill in the art based on this disclosure and knowledge in the field, and are within the scope of this disclosure. Such additional exemplary suitable nuclease-inactive Cas9 domains include, but are not limited to, D10A/H840A, D10A/D839A/H840A, D10A/D839A/H840A/N863A mutant domains in SEQ ID NO: 1 (See, e.g., Prashant et al., Nature Biotechnology. 2013; 31(9): 833-838, which is incorporated herein by reference), or K603R (See, e.g., Chavez et al., Nature Methods 12, 326-328, 2015, which is incorporated herein by reference).

In some embodiments, the nucleobase editors described herein comprise a Cas9 domain with decreased electrostatic interactions between the Cas9 domain and a sugar-phosphate backbone of a DNA, as compared to a wild-type Cas9 domain. In some embodiments, a Cas9 domain comprises one or more mutations that decreases the association between the Cas9 domain and a sugar-phosphate backbone of a DNA. In some embodiments, the nucleobase editors described herein comprises a dCas9 (e.g., with D10A and H840A mutations in SEQ ID NO: 1) or a Cas9 nickase (e.g., with D10A mutation in SEQ ID NO: 1), wherein the dCas9 or the Cas9 nickase further comprises one or more of a N497X, a R661X, a Q695X, and/or a Q926X mutation of the amino acid sequence provided in SEQ ID NO: 10, or a corresponding mutation in any of the amino acid sequences provided in SEQ ID NOs: 11-260, wherein X is any amino acid. In some embodiments, the nucleobase editors described herein comprises a dCas9 (e.g., with D10A and H840A mutations in SEQ ID NO: 1) or a Cas9 nickase (e.g., with D10A mutation in SEQ ID NO: 1), wherein the dCas9 or the Cas9 nickase further comprises one or more of a N497A, a R661A, a Q695A, and/or a Q926A mutation of the amino acid sequence provided in SEQ ID NO: 10, or a corresponding mutation in any of the amino acid sequences provided in SEQ ID NOs: 11-260. In some embodiments, the Cas9 domain (e.g., of any of the nucleobase editors provided herein) comprises the amino acid sequence as set forth in SEQ ID NO: 720. In some embodiments, the nucleobase editor comprises the amino acid sequence as set forth in SEQ ID NO: 721. Cas9 domains with high fidelity are known in the art and would be apparent to the skilled artisan. For example, Cas9 domains with high fidelity have been described in Kleinstiver, B. P., et al. “High-fidelity CRISPR-Cas9 nucleases with no detectable genome-wide off-target effects.” Nature 529, 490-495 (2016); and Slaymaker, I. M., et al. “Rationally engineered Cas9 nucleases with improved specificity.” Science 351, 84-88 (2015); the entire contents of each are incorporated herein by reference.

Cas9 variant with decreased electrostatic interactions
between the Cas9 and DNA backbone
(SEQ ID NO: 720)
DKKYSIGLAIGTNSVGWAVITDEYKVPSKKFKVLGNTDRHSIKKNLIGALLFDSGETA
EATRLKRTARRRYTRRKNRICYLQEIFSNEMAKVDDSFFHRLEESFLVEEDKKHERH
PIFGNIVDEVAYHEKYPTIYHLRKKLVDSTDKADLRLIYLALAHMIKFRGHFLIEGDL
NPDNSDVDKLFIQLVQTYNQLFEENPINASGVDAKAILSARLSKSRRLENLIAQLPGE
KKNGLFGNLIALSLGLTPNFKSNFDLAEDAKLQLSKDTYDDDLDNLLAQIGDQYADL
FLAAKNLSDAILLSDILRVNTEITKAPLSASMIKRYDEHHQDLTLLKALVRQQLPEKY
KEIFFDQSKNGYAGYIDGGASQEEFYKFIKPILEKMDGTEELLVKLNREDLLRKQRTF
DNGSIPHQIHLGELHAILRRQEDFYPFLKDNREKIEKILTFRIPYYVGPLARGNSRFAW
MTRKSEETITPWNFEEVVDKGASAQSFIERMTAFDKNLPNEKVLPKHSLLYEYFTVY
NELTKVKYVTEGMRKPAFLSGEQKKAIVDLLFKTNRKVTVKQLKEDYFKKIECFDS
VEISGVEDRFNASLGTYHDLLKIIKDKDFLDNEENEDILEDIVLTLTLFEDREMIEERL
KTYAHLFDDKVMKQLKRRRYTGWGALSRKLINGIRDKQSGKTILDFLKSDGFANRN
FMALIHDDSLTFKEDIQKAQVSGQGDSLHEHIANLAGSPAIKKGILQTVKVVDELVK
VMGRHKPENIVIEMARENQTTQKGQKNSRERMKRIEEGIKELGSQILKEHPVENTQL
QNEKLYLYYLQNGRDMYVDQELDINRLSDYDVDHIVPQSFLKDDSIDNKVLTRSDK
NRGKSDNVPSEEVVKKMKNYWRQLLNAKLITQRKFDNLTKAERGGLSELDKAGFIK
RQLVETRAITKHVAQILDSRMNTKYDENDKLIREVKVITLKSKLVSDFRKDFQFYKV
REINNYHHAHDAYLNAVVGTALIKKYPKLESEFVYGDYKVYDVRKMIAKSEQEIGK
ATAKYFFYSNIMNFFKTEITLANGEIRKRPLIETNGETGEIVWDKGRDFATVRKVLSM
PQVNIVKKTEVQTGGFSKESILPKRNSDKLIARKKDWDPKKYGGFDSPTVAYSVLVV
AKVEKGKSKKLKSVKELLGITIMERSSFEKNPIDFLEAKGYKEVKKDLIIKLPKYSLFE
LENGRKRMLASAGELQKGNELALPSKYVNFLYLASHYEKLKGSPEDNEQKQLFVEQ
HKHYLDEIIEQISEFSKRVILADANLDKVLSAYNKHRDKPIREQAENIIHLFTLTNLGA
PAAFKYFDTTIDRKRYTSTKEVLDATLIHQSITGLYETRIDLSQLGGD
High fidelity nucleobase editor
(SEQ ID NO: 721)
MSSETGPVAVDPTLRRRIEPHEFEVFFDPRELRKETCLLYEINWGGRHSIWRHTSQNT
NKHVEVNFIEKFTTERYFCPNTRCSITWFLSWSPCGECSRAITEFLSRYPHVTLFIYIAR
LYHHADPRNRQGLRDLISSGVTIQIMTEQESGYCWRNFVNYSPSNEAHWPRYPHLW
VRLYVLELYCIILGLPPCLNILRRKQPQLTFFTIALQSCHYQRLPPHILWATGLKSGSET
PGTSESATPESDKKYSIGLAIGTNSVGWAVITDEYKVPSKKFKVLGNTDRHSIKKNLI
GALLFDSGETAEATRLKRTARRRYTRRKNRICYLQEIFSNEMAKVDDSFFHRLEESFL
VEEDKKHERHPIFGNIVDEVAYHEKYPTIYHLRKKLVDSTDKADLRLIYLALAHMIK
FRGHFLIEGDLNPDNSDVDKLFIQLVQTYNQLFEENPINASGVDAKAILSARLSKSRR
LENLIAQLPGEKKNGLFGNLIALSLGLTPNFKSNFDLAEDAKLQLSKDTYDDDLDNL
LAQIGDQYADLFLAAKNLSDAILLSDILRVNTEITKAPLSASMIKRYDEHHQDLTLLK
ALVRQQLPEKYKEIFFDQSKNGYAGYIDGGASQEEFYKFIKPILEKMDGTEELLVKLN
REDLLRKQRTFDNGSIPHQIHLGELHAILRRQEDFYPFLKDNREKIEKILTFRIPYYVGP
LARGNSRFAWMTRKSEETITPWNFEEVVDKGASAQSFIERMTAFDKNLPNEKVLPK
HSLLYEYFTVYNELTKVKYVTEGMRKPAFLSGEQKKAIVDLLFKTNRKVTVKQLKE
DYFKKIECFDSVEISGVEDRFNASLGTYHDLLKIIKDKDFLDNEENEDILEDIVLTLTLF
EDREMIEERLKTYAHLFDDKVMKQLKRRRYTGWGALSRKLINGIRDKQSGKTILDFL
KSDGFANRNFMALIHDDSLTFKEDIQKAQVSGQGDSLHEHIANLAGSPAIKKGILQTV
KVVDELVKVMGRHKPENIVIEMARENQTTQKGQKNSRERMKRIEEGIKELGSQILKE
HPVENTQLQNEKLYLYYLQNGRDMYVDQELDINRLSDYDVDHIVPQSFLKDDSIDN
KVLTRSDKNRGKSDNVPSEEVVKKMKNYWRQLLNAKLITQRKFDNLTKAERGGLS
ELDKAGFIKRQLVETRAITKHVAQILDSRMNTKYDENDKLIREVKVITLKSKLVSDFR
KDFQFYKVREINNYHHAHDAYLNAVVGTALIKKYPKLESEFVYGDYKVYDVRKMI
AKSEQEIGKATAKYFFYSNIMNFFKTEITLANGEIRKRPLIETNGETGEIVWDKGRDFA
TVRKVLSMPQVNIVKKTEVQTGGFSKESILPKRNSDKLIARKKDWDPKKYGGFDSPT
VAYSVLVVAKVEKGKSKKLKSVKELLGITIMERSSFEKNPIDFLEAKGYKEVKKDLII
KLPKYSLFELENGRKRMLASAGELQKGNELALPSKYVNFLYLASHYEKLKGSPEDN
EQKQLFVEQHKHYLDEIIEQISEFSKRVILADANLDKVLSAYNKHRDKPIREQAENIIH
LFTLTNLGAPAAFKYFDTTIDRKRYTSTKEVLDATLIHQSITGLYETRIDLSQLGGD

The Cas9 protein recognizes a short motif (PAM motif) within the target DNA sequence, which is required for the Cas9-DNA interaction but that is not determined by complementarity to the guide RNA nucleotide sequence. A “PAM motif” or “protospacer adjacent motif,” as used herein, refers to a DNA sequence adjacent to the 5′- or 3′-immediately following the DNA sequence that is complementary to the guide RNA oligonucleotide sequence. Cas9 will not successfully bind to, cleave, or nick the target DNA sequence if it is not followed by an appropriate PAM sequence. Without wishing to be bound by any particular theory, specific amino acid residues in the Cas9 enzyme are responsible for interacting with the bases of the PAM and determine the PAM specificity. Therefore, changes in these residues or nearby residues leads to a different or relaxed PAM specificity. Changing or relaxing the PAM specificity may shift the places where Cas9 can bind, as will be apparent to those of skill in the art based on the instant disclosure.

Wild-type Streptococcus pyogenes Cas9 recognizes a canonical PAM sequence (5′-NGG-3′). Other Cas9 nucleases (e.g., Cas9 from Streptococcus thermophiles, Staphylococcus aureus, Neisseria meningitidis, or Treponema denticolaor) and Cas9 variants thereof have been described in the art to have different, or more relaxed PAM requirements. For example, in Kleinstiver et al., Nature 523, 481-485, 2015; Klenstiver et al., Nature 529, 490-495, 2016; Ran et al., Nature, April 9; 520(7546): 186-191, 2015; Kleinstiver et al., Nat Biotechnol, 33(12):1293-1298, 2015; Hou et al., Proc Natl Acad Sci USA, 110(39):15644-9, 2014; Prykhozhij et al., PLoS One, 10(3): e0119372, 2015; Zetsche et al., Cell 163, 759-771, 2015; Gao et al., Nature Biotechnology, doi:10.1038/nbt.3547, 2016; Want et al., Nature 461, 754-761, 2009; Chavez et al., doi: dx.doi dot org/10.1101/058974; Fagerlund et al., Genome Biol. 2015; 16: 25, 2015; Zetsche et al., Cell, 163, 759-771, 2015; and Swarts et al., Nat Struct Mol Biol, 21(9):743-53, 2014, each of which is incorporated herein by reference.

Thus, the guide nucleotide sequence-programmable DNA-binding protein of the present disclosure may recognize a variety of PAM sequences including, without limitation PAM sequences that are on the 3′ or the 5′ end of the DNA sequence determined by the guide RNA. For example, the sequence may be: NGG, NGAN (SEQ ID NO: 741), NGNG (SEQ ID NO: 742), NGAG (SEQ ID NO: 743), NGCG (SEQ ID NO: 744), NNGRRT (SEQ ID NO: 745), NGRRN (SEQ ID NO: 746), NNNRRT (SEQ ID NO: 747), NNNGATT (SEQ ID NO: 748), NNAGAAW (SEQ ID NO: 749), NAAAC (SEQ ID NO: 750), TTN, TTTN (SEQ ID NO: 751), and YTN, wherein Y is a pyrimidine, R is a purine, and N is any nucleobase.

Some aspects of the disclosure provide RNA-programmable DNA binding proteins, which may be used to guide a protein, such as a base editor, to a specific nucleic acid (e.g., DNA or RNA) sequence. Nucleic acid programmable DNA binding proteins include, without limitation, Cas9 (e.g., dCas9 and nCas9), CasX, CasY, Cpf1, C2c1, C2c2, C2C3, and Argonaute. One example of an RNA-programmable DNA-binding protein that has different PAM specificity is Clustered Regularly Interspaced Short Palindromic Repeats from Prevotella and Francisella 1 (Cpf1). Similar to Cas9, Cpf1 is also a class 2 CRISPR effector. It has been shown that Cpf1 mediates robust DNA interference with features distinct from Cas9. Cpf1 is a single RNA-guided endonuclease lacking tracrRNA, and it may utilize a T-rich protospacer-adjacent motif (e.g., TTN, TTTN (SEQ ID NO: 751), or YTN), which is on the 5′-end of the DNA sequence determined by the guide RNA. Moreover, Cpf1 cleaves DNA via a staggered DNA double-stranded break. Out of 16 Cpf1-family proteins, two enzymes from Acidaminococcus and Lachnospiraceae are shown to have efficient genome-editing activity in human cells. Cpf1 proteins are known in the art and have been described previously, for example Yamano et al., “Crystal structure of Cpf1 in complex with guide RNA and target DNA.” Cell (165) 2016, p. 949-962; the entire contents of which is hereby incorporated by reference.

Also useful in the present compositions and methods are nuclease-inactive Cpf1 (dCpf1) variants that may be used as a guide nucleotide sequence-programmable DNA-binding protein domain. The Cpf1 protein has a RuvC-like endonuclease domain that is similar to the RuvC domain of Cas9 but does not have a HNH endonuclease domain, and the N-terminal of Cpf1 does not have the alfa-helical recognition lobe of Cas9. It was shown in Zetsche et al., Cell, 163, 759-771, 2015 (which is incorporated herein by reference) that, the RuvC-like domain of Cpf1 is responsible for cleaving both DNA strands and inactivation of the RuvC-like domain inactivates Cpf1 nuclease activity. For example, mutations corresponding to D917A, E1006A, or D1255A in Francisella novicida Cpf1 (SEQ ID NO: 714) inactivate Cpf1 nuclease activity. In some embodiments, the dCpf1 of the present disclosure may comprise mutations corresponding to D917A, E1006A, D1255A, D917A/E1006A, D917A/D1255A, E1006A/D1255A, or D917A/E1006A/D1255A in SEQ ID NO: 714. In other embodiments, the Cpf1 nickase of the present disclosure may comprise mutations corresponding to D917A, E1006A, D1255A, D917A/E1006A, D917A/D1255A, E1006A/D1255A, or D917A/E1006A/D1255A in SEQ ID NO: 714. A Cpf1 nickase useful for the embodiments of the instant disclosure may comprise other mutations and/or further mutations known in the field. It is to be understood that any mutations, e.g., substitution mutations, deletions, or insertions that fully or partially inactivates the RuvC domain of Cpf1 may be used in accordance with the present disclosure, and that these mutations of Cpf1 may result in, for example, a dCpf1 or Cpf1 nickase.

Thus, in some embodiments, the guide nucleotide sequence-programmable DNA binding protein is a nuclease inactive Cpf1 (dCpf1). In some embodiments, the dCpf1 comprises an amino acid sequence of any one SEQ ID NOs: 714-717. In some embodiments, the dCpf1 comprises an amino acid sequence that is at least 85%, at least 90%, at least 91%, at least 92%, at least 93%, at least 94%, at least 95%, at least 96%, at least 97%, at least 98%, at least 99%, or at ease 99.5% identical to any one of SEQ ID NOs: 714-717, and comprises mutations corresponding to D917A, E1006A, D1255A, D917A/E1006A, D917A/D1255A, E1006A/D1255A, or D917A/E1006A/D1255A in SEQ ID NO: 714. Cpf1 from other bacterial species may also be used in accordance with the present disclosure, as a dCpf1 or Cpf1 nickase.

Wild type Francisella novicida Cpf1 (D917, E1006, and D1255
are bolded and underlined)
(SEQ ID NO: 714)
MSIYQEFVNKYSLSKTLRFELIPQGKTLENIKARGLILDDEKRAKDYKKAKQIIDKYH
QFFIEEILSSVCISEDLLQNYSDVYFKLKKSDDDNLQKDFKSAKDTIKKQISEYIKDSE
KFKNLFNQNLIDAKKGQESDLILWLKQSKDNGIELFKANSDITDIDEALEIIKSFKGWT
TYFKGFHENRKNVYSSNDIPTSIIYRIVDDNLPKFLENKAKYESLKDKAPEAINYEQIK
KDLAEELTFDIDYKTSEVNQRVFSLDEVFEIANFNNYLNQSGITKFNTIIGGKFVNGEN
TKRKGINEYINLYSQQINDKTLKKYKMSVLFKQILSDTESKSFVIDKLEDDSDVVTTM
QSFYEQIAAFKTVEEKSIKETLSLLFDDLKAQKLDLSKIYFKNDKSLTDLSQQVFDDY
SVIGTAVLEYITQQIAPKNLDNPSKKEQELIAKKTEKAKYLSLETIKLALEEFNKHRDI
DKQCRFEEILANFAAIPMIFDEIAQNKDNLAQISIKYQNQGKKDLLQASAEDDVKAIK
DLLDQTNNLLHKLKIFHISQSEDKANILDKDEHFYLVFEECYFELANIVPLYNKIRNYI
TQKPYSDEKFKLNFENSTLANGWDKNKEPDNTAILFIKDDKYYLGVMNKKNNKIFD
DKAIKENKGEGYKKIVYKLLPGANKMLPKVFFSAKSIKFYNPSEDILRIRNHSTHTKN
GSPQKGYEKFEFNIEDCRKFIDFYKQSISKHPEWKDFGFRFSDTQRYNSIDEFYREVE
NQGYKLTFENISESYIDSVVNQGKLYLFQIYNKDFSAYSKGRPNLHTLYWKALFDER
NLQDVVYKLNGEAELFYRKQSIPKKITHPAKEAIANKNKDNPKKESVFEYDLIKDKR
FTEDKFFFHCPITINFKSSGANKFNDEINLLLKEKANDVHILSIDRGERHLAYYTLVDG
KGNIIKQDTFNIIGNDRMKTNYHDKLAAIEKDRDSARKDWKKINNIKEMKEGYLSQV
VHEIAKLVIEYNAIVVFEDLNFGFKRGRFKVEKQVYQKLEKMLIEKLNYLVFKDNEF
DKTGGVLRAYQLTAPFETFKKMGKQTGIIYYVPAGFTSKICPVTGFVNQLYPKYESV
SKSQEFFSKFDKICYNLDKGYFEFSFDYKNFGDKAAKGKWTIASFGSRLINFRNSDKN
HNWDTREVYPTKELEKLLKDYSIEYGHGECIKAAICGESDKKFFAKLTSVLNTILQM
RNSKTGTELDYLISPVADVNGNFFDSRQAPKNMPQDADANGAYHIGLKGLMLLGRI
KNNQEGKKLNLVIKNEEYFEFVQNRNN
Francisella novicida Cpf1 D917A
(SEQ ID NO: 715)
MSIYQEFVNKYSLSKTLRFELIPQGKTLENIKARGLILDDEKRAKDYKKAKQIIDKYH
QFFIEEILSSVCISEDLLQNYSDVYFKLKKSDDDNLQKDFKSAKDTIKKQISEYIKDSE
KFKNLFNQNLIDAKKGQESDLILWLKQSKDNGIELFKANSDITDIDEALEIIKSFKGWT
TYFKGFHENRKNVYSSNDIPTSIIYRIVDDNLPKFLENKAKYESLKDKAPEAINYEQIK
KDLAEELTFDIDYKTSEVNQRVFSLDEVFEIANFNNYLNQSGITKFNTIIGGKFVNGEN
TKRKGINEYINLYSQQINDKTLKKYKMSVLFKQILSDTESKSFVIDKLEDDSDVVTTM
QSFYEQIAAFKTVEEKSIKETLSLLFDDLKAQKLDLSKIYFKNDKSLTDLSQQVFDDY
SVIGTAVLEYITQQIAPKNLDNPSKKEQELIAKKTEKAKYLSLETIKLALEEFNKHRDI
DKQCRFEEILANFAAIPMIFDEIAQNKDNLAQISIKYQNQGKKDLLQASAEDDVKAIK
DLLDQTNNLLHKLKIFHISQSEDKANILDKDEHFYLVFEECYFELANIVPLYNKIRNYI
TQKPYSDEKFKLNFENSTLANGWDKNKEPDNTAILFIKDDKYYLGVMNKKNNKIFD
DKAIKENKGEGYKKIVYKLLPGANKMLPKVFFSAKSIKFYNPSEDILRIRNHSTHTKN
GSPQKGYEKFEFNIEDCRKFIDFYKQSISKHPEWKDFGFRFSDTQRYNSIDEFYREVE
NQGYKLTFENISESYIDSVVNQGKLYLFQIYNKDFSAYSKGRPNLHTLYWKALFDER
NLQDVVYKLNGEAELFYRKQSIPKKITHPAKEAIANKNKDNPKKESVFEYDLIKDKR
FTEDKFFFHCPITINFKSSGANKFNDEINLLLKEKANDVHILSIARGERHLAYYTLVDG
KGNIIKQDTFNIIGNDRMKTNYHDKLAAIEKDRDSARKDWKKINNIKEMKEGYLSQV
VHEIAKLVIEYNAIVVFEDLNFGFKRGRFKVEKQVYQKLEKMLIEKLNYLVFKDNEF
DKTGGVLRAYQLTAPFETFKKMGKQTGIIYYVPAGFTSKICPVTGFVNQLYPKYESV
SKSQEFFSKFDKICYNLDKGYFEFSFDYKNFGDKAAKGKWTIASFGSRLINFRNSDKN
HNWDTREVYPTKELEKLLKDYSIEYGHGECIKAAICGESDKKFFAKLTSVLNTILQM
RNSKTGTELDYLISPVADVNGNFFDSRQAPKNMPQDADANGAYHIGLKGLMLLGRI
KNNQEGKKLNLVIKNEEYFEFVQNRNN
Francisella novicida Cpf1 E1006A
(SEQ ID NO: 716)
MSIYQEFVNKYSLSKTLRFELIPQGKTLENIKARGLILDDEKRAKDYKKAKQIIDKYH
QFFIEEILSSVCISEDLLQNYSDVYFKLKKSDDDNLQKDFKSAKDTIKKQISEYIKDSE
KFKNLFNQNLIDAKKGQESDLILWLKQSKDNGIELFKANSDITDIDEALEIIKSFKGWT
TYFKGFHENRKNVYSSNDIPTSIIYRIVDDNLPKFLENKAKYESLKDKAPEAINYEQIK
KDLAEELTFDIDYKTSEVNQRVFSLDEVFEIANFNNYLNQSGITKFNTIIGGKFVNGEN
TKRKGINEYINLYSQQINDKTLKKYKMSVLFKQILSDTESKSFVIDKLEDDSDVVTTM
QSFYEQIAAFKTVEEKSIKETLSLLFDDLKAQKLDLSKIYFKNDKSLTDLSQQVFDDY
SVIGTAVLEYITQQIAPKNLDNPSKKEQELIAKKTEKAKYLSLETIKLALEEFNKHRDI
DKQCRFEEILANFAAIPMIFDEIAQNKDNLAQISIKYQNQGKKDLLQASAEDDVKAIK
DLLDQTNNLLHKLKIFHISQSEDKANILDKDEHFYLVFEECYFELANIVPLYNKIRNYI
TQKPYSDEKFKLNFENSTLANGWDKNKEPDNTAILFIKDDKYYLGVMNKKNNKIFD
DKAIKENKGEGYKKIVYKLLPGANKMLPKVFFSAKSIKFYNPSEDILRIRNHSTHTKN
GSPQKGYEKFEFNIEDCRKFIDFYKQSISKHPEWKDFGFRFSDTQRYNSIDEFYREVE
NQGYKLTFENISESYIDSVVNQGKLYLFQIYNKDFSAYSKGRPNLHTLYWKALFDER
NLQDVVYKLNGEAELFYRKQSIPKKITHPAKEAIANKNKDNPKKESVFEYDLIKDKR
FTEDKFFFHCPITINFKSSGANKFNDEINLLLKEKANDVHILSIDRGERHLAYYTLVDG
KGNIIKQDTFNIIGNDRMKTNYHDKLAAIEKDRDSARKDWKKINNIKEMKEGYLSQV
VHEIAKLVIEYNAIVVFADLNFGFKRGRFKVEKQVYQKLEKMLIEKLNYLVFKDNEF
DKTGGVLRAYQLTAPFETFKKMGKQTGIIYYVPAGFTSKICPVTGFVNQLYPKYESV
SKSQEFFSKFDKICYNLDKGYFEFSFDYKNFGDKAAKGKWTIASFGSRLINFRNSDKN
HNWDTREVYPTKELEKLLKDYSIEYGHGECIKAAICGESDKKFFAKLTSVLNTILQM
RNSKTGTELDYLISPVADVNGNFFDSRQAPKNMPQDADANGAYHIGLKGLMLLGRI
KNNQEGKKLNLVIKNEEYFEFVQNRNN
Francisella novicida Cpf1 D1255A
(SEQ ID NO: 717)
MSIYQEFVNKYSLSKTLRFELIPQGKTLENIKARGLILDDEKRAKDYKKAKQIIDKYH
QFFIEEILSSVCISEDLLQNYSDVYFKLKKSDDDNLQKDFKSAKDTIKKQISEYIKDSE
KFKNLFNQNLIDAKKGQESDLILWLKQSKDNGIELFKANSDITDIDEALEIIKSFKGWT
TYFKGFHENRKNVYSSNDIPTSIIYRIVDDNLPKFLENKAKYESLKDKAPEAINYEQIK
KDLAEELTFDIDYKTSEVNQRVFSLDEVFEIANFNNYLNQSGITKFNTIIGGKFVNGEN
TKRKGINEYINLYSQQINDKTLKKYKMSVLFKQILSDTESKSFVIDKLEDDSDVVTTM
QSFYEQIAAFKTVEEKSIKETLSLLFDDLKAQKLDLSKIYFKNDKSLTDLSQQVFDDY
SVIGTAVLEYITQQIAPKNLDNPSKKEQELIAKKTEKAKYLSLETIKLALEEFNKHRDI
DKQCRFEEILANFAAIPMIFDEIAQNKDNLAQISIKYQNQGKKDLLQASAEDDVKAIK
DLLDQTNNLLHKLKIFHISQSEDKANILDKDEHFYLVFEECYFELANIVPLYNKIRNYI
TQKPYSDEKFKLNFENSTLANGWDKNKEPDNTAILFIKDDKYYLGVMNKKNNKIFD
DKAIKENKGEGYKKIVYKLLPGANKMLPKVFFSAKSIKFYNPSEDILRIRNHSTHTKN
GSPQKGYEKFEFNIEDCRKFIDFYKQSISKHPEWKDFGFRFSDTQRYNSIDEFYREVE
NQGYKLTFENISESYIDSVVNQGKLYLFQIYNKDFSAYSKGRPNLHTLYWKALFDER
NLQDVVYKLNGEAELFYRKQSIPKKITHPAKEAIANKNKDNPKKESVFEYDLIKDKR
FTEDKFFFHCPITINFKSSGANKFNDEINLLLKEKANDVHILSIDRGERHLAYYTLVDG
KGNIIKQDTFNIIGNDRMKTNYHDKLAAIEKDRDSARKDWKKINNIKEMKEGYLSQV
VHEIAKLVIEYNAIVVFEDLNFGFKRGRFKVEKQVYQKLEKMLIEKLNYLVFKDNEF
DKTGGVLRAYQLTAPFETFKKMGKQTGIIYYVPAGFTSKICPVTGFVNQLYPKYESV
SKSQEFFSKFDKICYNLDKGYFEFSFDYKNFGDKAAKGKWTIASFGSRLINFRNSDKN
HNWDTREVYPTKELEKLLKDYSIEYGHGECIKAAICGESDKKFFAKLTSVLNTILQM
RNSKTGTELDYLISPVADVNGNFFDSRQAPKNMPQDAAANGAYHIGLKGLMLLGRI
KNNQEGKKLNLVIKNEEYFEFVQNRNN

In addition to Cas9 and Cpf1, three distinct Class 2 CRISPR-Cas systems (C2c1, C2c2, and C2c3) have been described by Shmakov et al., “Discovery and Functional Characterization of Diverse Class 2 CRISPR Cas Systems”, Mol. Cell, 2015 November 5; 60(3): 385-397, the entire contents of which is hereby incorporated by reference. Effectors of two of the systems, C2c1 and C2c3, contain RuvC-like endonuclease domains related to Cpf1. A third system, C2c2 contains an effector with two predicated HEPN RNase domains. Production of mature CRISPR RNA is tracrRNA-independent, unlike production of CRISPR RNA by C2c1. C2c1 depends on both CRISPR RNA and tracrRNA for DNA cleavage. Bacterial C2c2 has been shown to possess a unique RNase activity for CRISPR RNA maturation distinct from its RNA-activated single-stranded RNA degradation activity. These RNase functions are different from each other and from the CRISPR RNA-processing behavior of Cpf1. See, e.g., East-Seletsky, et al., “Two distinct RNase activities of CRISPR-C2c2 enable guide-RNA processing and RNA detection”, Nature, 2016 Oct. 13; 538(7624):270-273, the entire contents of which are hereby incorporated by reference. In vitro biochemical analysis of C2c2 in Leptotrichia shahii has shown that C2c2 is guided by a single CRISPR RNA and can be programed to cleave ssRNA targets carrying complementary protospacers. Catalytic residues in the two conserved HEPN domains mediate cleavage. Mutations in the catalytic residues generate catalytically inactive RNA-binding proteins. See e.g., Abudayyeh et al., “C2c2 is a single-component programmable RNA-guided RNA-targeting CRISPR effector”, Science, 2016 Aug. 5; 353(6299), the entire contents of which are hereby incorporated by reference.

The crystal structure of Alicyclobaccillus acidoterrastris C2c1 (AacC2c1) has been reported in complex with a chimeric single-molecule guide RNA (sgRNA). See, e.g., Liu et al., “C2c1-sgRNA Complex Structure Reveals RNA-Guided DNA Cleavage Mechanism”, Mol. Cell, 2017 Jan. 19; 65(2):310-322, the entire contents of which are hereby incorporated by reference. The crystal structure has also been reported in Alicyclobacillus acidoterrestris C2c1 bound to target DNAs as ternary complexes. See, e.g., Yang et al., “PAM-dependent Target DNA Recognition and Cleavage by C2C1 CRISPR-Cas endonuclease”, Cell, 2016 Dec. 15; 167(7):1814-1828, the entire contents of which are hereby incorporated by reference. Catalytically competent conformations of AacC2c1, both with target and non-target DNA strands, have been captured independently positioned within a single RuvC catalytic pocket, with C2c1-mediated cleavage resulting in a staggered seven-nucleotide break of target DNA. Structural comparisons between C2c1 ternary complexes and previously identified Cas9 and Cpf1 counterparts demonstrate the diversity of mechanisms used by CRISPR-Cas9 systems.

In some embodiments, the guide nucleotide sequence-programmable DNA-binding protein of any of the fusion proteins provided herein may be a C2c1, a C2c2, or a C2c3 protein. In some embodiments, the guide nucleotide sequence-programmable DNA-binding protein is a C2c1 protein. In some embodiments, the guide nucleotide sequence-programmable DNA-binding protein is a C2c2 protein. In some embodiments, the guide nucleotide sequence-programmable DNA-binding protein is a C2c3 protein. In some embodiments, the guide nucleotide sequence-programmable DNA-binding protein comprises an amino acid sequence that is at least 85%, at least 90%, at least 91%, at least 92%, at least 93%, at least 94%, at least 95%, at least 96%, at least 97%, at least 98%, at least 99%, or at least 99.5% identical to a naturally-occurring C2c1, C2c2, or C2c3 protein. In some embodiments, the guide nucleotide sequence-programmable DNA-binding protein is a naturally-occurring C2c1, C2c2, or C2c3 protein. In some embodiments, the guide nucleotide sequence-programmable DNA-binding protein comprises an amino acid sequence that is at least 85%, at least 90%, at least 91%, at least 92%, at least 93%, at least 94%, at least 95%, at least 96%, at least 97%, at least 98%, at least 99%, or at least 99.5% identical to any of the C2c1, C2c2, or C2c3 proteins described herein. In some embodiments, the guide nucleotide sequence-programmable DNA-binding protein comprises an amino acid sequence of any one of the C2c1, C2c2, or C2c3 proteins described herein. It should be appreciated that C2c1, C2c2, or C2c3 from other bacterial species may also be used in accordance with the present disclosure.

C2c1 (uniprot.org/uniprot/T0D7A2#)
sp|T0D7A2|C2C1_ALIAG CRISPR-associated
endonuclease C2c1 OS = Alicyclobacillus
acidoterrestris (strain ATCC 49025/DSM 3922/
CIP 6132/NCIMB 13137/GD3B) GN = c2c1
PE = 1 SV = 1
(SEQ ID NO: 762)
MAVKSIKVKLRLDDMPEIRAGLWKLHKEVNAGVRYYTEWLSLLRQENLYR
RSPNGDGEQECDKTAEECKAELLERLRARQVENGHRGPAGSDDELLQLAR
QLYELLVPQAIGAKGDAQQIARKFLSPLADKDAVGGLGIAKAGNKPRWVR
MREAGEPGWEEEKEKAETRKSADRTADVLRALADFGLKPLMRVYTDSEMS
SVEWKPLRKGQAVRTWDRDMFQQAIERMMSWESWNQRVGQEYAKLVEQKN
RFEQKNFVGQEHLVHLVNQLQQDMKEASPGLESKEQTAHYVTGRALRGSD
KVFEKWGKLAPDAPFDLYDAEIKNVQRRNTRRFGSHDLFAKLAEPEYQAL
WREDASFLTRYAVYNSILRKLNHAKMFATFTLPDATAHPIWTRFDKLGGN
LHQYTFLFNEFGERRHAIRFHKLLKVENGVAREVDDVTVPISMSEQLDNL
LPRDPNEPIALYFRDYGAEQHFTGEFGGAKIQCRRDQLAHMHRRRGARDV
YLNVSVRVQSQSEARGERRPPYAAVFRLVGDNHRAFVHFDKLSDYLAEHP
DDGKLGSEGLLSGLRVMSVDLGLRTSASISVFRVARKDELKPNSKGRVPF
FFPIKGNDNLVAVHERSQLLKLPGETESKDLRAIREERQRTLRQLRTQLA
YLRLLVRCGSEDVGRRERSWAKLIEQPVDAANHMTPDWREAFENELQKLK
SLHGICSDKEWMDAVYESVRRVWRHMGKQVRDWRKDVRSGERPKIRGYAK
DVVGGNSIEQIEYLERQYKFLKSWSFFGKVSGQVIRAEKGSRFAITLREH
IDHAKEDRLKKLADRIIMEALGYVYALDERGKGKWVAKYPPCQLILLEEL
SEYQFNNDRPPSENNQLMQWSHRGVFQELINQAQVHDLLVGTMYAAFSSR
FDARTGAPGIRCRRVPARCTQEHNPEPFPWWLNKFVVEHTLDACPLRADD
LIPTGEGEIFVSPFSAEEGDFHQIHADLNAAQNLQQRLWSDFDISQIRLR
CDWGEVDGELVLIPRLTGKRTADSYSNKVFYTNTGVTYYERERGKKRRKV
FAQEKLSEEEAELLVEADEAREKSVVLMRDPSGIINRGNWTRQKEFWSMV
NQRIEGYLVKQIRSRVPLQDSACENTGDI
C2c2 (uniprot.org/uniprot/P0DOC6)
>sp|P0DOC6|C2C2_LEPSD CRISPR-
associated endoribonuclease C2c2 OS =
Leptotrichiashahii (strain DSM 19757/CCUG 47503/
CIP 107916/JCM 16776/LB37) GN = c2c2
PE = 1 SV = 1
(SEQ ID NO: 764)
MGNLFGHKRWYEVRDKKDFKIKRKVKVKRNYDGNKYILNINENNNKEKID
NNKFIRKYINYKKNDNILKEFTRKFHAGNILFKLKGKEGIIRIENNDDFL
ETEEVVLYIEAYGKSEKLKALGITKKKIIDEAIRQGITKDDKKIEIKRQE
NEEEIEIDIRDEYTNKTLNDCSIILRIIENDELETKKSIYEIFKNINMSL
YKIIEKIIENETEKVFENRYYEEHLREKLLKDDKIDVILTNFMEIREKIK
SNLEILGFVKFYLNVGGDKKKSKNKKMLVEKILNINVDLTVEDIADFVIK
ELEFWNITKRIEKVKKVNNEFLEKRRNRTYIKSYVLLDKHEKFKIERENK
KDKIVKFFVENIKNNSIKEKIEKILAEFKIDELIKKLEKELKKGNCDTEI
FGIFKKHYKVNFDSKKFSKKSDEEKELYKIIYRYLKGRIEKILVNEQKVR
LKKMEKIEIEKILNESILSEKILKRVKQYTLEHIMYLGKLRHNDIDMTTV
NTDDFSRLHAKEELDLELITFFASTNMELNKIFSRENINNDENIDFFGGD
REKNYVLDKKILNSKIKIIRDLDFIDNKNNITNNFIRKFTKIGTNERNRI
LHAISKERDLQGTQDDYNKVINIIQNLKISDEEVSKALNLDVVFKDKKNI
ITKINDIKISEENNNDIKYLPSFSKVLPEILNLYRNNPKNEPFDTIETEK
IVLNALIYVNKELYKKLILEDDLEENESKNIFLQELKKTLGNIDEIDENI
IENYYKNAQISASKGNNKAIKKYQKKVIECYIGYLRKNYEELFDFSDFKM
NIQEIKKQIKDINDNKTYERITVKTSDKTIVINDDFEYIISIFALLNSNA
VINKIRNRFFATSVWLNTSEYQNIIDILDEIMQLNTLRNECITENWNLNL
EEFIQKMKEIEKDFDDFKIQTKKEIFNNYYEDIKNNILTEFKDDINGCDV
LEKKLEKIVIFDDETKFEIDKKSNILQDEQRKLSNINKKDLKKKVDQYIK
DKDQEIKSKILCRIIFNSDFLKKYKKEIDNLIEDMESENENKFQEIYYPK
ERKNELYIYKKNLFLNIGNPNFDKIYGLISNDIKMADAKFLFNIDGKNIR
KNKISEIDAILKNLNDKLNGYSKEYKEKYIKKLKENDDFFAKNIQNKNYK
SFEKDYNRVSEYKKIRDLVEFNYLNKIESYLIDINWKLAIQMARFERDMH
YIVNGLRELGIIKLSGYNTGISRAYPKRNGSDGFYTTTAYYKFFDEESYK
KFEKICYGFGIDLSENSEINKPENESIRNYISHFYIVRNPFADYSIAEQI
DRVSNLLSYSTRYNNSTYASVFEVFKKDVNLDYDELKKKFKLIGNNDILE
RLMKPKKVSVLELESYNSDYIKNLIIELLTKIENTNDTL

In some embodiments, the guide nucleotide sequence-programmable DNA-binding protein domain of the present disclosure has no requirements for a PAM sequence. One example of such a guide nucleotide sequence-programmable DNA-binding protein may be an Argonaute protein from Natronobacterium gregoryi (NgAgo). NgAgo is a ssDNA-guided endonuclease. NgAgo binds 5′ phosphorylated ssDNA of ˜-24 nucleotides (gDNA) to guide it to its target site and will make DNA double-strand breaks at the gDNA site. In contrast to Cas9, the NgAgo-gDNA system does not require a protospacer-adjacent motif (PAM). Using a nuclease inactive NgAgo (dNgAgo) can greatly expand the codons that may be targeted. The characterization and use of NgAgo have been described in Gao et al., Nat Biotechnol., 2016 July; 34(7):768-73. PubMed PMID: 27136078; Swarts et al., Nature. 507(7491) (2014):258-61; and Swarts et al., Nucleic Acids Res. 43(10) (2015):5120-9, each of which is incorporated herein by reference. The sequence of Natronobacterium gregoryi Argonaute is provided in SEQ ID NO: 718.

Wild type Natronobacterium gregoryi Argonaute
(SEQ ID NO: 718)
MTVIDLDSTTTADELTSGHTYDISVTLTGVYDNTDEQHPRMSLAFEQDNG
ERRYITLWKNTTPKDVFTYDYATGSTYIFTNIDYEVKDGYENLTATYQTT
VENATAQEVGTTDEDETFAGGEPLDHHLDDALNETPDDAETESDSGHVMT
SFASRDQLPEWTLHTYTLTATDGAKTDTEYARRTLAYTVRQELYTDHDAA
PVATDGLMLLTPEPLGETPLDLDCGVRVEADETRTLDYTTAKDRLLAREL
VEEGLKRSLWDDYLVRGIDEVLSKEPVLTCDEFDLHERYDLSVEVGHSGR
AYLHINFRHRFVPKLTLADIDDDNIYPGLRVKTTYRPRRGHIVWGLRDEC
ATDSLNTLGNQSVVAYHRNNQTPINTDLLDAIEAADRRVVETRRQGHGDD
AVSFPQELLAVEPNTHQIKQFASDGFHQQARSKTRLSASRCSEKAQAFAE
RLDPVRLNGSTVEFSSEFFTGNNEQQLRLLYENGESVLTFRDGARGAHPD
ETFSKGIVNPPESFEVAVVLPEQQADTCKAQWDTMADLLNQAGAPPTRSE
TVQYDAFSSPESISLNVAGAIDPSEVDAAFVVLPPDQEGFADLASPTETY
DELKKALANMGIYSQMAYFDRFRDAKIFYTRNVALGLLAAAGGVAFTTEH
AMPGDADMFIGIDVSRSYPEDGASGQINIAATATAVYKDGTILGHSSTRP
QLGEKLQSTDVRDIMKNAILGYQQVTGESPTHIVIHRDGFMNEDLDPATE
FLNEQGVEYDIVEIRKQPQTRLLAVSDVQYDTPVKSIAAINQNEPRATVA
TFGAPEYLATRDGGGLPRPIQIERVAGETDIETLTRQVYLLSQSHIQVHN
STARLPITTAYADQASTHATKGYLVQTGAFESNVGFL

Also provided herein are Cas9 variants that have relaxed PAM requirements (PAMless Cas9). PAMless Cas9 exhibits an increased activity on a target sequence that does not include a canonical PAM (e.g., NGG) sequence at its 3′-end as compared to Streptococcus pyogenes Cas9 as provided by SEQ ID NO: 1, e.g., increased activity by at least 5-fold, at least 10-fold, at least 50-fold, at least 100-fold, at least 500-fold, at least 1,000-fold, at least 5,000-fold, at least 10,000-fold, at least 50,000-fold, at least 100,000-fold, at least 500,000-fold, or at least 1,000,000-fold. Such Cas9 variants that have relaxed PAM requirements are described in US Provisional Applications, U.S. Ser. No. 62/245,828, filed Oct. 23, 2015; 62/279,346, filed Jan. 15, 2016; 62/311,763, filed Mar. 22, 2016; 62/322,178, filed Apr. 13, 2016; and 62/357,332, filed Jun. 30, 2016, each of which is incorporated herein by reference. In some embodiments, the dCas9 or Cas9 nickase useful in the present disclosure may further comprise mutations that relax the PAM requirements, e.g., mutations that correspond to A262T, K294R, S409I, E480K, E543D, M694I, or E1219V in SEQ ID NO: 1.

The “-” used in the general architecture discussed herein may indicate the presence of an optional linker. The term “linker,” as used herein, refers to a chemical group or a molecule linking two molecules or moieties, e.g., two domains of a fusion protein, such as, for example, a guide nucleotide sequence-programmable DNA binding protein domain and a recombinase catalytic domain. Typically, the linker is positioned between, or flanked by, two groups, molecules, or other moieties and connected to each one via a covalent bond, thus connecting the two. In some embodiments, the linker is an amino acid or a plurality of amino acids (e.g., a peptide or protein). In some embodiments, the linker is an organic molecule, group, polymer, or chemical moiety. In some embodiments, the linker is 5-100 amino acids in length, for example, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, 90-100, 100-150, or 150-200 amino acids in length. Longer or shorter linkers are also contemplated. Linkers may be of any form known in the art. For example, the linker may be a linker from a website such as www[dot]ibi[dot]vu[dot]nl/programs/linkerdbwww/or from www[dot] ibi[dot]vu[dot]nl/programs/linkerdbwww/src/database.txt. The linkers may also be unstructured, structured, helical, or extended.

In some embodiments, the guide nucleotide sequence-programmable DNA binding protein domain and the recombinase catalytic domain are fused to each other via a linker. Various linker lengths and flexibilities between the guide nucleotide sequence-programmable DNA binding protein domain and the recombinase catalytic domain can be employed (e.g., ranging from flexible linkers of the form (GGGS)n (SEQ ID NO: 759), (GGGGS)n (SEQ ID NO: 722), (GGS)n, and (G)n to more rigid linkers of the form (EAAAK)n (SEQ ID NO: 723), SGSETPGTSESATPES (SEQ ID NO: 724) (see, e.g., Guilinger et al., Nat. Biotechnol. 2014; 32(6): 577-82; the entire contents of which is incorporated herein by reference), (XP)n, or a combination of any of these, wherein X is any amino acid, and n is independently an integer between 1 and 30, in order to achieve the optimal length for activity for the specific application. In some embodiments, n is independently 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30, or, if more than one linker or more than one linker motif is present, any combination thereof. In some embodiments, the linker comprises a (GGS)n motif, wherein n is 1, 2, 3, 4, 5, 6, 7, 8, 9, 10, 11, 12, 13, 14 or 15. In some embodiments, the linker comprises a (GGS)n motif, wherein n is 1, 3, or 7. In some embodiments, the linker comprises an XTEN linker. The XTEN linker may have the sequence SGSETPGTSESATPES (SEQ ID NO: 7), SGSETPGTSESA (SEQ ID NO: 8), or SGSETPGTSESATPEGGSGGS (SEQ ID NO: 9). In some embodiments, the linker comprises an amino acid sequence chosen from the group including, but not limited to, AGVF (SEQ ID NO: 772), GFLG (SEQ ID NO: 773), FK, AL, ALAL (SEQ ID NO: 774), and ALALA (SEQ ID NO: 775). In some embodiments, suitable linker motifs and configurations include those described in Chen et al., Fusion protein linkers: property, design and functionality. Adv Drug Deliv Rev. 2013; 65(10):1357-69, which is incorporated herein by reference. In some embodiments, the linker may comprise any of the following amino acid sequences: VPFLLEPDNINGKTC (SEQ ID NO: 10), GSAGSAAGSGEF (SEQ ID NO: 11), SIVAQLSRPDPA (SEQ ID NO: 12), MKIIEQLPSA (SEQ ID NO: 13), VRHKLKRVGS (SEQ ID NO: 14), GHGTGSTGSGSS (SEQ ID NO: 15), MSRPDPA (SEQ ID NO: 16), GSAGSAAGSGEF (SEQ ID NO: 7), SGSETPGTSESA (SEQ ID NO: 8), SGSETPGTSESATPEGGSGGS (SEQ ID NO: 9), and GGSM (SEQ ID NO: 17).

Additional suitable linker sequences will be apparent to those of skill in the art based on the instant disclosure. In certain embodiments, the linker may have a length of about 33 angstroms to about 81 angstroms. In another embodiment, the linker may have a length of about 54 angstroms to about 81 angstroms. In a further embodiment, the linker may have a length of about 63 to about 81 angstroms. In another embodiment, the linker may have a length of about 65 angstroms to about 75 angstroms. In some embodiments, the linker may have a weight of about 1.20 kDa to about 1.85 kDa. In certain embodiments, the linker may have a weight of about 1.40 kDa to about 1.85 kDa. In certain embodiments, the linker may have a weight of about 1.60 kDa to about 1.7 kDa. In some embodiments, the linker is an amino acid or a plurality of amino acids (e.g., a peptide or protein). In some embodiments, the linker is an organic molecule, group, polymer, or chemical moiety. In some embodiments, the linker is a peptide linker. In some embodiments, the peptide linker is any stretch of amino acids having at least 1, at least 2, at least 3, at least 4, at least 5, at least 6, at least 7, at least 8, at least 9, at least 10, at least 15, at least 20, at least 25, at least 30, at least 40, at least 50, or more amino acids. In certain embodiments, the peptide linker is from 18 to 27 amino acids long. In a specific embodiment, the peptide linker is 24 amino acids long. In some embodiments, the peptide linker comprises repeats of the tri-peptide Gly-Gly-Ser, e.g., comprising the sequence (GGS)ₙ, wherein n represents at least 1, 2, 3, 4, 5, 6, 7, 8, 9, 10, or more repeats. In some embodiments, the linker comprises the sequence (GGS)₆ (SEQ ID NO: 6). In some embodiments, the peptide linker is the 16 residue “XTEN” linker, or a variant thereof (See, e.g., the Examples; and Schellenberger et al. A recombinant polypeptide extends the in vivo half-life of peptides and proteins in a tunable manner. Nat. Biotechnol. 27, 1186-1190 (2009)). In some embodiments, the XTEN linker comprises the sequence SGSETPGTSESATPES (SEQ ID NO: 7), SGSETPGTSESA (SEQ ID NO: 8), or SGSETPGTSESATPEGGSGGS (SEQ ID NO: 9). In some embodiments, the peptide linker is selected from VPFLLEPDNINGKTC (SEQ ID NO: 10), GSAGSAAGSGEF (SEQ ID NO: 11), SIVAQLSRPDPA (SEQ ID NO: 12), MKIIEQLPSA (SEQ ID NO: 13), VRHKLKRVGS (SEQ ID NO: 14), GHGTGSTGSGSS (SEQ ID NO: 15), MSRPDPA (SEQ ID NO: 16); or GGSM (SEQ ID NO: 17). In some embodiments, the linker is a non-peptide linker. In certain embodiments, the non-peptide linker comprises one or more of polyethylene glycol (PEG), polypropylene glycol (PPG), co-poly(ethylene/propylene) glycol, polyoxyethylene (POE), polyurethane, polyphosphazene, polysaccharides, dextran, polyvinyl alcohol, polyvinylpyrrolidones, polyvinyl ethyl ether, polyacryl amide, polyacrylate, polycyanoacrylates, lipid polymers, chitins, hyaluronic acid, heparin, or an alkyl linker. In one embodiment, the alkyl linker has the formula —NH—(CH₂)ₛ—C(O)—, wherein s may be any integer. In a further embodiment, s may be any integer from 1-20.

### Recombinase Catalytic Domain

The recombinase catalytic domain for use in the compositions and methods of the instant disclosure may be from any recombinase. Suitable recombinases catalytic domains for use in the disclosed methods and compositions may be obtained from, for example, and without limitation, tyrosine recombinases and serine recombinases. Some exemplary suitable recombinases provided herein include, for example, and without limitation, Gin recombinase (acting on gix sites), Hin recombinase (acting on hix sites), β recombinase (acting on six sites), Sin recombinase (acting on resH sites), Tn3 recombinase (acting on res sites), γδ recombinase (acting on res sites), Cre recombinase from bacteriophage P1 (acting on LoxP sites); FLP recombinases of fungal origin (acting on FTR sites); and phiC31 integrase (acting on att sites). Non-limiting sequences of exemplary suitable recombinases may be found below.

Cre recombinase sequence
(SEQ ID NO: 725)
MSNLLTVHQNLPALPVDATSDEVRKNLMDMFRDRQAFSEHTWKMLLSVCR
SWAAWCKLNNRKWFPAEPEDVRDYLLYLQARGLAVKTIQQHLGQLNMLHR
RSGLPRPSDSNAVSLVMRRIRKENVDAGERAKQALAFERTDFDQVRSLME
NSDRCQDIRNLAFLGIAYNTLLRIAEIARIRVKDISRTDGGRMLIHIGRT
KTLVSTAGVEKALSLGVTKLVERWISVSGVADDPNNYLFCRVRKNGVAAP
SATSQLSTRALEGIFEATHRLIYGAKDDSGQRYLAWSGHSARVGAARDMA
RAGVSIPEIMQAGGWTNVNIVMNYIRNLDSETGAMVRLLEDGD
FLP recombinase
(SEQ ID NO: 726)
MPQFGILCKTPPKVLVRQFVERFERPSGEKIALCAAELTYLCWMITHNGT
AIKRATFMSYNTIISNSLSFDIVNKSLQFKYKTQKATILEASLKKLIPAW
EFTIIPYYGQKHQSDITDIVSSLQLQFESSEEADKGNSHSKKMLKALLSE
GESIWEITEKILNSFEYTSRFTKTKTLYQFLFLATFINCGRFSDIKNVDP
KSFKLVQNKYLGVIIQCLVTETKTSVSRHIYFFSARGRIDPLVYLDEFLR
NSEPVLKRVNRTGNSSSNKQEYQLLKDNLVRSYNKALKKNAPYSIFAIKN
GPKSHIGRHLMTSFLSMKGLTELTNVVGNWSDKRASAVARTTYTHQITAI
PDHYFALVSRYYAYDPISKEMIALKDETNPIEEWQHIEQLKGSAEGSIRY
PAWNGIISQEVLDYLSSYINRRI
γδ recombinase (Gamma Delta resolvase)
(SEQ ID NO: 727)
MRLFGYARVSTSQQSLDIQVRALKDAGVKANRIFTDKASGSSSDRKGLDL
LRMKVEEGDVILVKKLDRLGRDTADMIQLIKEFDAQGVSIRFIDDGISTD
GEMGKMVVTILSAVAQAERQRILERTNEGRQEAMAKGVVFGRKR
γδ recombinase (E124Q mutation)
(SEQ ID NO: 728)
MRLFGYARVSTSQQSLDIQVRALKDAGVKANRIFTDKASGSSSDRKGLDL
LRMKVEEGDVILVKKLDRLGRDTADMIQLIKEFDAQGVSIRFIDDGISTD
GEMGKMVVTILSAVAQAERQRILQRTNEGRQEAMAKGVVFGRKR
γδ recombinase (E102Y/E124Q mutation)
(SEQ ID NO: 729)
MRLFGYARVSTSQQSLDIQVRALKDAGVKANRIFTDKASGSSSDRKGLDL
LRMKVEEGDVILVKKLDRLGRDTADMIQLIKEFDAQGVSIRFIDDGISTD
GYMGKMVVTILSAVAQAERQRILQRTNEGRQEAMAKGVVFGRKR
β recombinase
(SEQ ID NO: 730)
MAKIGYARVSSKEQNLDRQLQALQGVSKVFSDKLSGQSVERPQLQAMLNY
IREGDIVVVTELDRLGRNNKELTELMNAIQQKGATLEVLDLPSMNGIEDE
NLRRLINNLVIELYKYQAESERKRIKERQAQGIEIAKSKGKFKGRQH
β recombinase (N95D mutation)
(SEQ ID NO: 731)
MAKIGYARVSSKEQNLDRQLQALQGVSKVFSDKLSGQSVERPQLQAMLNY
IREGDIVVVTELDRLGRNNKELTELMNAIQQKGATLEVLDLPSMDGIEDE
NLRRLINNLVIELYKYQAESERKRIKERQAQGIEIAKSKGKFKGRQH
Sin recombinase
(SEQ ID NO: 732)
MIIGYARVSSLDQNLERQLENLKTFGAEKIFTEKQSGKSIENRPILQKAL
NFVRMGDRFIVESIDRLGRNYNEVIHTVNYLKDKEVQLMITSLPMMNEVI
GNPLLDKFMKDLIIQILAMVSEQERNESKRRQAQGIQVAKEKGVYKGRPL
Sin recombinase (Q87R/Q115R mutations)
(SEQ ID NO: 733)
MIIGYARVSSLDQNLERQLENLKTFGAEKIFTEKQSGKSIENRPILQKAL
NFVRMGDRFIVESIDRLGRNYNEVIHTVNYLKDKEVRLMITSLPMMNEVI
GNPLLDKFMKDLIIRILAMVSEQERNESKRRQAQGIQVAKEKGVYKGRPL
Tn3 recombinase
(SEQ ID NO: 734)
MRLFGYARVSTSQQSLDLQVRALKDAGVKANRIFTDKASGSST
DREGLDLLRMKVKEGDVILVKKLDRLGRDTADMLQLIKEFDAQGVAV
RFIDDGISTDGDMGQMVVTILSAVAQAERRRILERTNEGRQEAK
LKGIKFGRRR
Tn3 recombinase (G70S/D102Y, E124Q mutations)
(SEQ ID NO: 735)
MRLFGYARVSTSQQSLDLQVRALKDAGVKANRIFTDKASGSSTDREGLDL
LRMKVKEGDVILVKKLDRLSRDTADMLQLIKEFDAQGVAVRFIDDGISTD
GYMGQMVVTILSAVAQAERRRILQRTNEGRQEAKLKGIKFGRRR
Hin recombinase
(SEQ ID NO: 736)
MATIGYIRVSTIDQNIDLQRNALTSANCDRIFED
RISGKIANRPGLKRALKYVNKGDTLVVWKLDRLGRSVKNLVALISELHER
GAHFHSLTDSIDTSSAMGRFFFHVMSALAEMERELIVERTLAGLAAARAQ
GRLGGRPV
Hin recombinase (H107Y mutation)
(SEQ ID NO: 737)
MATIGYIRVSTIDQNIDLQRNALTSANCDRIFEDRISGKIANRPGLKRAL
KYVNKGDTLVVWKLDRLGRSVKNLVALISELHERGAHFHSLTDSIDTSSA
MGRFFFYVMSALAEMERELIVERTLAGLAAARAQGRLGGRPV
PhiC31 recombinase
(SEQ ID NO: 738)
MDTYAGAYDRQSRERENSSAASPATQRSANEDKAADLQREVERDGGRFRF
VGHFSEAPGTSAFGTAERPEFERILNECRAGRLNMIIVYDVSRFSRLKVM
DAIPIVSELLALGVTIVSTQEGVFRQGNVMDLIHLIMRLDASHKESSLKS
AKILDTKNLQRELGGYVGGKAPYGFELVSETKEITRNGRMVNVVINKLAH
STTPLTGPFEFEPDVIRWWWREIKTHKHLPFKPGSQAAIHPGSITGLCKR
MDADAVPTRGETIGKKTASSAWDPATVMRILRDPRIAGFAAEVIYKKKPD
GTPTTKIEGYRIQRDPITLRPVELDCGPIIEPAEWYELQAWLDGRGRGKG
LSRGQAILSAMDKLYCECGAVMTSKRGEESIKDSYRCRRRKVVDPSAPGQ
HEGTCNVSMAALDKFVAERIFNKIRHAEGDEETLALLWEAARRFGKLTEA
PEKSGERANLVAERADALNALEELYEDRAAGAYDGPVGRKHFRKQQAALT
LRQQGAEERLAELEAAEAPKLPLDQWFPEDADADPTGPKSWWGRASVDDK
RVFVGLFVDKIVVTKSTTGRGQGTPIEKRASITWAKPPTDDDEDDAQDGT
EDVAATGA

Recombinases for use with the disclosed compositions and methods may also include further mutations. Some aspects of this disclosure provide recombinases comprising an amino acid sequence that is at least 70%, at least 80%, at least 90%, at least 95%, or at least 97% identical to the sequence of the recombinase sequence discussed herein, wherein the amino acid sequence of the recombinase comprises at least one mutation as compared to the sequence of the recombinase sequence discussed herein. In some embodiments, the amino acid sequence of the recombinase comprises at least 2, at least 3, at least 4, at least 5, at least 6, at least 7, at least 8, at least 9, at least 10, at least 11, at least 12, at least 13, at least 14, or at least 15 mutations as compared to the sequence of the recombinase sequence discussed herein.

For example, the γδ recombinase may comprise one or more mutations from the list: R2A, E56K, G101S, E102Y, M103I, or E124Q. In one embodiment, the γδ recombinase may comprise an E102Y mutation, an E124Q mutation, or both an E102Y and E124Q mutation. In another embodiment, the β recombinase may comprise one or more mutations including, but not limited to N95D. See, for example, Sirk et al., “Expanding the zinc-finger recombinase repertoire: directed evolution and mutational analysis of serine recombinase specificity determinants” Nucl Acids Res (2014) 42 (7): 4755-4766. In another embodiment, the Sin recombinase may have one or more mutations including, but not limited to: Q87R, Q115R, or Q87R and Q115R. In another embodiment, the Tn3 recombinase may have one or more mutations including, but not limited to: G70S, D102Y, E124Q, and any combination thereof. In another embodiment, the Hin recombinase may have one or more mutations including, but not limited to: H107Y. In another embodiment, the Sin recombinase may have one or more mutations including, but not limited to: H107Y. Any of the recombinase catalytic domains for use with the disclosed compositions and methods may have greater than 85%, 90%, 95%, 98%, or 99% sequence identity with the native (or wild type) amino acid sequence. For example, in certain embodiments, the Gin recombinase catalytic domain has greater than 85%, 90%, 95%, 98%, or 99% sequence identity with the amino acid sequence shown in SEQ ID NO: 713. In another embodiment, the amino acid sequence of the Gin recombinase catalytic domain comprises a mutation corresponding to H106Y, and/or I127L, and/or I136R and/or G137F. In yet another embodiment, the amino acid sequence of the Gin recombinase catalytic domain comprises a mutation corresponding to H106Y, I127L, I136R, and G137F. In a further embodiment, the amino acid sequence of the Gin recombinase has been further mutated. In a specific embodiment, the amino acid sequence of the Gin recombinase catalytic domain comprises SEQ ID NO: 713.

The recombinase catalytic domain for use in the compositions and methods of the instant disclosure may be from an evolved recombinase. As used herein, the term “evolved recombinase” refers to a recombinase that has been altered (e.g., through mutation) to recognize non-native DNA target sequences.

Suitable recombinases that can be evolved include, for example, and without limitation, tyrosine recombinases and serine recombinases (e.g., any of the recombinases discussed herein). Some exemplary suitable recombinases that can be evolved by the methods and strategies provided herein include, for example, and without limitation, Gin recombinase (acting on gix sites), Hin recombinase (acting on hix sites), β recombinase (acting on six sites), Sin recombinase (acting on resH sites), Tn3 recombinase (acting on res sites), γδ recombinase (acting on res sites), Cre recombinase from bacteriophage P1 (acting on LoxP sites); λ phage integrase (acting on att sites); FLP recombinases of fungal origin (acting on FTR sites); phiC31 integrase; Dre recombinase, BxB1; and prokaryotic β-recombinase.

For example, the evolved recombinase for use with the compositions and methods of the instant disclosure may have been altered to interact with (e.g., bind and recombine) a non-canonical recombinase target sequence. As a non-limiting example, the non-canonical recombinase target sequence may be naturally occurring, such as, for example, sequences within a “safe harbor” genomic locus in a mammalian genome, e.g., a genomic locus that is known to be tolerant to genetic modification without any undesired effects. Recombinases targeting such sequences allow, e.g., for the targeted insertion of nucleic acid constructs at a specific genomic location without the need for conventional time- and labor-intensive gene targeting procedures, e.g., via homologous recombination technology. In addition, the directed evolution strategies provided herein can be used to evolve recombinases with an altered activity profile, e.g., recombinases that favor integration of a nucleic acid sequence over excision of that sequence or vice versa.

Evolved recombinases exhibit altered target sequence preferences as compared to their wild type counterparts, can be used to target virtually any target sequence for recombinase activity. Accordingly, the evolved recombinases can be used to modify, for example, any sequence within the genome of a cell or subject. Because recombinases can effect an insertion of a heterologous nucleic acid molecule into a target nucleic acid molecule, an excision of a nucleic acid sequence from a nucleic acid molecule, an inversion, or a replacement of nucleic acid sequences, the technology provided herein enables the efficient modification of genomic targets in a variety of ways (e.g., integration, deletion, inversion, exchange of nucleic acid sequences).

Catalytic domains from evolved recombinases for use with the methods and compositions of the instant disclosure comprise an amino acid sequence that is at least 70%, at least 80%, at least 90%, at least 95%, or at least 97% identical to the sequence of a wild-type recombinase, wherein the amino acid sequence of the evolved recombinase comprises at least one mutation as compared to the sequence of the wild-type recombinase, and wherein the evolved recombinase recognizes a DNA recombinase target sequence that differs from the canonical recombinase target sequence by at least one nucleotide. In some embodiments, the evolved recombinase recognizes a DNA recombinase target sequence that differs from the canonical recombinase target sequence (e.g., a res, gix, hix, six, resH, LoxP, FTR, or att core or related core sequence) by at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20 at least 25, or at least 30 nucleotides. In some embodiments, the evolved recombinase recognizes a DNA recombinase target sequence that differs from the canonical recombinase target sequence by 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides.

In some embodiments, only a portion of the recombinase is used in the fusion proteins and methods described herein. As a non-limiting embodiment, only the C-terminal portion of the recombinase may be used in the fusion proteins and methods described herein. In a specific embodiment, the 25 kDa carboxy-terminal domain of Cre recombinase may be used in the compositions and methods. See, for example, Hoess et al, “DNA Specificity of the Cre Recombinase Resides in the 25 kDa Carboxyl Domain of the Protein,” J. Mol. Bio. 1990 Dec. 20, 216(4):873-82, which is incorporated by reference herein for all purposes. The 25 kDa carboxy-terminal domain of Cre recombinase is the portion stretching from R118 to the carboxy terminus of the protein. In some embodiments, the 25 kDa carboxy-terminal domain of Cre recombinase for use in the instant fusion proteins and methods may differ from the canonical 25 kDa carboxy-terminal domain of Cre recombinase by at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 amino acids. In some embodiments, the 25 kDa carboxy-terminal domain of Cre recombinase for use in the instant fusion proteins and methods may differ from the canonical 25 kDa carboxy-terminal domain of Cre recombinase by 1, 2, 3, 4, 5, 6, 7, 8, 9, 10, 11, 12, 13, 14, 15, 16, 17, 18, 19, or 20 amino acids. In certain embodiments, only a portion of the 25 kDa carboxy-terminal domain of Cre recombinase may be used in the fusion proteins and methods described herein. For example, the portion of Cre recombinase used may be R130 to the carboxy terminus of the protein, T140 to the carboxy terminus of the protein, E150 to the carboxy terminus of the protein, N160 to the carboxy terminus of the protein, T170 to the carboxy terminus of the protein, 1180 to the carboxy terminus of the protein, G190 to the carboxy terminus of the protein, T200 to the carboxy terminus of the protein, E210 to the carboxy terminus of the protein, L220 to the carboxy terminus of the protein, V230 to the carboxy terminus of the protein, C240 to the carboxy terminus of the protein, P250 to the carboxy terminus of the protein, A260 to the carboxy terminus of the protein, R270 to the carboxy terminus of the protein, G280 to the carboxy terminus of the protein, S290 to the carboxy terminus of the protein, A300 to the carboxy terminus of the protein, or M310 to the carboxy terminus of the protein. As another set of non-limiting examples, the portion of Cre recombinase used may be R118-E340, R118-S330, R118-I320, R118-M310, R118-A300, R118-S290, R118-G280, R118-R270, R118-A260, R118-P250, R118-C240, R118-V230, R118-L220, or R118-E210. As a further set of non-limiting examples, the portion of Cre recombinase used may be R118-E210, G190-R270, E210-S290, P250-M310, or R270 to the carboxy terminus of the protein.

In some embodiments, the Cre recombinase used in the fusion proteins and methods described herein may be truncated at any position. In a specific embodiment, the Cre recombinase used in the fusion proteins and methods described herein may be truncated such that it begins with amino acid R118, A127, E138, or R154) (preceded in each case by methionine). In another set of non-limiting embodiments, the Cre recombinase used in the fusion proteins and methods described herein may be truncated within 10 amino acids, 9 amino acids, 8 amino acids, 7 amino acids, 6 amino acids, 5 amino acids, 4 amino acids, 3 amino acids, 2 amino acids, or 1 amino acid of R118, A127, E138, or R154.

In some embodiments, the recombinase target sequence is between 10-50 nucleotides long. In some embodiments, the recombinase is a Cre recombinase, a Hin recombinase, or a FLP recombinase. In some embodiments, the canonical recombinase target sequence is a LoxP site (5′-ATAACTTCGTATA GCATACAT TATACGAAGTTAT-3′ (SEQ ID NO: 739). In some embodiments, the canonical recombinase target sequence is an FRT site (5′-GAAGTTCCTATTCTCTAGAAA GTATAGGAACTTC-3′) (SEQ ID NO: 740). In some embodiments, the amino acid sequence of the evolved recombinase comprises at least 2, at least 3, at least 4, at least 5, at least 6, at least 7, at least 8, at least 9, at least 10, at least 11, at least 12, at least 13, at least 14, or at least 15 mutations as compared to the sequence of the wild-type recombinase. In some embodiments, the evolved recombinase recognizes a DNA recombinase target sequence that comprises a left half-site, a spacer sequence, and a right half-site, and wherein the left half-site is not a palindrome of the right half-site.

In some embodiments, the evolved recombinase recognizes a DNA recombinase target sequence that comprises a naturally occurring sequence. In some embodiments, the evolved recombinase recognizes a DNA recombinase target sequence that is comprised in the genome of a mammal. In some embodiments, the evolved recombinase recognizes a DNA recombinase target sequence comprised in the genome of a human. In some embodiments, the evolved recombinase recognizes a DNA recombinase target sequence that occurs only once in the genome of a mammal. In some embodiments, the evolved recombinase recognizes a DNA recombinase target sequence in the genome of a mammal that differs from any other site in the genome by at least 1, at least 2, at least 3, at least 4, at least 5, at least 6, at least 7, at least 8, at least 9, at least 10, at least 11, at least 12, at least 13, at least 14, or at least 15 nucleotide(s). In some embodiments, the evolved recombinase recognizes a DNA recombinase target sequence located in a safe harbor genomic locus. In some embodiments, the safe harbor genomic locus is a Rosa26 locus. In some embodiments, the evolved recombinase recognizes a DNA recombinase target sequence located in a genomic locus associated with a disease or disorder.

In certain embodiments, the evolved recombinase may target a site in the Rosa locus of the human genome (e.g., 36C6). A non-limiting set of such recombinases may be found, for example, in International PCT Publication, WO 2017/015545A1, published Jan. 26, 2017, entitled “Evolution of Site Specific Recombinases,” which is incorporated by reference herein for this purpose. In some embodiments, the amino acid sequence of the evolved recombinase comprises at least 2, at least 3, at least 4, at least 5, at least 6, at least 7, at least 8, at least 9, at least 10, at least 11, at least 12, at least 13, at least 14, or at least 15 mutations as compared to the sequence of the wild-type recombinase. The nucleotide sequence encoding 36C6 is shown below in bold; those encoding GGS linkers are shown in italics; those encoding dCas9 linkers are black; those encoding the FLAG tag and NLS are underlined and in lowercase, respectively.

dCas9-36C6 (nucleotide)
(SEQ ID NO: 765)
ATGTCCAACCTCCTTACCGTCCACCAGAATCTCCCTGCCCTTCCGGTGGATGCCACCTCTGATGAAGTGCGAAAA
AACCTGATGGATATGTTTCGCGATAGGCAAGCTTTTTCTGAACACACGTGGAAGATGCTCCTGTCAGTGTGTAGA
AGCTGGGCAGCTTGGTGCAAGTTGAACAACCGAAAATGGTTTCCTGCCGAACCCGAAGATGTGAGAGACTACCTC
CTCTACCTGCAGGCTCGAGGGCTCGCCGTGAAAACAATCCAACAACACTTGGGTCAGCTCAACATGCTGCACAGG
AGATCTGGGCTGCCCCGGCCGAGTGACTCTAATGCCGTTAGTCTCGTAATGCGGCGCATTCGCAAAGAGAATGTG
GATGCTGGAGAACGGGCGAAACAGGCACTGGCTTTTGAACGGACCGACTTCGATCAGGTGCGGAGTCTTATGGAG
AATAGTGACAGATGCCAGGACATTCGGAACCTTGCATTCCTGGGTATCGCGTATAATACCCTGCTGAGAATCGCT
GAGATCGCCAGAATCAGGGTAAAGGATATTTCTCGAACGGACGGGGGACGGATGTTGATTCATATCGGTCGCACT
AAAACACTTGTGAGTACCGCCGGGGTAGAGAAAGCCCTGAGCCTTGGAGTTACTAAACTGGTGGAGCGGTGGATT
AGCGTGTCCGGCGTGGCGGATGACCCAAACAATTACTTGTTTTGTAGGGTGCGGAAAAATGGTGTAGCCGCTCCA
TCCGCTACCTCACAGTTGAGTACACGCGCGTTGGAGGGGATTTTCGAAGCCACACATCGCTTGATCTACGGCGCC
AAGGACGATTCAGGCCAGCGATATCTTGCCTGGAGCGGGCATAGTGCCCGGGTGGGTGCCGCCCGAGACATGGCA
AGGGCTGGCGTGTCAATTCCTGAAATCATGCAGGCCGGCGGGTGGACCAACGTGAACATTGTGATGAACTATATC
CGGAACCTGGATAGCGAGACCGGAGCAATGGTCAGACTGCTTGAGGATGGCGACGGTGGATCCGGAGGGTCCGGA
GGTAGTGGCGGCAGCGGTGGTTCAGGTGGCAGCGGAGGGTCAGGAGGCTCTGATAAAAAGTATTCTATTGGTTTA
GCTATCGGCACTAATTCCGTTGGATGGGCTGTCATAACCGATGAATACAAAGTACCTTCAAAGAAATTTAAGGTG
TTGGGGAACACAGACCGTCATTCGATTAAAAAGAATCTTATCGGTGCCCTCCTATTCGATAGTGGCGAAACGGCA
GAGGCGACTCGCCTGAAACGAACCGCTCGGAGAAGGTATACACGTCGCAAGAACCGAATATGTTACTTACAAGAA
ATTTTTAGCAATGAGATGGCCAAAGTTGACGATTCTTTCTTTCACCGTTTGGAAGAGTCCTTCCTTGTCGAAGAG
GACAAGAAACATGAACGGCACCCCATCTTTGGAAACATAGTAGATGAGGTGGCATATCATGAAAAGTACCCAACG
ATTTATCACCTCAGAAAAAAGCTAGTTGACTCAACTGATAAAGCGGACCTGAGGTTAATCTACTTGGCTCTTGCC
CATATGATAAAGTTCCGTGGGCACTTTCTCATTGAGGGTGATCTAAATCCGGACAACTCGGATGTCGACAAACTG
TTCATCCAGTTAGTACAAACCTATAATCAGTTGTTTGAAGAGAACCCTATAAATGCAAGTGGCGTGGATGCGAAG
GCTATTCTTAGCGCCCGCCTCTCTAAATCCCGACGGCTAGAAAACCTGATCGCACAATTACCCGGAGAGAAGAAA
AATGGGTTGTTCGGTAACCTTATAGCGCTCTCACTAGGCCTGACACCAAATTTTAAGTCGAACTTCGACTTAGCT
GAAGATGCCAAATTGCAGCTTAGTAAGGACACGTACGATGACGATCTCGACAATCTACTGGCACAAATTGGAGAT
CAGTATGCGGACTTATTTTTGGCTGCCAAAAACCTTAGCGATGCAATCCTCCTATCTGACATACTGAGAGTTAAT
ACTGAGATTACCAAGGCGCCGTTATCCGCTTCAATGATCAAAAGGTACGATGAACATCACCAAGACTTGACACTT
CTCAAGGCCCTAGTCCGTCAGCAACTGCCTGAGAAATATAAGGAAATATTCTTTGATCAGTCGAAAAACGGGTAC
GCAGGTTATATTGACGGCGGAGCGAGTCAAGAGGAATTCTACAAGTTTATCAAACCCATATTAGAGAAGATGGAT
GGGACGGAAGAGTTGCTTGTAAAACTCAATCGCGAAGATCTACTGCGAAAGCAGCGGACTTTCGACAACGGTAGC
ATTCCACATCAAATCCACTTAGGCGAATTGCATGCTATACTTAGAAGGCAGGAGGATTTTTATCCGTTCCTCAAA
GACAATCGTGAAAAGATTGAGAAAATCCTAACCTTTCGCATACCTTACTATGTGGGACCCCTGGCCCGAGGGAAC
TCTCGGTTCGCATGGATGACAAGAAAGTCCGAAGAAACGATTACTCCATGGAATTTTGAGGAAGTTGTCGATAAA
GGTGCGTCAGCTCAATCGTTCATCGAGAGGATGACCAACTTTGACAAGAATTTACCGAACGAAAAAGTATTGCCT
AAGCACAGTTTACTTTACGAGTATTTCACAGTGTACAATGAACTCACGAAAGTTAAGTATGTCACTGAGGGCATG
CGTAAACCCGCCTTTCTAAGCGGAGAACAGAAGAAAGCAATAGTAGATCTGTTATTCAAGACCAACCGCAAAGTG
ACAGTTAAGCAATTGAAAGAGGACTACTTTAAGAAAATTGAATGCTTCGATTCTGTCGAGATCTCCGGGGTAGAA
GATCGATTTAATGCGTCACTTGGTACGTATCATGACCTCCTAAAGATAATTAAAGATAAGGACTTCCTGGATAAC
GAAGAGAATGAAGATATCTTAGAAGATATAGTGTTGACTCTTACCCTCTTTGAAGATCGGGAAATGATTGAGGAA
AGACTAAAAACATACGCTCACCTGTTCGACGATAAGGTTATGAAACAGTTAAAGAGGCGTCGCTATACGGGCTGG
GGACGATTGTCGCGGAAACTTATCAACGGGATAAGAGACAAGCAAAGTGGTAAAACTATTCTCGATTTTCTAAAG
AGCGACGGCTTCGCCAATAGGAACTTTATGCAGCTGATCCATGATGACTCTTTAACCTTCAAAGAGGATATACAA
AAGGCACAGGTTTCCGGACAAGGGGACTCATTGCACGAACATATTGCGAATCTTGCTGGTTCGCCAGCCATCAAA
AAGGGCATACTCCAGACAGTCAAAGTAGTGGATGAGCTAGTTAAGGTCATGGGACGTCACAAACCGGAAAACATT
GTAATCGAGATGGCACGCGAAAATCAAACGACTCAGAAGGGGCAAAAAAACAGTCGAGAGCGGATGAAGAGAATA
GAAGAGGGTATTAAAGAACTGGGCAGCCAGATCTTAAAGGAGCATCCTGTGGAAAATACCCAATTGCAGAACGAG
AAACTTTACCTCTATTACCTACAAAATGGAAGGGACATGTATGTTGATCAGGAACTGGACATAAACCGTTTATCT
GATTACGACGTCGATGCCATTGTACCCCAATCCTTTTTGAAGGACGATTCAATCGACAATAAAGTGCTTACACGC
TCGGATAAGAACCGAGGGAAAAGTGACAATGTTCCAAGCGAGGAAGTCGTAAAGAAAATGAAGAACTATTGGCGG
CAGCTCCTAAATGCGAAACTGATAACGCAAAGAAAGTTCGATAACTTAACTAAAGCTGAGAGGGGTGGCTTGTCT
GAACTTGACAAGGCCGGATTTATTAAACGTCAGCTCGTGGAAACCCGCCAAATCACAAAGCATGTTGCACAGATA
CTAGATTCCCGAATGAATACGAAATACGACGAGAACGATAAGCTGATTCGGGAAGTCAAAGTAATCACTTTAAAG
TCAAAATTGGTGTCGGACTTCAGAAAGGATTTTCAATTCTATAAAGTTAGGGAGATAAATAACTACCACCATGCG
CACGACGCTTATCTTAATGCCGTCGTAGGGACCGCACTCATTAAGAAATACCCGAAGCTAGAAAGTGAGTTTGTG
TATGGTGATTACAAAGTTTATGACGTCCGTAAGATGATCGCGAAAAGCGAACAGGAGATAGGCAAGGCTACAGCC
AAATACTTCTTTTATTCTAACATTATGAATTTCTTTAAGACGGAAATCACTCTGGCAAACGGAGAGATACGCAAA
CGACCTTTAATTGAAACCAATGGGGAGACAGGTGAAATCGTATGGGATAAGGGCCGGGACTTCGCGACGGTGAGA
AAAGTTTTGTCCATGCCCCAAGTCAACATAGTAAAGAAAACTGAGGTGCAGACCGGAGGGTTTTCAAAGGAATCG
ATTCTTCCAAAAAGGAATAGTGATAAGCTCATCGCTCGTAAAAAGGACTGGGACCCGAAAAAGTACGGTGGCTTC
GATAGCCCTACAGTTGCCTATTCTGTCCTAGTAGTGGCAAAAGTTGAGAAGGGAAAATCCAAGAAACTGAAGTCA
GTCAAAGAATTATTGGGGATAACGATTATGGAGCGCTCGTCTTTTGAAAAGAACCCCATCGACTTCCTTGAGGCG
AAAGGTTACAAGGAAGTAAAAAAGGATCTCATAATTAAACTACCAAAGTATAGTCTGTTTGAGTTAGAAAATGGC
CGAAAACGGATGTTGGCTAGCGCCGGAGAGCTTCAAAAGGGGAACGAACTCGCACTACCGTCTAAATACGTGAAT
TTCCTGTATTTAGCGTCCCATTACGAGAAGTTGAAAGGTTCACCTGAAGATAACGAACAGAAGCAACTTTTTGTT
GAGCAGCACAAACATTATCTCGACGAAATCATAGAGCAAATTTCGGAATTCAGTAAGAGAGTCATCCTAGCTGAT
GCCAATCTGGACAAAGTATTAAGCGCATACAACAAGCACAGGGATAAACCCATACGTGAGCAGGCGGAAAATATT
ATCCATTTGTTTACTCTTACCAACCTCGGCGCTCCAGCCGCATTCAAGTATTTTGACACAACGATAGATCGCAAA
CGATACACTTCTACCAAGGAGGTGCTAGACGCGACACTGATTCACCAATCCATCACGGGATTATATGAAACTCGG
ATAGATTTGTCACAGCTTGGGGGTGACGGTGGCTCCGATTATAAGGATGATGACGACAAGGGAGGTTCCccaaag
aagaaaaggaaggtcTGA
dCas9-36C6 (amino acid)
(SEQ ID NO: 766)
MSNLLTVHQNLPALPVDATSDEVRKNLMDMFRDRQAFSEHTWKMLLSVCRSWAAWCKLNNRKWFPAEPEDVRDYL
LYLQARGLAVKTIQQHLGQLNMLHRRSGLPRPSDSNAVSLVMRRIRKENVDAGERAKQALAFERTDFDQVRSLME
NSDRCQDIRNLAFLGIAYNTLLRIAEIARIRVKDISRTDGGRMLIHIGRTKTLVSTAGVEKALSLGVTKLVERWI
SVSGVADDPNNYLFCRVRKNGVAAPSATSQLSTRALEGIFEATHRLIYGAKDDSGQRYLAWSGHSARVGAARDMA
RAGVSIPEIMQAGGWTNVNIVMNYIRNLDSETGAMVRLLEDGDGGSGGSGGSGGSGGSGGSGGSGGSDKKYSIGL
AIGTNSVGWAVITDEYKVPSKKFKVLGNTDRHSIKKNLIGALLFDSGETAEATRLKRTARRRYTRRKNRICYLQE
IFSNEMAKVDDSFFHRLEESFLVEEDKKHERHPIFGNIVDEVAYHEKYPTIYHLRKKLVDSTDKADLRLIYLALA
HMIKFRGHFLIEGDLNPDNSDVDKLFIQLVQTYNQLFEENPINASGVDAKAILSARLSKSRRLENLIAQLPGEKK
NGLFGNLIALSLGLTPNFKSNFDLAEDAKLQLSKDTYDDDLDNLLAQIGDQYADLFLAAKNLSDAILLSDILRVN
TEITKAPLSASMIKRYDEHHQDLTLLKALVRQQLPEKYKEIFFDQSKNGYAGYIDGGASQEEFYKFIKPILEKMD
GTEELLVKLNREDLLRKQRTFDNGSIPHQIHLGELHAILRRQEDFYPFLKDNREKIEKILTFRIPYYVGPLARGN
SRFAWMTRKSEETITPWNFEEVVDKGASAQSFIERMTNFDKNLPNEKVLPKHSLLYEYFTVYNELTKVKYVTEGM
RKPAFLSGEQKKAIVDLLFKTNRKVTVKQLKEDYFKKIECFDSVEISGVEDRFNASLGTYHDLLKIIKDKDFLDN
EENEDILEDIVLTLTLFEDREMIEERLKTYAHLFDDKVMKQLKRRRYTGWGRLSRKLINGIRDKQSGKTILDFLK
SDGFANRNFMQLIHDDSLTFKEDIQKAQVSGQGDSLHEHIANLAGSPAIKKGILQTVKVVDELVKVMGRHKPENI
VIEMARENQTTQKGQKNSRERMKRIEEGIKELGSQILKEHPVENTQLQNEKLYLYYLQNGRDMYVDQELDINRLS
DYDVDAIVPQSFLKDDSIDNKVLTRSDKNRGKSDNVPSEEVVKKMKNYWRQLLNAKLITQRKFDNLTKAERGGLS
ELDKAGFIKRQLVETRQITKHVAQILDSRMNTKYDENDKLIREVKVITLKSKLVSDFRKDFQFYKVREINNYHHA
HDAYLNAVVGTALIKKYPKLESEFVYGDYKVYDVRKMIAKSEQEIGKATAKYFFYSNIMNFFKTEITLANGEIRK
RPLIETNGETGEIVWDKGRDFATVRKVLSMPQVNIVKKTEVQTGGFSKESILPKRNSDKLIARKKDWDPKKYGGF
DSPTVAYSVLVVAKVEKGKSKKLKSVKELLGITIMERSSFEKNPIDFLEAKGYKEVKKDLIIKLPKYSLFELENG
RKRMLASAGELQKGNELALPSKYVNFLYLASHYEKLKGSPEDNEQKQLFVEQHKHYLDEIIEQISEFSKRVILAD
ANLDKVLSAYNKHRDKPIREQAENIIHLFTLTNLGAPAAFKYFDTTIDRKRYTSTKEVLDATLIHQSITGLYETR
IDLSQLGGDGGSDYKDDDDKGGSpkkkrkv Stop

Some aspects of this disclosure provide evolved recombinases (e.g., a Cre recombinase) comprising an amino acid sequence that is at least 70%, at least 80%, at least 90%, at least 95%, or at least 97% identical to the sequence of the recombinase sequence (e.g., a Cre recombinase) discussed herein, wherein the amino acid sequence of the recombinase (e.g., a Cre recombinase) comprises at least one mutation as compared to the sequence of the recombinase (e.g., a Cre recombinase) sequence discussed herein, and wherein the recombinase (e.g., a Cre recombinase) recognizes a DNA recombinase target sequence that differs from the canonical LoxP site 5′-ATAACTTCGTATA GCATACAT TATACGAAGTTAT-3′ (SEQ ID NO: 739) in at least one nucleotide.

In some embodiments, the amino acid sequence of the evolved recombinase (e.g., a Cre recombinase) comprises at least 2, at least 3, at least 4, at least 5, at least 6, at least 7, at least 8, at least 9, at least 10, at least 11, at least 12, at least 13, at least 14, or at least 15 mutations as compared to the sequence of the recombinase (e.g., a Cre recombinase) sequence discussed herein and recognizes a DNA recombinase target sequence that differs from the canonical target site (e.g., a LoxP site) in at least 1, at least 2, at least 3, at least 4, at least 5, at least 6, at least 7, at least 8, at least 9, at least 10, at least 11, at least 12, at least 13, at least 14, or at least 15 nucleotides.

In some embodiments, the evolved Cre recombinase recognizes a DNA recombinase target sequence that comprises a left half-site, a spacer sequence, and a right half-site, wherein the left half-site is not a palindrome of the right half-site. In some embodiments, the evolved Cre recombinase recognizes a DNA recombinase target sequence that comprises a naturally occurring sequence. In some embodiments, the evolved Cre recombinase recognizes a DNA recombinase target sequence that is comprised in the genome of a mammal.

In some embodiments, the evolved Cre recombinase recognizes a DNA recombinase target sequence that is comprised in the genome of a human. In some embodiments, the evolved Cre recombinase recognizes a DNA recombinase target sequence that is comprised only once in the genome of a mammal. In some embodiments, the evolved Cre recombinase recognizes a DNA recombinase target sequence in the genome of a mammal that differs from any other site in the genome by at least 1, at least 2, at least 3, at least 4, at least 5, at least 6, at least 7, at least 8, at least 9, at least 10, at least 11, at least 12, at least 13, at least 14, or at least 15 nucleotide(s). In some embodiments, the evolved Cre recombinase recognizes a DNA recombinase target sequence located in a safe harbor genomic locus. In some embodiments, the safe harbor genomic locus is a Rosa26 locus. In some embodiments, the evolved Cre recombinase recognizes a DNA recombinase target sequence located in a genomic locus associated with a disease or disorder.

Additional evolved recombinases (and methods for making the same) for use with the instant methods and compositions may be found in, for example, U.S. patent application Ser. No. 15/216,844, which is incorporated herein by reference.

Additional suitable recombinases will be apparent to those of skill in the art for both providing recombinase catalytic domains or evolved recombinase catalytic domains, and such suitable recombinases include, without limitation, those disclosed in Hirano et al., Site-specific recombinases as tools for heterologous gene integration. Appl Microbiol Biotechnol. 2011 October; 92(2):227-39; Fogg et al., New applications for phage integrases. J Mol Biol. 2014 Jul. 29; 426(15):2703; Brown et al., Serine recombinases as tools for genome engineering. Methods. 2011 April; 53(4):372-9; Smith et al., Site-specific recombination by phiC31 integrase and other large serine recombinases. Biochem Soc Trans. 2010 April; 38(2):388-94; Grindley et al., Mechanisms of site-specific recombination. Annu Rev Biochem. 2006; 75:567-605; Smith et al., Diversity in the serine recombinases. Mol Microbiol. 2002 April; 44(2):299-307; Grainge et al., The integrase family of recombinase: organization and function of the active site. Mol Microbiol. 1999 August; 33(3):449-56; Gopaul et al., Structure and mechanism in site-specific recombination. Curr Opin Struct Biol. 1999 February; 9(1):14-20; Cox et al., Conditional gene expression in the mouse inner ear using Cre-loxP. J Assoc Res Otolaryngol. 2012 June; 13(3):295-322; Birling et al., Site-specific recombinases for manipulation of the mouse genome. Methods Mol Biol. 2009; 561:245-63; and Mishina M, Sakimura K. Conditional gene targeting on the pure C57BL/6 genetic background. Neurosci Res. 2007 June; 58(2):105-12; the entire contents of each of which are incorporated herein by reference.

### Structure of the Fusion Protein

The fusion protein of the instant disclosure may be any combination and order of the elements described herein. Exemplary fusion proteins include, but are not limited to, any of the following structures: NH₂-[recombinase catalytic domain]-[linker sequence]-[guide nucleotide sequence-programmable DNA binding protein domain]-[optional linker sequence]-[optional NLS domain]-[optional linker sequence]-[optional affinity tag]-COOH. In another embodiment, the fusion protein has the structure NH₂-[recombinase catalytic domain]-[linker sequence]-[guide nucleotide sequence-programmable DNA binding protein domain]-[optional linker sequence]-[NLS domain]-[optional linker sequence]-[optional affinity tag]-COOH. In another embodiment, the fusion protein has the structure NH₂-[recombinase catalytic domain]-[linker sequence]-[guide nucleotide sequence-programmable DNA binding protein domain]-[optional linker sequence]-[NLS domain]-[optional linker sequence]-[affinity tag]-COOH. In another embodiment, the fusion protein has the structure NH₂-[recombinase catalytic domain]-[linker sequence]-[guide nucleotide sequence-programmable DNA binding protein domain]-[linker sequence]-[NLS domain]-[linker sequence]-[affinity tag]-COOH.

In another embodiment, the fusion protein has the structure NH₂-[recombinase catalytic domain]-[optional linker sequence]-[guide nucleotide sequence-programmable DNA binding protein domain]-[optional linker sequence]-[NLS domain]-[optional linker sequence]-[affinity tag]-COOH, NH₂-[guide nucleotide sequence-programmable DNA binding protein domain]-[linker sequence]-[recombinase catalytic domain]-[optional linker sequence]-[NLS domain]-[optional linker sequence]-[optional affinity tag]-COOH, NH₂-[N-terminal portion of a bifurcated or circularly permuted guide nucleotide sequence-programmable DNA binding protein domain]-[optional linker sequence]-[recombinase catalytic domain]-[optional linker sequence]-[C-terminal portion of a bifurcated or circularly permuted guide nucleotide sequence-programmable DNA binding protein domain]-[optional linker sequence]-[NLS domain]-[optional linker sequence]-[optional affinity tag]-COOH, NH₂-[affinity tag]-[optional linker sequence]-[recombinase catalytic domain]-[linker sequence]-[guide nucleotide sequence-programmable DNA binding protein domain]-[optional linker sequence]-[NLS domain]-COOH, NH₂-[affinity tag]-[optional linker sequence]-[guide nucleotide sequence-programmable DNA binding protein domain]-[linker sequence]-[recombinase catalytic domain]-[optional linker sequence]-[NLS domain]-COOH, or NH₂-[affinity tag]-[optional linker sequence]-[N-terminal portion of a bifurcated or circularly permuted guide nucleotide sequence-programmable DNA binding protein domain]-[optional linker sequence]-[recombinase catalytic domain]-[optional linker sequence]-[C-terminal portion of a bifurcated or circularly permuted guide nucleotide sequence-programmable DNA binding protein domain]-[optional linker sequence]-[NLS domain]-COOH.

In another embodiment, the fusion protein has the structure: NH₂-[guide nucleotide sequence-programmable DNA binding protein domain]-[linker sequence]-[recombinase catalytic domain]-[optional linker sequence]-[optional NLS domain]-[optional linker sequence]-[optional affinity tag]-COOH. In one embodiment, the fusion protein comprises the structure NH₂-[guide nucleotide sequence-programmable DNA binding protein domain]-[linker sequence]-[recombinase catalytic domain]-[optional linker sequence]-[NLS domain]-[optional linker sequence]-[optional affinity tag]-COOH. In one embodiment, the fusion protein comprises the structure NH₂-[guide nucleotide sequence-programmable DNA binding protein domain]-[linker sequence]-[recombinase catalytic domain]-[optional linker sequence]-[NLS domain]-[optional linker sequence]-[affinity tag]-COOH. In one embodiment, the fusion protein comprises the structure NH₂-[guide nucleotide sequence-programmable DNA binding protein domain]-[linker sequence]-[recombinase catalytic domain]-[linker sequence]-[NLS domain]-[linker sequence]-[affinity tag]-COOH.

In another embodiment, the fusion protein has the structure NH₂-[N-terminal portion of a bifurcated or circularly permuted guide nucleotide sequence-programmable DNA binding protein domain]-[optional linker sequence]-[recombinase catalytic domain]-[optional linker sequence]-[C-terminal portion of a bifurcated or circularly permuted guide nucleotide sequence-programmable DNA binding protein domain]-[optional linker sequence]-[optional NLS domain]-[optional linker sequence]-[optional affinity tag]-COOH. In another embodiment, the fusion protein comprises the structure NH₂-[N-terminal portion of a bifurcated or circularly permuted guide nucleotide sequence-programmable DNA binding protein domain]-[optional linker sequence]-[recombinase catalytic domain]-[optional linker sequence]-[C-terminal portion of a bifurcated or circularly permuted guide nucleotide sequence-programmable DNA binding protein domain]-[optional linker sequence]-[NLS domain]-[optional linker sequence]-[optional affinity tag]-COOH. In another embodiment, the fusion protein comprises the structure NH₂-[N-terminal portion of a bifurcated or circularly permuted guide nucleotide sequence-programmable DNA binding protein domain]-[optional linker sequence]-[recombinase catalytic domain]-[optional linker sequence]-[C-terminal portion of a bifurcated or circularly permuted guide nucleotide sequence-programmable DNA binding protein domain]-[optional linker sequence]-[NLS domain]-[optional linker sequence]-[affinity tag]-COOH. In another embodiment, the fusion protein comprises the structure NH₂-[N-terminal portion of a bifurcated or circularly permuted guide nucleotide sequence-programmable DNA binding protein domain]-[optional linker sequence]-[recombinase catalytic domain]-[optional linker sequence]-[C-terminal portion of a bifurcated or circularly permuted guide nucleotide sequence-programmable DNA binding protein domain]-[linker sequence]-[NLS domain]-[linker sequence]-[affinity tag]-COOH.

In another embodiment, the fusion protein has the structure NH₂-[N-terminal portion of a bifurcated or circularly permuted guide nucleotide sequence-programmable DNA binding protein domain]-[linker sequence]-[recombinase catalytic domain]-[optional linker sequence]-[C-terminal portion of a bifurcated or circularly permuted guide nucleotide sequence-programmable DNA binding protein domain]-[optional linker sequence]-[optional NLS domain]-[optional linker sequence]-[optional affinity tag]-COOH. In another embodiment, the fusion protein comprises the structure NH₂-[N-terminal portion of a bifurcated or circularly permuted guide nucleotide sequence-programmable DNA binding protein domain]-[linker sequence]-[recombinase catalytic domain]-[optional linker sequence]-[C-terminal portion of a bifurcated or circularly permuted guide nucleotide sequence-programmable DNA binding protein domain]-[optional linker sequence]-[NLS domain]-[optional linker sequence]-[optional affinity tag]-COOH. In another embodiment, the fusion protein comprises the structure NH₂-[N-terminal portion of a bifurcated or circularly permuted guide nucleotide sequence-programmable DNA binding protein domain]-[linker sequence]-[recombinase catalytic domain]-[optional linker sequence]-[C-terminal portion of a bifurcated or circularly permuted guide nucleotide sequence-programmable DNA binding protein domain]-[optional linker sequence]-[NLS domain]-[optional linker sequence]-[affinity tag]-COOH. In another embodiment, the fusion protein comprises the structure NH₂-[N-terminal portion of a bifurcated or circularly permuted guide nucleotide sequence-programmable DNA binding protein domain]-[linker sequence]-[recombinase catalytic domain]-[optional linker sequence]-[C-terminal portion of a bifurcated or circularly permuted guide nucleotide sequence-programmable DNA binding protein domain]-[linker sequence]-[NLS domain]-[linker sequence]-[affinity tag]-COOH.

In another embodiment, the fusion protein has the structure NH₂-[N-terminal portion of a bifurcated or circularly permuted guide nucleotide sequence-programmable DNA binding protein domain]-[optional linker sequence]-[recombinase catalytic domain]-[linker sequence]-[C-terminal portion of a bifurcated or circularly permuted guide nucleotide sequence-programmable DNA binding protein domain]-[optional linker sequence]-[optional NLS domain]-[optional linker sequence]-[optional affinity tag]-COOH. In another embodiment, the fusion protein comprises the structure NH₂-[N-terminal portion of a bifurcated or circularly permuted guide nucleotide sequence-programmable DNA binding protein domain]-[optional linker sequence]-[recombinase catalytic domain]-[linker sequence]-[C-terminal portion of a bifurcated or circularly permuted guide nucleotide sequence-programmable DNA binding protein domain]-[optional linker sequence]-[NLS domain]-[optional linker sequence]-[optional affinity tag]-COOH. In another embodiment, the fusion protein comprises the structure NH₂-[N-terminal portion of a bifurcated or circularly permuted guide nucleotide sequence-programmable DNA binding protein domain]-[optional linker sequence]-[recombinase catalytic domain]-[linker sequence]-[C-terminal portion of a bifurcated or circularly permuted guide nucleotide sequence-programmable DNA binding protein domain]-[optional linker sequence]-[NLS domain]-[optional linker sequence]-[affinity tag]-COOH. In another embodiment, the fusion protein comprises the structure NH₂-[N-terminal portion of a bifurcated or circularly permuted guide nucleotide sequence-programmable DNA binding protein domain]-[optional linker sequence]-[recombinase catalytic domain]-[linker sequence]-[C-terminal portion of a bifurcated or circularly permuted guide nucleotide sequence-programmable DNA binding protein domain]-[linker sequence]-[NLS domain]-[linker sequence]-[affinity tag]-COOH.

In another embodiment, the fusion protein has the structure NH₂-[N-terminal portion of a bifurcated or circularly permuted guide nucleotide sequence-programmable DNA binding protein domain]-[linker sequence]-[recombinase catalytic domain]-[linker sequence]-[C-terminal portion of a bifurcated or circularly permuted guide nucleotide sequence-programmable DNA binding protein domain]-[optional linker sequence]-[optional NLS domain]-[optional linker sequence]-[optional affinity tag]-COOH. In another embodiment, the fusion protein comprises the structure NH₂-[N-terminal portion of a bifurcated or circularly permuted guide nucleotide sequence-programmable DNA binding protein domain]-[linker sequence]-[recombinase catalytic domain]-[linker sequence]-[C-terminal portion of a bifurcated or circularly permuted guide nucleotide sequence-programmable DNA binding protein domain]-[optional linker sequence]-[NLS domain]-[optional linker sequence]-[optional affinity tag]-COOH. In another embodiment, the fusion protein comprises the structure NH₂-[N-terminal portion of a bifurcated or circularly permuted guide nucleotide sequence-programmable DNA binding protein domain]-[linker sequence]-[recombinase catalytic domain]-[linker sequence]-[C-terminal portion of a bifurcated or circularly permuted guide nucleotide sequence-programmable DNA binding protein domain]-[optional linker sequence]-[NLS domain]-[optional linker sequence]-[affinity tag]-COOH. In another embodiment, the fusion protein comprises the structure NH₂-[N-terminal portion of a bifurcated or circularly permuted guide nucleotide sequence-programmable DNA binding protein domain]-[linker sequence]-[recombinase catalytic domain]-[linker sequence]-[C-terminal portion of a bifurcated or circularly permuted guide nucleotide sequence-programmable DNA binding protein domain]-[linker sequence]-[NLS domain]-[linker sequence]-[affinity tag]-COOH.

In one embodiment, the fusion protein has the structure NH₂-[optional affinity tag]-[optional linker sequence]-[optional NLS domain]-[optional linker sequence]-[recombinase catalytic domain]-[linker sequence]-[guide nucleotide sequence-programmable DNA binding protein domain]-COOH. In one embodiment, the fusion protein comprises the structure NH₂-[optional affinity tag]-[optional linker sequence]-[NLS domain]-[optional linker sequence]-[recombinase catalytic domain]-[linker sequence]-[guide nucleotide sequence-programmable DNA binding protein domain]-COOH. In one embodiment, the fusion protein comprises the structure NH₂-[affinity tag]-[optional linker sequence]-[NLS domain]-[optional linker sequence]-[recombinase catalytic domain]-[linker sequence]-[guide nucleotide sequence-programmable DNA binding protein domain]-COOH. In one embodiment, the fusion protein comprises the structure NH₂-[affinity tag]-[linker sequence]-[NLS domain]-[linker sequence]-[recombinase catalytic domain]-[linker sequence]-[guide nucleotide sequence-programmable DNA binding protein domain]-COOH.

In one embodiment, the fusion protein has the structure NH₂-[optional affinity tag]-[optional linker sequence]-[optional NLS domain]-[optional linker sequence]-[guide nucleotide sequence-programmable DNA binding protein domain]-[linker sequence]-[recombinase catalytic domain]-COOH. In one embodiment, the fusion protein comprises the structure NH₂-[optional affinity tag]-[optional linker sequence]-[NLS domain]-[optional linker sequence]-[guide nucleotide sequence-programmable DNA binding protein domain]-[linker sequence]-[recombinase catalytic domain]-COOH. In one embodiment, the fusion protein comprises the structure NH₂-[affinity tag]-[optional linker sequence]-[NLS domain]-[optional linker sequence]-[guide nucleotide sequence-programmable DNA binding protein domain]-[linker sequence]-[recombinase catalytic domain]-COOH. In one embodiment, the fusion protein comprises the structure NH₂-[affinity tag]-[linker sequence]-[NLS domain]-[linker sequence]-[guide nucleotide sequence-programmable DNA binding protein domain]-[linker sequence]-[recombinase catalytic domain]-COOH.

In another embodiment, the fusion protein has the structure NH₂-[optional affinity tag]-[optional linker sequence]-[optional NLS domain]-[optional linker sequence]-[N-terminal portion of a bifurcated or circularly permuted guide nucleotide sequence-programmable DNA binding protein domain]-[optional linker sequence]-[recombinase catalytic domain]-[optional linker sequence]-[C-terminal portion of a bifurcated or circularly permuted guide nucleotide sequence-programmable DNA binding protein domain]-COOH. In another embodiment, the fusion protein comprises the structure NH₂-[optional affinity tag]-[optional linker sequence]-[NLS domain]-[optional linker sequence]-[N-terminal portion of a bifurcated or circularly permuted guide nucleotide sequence-programmable DNA binding protein domain]-[optional linker sequence]-[recombinase catalytic domain]-[optional linker sequence]-[C-terminal portion of a bifurcated or circularly permuted guide nucleotide sequence-programmable DNA binding protein domain]-COOH. In another embodiment, the fusion protein comprises the structure NH₂-[affinity tag]-[optional linker sequence]-[NLS domain]-[optional linker sequence]-[N-terminal portion of a bifurcated or circularly permuted guide nucleotide sequence-programmable DNA binding protein domain]-[optional linker sequence]-[recombinase catalytic domain]-[optional linker sequence]-[C-terminal portion of a bifurcated or circularly permuted guide nucleotide sequence-programmable DNA binding protein domain]-COOH. In another embodiment, the fusion protein comprises the structure NH₂-[affinity tag]-[linker sequence]-[NLS domain]-[linker sequence]-[N-terminal portion of a bifurcated or circularly permuted guide nucleotide sequence-programmable DNA binding protein domain]-[optional linker sequence]-[recombinase catalytic domain]-[optional linker sequence]-[C-terminal portion of a bifurcated or circularly permuted guide nucleotide sequence-programmable DNA binding protein domain]-COOH.

In another embodiment, the fusion protein has the structure NH₂-[optional affinity tag]-[optional linker sequence]-[optional NLS domain]-[optional linker sequence]-[N-terminal portion of a bifurcated or circularly permuted guide nucleotide sequence-programmable DNA binding protein domain]-[linker sequence]-[recombinase catalytic domain]-[optional linker sequence]-[C-terminal portion of a bifurcated or circularly permuted guide nucleotide sequence-programmable DNA binding protein domain]-COOH. In another embodiment, the fusion protein comprises the structure NH₂-[optional affinity tag]-[optional linker sequence]-[NLS domain]-[optional linker sequence]-[N-terminal portion of a bifurcated or circularly permuted guide nucleotide sequence-programmable DNA binding protein domain]-[linker sequence]-[recombinase catalytic domain]-[optional linker sequence]-[C-terminal portion of a bifurcated or circularly permuted guide nucleotide sequence-programmable DNA binding protein domain]-COOH. In another embodiment, the fusion protein comprises the structure NH₂-[affinity tag]-[optional linker sequence]-[NLS domain]-[optional linker sequence]-[N-terminal portion of a bifurcated or circularly permuted guide nucleotide sequence-programmable DNA binding protein domain]-[linker sequence]-[recombinase catalytic domain]-[optional linker sequence]-[C-terminal portion of a bifurcated or circularly permuted guide nucleotide sequence-programmable DNA binding protein domain]-COOH. In another embodiment, the fusion protein comprises the structure NH₂-[affinity tag]-[linker sequence]-[NLS domain]-[linker sequence]-[N-terminal portion of a bifurcated or circularly permuted guide nucleotide sequence-programmable DNA binding protein domain]-[linker sequence]-[recombinase catalytic domain]-[optional linker sequence]-[C-terminal portion of a bifurcated or circularly permuted guide nucleotide sequence-programmable DNA binding protein domain]-COOH.

In another embodiment, the fusion protein has the structure NH₂-[optional affinity tag]-[optional linker sequence]-[optional NLS domain]-[optional linker sequence]-[N-terminal portion of a bifurcated or circularly permuted guide nucleotide sequence-programmable DNA binding protein domain]-[optional linker sequence]-[recombinase catalytic domain]-[linker sequence]-[C-terminal portion of a bifurcated or circularly permuted guide nucleotide sequence-programmable DNA binding protein domain]-COOH. In another embodiment, the fusion protein comprises the structure NH₂-[optional affinity tag]-[optional linker sequence]-[NLS domain]-[optional linker sequence]-[N-terminal portion of a bifurcated or circularly permuted guide nucleotide sequence-programmable DNA binding protein domain]-[optional linker sequence]-[recombinase catalytic domain]-[linker sequence]-[C-terminal portion of a bifurcated or circularly permuted guide nucleotide sequence-programmable DNA binding protein domain]-COOH. In another embodiment, the fusion protein comprises the structure NH₂-[affinity tag]-[optional linker sequence]-[NLS domain]-[optional linker sequence]-[N-terminal portion of a bifurcated or circularly permuted guide nucleotide sequence-programmable DNA binding protein domain]-[optional linker sequence]-[recombinase catalytic domain]-[linker sequence]-[C-terminal portion of a bifurcated or circularly permuted guide nucleotide sequence-programmable DNA binding protein domain]-COOH. In another embodiment, the fusion protein comprises the structure NH₂-[affinity tag]-[linker sequence]-[NLS domain]-[linker sequence]-[N-terminal portion of a bifurcated or circularly permuted guide nucleotide sequence-programmable DNA binding protein domain]-[optional linker sequence]-[recombinase catalytic domain]-[linker sequence]-[C-terminal portion of a bifurcated or circularly permuted guide nucleotide sequence-programmable DNA binding protein domain]-COOH.

In another embodiment, the fusion protein has the structure NH₂-[optional affinity tag]-[optional linker sequence]-[optional NLS domain]-[optional linker sequence]-[N-terminal portion of a bifurcated or circularly permuted guide nucleotide sequence-programmable DNA binding protein domain]-[linker sequence]-[recombinase catalytic domain]-[linker sequence]-[C-terminal portion of a bifurcated or circularly permuted guide nucleotide sequence-programmable DNA binding protein domain]-COOH. In another embodiment, the fusion protein comprises the structure NH₂-[optional affinity tag]-[optional linker sequence]-[NLS domain]-[optional linker sequence]-[N-terminal portion of a bifurcated or circularly permuted guide nucleotide sequence-programmable DNA binding protein domain]-[linker sequence]-[recombinase catalytic domain]-[linker sequence]-[C-terminal portion of a bifurcated or circularly permuted guide nucleotide sequence-programmable DNA binding protein domain]-COOH. In another embodiment, the fusion protein comprises the structure NH₂-[affinity tag]-[optional linker sequence]-[NLS domain]-[optional linker sequence]-[N-terminal portion of a bifurcated or circularly permuted guide nucleotide sequence-programmable DNA binding protein domain]-[linker sequence]-[recombinase catalytic domain]-[linker sequence]-[C-terminal portion of a bifurcated or circularly permuted guide nucleotide sequence-programmable DNA binding protein domain]-COOH. In another embodiment, the fusion protein comprises the structure NH₂-[affinity tag]-[linker sequence]-[NLS domain]-[linker sequence]-[N-terminal portion of a bifurcated or circularly permuted guide nucleotide sequence-programmable DNA binding protein domain]-[linker sequence]-[recombinase catalytic domain]-[linker sequence]-[C-terminal portion of a bifurcated or circularly permuted guide nucleotide sequence-programmable DNA binding protein domain]-COOH.

The fusion protein may further comprise one or more affinity tags. Suitable affinity tags provided herein include, but are not limited to, biotin carboxylase carrier protein (BCCP) tags, myc-tags, calmodulin-tags, FLAG-tags, hemagglutinin (HA)-tags, polyhistidine tags, also referred to as histidine tags or His-tags, polyarginine (poly-Arg) tags, maltose binding protein (MBP)-tags, nus-tags, glutathione-S-transferase (GST)-tags, green fluorescent protein (GFP)-tags, thioredoxin-tags, S-tags, Softags (e.g., Softag 1, Softag 3), strep-tags, biotin ligase tags, FlAsH tags, V5 tags, and SBP-tags. Additional suitable sequences will be apparent to those of skill in the art. The FLAG tag may have the sequence PKKKRKV (SEQ ID NO: 702). The one or more affinity tags are bound to the guide nucleotide sequence-programmable DNA binding protein domain, the recombinase catalytic domain, or the NLS domain via one or more third linkers. The third linker may be any peptide linker described herein. For example, the third linker may be a peptide linker.

As a non-limiting set of examples, the third linker may comprise an XTEN linker SGSETPGTSESATPES (SEQ ID NO: 7), SGSETPGTSESA (SEQ ID NO: 8), or SGSETPGTSESATPEGGSGGS (SEQ ID NO: 9), an amino acid sequence comprising one or more repeats of the tri-peptide GGS, or any of the following amino acid sequences: VPFLLEPDNINGKTC (SEQ ID NO: 10), GSAGSAAGSGEF (SEQ ID NO: 11), SIVAQLSRPDPA (SEQ ID NO: 12), MKIIEQLPSA (SEQ ID NO: 13), VRHKLKRVGS (SEQ ID NO: 14), GHGTGSTGSGSS (SEQ ID NO: 15), MSRPDPA (SEQ ID NO; 16), or GGSM (SEQ ID NO: 17). In certain embodiments, the third linker comprises one or more repeats of the tri-peptide GGS. In an embodiment, the third linker comprises from one to five repeats of the tri-peptide GGS. In another embodiment, the third linker comprises one repeat of the tri-peptide GGS. In a specific embodiment, the third linker has the sequence GGS.

The third linker may also be a non-peptide linker. In certain embodiments, the non-peptide linker comprises polyethylene glycol (PEG), polypropylene glycol (PPG), co-poly(ethylene/propylene) glycol, polyoxyethylene (POE), polyurethane, polyphosphazene, polysaccharides, dextran, polyvinyl alcohol, polyvinylpyrrolidones, polyvinyl ethyl ether, polyacryl amide, polyacrylate, polycyanoacrylates, lipid polymers, chitins, hyaluronic acid, heparin, or an alkyl linker. In other embodiments, the alkyl linker has the formula: —NH—(CH₂)ₛ—C(O)—, wherein s may be any integer between 1 and 100, inclusive. In a specific embodiment, s is any integer between 1 and 20, inclusive.

The fusion protein of the instant disclosure has greater than 90%, 95%, or 99% sequence identity with the amino acid sequence shown in amino acids 1-1544 of SEQ ID NO: 185, which is identical to the sequence shown in SEQ ID NO: 719.

(SEQ ID NO: 719)
MLIGYVRVSTNDQNTDLQRNALVCAGCEQIFEDKLSGTRTDRPGLKRALK
RLQKGDTLVVWKLDRLGRSMKHLISLVGELRERGINFRSLTDSIDTSSPM
GRFFFYVMGALAEMERELIIERTMAGLAAARNKGRRFGRPPKGGSGGSGG
SGGSGGSGGSGGSGGSDKKYSIGLAIGTNSVGWAVITDEYKVPSKKFKVL
GNTDRHSIKKNLIGALLFDSGETAEATRLKRTARRRYTRRKNRICYLQEI
FSNEMAKVDDSFFHRLEESFLVEEDKKHERHPIFGNIVDEVAYHEKYPTI
YHLRKKLVDSTDKADLRLIYLALAHMIKFRGHFLIEGDLNPDNSDVDKLF
IQLVQTYNQLFEENPINASGVDAKAILSARLSKSRRLENLIAQLPGEKKN
GLFGNLIALSLGLTPNFKSNFDLAEDAKLQLSKDTYDDDLDNLLAQIGDQ
YADLFLAAKNLSDAILLSDILRVNTEITKAPLSASMIKRYDEHHQDLTLL
KALVRQQLPEKYKEIFFDQSKNGYAGYIDGGASQEEFYKFIKPILEKMDG
TEELLVKLNREDLLRKQRTFDNGSIPHQIHLGELHAILRRQEDFYPFLKD
NREKIEKILTFRIPYYVGPLARGNSRFAWMTRKSEETITPWNFEEVVDKG
ASAQSFIERMTNFDKNLPNEKVLPKHSLLYEYFTVYNELTKVKYVTEGMR
KPAFLSGEQKKAIVDLLFKTNRKVTVKQLKEDYFKKIECFDSVEISGVED
RFNASLGTYHDLLKIIKDKDFLDNEENEDILEDIVLTLTLFEDREMIEER
LKTYAHLFDDKVMKQLKRRRYTGWGRLSRKLINGIRDKQSGKTILDFLKS
DGFANRNFMQLIHDDSLTFKEDIQKAQVSGQGDSLHEHIANLAGSPAIKK
GILQTVKVVDELVKVMGRHKPENIVIEMARENQTTQKGQKNSRERMKRIE
EGIKELGSQILKEHPVENTQLQNEKLYLYYLQNGRDMYVDQELDINRLSD
YDVDAIVPQSFLKDDSIDNKVLTRSDKNRGKSDNVPSEEVVKKMKNYWRQ
LLNAKLITQRKFDNLTKAERGGLSELDKAGFIKRQLVETRQITKHVAQIL
DSRMNTKYDENDKLIREVKVITLKSKLVSDFRKDFQFYKVREINNYHHAH
DAYLNAVVGTALIKKYPKLESEFVYGDYKVYDVRKMIAKSEQEIGKATAK
YFFYSNIMNFFKTEITLANGEIRKRPLIETNGETGEIVWDKGRDFATVRK
VLSMPQVNIVKKTEVQTGGFSKESILPKRNSDKLIARKKDWDPKKYGGFD
SPTVAYSVLVVAKVEKGKSKKLKSVKELLGITIMERSSFEKNPIDFLEAK
GYKEVKKDLIIKLPKYSLFELENGRKRMLASAGELQKGNELALPSKYVNF
LYLASHYEKLKGSPEDNEQKQLFVEQHKHYLDEIIEQISEFSKRVILADA
NLDKVLSAYNKHRDKPIREQAENIIHLFTLTNLGAPAAFKYFDTTIDRKR
YTSTKEVLDATLIHQSITGLYETRIDLSQLGGDGGSDYKDDDDK Stop

In the context of proteins that dimerize (or multimerize) such as, for example, fusions between a nuclease-inactivated Cas9 (or a Cas9 gRNA binding domain) and a recombinase (or catalytic domain of a recombinase), a target site typically comprises a left-half site (bound by one protein), a right-half site (bound by the second protein), and a spacer sequence between the half sites in which the recombination is made. In some embodiments, either the left-half site or the right half-site (and not the spacer sequence) is recombined. In other embodiments, the spacer sequence is recombined. This structure ([left-half site]-[spacer sequence]-[right-half site]) is referred to herein as an LSR structure. In some embodiments, the left-half site and/or the right-half site correspond to an RNA-guided target site (e.g., a Cas9 target site). In some embodiments, either or both half-sites are shorter or longer than e.g., a typical region targeted by Cas9, for example shorter or longer than 20 nucleotides. In some embodiments, the left and right half sites comprise different nucleic acid sequences. In some embodiments, the spacer sequence is at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, at least 50, at least 60, at least 70, at least 80, at least 90, at least 100, at least 125, at least 150, at least 175, at least 200, or at least 250 bp long. In some embodiments, the spacer sequence is between approximately 15 bp and approximately 25 bp long. In some embodiments, the spacer sequence is approximately 15 bp long. In some embodiments, the spacer sequence is approximately 25 bp long.

### EXAMPLES

### Example 1: A Programmable Cas9-Serine Recombinase Fusion Protein that Operates on DNA Sequences in Mammalian Cells

### Materials and Methods

### Oligonucleotides and PCR

All oligonucleotides were purchased from Integrated DNA Technologies (IDT, Coralville, CA) and are listed in Tables 1-5. Enzymes, unless otherwise noted, were purchased from New England Biolabs (Ipswich, MA). Plasmid Safe ATP-dependent DNAse was purchased from Epicentre (Madison, WI). All assembled vectors were transformed into One Shot Mach1-T1 phage-resistant chemically competent cells (Fisher Scientific, Waltham, MA). Unless otherwise noted, all PCR reactions were performed with Q5 Hot Start High-Fidelity 2× Master Mix. Phusion polymerase was used for circular polymerase extension cloning (CPEC) assemblies.

TABLE 1
Oligonucleotides for gRNA construction

| | | SEQ ID |
|---|---|---|
| Oligonucleotide Name | Sequence | NO: |
| R.pHU6.TSS(−1).univ | GGTGTTTCGTCCTTTCCACAAG | 20 |
| F.non-target | GCACACTAGTTAGGGATAACAGTTTTAG | 21 |
| | AGCTAGAAATAGC | |
| F.Chr10-1 | GCCCATGACCCTTCTCCTCTGTTTTAGAG | 22 |
| | CTAGAAATAGC | |
| F.Chr10-1-rev | GCTCAGGGCCTGTGATGGGAGGTTTTAG | 23 |
| | AGCTAGAAATAGC | |
| F.Chr10-2 | GGCCCATGACCCTTCTCCTCGTTTTAGAG | 24 |
| | CTAGAAATAGC | |
| F.Chr10-2rev | GCCTCAGGGCCTGTGATGGGAGTTTTAG | 25 |
| | AGCTAGAAATAGC | |
| F.Centromere_Chr_1_5_19- | GACTTGAAACACTCTTTTTCGTTTTAGAG | 26 |
| gRNA-for | CTAGAAATAGC | |
| F.Centromere_Chr_1_5_19- | GAGTTGAAGACACACAACACAGTTTTAG | 27 |
| gRNA-rev | AGCTAGAAATAGC | |
| F.Ch5_155183064-gRNA-for | GGAACTCATGTGATTAACTGGTTTTAGA | 28 |
| | GCTAGAAATAGC | |
| F.Ch5_155183064-gRNA-rev-1 | GTCTACCTCTCATGAGCCGGTGTTTTAGA | 29 |
| | GCTAGAAATAGC | |
| F.Ch5_169395198-gRNA-for | GTTTCCCGCAGGATGTGGGATGTTTTAG | 30 |
| | AGCTAGAAATAGC | |
| F.Ch5_169395198-gRNA-rev | GCCTGGGGATTTATGTTCTTAGTTTTAGA | 31 |
| | GCTAGAAATAGC | |
| F.Ch12_62418577-gRNA-for | GAAATAGCACAATGAATGGAAGTTTTAG | 32 |
| | AGCTAGAAATAGC | |
| F.Ch12_62418577-gRNA-rev | GACTTTTTGGGGGAGAGGGAGGTTTTAG | 33 |
| | AGCTAGAAATAGC | |
| F.Ch13_102010574-gRNA-for | GGAGACTTAAGTCCAAAACCGTTTTAGA | 34 |
| | GCTAGAAATAGC | |
| F.Ch13_102010574-gRNA- | GTCAGCTATGATCACTTCCCTGTTTTAGA | 35 |
| rev | GCTAGAAATAGC | |

TABLE 2
Oligonucleotides and gBlocks for reporter construction

| | | SEQ ID |
|---|---|---|
| Construct Name | Sequence | NO: |
| 1-0bp-for | TCGTCTCGGCGTCCCCAATTTTCCCAAACAGAG | 36 |
| | GTCTGTAAACCGAGGTGAGACGG | |
| 1-0bp-rev | CCGTCTCACCTCGGTTTACAGACCTCTGTTTGG | 37 |
| | GAAAATTGGGGACGCCGAGACGA | |
| 1-1bp-for | TCGTCTCGGCGTCCCCAATTTTCCCAAACAGAG | 38 |
| | GTtCTGTAAACCGAGGTGAGACGG | |
| 1-1bp-rev | CCGTCTCACCTCGGTTTACAGaACCTCTGTTTGG | 39 |
| | GAAAATTGGGGACGCCGAGACGA | |
| 1-2bp-for | TCGTCTCGGCGTCCCCAATTTTCCCAAACAGAG | 40 |
| | GTatCTGTAAACCGAGGTGAGACGG | |
| 1-2bp-rev | CCGTCTCACCTCGGTTTACAGatACCTCTGTTTG | 41 |
| | GGAAAATTGGGGACGCCGAGACGA | |
| 1-3bp-for | TCGTCTCGGCGTCCCCAATTTTCCCAAACAGAG | 42 |
| | GTaatCTGTAAACCGAGGTGAGACGG | |
| 1-3bp-rev | CCGTCTCACCTCGGTTTACAGattACCTCTGTTTG | 43 |
| | GGAAAATTGGGGACGCCGAGACGA | |
| 1-4bp-for | TCGTCTCGGCGTCCCCAATTTTCCCAAACAGAG | 44 |
| | GTaaatCTGTAAACCGAGGTGAGACGG | |
| 1-4bp-rev | CCGTCTCACCTCGGTTTACAGatttACCTCTGTTT | 45 |
| | GGGAAAATTGGGGACGCCGAGACGA | |
| 1-5bp-for | TCGTCTCGGCGTCCCCAATTTTCCCAAACAGAG | 46 |
| | GTgaaatCTGTAAACCGAGGTGAGACGG | |
| 1-5bp-rev | CCGTCTCACCTCGGTTTACAGatttcACCTCTGTTT | 47 |
| | GGGAAAATTGGGGACGCCGAGACGA | |
| 1-6bp-for | TCGTCTCGGCGTCCCCAATTTTCCCAAACAGAG | 48 |
| | GTcgaaatCTGTAAACCGAGGTGAGACGG | |
| 1-6bp-rev | CCGTCTCACCTCGGTTTACAGatttcgACCTCTGTT | 49 |
| | TGGGAAAATTGGGGACGCCGAGACGA | |
| 1-7bp-for | TCGTCTCGGCGTCCCCAATTTTCCCAAACAGAG | 50 |
| | GTtcgaaatCTGTAAACCGAGGTGAGACGG | |
| 1-7bp-rev | CCGTCTCACCTCGGTTTACAGatttcgaACCTCTGT | 51 |
| | TTGGGAAAATTGGGGACGCCGAGACGA | |
| 2-0bp-for | TCGTCTCGGAGGTTTTGGAACCTCTGTTTGGGA | 52 |
| | AAATTGGGGAGTCTGAGACGG | |
| 2-0bp-rev | CCGTCTCAGACTCCCCAATTTTCCCAAACAGAG | 53 |
| | GTTCCAAAACCTCCGAGACGA | |
| 2-1bp-for | TCGTCTCGGAGGTTTTGGACACCTCTGTTTGGG | 54 |
| | AAAATTGGGGAGTCTGAGACGG | |
| 2-1bp-rev | CCGTCTCAGACTCCCCAATTTTCCCAAACAGAG | 55 |
| | GTGTCCAAAACCTCCGAGACGA | |
| 2-2bp-for | TCGTCTCGGAGGTTTTGGACTACCTCTGTTTGG | 56 |
| | GAAAATTGGGGAGTCTGAGACGG | |
| 2-2bp-rev | CCGTCTCAGACTCCCCAATTTTCCCAAACAGAG | 57 |
| | GTAGTCCAAAACCTCCGAGACGA | |
| 2-3bp-for | TCGTCTCGGAGGTTTTGGACTTACCTCTGTTTG | 58 |
| | GGAAAATTGGGGAGTCTGAGACGG | |
| 2-3bp-rev | CCGTCTCAGACTCCCCAATTTTCCCAAACAGAG | 59 |
| | GTAAGTCCAAAACCTCCGAGACGA | |
| 2-4bp-for | TCGTCTCGGAGGTTTTGGACTTAACCTCTGTTT | 60 |
| | GGGAAAATTGGGGAGTCTGAGACGG | |
| 2-4bp-rev | CCGTCTCAGACTCCCCAATTTTCCCAAACAGAG | 61 |
| | GTTAAGTCCAAAACCTCCGAGACGA | |
| 2-5bp-for | TCGTCTCGGAGGTTTTGGACTTAGACCTCTGTT | 62 |
| | TGGGAAAATTGGGGAGTCTGAGACGG | |
| 2-5bp-rev | CCGTCTCAGACTCCCCAATTTTCCCAAACAGAG | 63 |
| | GTCTAAGTCCAAAACCTCCGAGACGA | |
| 2-6bp-for | TCGTCTCGGAGGTTTTGGACTTAGCACCTCTGT | 64 |
| | TTGGGAAAATTGGGGAGTCTGAGACGG | |
| 2-6bp-rev | CCGTCTCAGACTCCCCAATTTTCCCAAACAGAG | 65 |
| | GTGCTAAGTCCAAAACCTCCGAGACGA | |
| 2-7bp-for | TCGTCTCGGAGGTTTTGGACTTAGCTACCTCTG | 66 |
| | TTTGGGAAAATTGGGGAGTCTGAGACGG | |
| 2-7bp-rev | CCGTCTCAGACTCCCCAATTTTCCCAAACAGAG | 67 |
| | GTAGCTAAGTCCAAAACCTCCGAGACGA | |
| 4-0bp-for | TCGTCTCTGCACCCCCAATTTTCCCAAACAGAG | 68 |
| | GTCTGTAAACCGATGAGACGG | |
| 4-0bp-rev | CCGTCTCATCGGTTTACAGACCTCTGTTTGGGA | 69 |
| | AAATTGGGGGTGCAGAGACGA | |
| 4-1bp-for | TCGTCTCTGCACCCCCAATTTTCCCAAACAGAG | 70 |
| | GTtCTGTAAACCGATGAGACGG | |
| 4-1bp-rev | CCGTCTCATCGGTTTACAGaACCTCTGTTTGGG | 71 |
| | AAAATTGGGGGTGCAGAGACGA | |
| 4-2bp-for | TCGTCTCTGCACCCCCAATTTTCCCAAACAGAG | 72 |
| | GTatCTGTAAACCGATGAGACGG | |
| 4-2bp-rev | CCGTCTCATCGGTTTACAGatACCTCTGTTTGGG | 73 |
| | AAAATTGGGGGTGCAGAGACGA | |
| 4-3bp-for | TCGTCTCTGCACCCCCAATTTTCCCAAACAGAG | 74 |
| | GTaatCTGTAAACCGATGAGACGG | |
| 4-3bp-rev | CCGTCTCATCGGTTTACAGattACCTCTGTTTGGG | 75 |
| | AAAATTGGGGGTGCAGAGACGA | |
| 4-4bp-for | TCGTCTCTGCACCCCCAATTTTCCCAAACAGAG | 76 |
| | GTaaatCTGTAAACCGATGAGACGG | |
| 4-4bp-rev | CCGTCTCATCGGTTTACAGatttACCTCTGTTTGG | 77 |
| | GAAAATTGGGGGTGCAGAGACGA | |
| 4-5bp-for | TCGTCTCTGCACCCCCAATTTTCCCAAACAGAG | 78 |
| | GTgaaatCTGTAAACCGATGAGACGG | |
| 4-5bp-rev | CCGTCTCATCGGTTTACAGatttcACCTCTGTTTGG | 79 |
| | GAAAATTGGGGGTGCAGAGACGA | |
| 4-6bp-for | TCGTCTCTGCACCCCCAATTTTCCCAAACAGAG | 80 |
| | GTcgaaatCTGTAAACCGATGAGACGG | |
| 4-6bp-rev | CCGTCTCATCGGTTTACAGatttcgACCTCTGTTTG | 81 |
| | GGAAAATTGGGGGTGCAGAGACGA | |
| 4-7bp-for | TCGTCTCTGCACCCCCAATTTTCCCAAACAGAG | 82 |
| | GTtcgaaatCTGTAAACCGATGAGACGG | |
| 4-7bp-rev | CCGTCTCATCGGTTTACAGatttcgaACCTCTGTTT | 83 |
| | GGGAAAATTGGGGGTGCAGAGACGA | |
| 5-0bp-for | TCGTCTCGCCGAGGTTTTGGAACCTCTGTTTGG | 84 |
| | GAAAATTGGGGCTCGTGAGACGG | |
| 5-0bp-rev | CCGTCTCACGAGCCCCAATTTTCCCAAACAGAG | 85 |
| | GTTCCAAAACCTCGGCGAGACGA | |
| 5-1bp-for | TCGTCTCGCCGAGGTTTTGGACACCTCTGTTTG | 86 |
| | GGAAAATTGGGGCTCGTGAGACGG | |
| 5-1bp-rev | CCGTCTCACGAGCCCCAATTTTCCCAAACAGAG | 87 |
| | GTGTCCAAAACCTCGGCGAGACGA | |
| 5-2bp-for | TCGTCTCGCCGAGGTTTTGGACTACCTCTGTTT | 88 |
| | GGGAAAATTGGGGCTCGTGAGACGG | |
| 5-2bp-rev | CCGTCTCACGAGCCCCAATTTTCCCAAACAGAG | 89 |
| | GTAGTCCAAAACCTCGGCGAGACGA | |
| 5-3bp-for | TCGTCTCGCCGAGGTTTTGGACTTACCTCTGTT | 90 |
| | TGGGAAAATTGGGGCTCGTGAGACGG | |
| 5-3bp-rev | CCGTCTCACGAGCCCCAATTTTCCCAAACAGAG | 91 |
| | GTAAGTCCAAAACCTCGGCGAGACGA | |
| 5-4bp-for | TCGTCTCGCCGAGGTTTTGGACTTAACCTCTGT | 92 |
| | TTGGGAAAATTGGGGCTCGTGAGACGG | |
| 5-4bp-rev | CCGTCTCACGAGCCCCAATTTTCCCAAACAGAG | 93 |
| | GTTAAGTCCAAAACCTCGGCGAGACGA | |
| 5-5bp-for | TCGTCTCGCCGAGGTTTTGGACTTAGACCTCTG | 94 |
| | TTTGGGAAAATTGGGGCTCGTGAGACGG | |
| 5-5bp-rev | CCGTCTCACGAGCCCCAATTTTCCCAAACAGAG | 95 |
| | GTCTAAGTCCAAAACCTCGGCGAGACGA | |
| 5-6bp-for | TCGTCTCGCCGAGGTTTTGGACTTAGCACCTCT | 96 |
| | GTTTGGGAAAATTGGGGCTCGTGAGACGG | |
| 5-6bp-rev | CCGTCTCACGAGCCCCAATTTTCCCAAACAGAG | 97 |
| | GTGCTAAGTCCAAAACCTCGGCGAGACGA | |
| 5-7bp-for | TCGTCTCGCCGAGGTTTTGGACTTAGCTACCTC | 98 |
| | TGTTTGGGAAAATTGGGGCTCGTGAGACGG | |
| 5-7bp-rev | CCGTCTCACGAGCCCCAATTTTCCCAAACAGAG | 99 |
| | GTAGCTAAGTCCAAAACCTCGGCGAGACGA | |
| 1-Chr10--54913298- | TCGTCTCGGCGTCCCCTCCCATCACAGGCCCTG | 100 |
| 54913376-for | AGGTTTAAGAGAAAACCTGAGACGG | |
| 1-Chr10-54913298- | CCGTCTCAGGTTTTCTCTTAAACCTCAGGGCCT | 101 |
| 54913376-rev | GTGATGGGAGGGGACGCCGAGACGA | |
| 2-Chr10--54913298- | TCGTCTCGAACCATGGTTTTGTGGGCCAGGCCC | 102 |
| 54913376-for | ATGACCCTTCTCCTCTGGGAGTCTGAGACGG | |
| 2-Chr10--54913298- | CCGTCTCAGACTCCCAGAGGAGAAGGGTCATG | 103 |
| 54913376-rev | GGCCTGGCCCACAAAACCATGGTTCGAGACGA | |
| 4-Chr10-54913298- | TCGTCTCTGCACCCCCTCCCATCACAGGCCCTG | 104 |
| 54913376-for | AGGTTTAAGAGAAAACCATTGAGACGG | |
| 4-Chr10-54913298- | CCGTCTCAATGGTTTTCTCTTAAACCTCAGGGC | 105 |
| 54913376-rev | CTGTGATGGGAGGGGGTGCAGAGACGA | |
| 5-Chr10-54913298- | TCGTCTCGCCATGGTTTTGTGGGCCAGGCCCAT | 106 |
| 54913376-for | GACCCTTCTCCTCTGGGCTCGTGAGACGG | |
| 5-Chr10-54913298- | CCGTCTCACGAGCCCAGAGGAGAAGGGTCATG | 107 |
| 54913376-rev | GGCCTGGCCCACAAAACCATGGCGAGACGA | |
| 3-for | ATCCGTCTCCAGTCGAGTCGGATTTGATCTGAT | 108 |
| | CAAGAGACAG | |
| 3-rev | AACCGTCTCGGTGCGTTCGGATTTGATCCAGAC | 109 |
| | ATGATAAGATAC | |
| Esp3I-insert-for | /Phos/CGCGTTGAGACGCTGCCATCCGTCTCGC | 110 |
| Esp3I-insert-rev | /Phos/TCGAGCGAGACGGATGGCAGCGTCTCAA | 111 |
| Centromere_Chr_1_5_19- | GTTGTTCGTCTCGGCGTCCTTGTGTTGTGTGTCT | 112 |
| 1_2* | TCAACTCACAGAGTTAAACGATGCTTTACACA | |
| | GAGTAGACTTGAAACACTCTTTTTCTGGAGTCT | |
| | GAGACGGTTCTGTTTTGGTGTGATTAGTTAT | |
| Centromere_Chr_1_5_19- | GTTGGTCGTCTCTGCACCCTTGTGTTGTGTGTCT | 113 |
| 4_5* | TCAACTCACAGAGTTAAACGATGCTTTACACA | |
| | GAGTAGACTTGAAACACTCTTTTTCTGGCTCGT | |
| | GAGACGGTTCTGTTTTGGTGTGATTAGTTAT | |
| Ch5_155183064- | GTTGTTCGTCTCGGCGTCCCACCGGCTCATGAG | 114 |
| 155183141-1_2* | AGGTAGAGCTAAGGTCCAAACCTAGGTTTATC | |
| | TGAGACCGGAACTCATGTGATTAACTGTGGAG | |
| | TCTGAGACGGTTCTGTTTTGGTGTGATTAGTTAT | |
| Ch5_155183064- | GTTGGTCGTCTCTGCACCCCACCGGCTCATGAG | 115 |
| 155183141-4_5* | AGGTAGAGCTAAGGTCCAAACCTAGGTTTATC | |
| | TGAGACCGGAACTCATGTGATTAACTGTGGCTC | |
| | GTGAGACGGTTCTGTTTTGGTGTGATTAGTTAT | |
| Ch5_169395198- | GTTGTTCGTCTCGGCGTCCTTAAGAACATAAAT | 116 |
| 169395274-1_2* | CCCCAGGAATTCACAGAAACCTTGGTTTGAGCT | |
| | TTGGATTTCCCGCAGGATGTGGGATAGGAGTCT | |
| | GAGACGGTTCTGTTTTGGTGTGATTAGTTAT | |
| Ch5_169395198- | GTTGGTCGTCTCTGCACCCTTAAGAACATAAAT | 117 |
| 169395274-4_5* | CCCCAGGAATTCACAGAAACCTTGGTTTGAGCT | |
| | TTGGATTTCCCGCAGGATGTGGGATAGGCTCGT | |
| | GAGACGGTTCTGTTTTGGTGTGATTAGTTAT | |
| Ch12_62418577- | GTTGTTCGTCTCGGCGTCCACTCCCTCTCCCCC | 118 |
| 62418652-1_2* | AAAAAGTAAAGGTAGAAAACCAAGGTTTACAG | |
| | GCAACAAATAGCACAATGAATGGAATGGAGTC | |
| | TGAGACGGTTCTGTTTTGGTGTGATTAGTTAT | |
| Ch12_62418577- | GTTGGTCGTCTCTGCACCCACTCCCTCTCCCCC | 119 |
| 62418652-4_5* | AAAAAGTAAAGGTAGAAAACCAAGGTTTACAG | |
| | GCAACAAATAGCACAATGAATGGAATGGCTCG | |
| | TGAGACGGTTCTGTTTTGGTGTGATTAGTTAT | |
| chr13_102010574- | GTTGTTCGTCTCGGCGTCCTAGGGAAGTGATCA | 120 |
| 102010650-1_2* | TAGCTGAGTTTCTGGAAAAACCTAGGTTTTAAA | |
| | GTTGAGGAGACTTAAGTCCAAAACCTGGAGTC | |
| | TGAGACGGTTCTGTTTTGGTGTGATTAGTTAT | |
| chr13_102010574- | GTTGGTCGTCTCTGCACCCTAGGGAAGTGATCA | 121 |
| 102010650-4_5* | TAGCTGAGTTTCTGGAAAAACCTAGGTTTTAAA | |
| | GTTGAGGAGACTTAAGTCCAAAACCTGGCTCG | |
| | TGAGACGGTTCTGTTTTGGTGTGATTAGTTAT | |

Oligonucleotide sequences were annealed to create the fragments shown in FIG. 1. The names correspond to the fragment number (1, 2, 4, or 5) and then to the number of base pair spacer nucleotides separating the Cas9 binding site from the gix core site.
*Double stranded gBlocks as described in the methods within the supporting material document.

TABLE 3
Oligonucleotides for recCas9 construction

| | | SEQ ID |
|---|---|---|
| Oligonucleotide Name | Sequence | NO: |
| 1GGS-link-for_BamHI | TTCATCGGATCCGATAAAAAGTATTCTATTG | 122 |
| | GTTTAGCTATCGGCAC | |
| 5GGS-link-for_BamHI | TTCATCGGATCCGGTGGTTCAGGTGGCAGC | 123 |
| | GGAG | |
| 8GGS-link-for_BamHI | TTCATCGGATCCGGAGGGTCCGGAGGTAGT | 124 |
| | GGCGGCAGCGGTGGTTCAGGTGGCAGCGGAG | |
| Cas9-rev-FLAG-NLS- | AATAACCGGTTCAGACCTTCCTTTTCTTCTT | 125 |
| AgeI | TGGGGAACCTCCCTTGTCGTCATCATCCTTA | |
| | TAATCGGAGCCACCGTCACCCCCAAGCTGT | |
| | GACAAATC | |
| 1GGS-rev-BamHI | TGATAAGGATCCACCCTTTGGTGGTCTTCCA | 126 |
| | AACCGCC | |
| 2GGS-rev-BamH | TGATAAGGATCCACCGCTACCACCCTTTGG | 127 |
| | TGGTCTTC | |
| Gin-for_NotI | AGATCCGCGGCCGCTAATAC | 128 |
| Esp3I-for-plasmid | TTGAGTcgtctcTATACTCTTCCTTTTTCAATAT | 129 |
| | TATTGAAGCATTTATCAGGG | |
| Esp3I-rev-plasmid | CTGGAAcgtctcACTGTCAGACCAAGTTTACTC | 130 |
| | ATATATACTTTAGATTG | |
| spec-Esp3I-for | GGTGTGcgtctcTACAGTTATTTGCCGACTACC | 131 |
| | TTGGTGATCTCGC | |
| spec-Esp3I-rev | ACACCAcgtctcTGTATGAGGGAAGCGGTGAT | 132 |
| | CGCC | |
| cpec assembly-for- | CATACTCTTCCTTTTTCAATATTATTGAAGC | 133 |
| plasmid | ATTTATCAGGG | |
| cpec assembly-rev- | CTGTCAGACCAAGTTTACTCATATATACTTT | 134 |
| plasmid | AGATTG | |
| cpec assembly-for-spec | CAATCTAAAGTATATATGAGTAAACTTGGT | 135 |
| | CTGACAGTTTGCCGACTACCTTGGTGATCTCG | |
| cpec assembly-for-spec2 | CAATCTAAAGTATATATGAGTAAACTTGGT | 136 |
| | CTGACAGTTATTTGCCGACTACCTTGGTGAT | |
| | CTCG | |
| cpec assembly-rev-spec | CCCTGATAAATGCTTCAATAATATTGAAAA | 137 |
| | AGGAAGAGTATG | |

TABLE 4
Custom sequencing oligonucleotides

| | | SEQ ID |
|---|---|---|
| Oligonucleotide Name | Sequence | NO: |
| Fwd CMV | CGCAAATGGGCGGTAGGCGTG | 138 |
| Cas9coRevE1 | CCGTGATGGATTGGTGAATC | 139 |
| Cas9coRevE2 | CCCATACGATTTCACCTGTC | 140 |
| Cas9coRevE3 | GGGTATTTTCCACAGGATGC | 141 |
| Cas9coRevE4 | CTTAGAAAGGCGGGTTTACG | 142 |
| Cas9coRevE5 | CTTACTAAGCTGCAATTTGG | 143 |
| Cas9coRevE6 | TGTATTCATCGGTTATGACAG | 144 |
| bGH_PArev seq1 | CAGGGTCAAGGAAGGCACG | 145 |
| pHU6-gRNA_for | GTTCCGCGCACATTTCC | 146 |
| pHU6-gRNA_rev | GCGGAGCCTATGGAAAAAC | 147 |
| pCALNL-for1 | GCCTTCTTCTTTTTCCTACAGC | 148 |
| pCALNL-for2 | CGCATCGAGCGAGCAC | 149 |

TABLE 5
Genomic PCR primers

| | | SEQ |
|---|---|---|
| Oligonucleotide | | ID |
| Name | Sequence | NO: |
| FAM19A2-F1 | TCAAGTAGCAAAAGAAGTAGGAGTCAG | 150 |
| FAM19A2-F2 | TTAGATGCATTCGTGCTTGAAG | 151 |
| FAM19A2-C1 | TTAATTTCTGCTGCTAGAACTAAATCTGG | 152 |
| FAM19A2-R1 | GGGAAGAAAACTGGATGGAGAATG | 153 |
| FAM19A2-R2 | CATAAATGACCTAGTGGAGCTG | 154 |
| FAM19A2-C2 | TGGTTATTTTGCCCATTAGTTGATGC | 155 |

### Reporter Construction

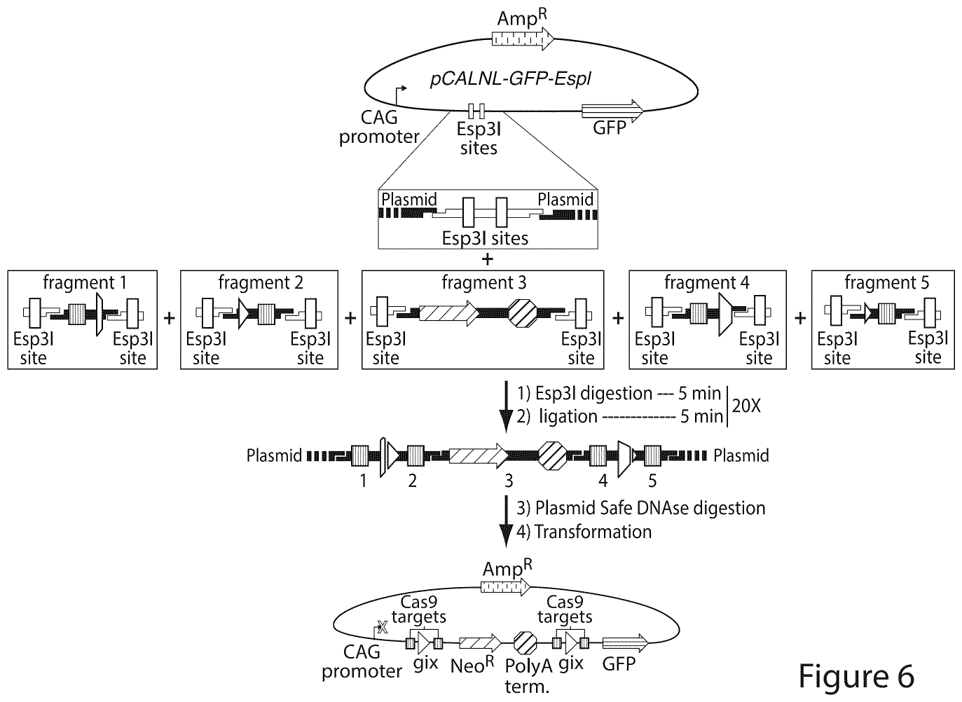

A five-piece Golden Gate assembly was used to construct reporters described below. Fragments 1-5 were flanked by Esp3J sites; Esp3J digestion created complementary 5′ overhangs specifying the order of fragment assembly (FIG. 6). Fragments 1, 2, 4, and 5 were created by annealing forward and reverse complementary oligonucleotides listed in Table 5. Fragments were annealed by mixing 10 μl of each oligonucleotide (100 μM) in 20 μl of molecular grade water, incubating at 95° C. for 3 minutes and reducing the temperature to 16° C. at a rate of −0.1° C./sec. Fragment 3 was created by PCR amplifying the region containing kanR and a PolyA stop codon with primers 3-for and 3-rev. These primers also appended Esp3J on the 5′ and 3′ ends of this sequence.

Annealed fragments 1, 2, 4 and 5 were diluted 12,000 fold and 0.625 μl of each fragment were added to a mixture containing the following:
1) 40-50 ng fragment 3
2) 100 ng pCALNL EGFP-Esp3I
3) 1 μL Tango Buffer (10×)
4) 1 μL DTT (10 mM)
5) 1 μL ATP (10 mM)
6) 0.25 uL T7 ligase (3,000 U/μL)
7) 0.75 uL Esp3I (10 U/μL)
8) H₂O up to 10 μL

Reactions were incubated in thermal cycler programmed for 20 cycles (37° C. for 5 min, 20° C.).

After completion of the Golden Gate reactions, 7 μL of each reaction was mixed with 1 μL of ATP (10 mM), 1 μL of 10× Plasmid Safe ATP-dependent DNAse buffer (10×), and 1 μL of Plasmid Safe ATP-dependent DNAse (10 U/μL) (Epicentre, Madison, WI) to remove linear DNA and reduce background. DNAse digestions were incubated at 37° C. for 30 min and heat killed at 70° C. for 30 min. Half (5 μL) of each reaction was transformed into Mach1-T1 cells. Colonies were analyzed by colony PCR and sequenced.

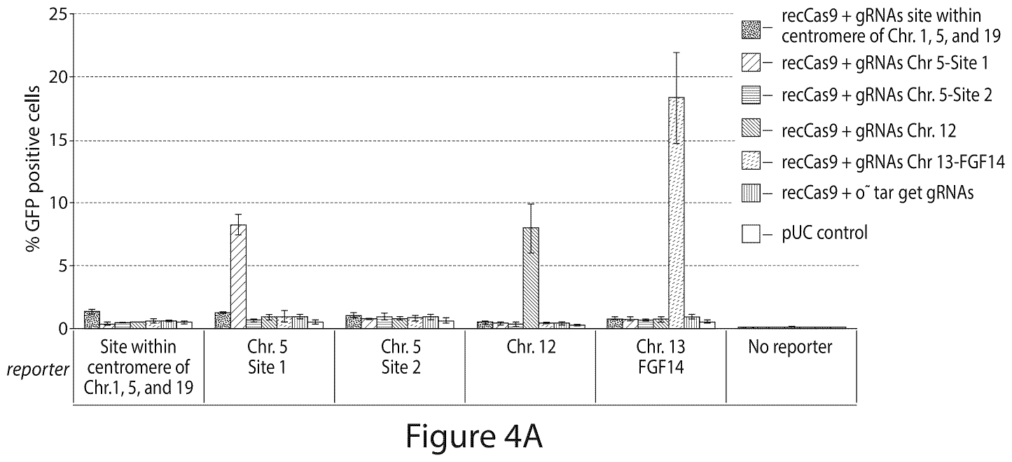

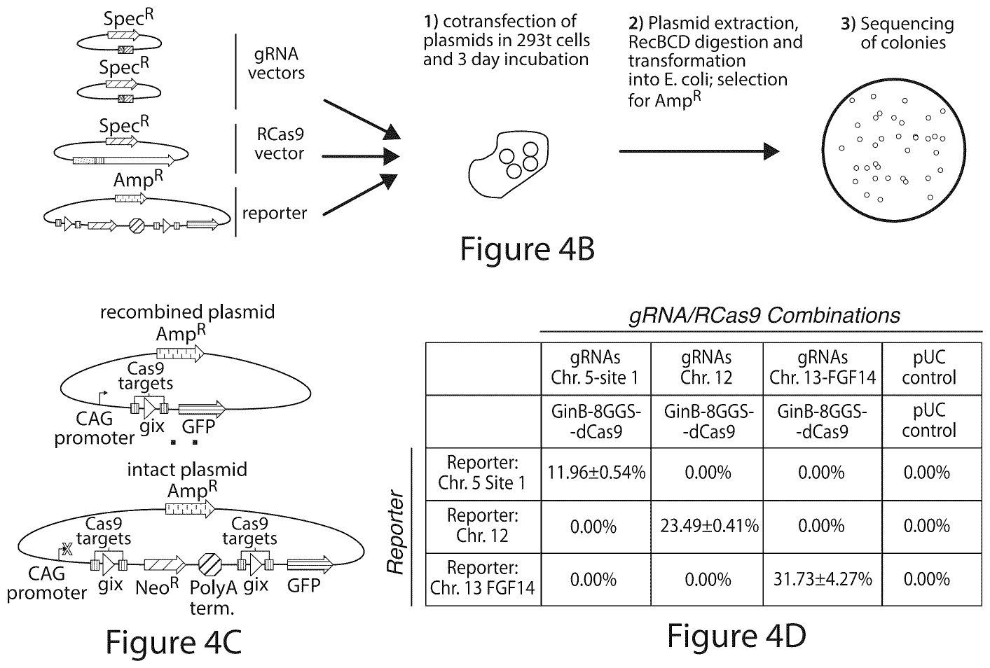

The protocol was modified for reporters used in FIG. 4. Two gBlocks, encoding target sites to the 5′ or 3′ of the PolyA terminator were used instead of fragments 1, 2, 4 and 5. These gBlocks (10 ng) were added to the MMX, which was cycled 10 times (37° C. for 5 min, 20° C.) and carried forward as described above.

### Plasmids

Unless otherwise stated, DNA fragments were isolated from agarose gels using QIAquick Gel Extraction Kit (Qiagen, Valencia, CA) and further purified using DNA Clean & Concentrator-5 (Zymo Research, Irvine, CA) or Qiaquick PCR purification kit (Qiagen, Valencia, CA). PCR fragments not requiring gel purification were isolated using one of the kits listed above.

The pCALNL-GFP subcloning vector, pCALNL-EGFP-Esp3I, was used to clone all recCas9 reporter plasmids and was based on the previously described pCALNL-GFP vector (Matsuda and Cepko, Controlled expression of transgenes introduced by in vivo electroporation. Proceedings of the National Academy of Sciences of the United States of America 104, 1027-1032 (2007), which is incorporated herein by reference). To create pCALNL-EGFP-Esp3I, pCALNL-GFP vectors were digested with XhoI and MluI and gel purified to remove the loxP sites, the kanamycin resistance marker, and the poly-A terminator. Annealed oligonucleotides formed an EspI-Insert, that contained inverted Esp3I sites as well as XhoI and MluI compatible overhangs; this insert was ligated into the XhoI and MluI digested plasmid and transformed.

pCALNL-GFP recCas9 reporter plasmids were created by Golden Gate assembly with annealed oligos and PCR products containing compatible Esp3I overhangs. Golden Gate reactions were set up and performed as described previously with Esp3I (ThermoFisher Scientific, Waltham, MA) (Sanjana et al., A transcription activator-like effector toolbox for genome engineering. Nature protocols 7, 171-192 (2012), the entire contents of which is hereby incorporated by reference). FIG. 6 outlines the general assembly scheme and relevant primers for reporter assembly as well as sequences for all recCas9 target sites are listed in Tables 2 and 6, respectively. A representative DNA sequence containing KanR (bold and underlined) and PolyA terminator (in italics and underlined) flanked by two recCas9 target sites is shown below. The target sites shown are both PAM_NT1-Obp-gix_core-0bp-NT1_PAM (see Table 6). Protoadjacent spacer motifs (PAMs) are in bold. Base pair spacers are lower case. Gix site or gix-related sites are in italics and dCas9 binding sites are underlined. For the genomic reporter plasmids used in the assays of FIG. 4, a G to T transversion was observed in the kanamycin resistance marker, denoted by a G/T in the sequence below. This was present in all the reporters used in this figure, and it is not expected to affect the results, as it is far removed from the PolyA terminator and recCas9 target sites.

(SEQ ID NO: 156)
ACGCGTCCCCAATTTTCCCAAACAGAGGTCTGTAAACCGAGGTTTTGGAA
CCTCTGTTTGGGAAAATTGGGGAGTCGAGTCGGATTTGATCTGATCAAGA
GACAGGATGAGGATCGTTTCGCATGATTGAACAAGATGGATTGCACGCAG
GTTCTCCGGCCGCTTGGGTGGAGAGGCTATTCGGCTATGACTGGGCACAA
CAGACAATCGGCTGCTCTGATGCCGCCGTGTTCCGGCTGTCAGTCGCAGG
GGCGCCCGGTTCTTTTTGTCAAGACCGACCTGTCCGGTGCCCTGAATGAA
CTGCAGGACGAGGCAGCGCGGCTATCGTGGCTGGCCACGACGGGCGTTCC
TTGCGCAGCTGTGCTCGACGTTGTCACTGAAGCGGGAAGGGACTGGCTGC
TATTGGGCGAAGTGCCGGGGCAGGATCTCCTGTCATCTCACCTTGCTCCT
GCCGAGAAAGTATCCATCATGGCTGATGCAATGCGGCGGCTGCATACGCT
TGATCCGGCTACCTGCCCATTCGACCACCAAGCGAAACATCGCATCGAGC
GAGCACGTACTCGGATGGAAGCCGGTCTTGTCGATCAGGATGATCTGGAC
GAAGAGCATCAGGGGCTCGCGCCAGCCGAACTGTTCGCCAGGCTCAAGGC
GCGCATGCCCGACGGCGAGGATCTCGTCGTGACCCATGGCGATGCCTGCT
TGCCGAATATCATGGTGGAAAATGGCCGCTTTTCTGGATTCATCGACTGT
GGCCGGCTGGGTGTGGCGGACCGCTATCAGGACATAGCGTTGGCTACCCG
TGATATTGCTGAAGAGCTTGGCGGCGAATGGGCTGACCGCTTCCTCGTGC
TTTACGGTATCGCCGCTCCCGATTCGCAGCGCATCGCCTTCTATCGCCTT
CTTGACGAGTTCTTCTGAGCGGGACTCTGGGGTTCGAAATGACCGACCAA
GCGACGCCCAACCTGCCATCACGAGATTTCGATTCCACCGCCGCCTTCTA
TGAAAGGTTGGGCTTCGGAATCGTTTTCCGGGACGCCGGCTGGATGATCC
TCCAGCGCGGGGATCTCATGCTGGAGTTCTTCGCCCACCCCATCGATAAC
TTGTTTATTGCAGCTTATAATGGTTACAAATAAAGCAATAGCATCACAAA
TTTCACAAATAAAGCATTTTTTTCACTGCATTCTAGTTGTGGTTTGTCCA
AACTCATCAATGTATCTTATCATGTCTGGATCAAATCCGAACGCACCCCC
AATTTTCCCAAACAGAGGTCTGTAAACCGAGGTTTTGGAACCTCTGTTTG
GGAAAATTGGGGCTCGAG

TABLE 6
List of target site sequences used in reporter assays

| | | SEQ ID |
|---|---|---|
| Target site name | Sequence | NO: |
| PAM_NT1-0bp- | CCCCAATTTTCCCAAACAGAGGTtCTGTAAACCGAG | 157 |
| gix_core-0bp- | GTTTTGGAACCTCTGTTTGGGAAAATTGGGG | |
| NT1_PAM | | |
| PAM_NT1-1bp- | CCCCAATTTTCCCAAACAGAGGTtCTGTAAACCGAG | 158 |
| gix_core-1bp- | GTTTTGGcAACCTCTGTTTGGGAAAATTGGGG | |
| NT1_PAM | | |
| PAM_NT1-2bp- | CCCCAATTTTCCCAAACAGAGGTatCTGTAAACCGA | 159 |
| gix_core-2bp- | GGTTTTGGctAACCTCTGTTTGGGAAAATTGGGG | |
| NT1_PAM | | |
| PAM_NT1-3bp- | CCCCAATTTTCCCAAACAGAGGTaatCTGTAAACCG | 160 |
| gix_core-3bp- | AGGTTTTGGcttAACCTCTGTTTGGGAAAATTGGGG | |
| NT1_PAM | | |
| PAM_NT1-4bp- | CCCCAATTTTCCCAAACAGAGGTaaatCTGTAAACCG | 161 |
| gix_core-4bp- | AGGTTTTGGcttaAACCTCTGTTTGGGAAAATTGGGG | |
| NT1_PAM | | |
| PAM_NT1-5bp- | CCCCAATTTTCCCAAACAGAGGTgaaatCTGTAAACC | 162 |
| gix_core-5bp- | GAGGTTTTGGcttagAACCTCTGTTTGGGAAAATTGG | |
| NT1_PAM | GG | |
| PAM_NT1-6bp- | CCCCAATTTTCCCAAACAGAGGTcgaaatCTGTAAAC | 163 |
| gix_core-6bp- | CGAGGTTTTGGcttagcAACCTCTGTTTGGGAAAATTG | |
| NT1_PAM | GGG | |
| PAM_NT1-7bp- | CCCCAATTTTCCCAAACAGAGGTtcgaaatCTGTAAAC | 164 |
| gix_core-7bp- | CGAGGTTTTGGcttagctAACCTCTGTTTGGGAAAATT | |
| NT1_PAM | GGGG | |
| PAM_NT1-6bp- | CCCCAATTTTCCCAAACAGAGGTtcgaaatCTGTAAAC | 165 |
| gix_core-0bp- | CGAGGTTTTGGAACCTCTGTTTGGGAAAATTGGGG | |
| NT1_PAM | | |
| PAM_NT1-6bp- | CCCCAATTTTCCCAAACAGAGGTtcgaaatCTGTAAAC | 166 |
| gix_core-1bp- | CGAGGTTTTGGcAACCTCTGTTTGGGAAAATTGGGG | |
| NT1_PAM | | |
| PAM_NT1-6bp- | CCCCAATTTTCCCAAACAGAGGTcgaaatCTGTAAAC | 167 |
| gix_core-2bp- | CGAGGTTTTGGctAACCTCTGTTTGGGAAAATTGGGG | |
| NT1_PAM | | |
| PAM_NT1-6bp- | CCCCAATTTTCCCAAACAGAGGTcgaaatCTGTAAAC | 168 |
| gix_core-4bp- | CGAGGTTTTGGcttaAACCTCTGTTTGGGAAAATTGG | |
| NT1_PAM | GG | |
| PAM_NT1-6bp- | CCCCAATTTTCCCAAACAGAGGTcgaaatCTGTAAAC | 169 |
| gix_core-5bp- | CGAGGTTTTGGcttagAACCTCTGTTTGGGAAAATTG | |
| NT1_PAM | GGG | |
| PAM_NT1-0bp- | CCCCAATTTTCCCAAACAGAGGTCTGTAAACCGAG | 170 |
| gix_core-6bp- | GTTTTGGcttagcAACCTCTGTTTGGGAAAATTGGGG | |
| NT1_PAM | | |
| PAM_NT1-1bp- | CCCCAATTTTCCCAAACAGAGGTtCTGTAAACCGAG | 171 |
| gix_core-6bp- | GTTTTGGcttagcAACCTCTGTTTGGGAAAATTGGGG | |
| NT1_PAM | | |
| PAM_NT1-2bp- | CCCCAATTTTCCCAAACAGAGGTatCTGTAAACCGA | 172 |
| gix_core-6bp- | GGTTTTGGcttagcAACCTCTGTTTGGGAAAATTGGGG | |
| NT1_PAM | | |
| PAM_NT1-3bp- | CCCCAATTTTCCCAAACAGAGGTaatCTGTAAACCG | 173 |
| gix_core-6bp- | AGGTTTTGGcttagcAACCTCTGTTTGGGAAAATTGG | |
| NT1_PAM | GG | |
| PAM_NT1-4bp- | CCCCAATTTTCCCAAACAGAGGTaaatCTGTAAACCG | 174 |
| gix_core-6bp- | AGGTTTTGGcttagcAACCTCTGTTTGGGAAAATTGG | |
| NT1_PAM | GG | |
| PAM_NT1-5bp- | CCCCAATTTTCCCAAACAGAGGTgaaatCTGTAAACC | 175 |
| gix_core-6bp- | GAGGTTTTGGcttagcAACCTCTGTTTGGGAAAATTGG | |
| NT1_PAM | GG | |
| Chromosome_10- | CCCCTCCCATCACAGGCCCTGAGgtttaaGAGAAAAC | 176 |
| 54913298-54913376* | CATGGTTTTGTGggccagGCCCATGACCCTTCTCCTCT | |
| | GGG | |
| Centromere_Chromosomes_1_5_19 | CCTTGTGTTGTGTGTCTTCAACTcacagAGTTAAACGA | 177 |
| | TGCTTTACACagagtaGACTTGAAACACTCTTTTTCTGG | |
| Chromosome_5_155183064- | CCACCGGCTCATGAGAGGTAGAGctaagGTCCAAAC | 178 |
| 155183141 | CTAGGTTTATCTgagaccGGAACTCATGTGATTAACTG | |
| (site 1) | TGG | |
| Chromosome_5_169395198- | CCTTAAGAACATAAATCCCCAGGaattcACAGAAACC | 179 |
| 169395274 | TTGGTTTGAGCtttggaTTTCCCGCAGGATGTGGGATA | |
| (site 2) | GG | |
| Chromosome_12_62418577- | CCACTCCCTCTCCCCCAAAAAGTaaaggTAGAAAACC | 180 |
| 62418652 | AAGGTTTACAGgcaacAAATAGCACAATGAATGGAA | |
| | TGG | |
| Chromosome_13_102010574- | CCTAGGGAAGTGATCATAGCTGAgtttctGGAAAAAC | 181 |
| 102010650 | CTAGGTTTTAAAgttgaGGAGACTTAAGTCCAAAACCT | |
| (FGF14) | GG | |

Protoadjacent spacer motifs (PAMs) are in bold. Base pair spacers are lower case. Gix site or gix-related sites are in italics and dCas9 binding sites are underlined.
*Chromosome_10 reporter contains two overlapping PAM sites and dCas9 binding sites on the 5′ and 3′ ends of the gix sites.

Plasmids containing the recCas9 gene were constructed by PCR amplification of a gBlock encoding an evolved, hyperactivated Gin variant (Ginβ) (Gaj et al., A comprehensive approach to zinc-finger recombinase customization enables genomic targeting in human cells. Nucleic acids research 41, 3937-3946 (2013), the entire contents of which is hereby incorporated by reference) with the oligonucleotides 1GGS-rev-BamHI or 2GGS-rev-BamHI (using linker SEQ ID NO: 182) and Gin-for-NotI. PCR fragments were digested with BamHI and NotI, purified and ligated into a previously described expression vector (Addgene plasmid 43861) (see, e.g., Fu et al., High-frequency off-target mutagenesis induced by CRISPR-Cas nucleases in human cells. Nature biotechnology 31, 822-826 (2013), the entire contents of which is hereby incorporated by reference) to produce subcloning vectors pGin-1GGS and pGIN-2GGS (using linker SEQ ID NO: 182). Oligonucleotides 1GGS-link-for-BamHI, 5GGS-link-for-BamHI (using linker SEQ ID NO: 701), or 8GGS-link-for-BamHI (using linker SEQ ID NO: 183) were used with Cas9-rev-FLAG-NLS-AgeI to construct PCR fragments encoding Cas9-FLAG-NLS with a 1, 5, or 8 GGS linker (see Table 3). For DNA sequences encoding the GGS amino acid linkers, see Table 7. PCR fragments and subcloning plasmids were digested with BamHI and AgeI and ligated to create plasmids pGinβ-2×GGS-dCas9-FLAG-NLS (using linker SEQ ID NO: 182), pGinβ-5×GGS-dCas9-FLAG-NLS (using linker SEQ ID NO: 701), and pGinβ-8×GGS-dCas9-FLAG-NLS (using linker SEQ ID NO: 183). For the DNA and amino acid sequence of the pGinβ-8×GGS-dCas9-FLAG-NLS (i.e., recCas9), see below. The sequence encoding Ginβ is shown in bold; those encoding GGS linkers are shown in italics; those encoding dCas9 linkers are black; those encoding the FLAG tag and NLS are underlined and in lowercase, respectively.

(SEQ ID NO: 184)
ATGCTCATTGGCTACGTGCGCGTCTCAACTAACGACCAGAATACCGATCTTC
AGAGGAACGCACTGGTTTGTGCAGGCTGCGAACAGATTTTCGAGGACAAAC
TCAGCGGGACACGGACGGACAGACCTGGCCTCAAGCGAGCACTCAAGAGGC
TGCAGAAAGGAGACACTCTGGTGGTCTGGAAATTGGACCGCCTGGGTCGAA
GCATGAAGCATCTCATTTCTCTGGTTGGCGAACTGCGAGAAAGGGGGATCA
ACTTTCGAAGTCTGACGGATTCCATAGATACAAGCAGCCCCATGGGCCGGT
TCTTCTTCTACGTGATGGGTGCACTGGCTGAAATGGAAAGAGAACTCATTAT
AGAGCGAACCATGGCAGGGCTTGCGGCTGCCAGGAATAAAGGCAGGCGGTT
TGGAAGACCACCAAAGGGTGGATCCGGAGGGTCCGGAGGTAGTGGCGGCAGCGG
TGGTTCAGGTGGCAGCGGAGGGTCAGGAGGCTCTGATAAAAAGTATTCTATTGGTT
TAGCTATCGGCACTAATTCCGTTGGATGGGCTGTCATAACCGATGAATACAAAGT
ACCTTCAAAGAAATTTAAGGTGTTGGGGAACACAGACCGTCATTCGATTAAAAA
GAATCTTATCGGTGCCCTCCTATTCGATAGTGGCGAAACGGCAGAGGCGACTCGC
CTGAAACGAACCGCTCGGAGAAGGTATACACGTCGCAAGAACCGAATATGTTAC
TTACAAGAAATTTTTAGCAATGAGATGGCCAAAGTTGACGATTCTTTCTTTCACC
GTTTGGAAGAGTCCTTCCTTGTCGAAGAGGACAAGAAACATGAACGGCACCCCA
TCTTTGGAAACATAGTAGATGAGGTGGCATATCATGAAAAGTACCCAACGATTTA
TCACCTCAGAAAAAAGCTAGTTGACTCAACTGATAAAGCGGACCTGAGGTTAAT
CTACTTGGCTCTTGCCCATATGATAAAGTTCCGTGGGCACTTTCTCATTGAGGGTG
ATCTAAATCCGGACAACTCGGATGTCGACAAACTGTTCATCCAGTTAGTACAAAC
CTATAATCAGTTGTTTGAAGAGAACCCTATAAATGCAAGTGGCGTGGATGCGAA
GGCTATTCTTAGCGCCCGCCTCTCTAAATCCCGACGGCTAGAAAACCTGATCGCA
CAATTACCCGGAGAGAAGAAAAATGGGTTGTTCGGTAACCTTATAGCGCTCTCAC
TAGGCCTGACACCAAATTTTAAGTCGAACTTCGACTTAGCTGAAGATGCCAAATT
GCAGCTTAGTAAGGACACGTACGATGACGATCTCGACAATCTACTGGCACAAAT
TGGAGATCAGTATGCGGACTTATTTTTGGCTGCCAAAAACCTTAGCGATGCAATC
CTCCTATCTGACATACTGAGAGTTAATACTGAGATTACCAAGGCGCCGTTATCCG
CTTCAATGATCAAAAGGTACGATGAACATCACCAAGACTTGACACTTCTCAAGGC
CCTAGTCCGTCAGCAACTGCCTGAGAAATATAAGGAAATATTCTTTGATCAGTCG
AAAAACGGGTACGCAGGTTATATTGACGGCGGAGCGAGTCAAGAGGAATTCTAC
AAGTTTATCAAACCCATATTAGAGAAGATGGATGGGACGGAAGAGTTGCTTGTA
AAACTCAATCGCGAAGATCTACTGCGAAAGCAGCGGACTTTCGACAACGGTAGC
ATTCCACATCAAATCCACTTAGGCGAATTGCATGCTATACTTAGAAGGCAGGAGG
ATTTTTATCCGTTCCTCAAAGACAATCGTGAAAAGATTGAGAAAATCCTAACCTT
TCGCATACCTTACTATGTGGGACCCCTGGCCCGAGGGAACTCTCGGTTCGCATGG
ATGACAAGAAAGTCCGAAGAAACGATTACTCCATGGAATTTTGAGGAAGTTGTC
GATAAAGGTGCGTCAGCTCAATCGTTCATCGAGAGGATGACCAACTTTGACAAG
AATTTACCGAACGAAAAAGTATTGCCTAAGCACAGTTTACTTTACGAGTATTTCA
CAGTGTACAATGAACTCACGAAAGTTAAGTATGTCACTGAGGGCATGCGTAAAC
CCGCCTTTCTAAGCGGAGAACAGAAGAAAGCAATAGTAGATCTGTTATTCAAGA
CCAACCGCAAAGTGACAGTTAAGCAATTGAAAGAGGACTACTTTAAGAAAATTG
AATGCTTCGATTCTGTCGAGATCTCCGGGGTAGAAGATCGATTTAATGCGTCACT
TGGTACGTATCATGACCTCCTAAAGATAATTAAAGATAAGGACTTCCTGGATAAC
GAAGAGAATGAAGATATCTTAGAAGATATAGTGTTGACTCTTACCCTCTTTGAAG
ATCGGGAAATGATTGAGGAAAGACTAAAAACATACGCTCACCTGTTCGACGATA
AGGTTATGAAACAGTTAAAGAGGCGTCGCTATACGGGCTGGGGACGATTGTCGC
GGAAACTTATCAACGGGATAAGAGACAAGCAAAGTGGTAAAACTATTCTCGATT
TTCTAAAGAGCGACGGCTTCGCCAATAGGAACTTTATGCAGCTGATCCATGATGA
CTCTTTAACCTTCAAAGAGGATATACAAAAGGCACAGGTTTCCGGACAAGGGGA
CTCATTGCACGAACATATTGCGAATCTTGCTGGTTCGCCAGCCATCAAAAAGGGC
ATACTCCAGACAGTCAAAGTAGTGGATGAGCTAGTTAAGGTCATGGGACGTCAC
AAACCGGAAAACATTGTAATCGAGATGGCACGCGAAAATCAAACGACTCAGAAG
GGGCAAAAAAACAGTCGAGAGCGGATGAAGAGAATAGAAGAGGGTATTAAAGA
ACTGGGCAGCCAGATCTTAAAGGAGCATCCTGTGGAAAATACCCAATTGCAGAA
CGAGAAACTTTACCTCTATTACCTACAAAATGGAAGGGACATGTATGTTGATCAG
GAACTGGACATAAACCGTTTATCTGATTACGACGTCGATGCCATTGTACCCCAAT
CCTTTTTGAAGGACGATTCAATCGACAATAAAGTGCTTACACGCTCGGATAAGAA
CCGAGGGAAAAGTGACAATGTTCCAAGCGAGGAAGTCGTAAAGAAAATGAAGA
ACTATTGGCGGCAGCTCCTAAATGCGAAACTGATAACGCAAAGAAAGTTCGATA
ACTTAACTAAAGCTGAGAGGGGTGGCTTGTCTGAACTTGACAAGGCCGGATTTAT
TAAACGTCAGCTCGTGGAAACCCGCCAAATCACAAAGCATGTTGCACAGATACT
AGATTCCCGAATGAATACGAAATACGACGAGAACGATAAGCTGATTCGGGAAGT
CAAAGTAATCACTTTAAAGTCAAAATTGGTGTCGGACTTCAGAAAGGATTTTCAA
TTCTATAAAGTTAGGGAGATAAATAACTACCACCATGCGCACGACGCTTATCTTA
ATGCCGTCGTAGGGACCGCACTCATTAAGAAATACCCGAAGCTAGAAAGTGAGT
TTGTGTATGGTGATTACAAAGTTTATGACGTCCGTAAGATGATCGCGAAAAGCGA
ACAGGAGATAGGCAAGGCTACAGCCAAATACTTCTTTTATTCTAACATTATGAAT
TTCTTTAAGACGGAAATCACTCTGGCAAACGGAGAGATACGCAAACGACCTTTA
ATTGAAACCAATGGGGAGACAGGTGAAATCGTATGGGATAAGGGCCGGGACTTC
GCGACGGTGAGAAAAGTTTTGTCCATGCCCCAAGTCAACATAGTAAAGAAAACT
GAGGTGCAGACCGGAGGGTTTTCAAAGGAATCGATTCTTCCAAAAAGGAATAGT
GATAAGCTCATCGCTCGTAAAAAGGACTGGGACCCGAAAAAGTACGGTGGCTTC
GATAGCCCTACAGTTGCCTATTCTGTCCTAGTAGTGGCAAAAGTTGAGAAGGGAA
AATCCAAGAAACTGAAGTCAGTCAAAGAATTATTGGGGATAACGATTATGGAGC
GCTCGTCTTTTGAAAAGAACCCCATCGACTTCCTTGAGGCGAAAGGTTACAAGGA
AGTAAAAAAGGATCTCATAATTAAACTACCAAAGTATAGTCTGTTTGAGTTAGAA
AATGGCCGAAAACGGATGTTGGCTAGCGCCGGAGAGCTTCAAAAGGGGAACGA
ACTCGCACTACCGTCTAAATACGTGAATTTCCTGTATTTAGCGTCCCATTACGAG
AAGTTGAAAGGTTCACCTGAAGATAACGAACAGAAGCAACTTTTTGTTGAGCAG
CACAAACATTATCTCGACGAAATCATAGAGCAAATTTCGGAATTCAGTAAGAGA
GTCATCCTAGCTGATGCCAATCTGGACAAAGTATTAAGCGCATACAACAAGCAC
AGGGATAAACCCATACGTGAGCAGGCGGAAAATATTATCCATTTGTTTACTCTTA
CCAACCTCGGCGCTCCAGCCGCATTCAAGTATTTTGACACAACGATAGATCGCAA
ACGATACACTTCTACCAAGGAGGTGCTAGACGCGACACTGATTCACCAATCCATC
ACGGGATTATATGAAACTCGGATAGATTTGTCACAGCTTGGGGGTGACGGTGGCT
CCGATTATAAGGATGATGACGACAAGGGAGGTTCCccaaagaagaaaaggaaggtcTGA
(SEQ ID NO: 185)
MLIGYVRVSTNDQNTDLQRNALVCAGCEQIFEDKLSGTRTDRPGLKRALKRLQ
KGDTLVVWKLDRLGRSMKHLISLVGELRERGINFRSLTDSIDTSSPMGRFFFYV
MGALAEMERELIIERTMAGLAAARNKGRRFGRPPKGGSGGSGGSGGSGGSGGSG
GSGGSDKKYSIGLAIGTNSVGWAVITDEYKVPSKKFKVLGNTDRHSIKKNLIGALLFD
SGETAEATRLKRTARRRYTRRKNRICYLQEIFSNEMAKVDDSFFHRLEESFLVEEDK
KHERHPIFGNIVDEVAYHEKYPTIYHLRKKLVDSTDKADLRLIYLALAHMIKFRGHFL
IEGDLNPDNSDVDKLFIQLVQTYNQLFEENPINASGVDAKAILSARLSKSRRLENLIAQ
LPGEKKNGLFGNLIALSLGLTPNFKSNFDLAEDAKLQLSKDTYDDDLDNLLAQIGDQ
YADLFLAAKNLSDAILLSDILRVNTEITKAPLSASMIKRYDEHHQDLTLLKALVRQQL
PEKYKEIFFDQSKNGYAGYIDGGASQEEFYKFIKPILEKMDGTEELLVKLNREDLLRK
QRTFDNGSIPHQIHLGELHAILRRQEDFYPFLKDNREKIEKILTFRIPYYVGPLARGNS
RFAWMTRKSEETITPWNFEEVVDKGASAQSFIERMTNFDKNLPNEKVLPKHSLLYEY
FTVYNELTKVKYVTEGMRKPAFLSGEQKKAIVDLLFKTNRKVTVKQLKEDYFKKIE
CFDSVEISGVEDRFNASLGTYHDLLKIIKDKDFLDNEENEDILEDIVLTLTLFEDREMIE
ERLKTYAHLFDDKVMKQLKRRRYTGWGRLSRKLINGIRDKQSGKTILDFLKSDGFA
NRNFMQLIHDDSLTFKEDIQKAQVSGQGDSLHEHIANLAGSPAIKKGILQTVKVVDE
LVKVMGRHKPENIVIEMARENQTTQKGQKNSRERMKRIEEGIKELGSQILKEHPVEN
TQLQNEKLYLYYLQNGRDMYVDQELDINRLSDYDVDAIVPQSFLKDDSIDNKVLTR
SDKNRGKSDNVPSEEVVKKMKNYWRQLLNAKLITQRKFDNLTKAERGGLSELDKA
GFIKRQLVETRQITKHVAQILDSRMNTKYDENDKLIREVKVITLKSKLVSDFRKDFQF
YKVREINNYHHAHDAYLNAVVGTALIKKYPKLESEFVYGDYKVYDVRKMIAKSEQ
EIGKATAKYFFYSNIMNFFKTEITLANGEIRKRPLIETNGETGEIVWDKGRDFATVRK
VLSMPQVNIVKKTEVQTGGFSKESILPKRNSDKLIARKKDWDPKKYGGFDSPTVAYS
VLVVAKVEKGKSKKLKSVKELLGITIMERSSFEKNPIDFLEAKGYKEVKKDLIIKLPK
YSLFELENGRKRMLASAGELQKGNELALPSKYVNFLYLASHYEKLKGSPEDNEQKQ
LFVEQHKHYLDEIIEQISEFSKRVILADANLDKVLSAYNKHRDKPIREQAENIIHLFTLT
NLGAPAAFKYFDTTIDRKRYTSTKEVLDATLIHQSITGLYETRIDLSQLGGDGGSDYK
DDDDKGGSpkkkrkv Stop

The Gin recombinase catalytic domain, which is amino acids 1-142 of SEQ ID NO: 185, is identical to the sequence of SEQ ID NO: 713. The dCas9 domain, in which is amino acids 167-1533 of SEQ ID NO: 185 is identical to the sequence of SEQ ID NO: 712.

(SEQ ID NO: 713)
MLIGYVRVSTNDQNTDLQRNALVCAGCEQIFEDKLSGTRTDRPGLKRALK
RLQKGDTLVVWKLDRLGRSMKHLISLVGELRERGINFRSLTDSIDTSSPM
GRFFFYVMGALAEMERELIIERTMAGLAAARNKGRRFGRPPK
(SEQ ID NO: 712)
DKKYSIGLAIGTNSVGWAVITDEYKVPSKKFKVLGNTDRHSIKKNLIGAL
LFDSGETAEATRLKRTARRRYTRRKNRICYLQEIFSNEMAKVDDSFFHRL
EESFLVEEDKKHERHPIFGNIVDEVAYHEKYPTIYHLRKKLVDSTDKADL
RLIYLALAHMIKFRGHFLIEGDLNPDNSDVDKLFIQLVQTYNQLFEENPI
NASGVDAKAILSARLSKSRRLENLIAQLPGEKKNGLFGNLIALSLGLTPN
FKSNFDLAEDAKLQLSKDTYDDDLDNLLAQIGDQYADLFLAAKNLSDAIL
LSDILRVNTEITKAPLSASMIKRYDEHHQDLTLLKALVRQQLPEKYKEIF
FDQSKNGYAGYIDGGASQEEFYKFIKPILEKMDGTEELLVKLNREDLLRK
QRTFDNGSIPHQIHLGELHAILRRQEDFYPFLKDNREKIEKILTFRIPYY
VGPLARGNSRFAWMTRKSEETITPWNFEEVVDKGASAQSFIERMTNFDKN
LPNEKVLPKHSLLYEYFTVYNELTKVKYVTEGMRKPAFLSGEQKKAIVDL
LFKTNRKVTVKQLKEDYFKKIECFDSVEISGVEDRFNASLGTYHDLLKII
KDKDFLDNEENEDILEDIVLTLTLFEDREMIEERLKTYAHLFDDKVMKQL
KRRRYTGWGRLSRKLINGIRDKQSGKTILDFLKSDGFANRNFMQLIHDDS
LTFKEDIQKAQVSGQGDSLHEHIANLAGSPAIKKGILQTVKVVDELVKVM
GRHKPENIVIEMARENQTTQKGQKNSRERMKRIEEGIKELGSQILKEHPV
ENTQLQNEKLYLYYLQNGRDMYVDQELDINRLSDYDVDAIVPQSFLKDDS
IDNKVLTRSDKNRGKSDNVPSEEVVKKMKNYWRQLLNAKLITQRKFDNLT
KAERGGLSELDKAGFIKRQLVETRQITKHVAQILDSRMNTKYDENDKLIR
EVKVITLKSKLVSDFRKDFQFYKVREINNYHHAHDAYLNAVVGTALIKKY
PKLESEFVYGDYKVYDVRKMIAKSEQEIGKATAKYFFYSNIMNFFKTEIT
LANGEIRKRPLIETNGETGEIVWDKGRDFATVRKVLSMPQVNIVKKTEVQ
TGGFSKESILPKRNSDKLIARKKDWDPKKYGGFDSPTVAYSVLVVAKVEK
GKSKKLKSVKELLGITIMERSSFEKNPIDFLEAKGYKEVKKDLIIKLPKY
SLFELENGRKRMLASAGELQKGNELALPSKYVNFLYLASHYEKLKGSPED
NEQKQLFVEQHKHYLDEIIEQISEFSKRVILADANLDKVLSAYNKHRDKP
IREQAENIIHLFTLTNLGAPAAFKYFDTTIDRKRYTSTKEVLDATLIHQS
ITGLYETRIDLSQLGGD

TABLE 7
DNA sequences encoding GGS linkers

| GGS | SEQ ID | | SEQ ID |
|---|---|---|---|
| linkers | NO: | DNA sequences for GGS linkers | NO: |
| 2XGGS | 182 | GGTGGTAGCGGTGGATCC | 186 |
| 5XGGS | 701 | GGTGGATCCGGTGGTTCAGGTGGCAGCGGAGGGTCAG | 187 |
| | | GAGGCTCT | |
| 8XGGS | 183 | GGTGGATCCGGAGGGTCCGGAGGTAGTGGCGGCAGC | 188 |
| | | GGTGGTTCAGGTGGCAGCGGAGGGTCAGGAGGCTCT | |

For plasmid sequencing experiments, the AmpR gene in pGinP-8×GGS-dCas9-FLAG-NLS (using linker SEQ ID NO: 183) was replaced with SpecR by golden gate cloning with PCR fragments. Esp3I sites were introduced into the pGinP-8×GGS-dCas9-FLAG-NLS (using linker SEQ ID NO: 183) plasmid at sites flanking the AmpR gene by PCR with Esp3I-for-plasmid and Esp3I-rev-plasmid. The primers spec-Esp3I-for and spec-Esp3I-rev were used to amplify the SpecR marker as well as introduce Esp3I sites and Esp3I generated overhangs compatible with those generated by the Esp3I-cleaved plasmid PCR product. Golden gate assembly was performed on the two fragments following the protocol used to generate the reporter plasmids as described herein.

The pHU6-NT1 guide RNA expression vector was based on the previously described pFYF1328 (Fu et al., High-frequency off-target mutagenesis induced by CRISPR-Cas nucleases in human cells. Nature biotechnology 31, 822-826 (2013), the entire contents of which is hereby incorporated by reference) altered to target a region within the bacterial luciferase gene LuxAB. Guide RNA expression vectors were created by PCR amplification of the entire vector with a universal primer R.pHU6.TSS(−1).univ and primers encoding unique guide RNA sequences (Table 1). A list of the guide RNA sequences is given in Table 8. These primers were phosphorylated with T4 polynucleotide kinase. The PCR reaction products and linear guide RNA expression vectors were blunt-end ligated and transformed. Guide RNA expression vectors used in initial optimizations, off target control guide RNA sequences and those targeting Chromosome 10 locus contained AmpR. All other plasmids described in this study contained specR to facilitate sequencing experiments. Spectinomycin resistance was initially introduced into guide RNA expression vectors via CPEC essentially as described (Quan et al., Circular polymerase extension cloning of complex gene libraries and pathways. PloS one 4, e6441 (2009); and Hillson (2010), vol. 2015, pp. CPEC protocol; each of which is incorporated herein by reference) and guide RNA plasmids were then constructed by PCR amplification of the vector, as described above. Reactions were incubated overnight at 37° C. with 40 U of DpnI, purified and transformed. Fragments for CPEC were generated by PCR amplification of a guide RNA expression vector with oligonucleotides cpec-assembly-for-spec2 and cpec assembly-rev. The specR fragment was generated by PCR amplification of the SpecR gene via the oligonucleotides cpec-assembly-for-spec and cpec-assembly-rev-spec. pUC19 (ThermoFisher Scientific, Waltham, MA) was similarly modified.

TABLE 8
List of gRNA sequences

| | | SEQ ID |
|---|---|---|
| gRNA name | gRNA-sequence | NO: |
| on-target_gRNA | ACCTCTGTTTGGGAAAATTG | 189 |
| non-target_gRNA | gCACACTAGTTAGGGATAACA | 190 |
| Chromosome_10-54913298- | gCCTCAGGGCCTGTGATGGGA | 191 |
| 54913376_gRNA-rev-5 | | |
| Chromosome_10-54913298- | gCTCAGGGCCTGTGATGGGAG | 192 |
| 54913376_gRNA-rev-6 | | |
| Chromosome_10-54913298- | GGCCCATGACCCTTCTCCTC | 193 |
| 54913376_gRNA-for-5 | | |
| Chromosome_10-54913298- | GCCCATGACCCTTCTCCTCT | 194 |
| 54913376_gRNA-for-6 | | |
| Centromere_Chromosomes_1_5_19- | GACTTGAAACACTCTTTTTC | 195 |
| gRNA-for | | |
| Centromere_Chromosomes_1_5_19- | gAGTTGAAGACACACAACACA | 196 |
| gRNA-rev | | |
| Chromosome_5_155183064- | GGAACTCATGTGATTAACTG | 197 |
| 155183141_(site 1)_gRNA-for | | |
| Chromosome_5_155183064- | gTCTACCTCTCATGAGCCGGT | 198 |
| 155183141_(site 1)_gRNA-rev | | |
| Chromosome_5_169395198- | gTTTCCCGCAGGATGTGGGAT | 199 |
| 169395274_(site 2)_gRNA-for | | |
| Chromosome_5_169395198- | gCCTGGGGATTTATGTTCTTA | 200 |
| 169395274_(site 2)_gRNA-rev | | |
| Chromosome_12_62418577- | gAAATAGCACAATGAATGGAA | 201 |
| 62418652_gRNA-for | | |
| Chromosome_12_62418577- | gACTTTTTGGGGGAGAGGGAG | 202 |
| 62418652_gRNA-rev | | |
| Chromosome_13_102010574- | GGAGACTTAAGTCCAAAACC | 203 |
| 102010650_(FGF14)_gRNA-for | | |
| Chromosome_13_102010574- | gTCAGCTATGATCACTTCCCT | 204 |
| 102010650_(FGF14)_gRNA-rev | | |
| Off target-for (CLTA) | GCAGATGTAGTGTTTCCACA | 205 |
| Off target-rev(VEGF) | GGGTGGGGGGAGTTTGCTCC | 206 |
| Chromosome_12_62098359- | gATATCCGTTTATCAGTGTCA | 207 |
| 62098434_(FAM19A2)_gRNA-rev | | |
| Chromosome_12_62098359- | gTTCCTAAGCTTGGGCTGCAG | 208 |
| 62098434_(FAM19A2)_gRNA-for | | |
| Chromosome_12_62112591- | gCCTAAAAGTGACTGGGAGAA | 209 |
| 62112668_(FAM19A2)_gRNA-rev | | |
| Chromosome_12_62112591- | gCACAGTCCCATATTTCTTGG | 210 |
| 62112668_(FAM19A2)_gRNA-for | | |

### Cell Culture and Transfection

HEK293T cells were purchased from the American Type Culture Collection (ATCC, Manassas, VA). Cells were cultured in Dulbecco's modified Eagle's medium (DMEM)+GlutaMAX-I (4.5 g/L D glucose+110 mg/mL sodium pyruvate) supplemented with 10% fetal bovine serum (FBS, Life Technologies, Carlsbad, CA). Cells were cultured at 37° C. at 5% CO₂ in a humidified incubator.

Plasmid used for transfections were isolated from PureYield Plasmid Miniprep System (Promega, Madison, WI). The night before transfections, HEK293T cells were seeded at a density of 3×10⁵ cells per well in 48 well collagen-treated plates (Corning, Corning, NY). Transfections reactions were prepared in 25 μL of Opti-MEM (ThermoFisher Scientific, Waltham, MA). For each transfection, 45 ng of each guide RNA expression vector, 9 ng of reporter plasmid, 9 ng of piRFP670-N1 (Addgene Plasmid 45457), and 160 ng of recCas9 expression vector were mixed, combined with 0.8 μL lipofectamine 2000 in Opti-MEM (ThermoFisher Scientific, Waltham, MA) and added to individual wells.

### Flow Cytometry

After 60-72 hours post-transfection, cells were washed with phosphate buffered saline and harvested with 50 μL of 0.05% trypsin-EDTA (Life Technologies, Carlsbad, CA) at 37° C. for 5-10 minutes. Cells were diluted in 250 μL culture media and run on a BD Fortessa analyzer. iRFP fluorescence was excited using a 635 nm laser and emission was collected using a 670/30 band pass filter. EGFP was excited using a 488 nM laser and emission fluorescence acquired with a 505 long pass and 530/30 band pass filters. Data was analyzed on FlowJo Software, gated for live and transfected events (expressing iRFP). Positive GFP-expressing cells were measured as a percentage of transfected cells gated from at least 6,000 live events. For optimization experiments, assay background was determined by measuring the percentage of transfected cells producing eGFP upon cotransfection with reporter plasmid and pUC, without recCas9 or guide RNA expression vectors. This background was then subtracted from percentage of eGFP-positive cells observed when the reporter plasmid was cotransfected with recCas9 and the on-target or non-target guide RNA expression vectors.

### Identification of Genomic Target Sites

Searching for appropriate target sites was done using Bioconductor, an open-source bioinformatics package using the R statistical programming (Fu et al., High-frequency off-target mutagenesis induced by CRISPR-Cas nucleases in human cells. Nature biotechnology 31, 822-826 (2013), the entire contents of which is hereby incorporated by reference). The latest release (GRCh38) of the human reference genome published by the Genome Reference Consortium was used to search for sites that matched both the PAM requirement of Cas9 and the evolved gix sequence as described in the text. With the genome loaded into R, each search pattern was represented as a Biostring, a container in R that allowed for string matching and manipulation Scanning both strands of DNA for the entire genome, using the stated parameters, reveals approximately 450 potential targets in the human genome when searching using the GRCh38 reference assembly (Table 9).

TABLE 9
recCas9 genomic targets identified in silico

| | | | | Pattern | SEQ ID |
|---|---|---|---|---|---|
| Chr. | Start | End | Sequence | ID | NO: |
| chr1 | 34169027 | 34169103 | CCTTTAGTGAAAAGTAGACAGCTCTGAATAT | 2 | 211 |
| | | | GAAAGGTAGGTTTTCATTTCTGGGAAAGAGA | | |
| | | | CGCCAAGTGATGTGG | | |
| chr1 | 51006703 | 51006780 | CCTCCAATAAATATGGGACTATGTGGAAAG | 1 | 212 |
| | | | ACCAAACCTACGTTTGATTGGTGTACCTGAA | | |
| | | | AGTGACGGGAAGAATGG | | |
| chr1 | 89229373 | 89229450 | CCATTCTGCCCGTCACTTTCAGGTACACCAA | 1 | 213 |
| | | | TCAAACGTAGGTTTAGTCTTTTCACATAGTC | | |
| | | | CCATATTTCTTGGAGG | | |
| chr1 | 115638077 | 115638154 | CCATTCTCCCCGTCACTTTCAGGTACAACAA | 1 | 214 |
| | | | TCAAACGTAGGTTTGGTCTTTTCACATAGTC | | |
| | | | CCATATTTCTTGGAGG | | |
| chr1 | 122552402 | 122552478 | CCTTGTAGTGTGTGTATTCAACTCACAGAGT | 2 | 215 |
| | | | TAAACGATCCTTTACACAGAGCAGACTTGAA | | |
| | | | ACACTCTTGTTGTGG | | |
| chr1 | 122609874 | 122609950 | CCTTGTAGTGTGTGTATTCAACTCACAGAGT | 2 | 216 |
| | | | TAAACGATCCTTTACACAGAGCATACTTGAA | | |
| | | | ACACTCTTTTTGTGG | | |
| chr1 | 122668677 | 122668753 | CCTTGTGTTGTGTGTATTCAACTCACAGAGT | 2 | 217 |
| | | | TAAACGATCCTTTACACAGAGCAGACTTGAA | | |
| | | | ACACTCTTTTTGTGG | | |
| chr1 | 123422419 | 123422495 | CCTTGTGTTGTGTTTATTCAACTCACAGAGTT | 2 | 218 |
| | | | AAACGATCCTTTACACAGAGCAGACTTGAA | | |
| | | | ATACTCTTTTTGTGG | | |
| chr1 | 123648614 | 123648690 | CCTTGTAGTGTGTGTATTCAACTCACAGAGT | 2 | 219 |
| | | | TAAACGATCCTTTACACAGAGCATACTTGAA | | |
| | | | ACACTCTTTTTGTGG | | |
| chr1 | 123806335 | 123806411 | CCTTGTATTGTGAGTATTCAACTCACAGAGT | 2 | 220 |
| | | | TAAACGATCCTTTACACAGAGCAGACTTGAA | | |
| | | | ACACTCTTTTTGTGG | | |
| chr1 | 124078228 | 124078304 | CCTTGTGTTGTGTGTCTTCAACTCACAGAGTT | 2 | 221 |
| | | | AAACGATGCTTTACACAGAGTAGACTTGAA | | |
| | | | ACACTCTTTTTCTGG | | |
| chr1 | 124231074 | 124231150 | CCTTGTGTTGTGTGTATTCAACTCACAGAGT | 2 | 222 |
| | | | TAAACGATCCTTTACACAGAGCAGACTTGTA | | |
| | | | ACACTCTTTTTGTGG | | |
| chr1 | 124232435 | 124232511 | CCTTGTGTTGTGTGTATTCAACTCACAGAGT | 2 | 223 |
| | | | TAAACGATCCTTTACACAGAGCAGACGTGA | | |
| | | | AACACTCTTTTTGTGG | | |
| chr1 | 124344781 | 124344857 | CCTTGTGTTGTGTGTATTCAACTCACAGAGT | 2 | 224 |
| | | | TAAACGATCCTTTACACAGAGCAGACTTGAA | | |
| | | | ACACTCTTTTTGTGG | | |
| chr1 | 124435716 | 124435792 | CCTTGTGTTGTGTGTATTCAACTCACAGAGT | 2 | 225 |
| | | | TAAACGATCCTTTACACAGAGGAGACTTGTA | | |
| | | | ACACTCTTTTTGTGG | | |
| chr1 | 158677186 | 158677262 | CCTGAGGTTTTCCAGGTTTTAAAAGGAAACC | 2 | 226 |
| | | | TAAAGGTAGGTTTAGCATTAAGTGTCTTGAA | | |
| | | | GTTTATTTTAAAAGG | | |
| chr1 | 167629479 | 167629554 | CCAAAATTCCCACAAAACCGAATGCATCAGT | 4 | 227 |
| | | | CAAAGCAAGGTTTGAAGAAAAGATTTACCA | | |
| | | | CTTCAGGGAGCTTGG | | |
| chr1 | 167783428 | 167783504 | CCTTTTCTGGATATCGTTGATGCTCTGTATGC | 3 | 228 |
| | | | AAAAGGTAGGTTTTTGGGTTATGTTGTTAAA | | |
| | | | CAGTGATTGAATGG | | |
| chr1 | 169409367 | 169409444 | CCTCCAAGAAATATGGAACTATGTGAAAAG | 1 | 229 |
| | | | ACCAAACCTACGTTTGATTGGTGTACCTGAA | | |
| | | | AGTGACAGAGAGAATGG | | |
| chr1 | 174145346 | 174145423 | CCTCCAAGAAATATGGGACTATGTGAGAAG | 1 | 230 |
| | | | ACCAAACCTACGTTTGATTGGTGTACCTGAA | | |
| | | | AGTGATGGGGAGAATGG | | |
| chr1 | 183750168 | 183750245 | CCATTCTCCCCATCGCTTTCAGGTACACCAA | 1 | 231 |
| | | | TCAAACGTAGGTTTGGTCTTTTCACATAGTT | | |
| | | | CCATATTCTTTGGAGG | | |
| chr1 | 200801540 | 200801617 | CCATTCTCCCCATCACTTTCAGGTGTACCGA | 1 | 232 |
| | | | TCAAACGTAGGTTTGGTCTTTTCACATAGTC | | |
| | | | CCATATTTCTTGGAGG | | |
| chr1 | 207589936 | 207590013 | CCTCCAAGAAATATGGGACTATGTGAAAAG | 1 | 233 |
| | | | ACCAAACCTACGTTTGATTGGTGTACCTGAA | | |
| | | | AGTGACGGGGAGAATGG | | |
| chr1 | 209768370 | 209768445 | CCTTCAGGGCAGAAACAGCTCTACTAGCAG | 4 | 234 |
| | | | AGAAAGCAAGCTTTCAATATTGTGCAATACA | | |
| | | | AAAACGAGAGCAGGG | | |
| chr1 | 218652378 | 218652455 | CCATTCTCCTCATCTCCTTCTGGTACTCCAAT | 1 | 235 |
| | | | CAAACGTAGGTTTGGTCTTTTCTCATAGTCTC | | |
| | | | ATATTTCTTGGAGG | | |
| chr1 | 222147250 | 222147327 | CCTCCAAGACATATAGGACTATGTGAAAATA | 1 | 236 |
| | | | CCAAACCTACGTTTGATTGGTGTACCTGAAA | | |
| | | | GTGACAGGGAGTATGG | | |
| chr1 | 245870710 | 245870785 | CCTGCCAGATACCAGTAGTCACTGTGAATTA | 4 | 237 |
| | | | CAAAGCTACGTTTCTTCCATAGGGAAAGTTT | | |
| | | | GGAGTCCAGCCAGG | | |
| chr2 | 2376037 | 2376114 | CCATTCTCCCTGTCACTTTCAGGTACACCAA | 1 | 238 |
| | | | TCAAACGTAGGTTTGGTCTTTTCACATAGTC | | |
| | | | CCATATTTCTTGGAGG | | |
| chr2 | 4119629 | 4119706 | CCATTCTCCCCACCACTTTCAGGTACACCAA | 1 | 239 |
| | | | TCAAACGTAGGTTTGGTCTTTTCACATAGTC | | |
| | | | CCATATTTCTTGTAGG | | |
| chr2 | 4909047 | 4909124 | CCTAACCAGAAACTAACTAATAGATATGGG | 1 | 240 |
| | | | CAGAAAGCATCCTTTCACTTTTGTTCTGGGA | | |
| | | | GAGGGAAGAAGCAAAGG | | |
| chr2 | 28984877 | 28984953 | CCATTTTGGGGAGGCCTTGATGGGAAGCTGG | 2 | 241 |
| | | | AAAAGGAAGCTTTCCTCCCAGTCCTGCTGAA | | |
| | | | GGCCTTGCCAGCTGG | | |
| chr2 | 31755833 | 31755910 | CCTCCAAGAAACACAGGACTATGTGAAAAG | 1 | 242 |
| | | | ATCAAACCTACGTTTGATTGGTGTTCCTGAA | | |
| | | | AGTGATGGGGAGAATGG | | |
| chr2 | 39829583 | 39829660 | CCATTCTCTTCATGACTTTCAGGTACACCATT | 1 | 243 |
| | | | GAAACGTAGGTTTGGTCTTTTCACATTGTCC | | |
| | | | CATATTTCTTGGAGG | | |
| chr2 | 60205947 | 60206024 | CCATTCTCCCCATCACTTTCAGGTACACCAA | 1 | 244 |
| | | | TCAAACGTAGGTTTGGTCTTTTCACATAGTC | | |
| | | | CCGTATTTCTTGGTGG | | |
| chr2 | 79082362 | 79082439 | CCATTCTCCCTGTCACTTTCAGGTACACCAA | 1 | 245 |
| | | | TCAAACGTAGGTTTGGTCTTTTCACATAGTC | | |
| | | | CCATATTTCTTGGGGG | | |
| chr2 | 79082362 | 79082438 | CCATTCTCCCTGTCACTTTCAGGTACACCAA | 3 | 246 |
| | | | TCAAACGTAGGTTTGGTCTTTTCACATAGTC | | |
| | | | CCATATTTCTTGGGG | | |
| chr2 | 108430915 | 108430992 | CCTCCAAGAAATATGAGATTATATGAAAAG | 1 | 247 |
| | | | ACCAAACCTACGTTTGATTGGTGTACTTTAA | | |
| | | | AGTGACGGGGAGAATGG | | |
| chr2 | 115893685 | 115893762 | CCATTCTCCCCGTCATTTTCAGGTACACCAA | 1 | 248 |
| | | | TCAAACGTAGGTTTGGTCTTTTCACATAGTC | | |
| | | | CCAAATTTCTTGGAGG | | |
| chr2 | 119620068 | 119620145 | CCCCCAAGAAATGTGGGACTATATGAAAAG | 1 | 249 |
| | | | ACCAAACCTACGTTTGACTGGTGTACCTAAA | | |
| | | | AGTGATGGGGAGAATGG | | |
| chr2 | 119620069 | 119620145 | CCCCAAGAAATGTGGGACTATATGAAAAGA | 2 | 250 |
| | | | CCAAACCTACGTTTGACTGGTGTACCTAAAA | | |
| | | | GTGATGGGGAGAATGG | | |
| chr2 | 128495068 | 128495144 | CCCATTGGTGCTGACCAGATGGTGAAGGAG | 2 | 251 |
| | | | GCAAAGGTTGCTTTGAATGACTGTGCTCTGG | | |
| | | | GGTGAGCCAGGCCTGG | | |
| chr2 | 133133559 | 133133634 | CCCTTTACAGAGGTGAGCTTTGTTATTAGTA | 4 | 252 |
| | | | AAAAGGTAGGTTTCCCTGTTTTTCTGAAGAA | | |
| | | | AAGCTGTGAGTGGG | | |
| chr2 | 134174983 | 134175060 | CCACTGCCCATTGACAGAGTGGCGAGGTGG | 1 | 253 |
| | | | GTGAAACCTTGCTTTCCTCCTGGCCCATGGG | | |
| | | | CAGGGTGGGGCTGTGGG | | |
| chr2 | 134174983 | 134175059 | CCACTGCCCATTGACAGAGTGGCGAGGTGG | 3 | 254 |
| | | | GTGAAACCTTGCTTTCCTCCTGGCCCATGGG | | |
| | | | CAGGGTGGGGCTGTGG | | |
| chr2 | 138069945 | 138070022 | CCATTCTCCCTGTCACTTTTAGATACACCAAT | 1 | 255 |
| | | | CAAACGTAGGTTTGGTCTTTTCACATAGTCC | | |
| | | | CATGTTTCTTGGAGG | | |
| chr2 | 138797420 | 138797496 | CCTCCAAGAAATATCAACTGTGTGAAAAGA | 2 | 256 |
| | | | CGAAACCTACGTTTGATTAATGTACCTGAAA | | |
| | | | GTGACAGGGAGAATGG | | |
| chr2 | 145212434 | 145212511 | CCATTCTCCCATTAACTTTCAAGTACACCAA | 1 | 257 |
| | | | TCAAAGGTAGGTTTGGTGTTTTCCCATAGTC | | |
| | | | CCGTATTTCTTGGAGG | | |
| chr2 | 147837842 | 147837919 | CCTTTTCATCATGCCCCTTTCACTTTAAGGTG | 1 | 258 |
| | | | AAAACCTTGCTTTACATGTCAGAGAAAAGA | | |
| | | | AGAGCCCTCAGCTGGG | | |
| chr2 | 147837842 | 147837918 | CCTTTTCATCATGCCCCTTTCACTTTAAGGTG | 3 | 259 |
| | | | AAAACCTTGCTTTACATGTCAGAGAAAAGA | | |
| | | | AGAGCCCTCAGCTGG | | |
| chr2 | 154152540 | 154152617 | CCATTCACCCCGTCACTTTCAGGTACACCAA | 1 | 260 |
| | | | TCAAACGTAGGTTTGGTCTTTTCACATAGTC | | |
| | | | CCATATTTCTTGGAGG | | |
| chr2 | 157705943 | 157706019 | CCTCCAAGAAATATGGGACTATGTGAAAAG | 3 | 261 |
| | | | ACCAAACCTACGTTTGATGGTGTACCCGAAA | | |
| | | | GTGACAGGGAGAATGG | | |
| chr2 | 158361152 | 158361229 | CCACCAAGAAATATGGGACTATGTGAAAAG | 1 | 262 |
| | | | ACCAAACCTACGTTTGATAGGTATACCTGAA | | |
| | | | AGTGACAGGGAGAATGG | | |
| chr2 | 161461006 | 161461083 | CCATTCTCCCCATCACTTTCAGGTGCACCAA | 1 | 263 |
| | | | TCAAACGTAGGTTTGGTCTTTTCACATAGTC | | |
| | | | CCATATTTCTTGGAGG | | |
| chr2 | 179077376 | 179077453 | CCCTCAAGAAATATGAGACTATGTGAAAAG | 1 | 264 |
| | | | ACCAAACCTACGTTTGACTGGTATACCTGAA | | |
| | | | AGTGACAGGGAGAATGG | | |
| chr2 | 179077377 | 179077453 | CCTCAAGAAATATGAGACTATGTGAAAAGA | 2 | 265 |
| | | | CCAAACCTACGTTTGACTGGTATACCTGAAA | | |
| | | | GTGACAGGGAGAATGG | | |
| chr2 | 181090699 | 181090776 | CCTCCAACAAATATGGGACTATGTGAAAAG | 1 | 266 |
| | | | ACCAAACCTACGTTTGATTGGTGTACCTGAA | | |
| | | | AGTGACGGGGATAATGG | | |
| chr2 | 182331957 | 182332034 | CCATTCTCTCCCTCACTTTCAAGTACACCAAT | 1 | 267 |
| | | | CAAACGTAGGTTTGGTCTTTTCACATAGTCT | | |
| | | | TATATTTCTTGGCGG | | |
| chr2 | 183620562 | 183620638 | CCATTCTCCCTGTCACTGTCAGTACACCAAT | 2 | 268 |
| | | | CAAACGTAGGTTTGGTCTCTTCACATAGTCC | | |
| | | | CATATTTCTTGGAGG | | |
| chr2 | 207345927 | 207346003 | CCTCCAAGAAATATGGGACTATGTGAACAG | 3 | 269 |
| | | | ACCAAACCTACGTTTGATTGGTGTACCTGAA | | |
| | | | AGTGATGGCAGAATGG | | |
| chr2 | 216652047 | 216652123 | CCACCATGCCTGGCCACCACACATTTTTTTCT | 2 | 270 |
| | | | AAAGCTTGGTTTTGGCCACAGTGAGAGTTTC | | |
| | | | TTGGGCTGTCAGGG | | |
| chr2 | 216652047 | 216652122 | CCACCATGCCTGGCCACCACACATTTTTTTCT | 4 | 271 |
| | | | AAAGCTTGGTTTTGGCCACAGTGAGAGTTTC | | |
| | | | TTGGGCTGTCAGG | | |
| chr2 | 223780040 | 223780116 | CCCACTAGGTGGCGATATCTGAGGGTCCAAT | 2 | 272 |
| | | | GAAACCATGCTTTTTACTCAGATCTTCCACT | | |
| | | | AACCACCTCCCCCGG | | |
| chr2 | 224486595 | 224486672 | CCTCTAAGAAATATGGGACTATGTGAAAAG | 1 | 273 |
| | | | ACCAAACCTACGTTTGACTGGTGTACCTGAA | | |
| | | | AGTGACGGGGAGAATGG | | |
| chr2 | 230526902 | 230526979 | CCTCCAAGAAATATGGGACTATGTGAAAAG | 1 | 274 |
| | | | ACCAAACCTACGTTTGATTAGTGTACCTGAA | | |
| | | | AGTGACGGGGAGAATGG | | |
| chr2 | 232036127 | 232036204 | CCATTCTCCCTGTCACTTTCAGGTACATCAAT | 1 | 275 |
| | | | CAAACGTAGGTTTGGTCTTTTCACATAGTCC | | |
| | | | CATATTTCTTGGAGG | | |
| chr3 | 4072812 | 4072889 | CCTCCAAGAAATATGGGACTATGTGAAAAG | 1 | 276 |
| | | | ACCAAACCTACGTTTGACTGGTGTACCTGAA | | |
| | | | AGGGATGGGGAGAATGG | | |
| chr3 | 9261677 | 9261754 | CCCCCAAGAAATATGAGACTATGTGAAAAG | 1 | 277 |
| | | | ACCAAACCTACGTTTGATTGGTGTACCTGAA | | |
| | | | AGTGACAGGGAGAATGG | | |
| chr3 | 9261678 | 9261754 | CCCCAAGAAATATGAGACTATGTGAAAAGA | 2 | 278 |
| | | | CCAAACCTACGTTTGATTGGTGTACCTGAAA | | |
| | | | GTGACAGGGAGAATGG | | |
| chr3 | 16732146 | 16732223 | CCTCTAAGAAATATGGGACTATGTGAAAAG | 1 | 279 |
| | | | ACCAAACCTACGTTTGATTGGTGTAACTGAA | | |
| | | | AGTGACAGGGAGAATGG | | |
| chr3 | 17450712 | 17450789 | CCTCCAAGAAATATGCGCCTATGTGAAAAG | 1 | 280 |
| | | | ACCAAACCTACGTTTGATTGGTATACCTGAA | | |
| | | | AGTGATGGAGAGAATGG | | |
| chr3 | 21559769 | 21559846 | CCATTCTCCCTGTCACTTTGAGGTACACCAA | 1 | 281 |
| | | | TCAAACGTAGGTTTGGTCTTTTCACATATTC | | |
| | | | GCATATTTCTTGGAGG | | |
| chr3 | 23416658 | 23416735 | CCATTCTCCCCGTCACTTTCAGGTACACCAA | 1 | 282 |
| | | | CCAAACGTTGGTTTGGTCTTTTCACATAGTC | | |
| | | | CCATATTTCTTGGAGG | | |
| chr3 | 29984019 | 29984096 | CCATTCTCCCTGTCACTTTCCAGTACACCAGT | 1 | 283 |
| | | | CAAACGTAGGTTTGGTCTTTTCACATACTCC | | |
| | | | CATATTTCTTGGAGG | | |
| chr3 | 38269551 | 38269627 | CCTGGCCTAATTTTTAATTCTTAGTTTGACTT | 2 | 284 |
| | | | AAACCTTGCTTTTAGTGTGATGGCGACAAAA | | |
| | | | GCTGAGCTGAAAGG | | |
| chr3 | 40515213 | 40515288 | CCAGTGCTTTTTGGTTTTAAAGGCAAGCCTC | 4 | 285 |
| | | | CAAACCTTCCTTTCTCCTGGATGCTGTGGTG | | |
| | | | GTTGCCATGCATGG | | |
| chr3 | 49233612 | 49233687 | CCCAACTCCTGCGAGAAGTAGCTCACCATGA | 4 | 286 |
| | | | CAAAGCTACCTTTGCTTTTATCGTTTTGCAAA | | |
| | | | ACAAAAAAGGGGG | | |
| chr3 | 66292894 | 66292971 | CCATTCTCCCCGTCACTTTGAGGTGTGCCAA | 1 | 287 |
| | | | TCAAACGTAGGTTTGGTCTTTTCACATAGTC | | |
| | | | CTATATTTCTTGGAGG | | |
| chr3 | 67541493 | 67541570 | CCTCCAAAAAATATGGGACTACGTAAAAAG | 1 | 288 |
| | | | ACCAAACCTACGTTTGATTGGTGTACCTGAA | | |
| | | | ACTGACAGGGAGAATGG | | |
| chr3 | 82273011 | 82273088 | CCATTCTCCCCGTCACTTTCAGGTACACCAA | 1 | 289 |
| | | | TCAAACGTAGGTTTGGTCTTTTCACATAGTT | | |
| | | | CCATATTTCTTGGAGG | | |
| chr3 | 98683349 | 98683426 | CCTACAAGATATATGGGACTATGTGAAAAG | 1 | 290 |
| | | | ACCAAACCTACGTTTTACTGGTGTGCCTGAA | | |
| | | | ACTGACGGGGAGAATGG | | |
| chr3 | 101923653 | 101923730 | CCATTCTCTCTGTCACTTTCAGGTACACCAAT | 1 | 291 |
| | | | CAAACGTAGGTTTGGTCTTTTCACATAGTCC | | |
| | | | CATATTTCTTGGAGG | | |
| chr3 | 114533467 | 114533544 | CCTCCAAGAAATATGGGACTATGTGAAAAG | 1 | 292 |
| | | | ACCAAACCTACGTTTCATTGGTGTACCTGAA | | |
| | | | AGTGATAGGGAGAATGG | | |
| chr3 | 132607602 | 132607679 | CCTCCAAAAAATATGGGATGATGTGAAAAG | 1 | 293 |
| | | | ACCAAACCTAGGTTTGACTGGTGTACCTGAA | | |
| | | | AATGATGGGGAGAATGG | | |
| chr3 | 137545176 | 137545253 | CCTCCAAGAAATATGAGACTATGTGAAAAG | 1 | 294 |
| | | | ACCAAACCTACGTTTGATTGGTGTACCTGAA | | |
| | | | AGTGACAGGGAGAATGG | | |
| chr3 | 137655679 | 137655756 | CCTCCAAGAAATATGGGACTACGTGAAAAG | 1 | 295 |
| | | | ATCAAACCTACGTTTGATTGTTGTACCTGAA | | |
| | | | AGTGATGGGGAGAATGG | | |
| chr3 | 137662040 | 137662117 | CCTCCAAGAAATATGGGACTATGTGAAAAG | 1 | 296 |
| | | | ACCAAACCTACGTTTGATTGTTGTACCTGAA | | |
| | | | AGTGATGGGGAGAATGG | | |
| chr3 | 142133796 | 142133873 | CCTCAAAAGTGTTCTGGTTTTGTTTTGTTTTT | 1 | 297 |
| | | | TAAACCATGGTTTTACCTCTGGCTTAGTGGG | | |
| | | | ACTAAAAATAGGAGG | | |
| chr3 | 146726949 | 146727026 | CCTCCAAGAAATATGGGACTATGTGAAAAG | 1 | 298 |
| | | | ACCAAACCTACGTTTGACTGGTGTACCTGAA | | |
| | | | AGTGATGGGGAAAATGG | | |
| chr3 | 152421096 | 152421173 | CCTCCAAGAAATATGGGACTGTGTGTAAAG | 1 | 299 |
| | | | ACCAAACCTACGTTTGATTGGTGTACCTCAA | | |
| | | | AGTGATGGGGAGAATGG | | |
| chr3 | 170620247 | 170620324 | CCATTCTCCCCATCACATTCAGGTACACCAA | 1 | 300 |
| | | | TCAAACGTAGGTTTGGTCTTTTCACATAGTC | | |
| | | | CCATATTTCTTGGAGG | | |
| chr3 | 181166873 | 181166949 | CCCCTGGAAAAGTTGGAGCATCACAGGAAA | 3 | 301 |
| | | | AGCAAACCAACCTTTTTTCTCCCCTAGGTAA | | |
| | | | ACTGGGGAGCCAGGGG | | |
| chr3 | 181166874 | 181166949 | CCCTGGAAAAGTTGGAGCATCACAGGAAAA | 4 | 302 |
| | | | GCAAACCAACCTTTTTTCTCCCCTAGGTAAA | | |
| | | | CTGGGGAGCCAGGGG | | |
| chr4 | 6604233 | 6604309 | CCTTCCCCAGTTGCAGCAGACAAGAGTCTCG | 2 | 303 |
| | | | AAAAGCTTGCTTTGGTTGCTGCAGTGGATGG | | |
| | | | GTTGGTAGGCACAGG | | |
| chr4 | 6626269 | 6626344 | CCCCCACCTCCCAAGCTGCTGGCTTCTCGAA | 4 | 304 |
| | | | TAAAGCTACCTTTCCTTTTACCAAAACTTGTC | | |
| | | | TCTCGAATGTCGG | | |
| chr4 | 8155396 | 8155472 | CCTTGGCCCTGGACAGCTGCTTTTCCTTCCCT | 2 | 305 |
| | | | AAACCTTGGTTTCCCCCTTTGTGCAGGTGGG | | |
| | | | TGGGTTTGGGCTGG | | |
| chr4 | 10386803 | 10386880 | CCTCTTCTAGTGAACCCATGGGGTTACCAAG | 1 | 306 |
| | | | GGAAAGCAACCTTTTGATAAATATTCCCATC | | |
| | | | TTTTTATGTTGTCTGG | | |
| chr4 | 20701579 | 20701656 | CCACTTGAAAGGGTTACCAAGGATAAGATTT | 1 | 307 |
| | | | TTAAAGCTTGCTTTCACAAACAACTCATGCT | | |
| | | | CCAGGCTTGTCAGTGG | | |
| chr4 | 29594286 | 29594363 | CCTTTCTCCCCATCACTTTCAGGTACACCAAT | 1 | 308 |
| | | | CAAACGTAGGTTTGATCTTTTCACATAGTCC | | |
| | | | CATATTTCTTGGAGG | | |
| chr4 | 53668422 | 53668499 | CCATTCTCCCCATCAATTTCAGTTACACCAA | 1 | 309 |
| | | | TGAAACGTAGGTTTGGCCTTTTCACATAGTC | | |
| | | | CCATATTTCTTAGAGG | | |
| chr4 | 74914802 | 74914879 | CCATTCTCCCTGTCACTCTCAGGTACACCAA | 1 | 310 |
| | | | TCAAACGTAGGTTTGGTCTTTTCATATAGTC | | |
| | | | CCATATTTCTTGGAGG | | |
| chr4 | 75332783 | 75332859 | CCTCCAAGAAAATTGGGACTATGTGAAAAA | 3 | 311 |
| | | | ACCAAACCTACGTTTGATTGATGTACCTGAA | | |
| | | | AGTGACAGGAGAATGG | | |
| chr4 | 88123643 | 88123720 | CCTTCAAGAAATATGGGACTATGTGAAAGG | 1 | 312 |
| | | | ACAAAACCTACGTTTTATTGGTGTACCTGAA | | |
| | | | AGTGACAGGGAGAATGG | | |
| chr4 | 89567192 | 89567269 | CCATTCTCCCCATCACTTTCAGGTACGCTAA | 1 | 313 |
| | | | TCAAACGTAGGTTTGATCTTTTCACATAGTC | | |
| | | | TTATATTTCTTGGAGG | | |
| chr4 | 93556577 | 93556654 | CCTCCAAGAAATATGGGACTATGTGAAAAG | 1 | 314 |
| | | | ACCAAACCTACGTTTGACTGGTGTACCTCAA | | |
| | | | TGTGACAGGGAGAATGG | | |
| chr4 | 100266379 | 100266456 | CCATTCTCCCTGTCACTTTTAGGTACACCAAT | 1 | 315 |
| | | | CAAACGTACGTTTGGTCTTTTCACATAGACC | | |
| | | | CATATTTCTTGGAGG | | |
| chr4 | 103486234 | 103486311 | CCTTCAAGAAATATGGGACTGTGTGAAAAG | 1 | 316 |
| | | | ACCAAAGCTAGGTTTGATTGGTGTACCTGAA | | |
| | | | AGTGATGGGGAGAATGG | | |
| chr4 | 105923129 | 105923204 | CCTACTATTCACAGAGTAATGCAGTTTGCTG | 4 | 317 |
| | | | AAAAGGTTGGTTTTTGCTGACCTCTGAGAGC | | |
| | | | TCACATTACAGTGG | | |
| chr4 | 106874711 | 106874788 | CCATTCTCTCTGTCACTTTCTGGTACACCAAT | 1 | 318 |
| | | | CAAACGTAGGTTTGCTCTTTTCACATAATCC | | |
| | | | CATATTTATTGAAGG | | |
| chr4 | 115805791 | 115805867 | CCATAACATGTATTTGCTGGTGCTAGACTCT | 3 | 319 |
| | | | CCAAAGCTAGGTTTCTTTCTACAACAATGGC | | |
| | | | TGGAAGTCTTCTTGG | | |
| chr4 | 122033277 | 122033354 | CCATTCTCCCCATCACTTTCAGGTACACCAA | 1 | 320 |
| | | | TCAAACGTAGGTTTGGTCTTCTCACACAGTC | | |
| | | | CCATATTTCTTGGAGG | | |
| chr4 | 129125132 | 129125209 | CCATTCTTCCCATTACTTTCAGGTACACCAAT | 1 | 321 |
| | | | CAAACGTAGGTTTGGTCTTTTCACATAGTCC | | |
| | | | CACATTTCTTGGAGG | | |
| chr4 | 135472562 | 135472639 | CCATTCTCCCCCTCACTTTCAGGTACACCAA | 1 | 322 |
| | | | TCAAACGTAGGTTTGGTCTTTTCACATTGTCC | | |
| | | | CATATTTCTTGGAGG | | |
| chr4 | 138507099 | 138507176 | CCATTCTCCCCAGCACTTACAGGTACACCAA | 1 | 323 |
| | | | TCAAACGTAGGTTTGGTCATTTCACATAGTC | | |
| | | | CCATATTTCTTGGAGG | | |
| chr4 | 144249093 | 144249170 | CCATTCTCCCTGTCACTTTCAGGTACAGCAA | 1 | 324 |
| | | | TCAAACGTAGGTTTGGTCTTTTCACATGGTC | | |
| | | | CCATATTTCTTGGAGG | | |
| chr4 | 144436406 | 144436483 | CCTCCAAGAAATATGAGACTATGTGAAAAG | 1 | 325 |
| | | | ACCAAACCTACGTTTGATTGGTGTACCTGAA | | |
| | | | AGTGACGGGGAAGATGG | | |
| chr4 | 154110259 | 154110336 | CCTCCAAGAAATATGAGACTATGTGAAAAG | 1 | 326 |
| | | | ACCAAACCTACGTTTGATTGGTGTACCTGAA | | |
| | | | AGTGACAGGGAGAATGG | | |
| chr4 | 154893438 | 154893515 | CCTCCAAGAGATATGAGACTATGTAAATAG | 1 | 327 |
| | | | ACCAAACCTACCTTTGATTGGTGTACGTGAA | | |
| | | | AGTGACAGGAAGAATGG | | |
| chr4 | 161116854 | 161116931 | CCATTCTCCCCATCACTTTCAGGTACACCAA | 1 | 328 |
| | | | CCAAACGTAGGTTTGGTCTTTTCACATAGTC | | |
| | | | TCATATTTCTTGGAGG | | |
| chr4 | 165140748 | 165140823 | CCTCCATTGACTACTCCTTATCATTGGCTAG | 4 | 329 |
| | | | AAAACCTACCTTTCAACCAGTTTCTAAGGCC | | |
| | | | AAGAAACTTGGAGG | | |
| chr4 | 181928508 | 181928585 | CCACCAAGAAATATGGGACTACGTGAAAAG | 1 | 330 |
| | | | ACCAAACCTACGTTTGATGGGTGTGCCTGAA | | |
| | | | AGTGACGGGAAGAATGG | | |
| chr4 | 187521958 | 187522035 | CCTCCAAGAAATAAGGGACTATGTGAAAAG | 1 | 331 |
| | | | ACCAAACCTACGTTTGATTGGTGTACCTGAA | | |
| | | | GGTGACAGGGAGAATGG | | |
| chr5 | 12675639 | 12675715 | CCAAAGGGCCTTTGTGATTCTACTTTGTAAT | 3 | 332 |
| | | | ATAAAGGATGGTTTCTTACTACGGTTGGTGT | | |
| | | | CCTTGCAGGAGTGGG | | |
| chr5 | 29271804 | 29271881 | CCTCCAAGAAATATGGGACTATGTGAAAAG | 1 | 333 |
| | | | ACCAAACCTACGTTTGATTGGTGTACCTGAA | | |
| | | | AGTGATGGGGAGAATGG | | |
| chr5 | 35352660 | 35352737 | CCATTCTCCCCGTTACTTTCAGGTACACCAA | 1 | 334 |
| | | | TAAAACCTAGGTTTGGTCTTTTCACATAGTC | | |
| | | | CCATATTTCTTGGAGG | | |
| chr5 | 38723235 | 38723310 | CCCATATCTCTGGCAAGGGCAGCTCTCTGGC | 4 | 335 |
| | | | TAAACCAAGCTTTCCTGTAGAGCTTGAGTTC | | |
| | | | CAAGGCAGCGTTGG | | |
| chr5 | 47358339 | 47358415 | CCTTGTAGTGTGTGTATTCAACTCACAGAGT | 2 | 336 |
| | | | TAAACGATCCTTTACACAGAGCAGACTTGAA | | |
| | | | ACACTCTTGTTGTGG | | |
| chr5 | 47415811 | 47415887 | CCTTGTAGTGTGTGTATTCAACTCACAGAGT | 2 | 337 |
| | | | TAAACGATCCTTTACACAGAGCATACTTGAA | | |
| | | | ACACTCTTTTTGTGG | | |
| chr5 | 47474614 | 47474690 | CCTTGTGTTGTGTGTATTCAACTCACAGAGT | 2 | 338 |
| | | | TAAACGATCCTTTACACAGAGCAGACTTGAA | | |
| | | | ACACTCTTTTTGTGG | | |
| chr5 | 48228356 | 48228432 | CCTTGTGTTGTGTTTATTCAACTCACAGAGTT | 2 | 339 |
| | | | AAACGATCCTTTACACAGAGCAGACTTGAA | | |
| | | | ATACTCTTTTTGTGG | | |
| chr5 | 48454551 | 48454627 | CCTTGTAGTGTGTGTATTCAACTCACAGAGT | 2 | 340 |
| | | | TAAACGATCCTTTACACAGAGCATACTTGAA | | |
| | | | ACACTCTTTTTGTGG | | |
| chr5 | 48612272 | 48612348 | CCTTGTATTGTGAGTATTCAACTCACAGAGT | 2 | 341 |
| | | | TAAACGATCCTTTACACAGAGCAGACTTGAA | | |
| | | | ACACTCTTTTTGTGG | | |
| chr5 | 48884165 | 48884241 | CCTTGTGTTGTGTGTCTTCAACTCACAGAGTT | 2 | 342 |
| | | | AAACGATGCTTTACACAGAGTAGACTTGAA | | |
| | | | ACACTCTTTTTCTGG | | |
| chr5 | 49037011 | 49037087 | CCTTGTGTTGTGTGTATTCAACTCACAGAGT | 2 | 343 |
| | | | TAAACGATCCTTTACACAGAGCAGACTTGTA | | |
| | | | ACACTCTTTTTGTGG | | |
| chr5 | 49038372 | 49038448 | CCTTGTGTTGTGTGTATTCAACTCACAGAGT | 2 | 344 |
| | | | TAAACGATCCTTTACACAGAGCAGACGTGA | | |
| | | | AACACTCTTTTTGTGG | | |
| chr5 | 49150718 | 49150794 | CCTTGTGTTGTGTGTATTCAACTCACAGAGT | 2 | 345 |
| | | | TAAACGATCCTTTACACAGAGCAGACTTGAA | | |
| | | | ACACTCTTTTTGTGG | | |
| chr5 | 49241653 | 49241729 | CCTTGTGTTGTGTGTATTCAACTCACAGAGT | 2 | 346 |
| | | | TAAACGATCCTTTACACAGAGGAGACTTGTA | | |
| | | | ACACTCTTTTTGTGG | | |
| chr5 | 88582714 | 88582790 | CCTTTTCATAAGAAGAAAATCGACTCATCAT | 3 | 347 |
| | | | TGAAACCAAGCTTTGGTACAATTTCATTGAT | | |
| | | | GTTTCCAGAAGCAGG | | |
| chr5 | 93497156 | 93497231 | CCCATAGACTATGATAGAAACAAAATAACC | 4 | 348 |
| | | | CAAAAGCTAGCTTTCTGATTGAGTTTCCATA | | |
| | | | AATGCAATGTGAAGG | | |
| chr5 | 94295029 | 94295105 | CCATTCACTTGTCACTTTCTGGTACACCAATC | 2 | 349 |
| | | | AAACGTAGGTTTGGTCTTTTCACATAGTCTC | | |
| | | | ATATTTCTTGGAGG | | |
| chr5 | 94956746 | 94956823 | CCTCCAAGAAATATGGGACTCTGTAAAGAG | 1 | 350 |
| | | | ACCAAACCTACGTTTGATTGGTGTACCTGAA | | |
| | | | AGTGAAGGGGAGAATGG | | |
| chr5 | 106003488 | 106003565 | CCATTCTCCCCGTCATTTTCAGGTACACCAA | 1 | 351 |
| | | | TCAAACCTAGGTTTGGTCTTTTTACATAGTCC | | |
| | | | CATATTTCTTGGAGG | | |
| chr5 | 118727905 | 118727982 | CCTCCACGAAACATGGGACTATGTGAAAAG | 1 | 352 |
| | | | ACCAAACCTACGTTTGATTGGTGTACCTGAA | | |
| | | | AGTGACAGGGAGAATGG | | |
| chr5 | 132156032 | 132156109 | CCAATTTCCCCCTCACTTTCAGATACACCAA | 1 | 353 |
| | | | TCAAACGTAGGTTTGGTCTTTTCACATAGTT | | |
| | | | CCATATTTCCTGGAGG | | |
| chr5 | 152037951 | 152038028 | CCATTCTCCCCATCACTTTCAGGTACACCAA | 1 | 354 |
| | | | TCAAACGTAGGTTTGGTCTTTTCACATATTCC | | |
| | | | CATATGTCTTGGAGG | | |
| chr5 | 155183064 | 155183141 | CCCACCGGCTCATGAGAGGTAGAGCTAAGG | 1 | 355 |
| | | | TCCAAACCTAGGTTTATCTGAGACCGGAACT | | |
| | | | CATGTGATTAACTGTGG | | |
| chr5 | 155183065 | 155183141 | CCACCGGCTCATGAGAGGTAGAGCTAAGGT | 2 | 356 |
| | | | CCAAACCTAGGTTTATCTGAGACCGGAACTC | | |
| | | | ATGTGATTAACTGTGG | | |
| chr5 | 163148211 | 163148288 | CCTTCAAGAAATATGGGACTATGTGAAGAG | 1 | 357 |
| | | | ACCAAACCTACGTTTGATTGGTGTAGCCAAA | | |
| | | | AGTGATGGGGAAAATGG | | |
| chr5 | 165889537 | 165889614 | CCTCAGATTAGATTTACTTGCAAAGAGACAT | 1 | 358 |
| | | | TTAAAGGATCGTTTTGATACTATTTTGAAAG | | |
| | | | TACTATACAAAGATGG | | |
| chr5 | 169395198 | 169395274 | CCTTAAGAACATAAATCCCCAGGAATTCACA | 2 | 359 |
| | | | GAAACCTTGGTTTGAGCTTTGGATTTCCCGC | | |
| | | | AGGATGTGGGATAGG | | |
| chr5 | 171021380 | 171021457 | CCATTCTCTCTGTCACTTTCAGGTACACCAAT | 1 | 360 |
| | | | CAAACGTAGGTTTGGTCTTTTCTCATAGTCC | | |
| | | | CATATTTCTTGGAGG | | |
| chr5 | 173059898 | 173059973 | CCATTTACCATCATTCTCTGTCATGGCAGGT | 4 | 361 |
| | | | GAAAGCAAGCTTTTATATAGACAATGTTCTA | | |
| | | | CTTAGTTTACAGGG | | |
| chr5 | 174102359 | 174102435 | CCCAAAGTTAATTTTACTCTTTTTCTGAATCA | 2 | 362 |
| | | | AAAGGAACCTTTCCTCCATGAGAAGAATCCT | | |
| | | | GCCATATTTCTAGG | | |
| chr5 | 180927811 | 180927888 | CCTCCAAGAAATATGGGACTATGTGAAAAG | 1 | 363 |
| | | | ACCAAACCTACGTTTGATTGCTATACATGAA | | |
| | | | AGTGACGGGGAGAATGG | | |
| chr6 | 1752363 | 1752440 | CCTTCAAGAAATATGGGACTATGTGAAAAG | 1 | 364 |
| | | | ACCAAACCTACCTTTGATTGGTGTACCTGAA | | |
| | | | AGTGATGGGAAGAATGG | | |
| chr6 | 20595279 | 20595356 | CCATTCTCCCCATCACTTTCAGGTACACCAA | 1 | 365 |
| | | | TCAAACGTAGGTTTGGTCTTTTCACATAGTC | | |
| | | | CCATAGTTCTTGGAGG | | |
| chr6 | 23431370 | 23431447 | CCATTCTCCCCGTCACTTTCAGGGACAACAA | 1 | 366 |
| | | | TCAAACGTAGGTTTGGCCTTTGCACATAGTC | | |
| | | | TTATATTTCTTGGAGG | | |
| chr6 | 29190624 | 29190701 | CCATTCTCCCCATCACTTTCAGGTACACCAA | 1 | 367 |
| | | | TCAAACGTAGGTTTGGTCTTTTCACATAGTC | | |
| | | | CCATATTTCTTGGAGG | | |
| chr6 | 61533266 | 61533343 | CCTCCAAAAAATATGGGACTATGTGAGAAG | 1 | 368 |
| | | | ACCAAACCTACGTTTTATTAGTGTACCTCAA | | |
| | | | AGTGACAGGGAGGATGG | | |
| chr6 | 101052764 | 101052841 | CCATTCTCCCCATCACTTTCAGGTACACCAA | 1 | 369 |
| | | | TGAAACGTAGGTTTGGCCTTTTCACATAGTT | | |
| | | | TCATATTTCTTGGAGG | | |
| chr6 | 117176355 | 117176432 | CCTCCAAGAAATATGGGACTATGTGAAAAG | 1 | 370 |
| | | | ACCAAACCTACGTTTGATTGGTGTACCTGAA | | |
| | | | AGTGATGGGGAGAATGG | | |
| chr6 | 117747073 | 117747149 | CCTACAAGAAATATGGAACTTGTAAAAAGA | 2 | 371 |
| | | | CCAAACCTACGTTTGATTGGTGTACCTGAAA | | |
| | | | GTGACGGGGAGAATGG | | |
| chr6 | 118422508 | 118422585 | CCTCCAAGAAATATGGGACAATGTGAAAAG | 1 | 372 |
| | | | GCCAAAGCTACGTTTGATTGGTGTACCTGAA | | |
| | | | AGTGACAGGGAGAATGG | | |
| chr6 | 122035019 | 122035096 | CCTTTCAAACTTAGAGGTAAACAAAAGTCCT | 1 | 373 |
| | | | GAAAACCTAGGTTTGACCATAAGTTGGGACC | | |
| | | | ATACGAGCATAGAAGG | | |
| chr6 | 134445210 | 134445287 | CCAAAAATAAAAAAAAATTGACTTATAAGT | 1 | 374 |
| | | | AAGAAAGGTTCGTTTTCTCACATTCAGAAAG | | |
| | | | AGAACCCACATGTTGGG | | |
| chr6 | 134445210 | 134445286 | CCAAAAATAAAAAAAAATTGACTTATAAGT | 3 | 375 |
| | | | AAGAAAGGTTCGTTTTCTCACATTCAGAAAG | | |
| | | | AGAACCCACATGTTGG | | |
| chr6 | 135154944 | 135155021 | CCATTCTCCCCATCACTTTCAGGTACACCAA | 1 | 376 |
| | | | TCAAACGTAGGTTTGGTCTTTTCACATAGTC | | |
| | | | CCATATTTCTTGGAGG | | |
| chr6 | 137889995 | 137890072 | CCATTCTCCCCGTCACTTTCAGGTACACCAA | 1 | 377 |
| | | | TCAAACGTTGGTTTAGTCTATTCACATAGTC | | |
| | | | CCATATTTCTTGGAGG | | |
| chr6 | 143993904 | 143993981 | CCGAAAAGAATAAGACTATCAGCTGAAGTC | 1 | 378 |
| | | | TTAAAACGATCCTTTGGCCCCCAGTACTCTA | | |
| | | | TATGCAGGATAGAAAGG | | |
| chr6 | 152610473 | 152610549 | CCTACAAAAATAGGGGACTATGTGATAAGA | 2 | 379 |
| | | | CCAAACCTACGTTTGATTGGTGTACCTGAAA | | |
| | | | GTGATGGGGAGAATGG | | |
| chr6 | 160372604 | 160372681 | CCATTCTACCCATCACTTTCAGGTACACCAA | 1 | 380 |
| | | | TCAAACGTAGGTTTGGCCTTTTCATATAGTC | | |
| | | | TCATATTTCTTGGAGG | | |
| chr6 | 169352478 | 169352555 | CCATTCTCCCCATCACTTTCTGGTATACCAAT | 1 | 381 |
| | | | CAAACGTAGGTTTGGTCTTTTCACATAGTCC | | |
| | | | CATATTTCTTAGAGG | | |
| chr6_GL000251v2_alt | 677196 | 677273 | CCATTCTCCCCATCACTTTCAGGTACACCAA | 1 | 382 |
| | | | TCAAACGTAGGTTTGGTCTTTTCACATAGTC | | |
| | | | CCATATTTCTTGGAGG | | |
| chr6_GL000252v2_alt | 456242 | 456319 | CCATTCTCCCCATCACTTTCAGGTACACCAA | 1 | 383 |
| | | | TCAAACGTAGGTTTGGTCTTTTCACATAGTC | | |
| | | | CCATATTTCTTGGAGG | | |
| chr6_GL000253v2_alt | 456279 | 456202 | CCATTCTCCCCATCACTTTCAGGTACACCAA | 1 | 384 |
| | | | TCAAACGTAGGTTTGGTCTTTTCACATAGTC | | |
| | | | CCATATTTCTTGGAGG | | |
| chr6_GL000254v2_alt | 456371 | 456448 | CCATTCTCCCCATCACTTTCAGGTACACCAA | 1 | 385 |
| | | | TCAAACGTAGGTTTGGTCTTTTCACATAGTC | | |
| | | | CCATATTTCTTGGAGG | | |
| chr6_GL000255v2_alt | 456225 | 456302 | CCATTCTCCCCATCACTTTCAGGTACACCAA | 1 | 386 |
| | | | TCAAACGTAGGTTTGGTCTTTTCACATAGTC | | |
| | | | CCATATTTCTTGGAGG | | |
| chr6_GL000256v2_alt | 500011 | 500088 | CCATTCTCCCCATCACTTTCAGGTACACCAA | 1 | 387 |
| | | | TCAAACGTAGGTTTGGTCTTTTCACATAGTC | | |
| | | | CCATATTTCTTGGAGG | | |
| chr7 | 5256551 | 5256627 | CCACCACACCCAGCCTTATGGGATGGTTTTC | 2 | 388 |
| | | | AAAAGCATCCTTTTTTAGAAGTGGATTCTGA | | |
| | | | TATATAATCGGATGG | | |
| chr7 | 7392583 | 7392660 | CCATTCTCAATGTCACTTTCAGGTACACCAA | 1 | 389 |
| | | | TCAAACGTAGGTTTGGTCTTTTCACATAGTC | | |
| | | | CCATATTTCTTGGAGG | | |
| chr7 | 8737741 | 8737818 | CCATTCTCTCTGTCACTTTCAGGTACACCAGT | 1 | 390 |
| | | | CAAAGGTAGGTTTGTTTTATTCACACGTTCA | | |
| | | | CATATTTCTTGGAGG | | |
| chr7 | 11352226 | 11352303 | CCATTCGCCCCATCACTTTCAGGTACACTAG | 1 | 391 |
| | | | TAAAACGTAGGTTTGGTCTTTTCACATAGTT | | |
| | | | CCATATTTCTTGGAGG | | |
| chr7 | 15519145 | 15519222 | CCTCCAAGAAATATGGGACTATGTGAAGAG | 1 | 392 |
| | | | ATCAAACCTAGGTTTGATTGTTGTACCTGAA | | |
| | | | AGTGATAAGAAGAATGG | | |
| chr7 | 19228341 | 19228418 | CCTCCAATAAATATGGGGCTATGTGAAAAG | 1 | 393 |
| | | | ACCAAACCTACGTTTGATTGGTGTACCTGAA | | |
| | | | AGTGACAGGGAGAATGG | | |
| chr7 | 23778445 | 23778522 | CCCTTTTCCCTGTCACTTTCAGGTACACCAGT | 1 | 394 |
| | | | CAAACGTAGGTTTGGTCTTTTCACATAGTCG | | |
| | | | AATATTTCTTCAAGG | | |
| chr7 | 23778446 | 23778522 | CCTTTTCCCTGTCACTTTCAGGTACACCAGTC | 2 | 395 |
| | | | AAACGTAGGTTTGGTCTTTTCACATAGTCGA | | |
| | | | ATATTTCTTCAAGG | | |
| chr7 | 26769065 | 26769142 | CCATTCTCCCTGTCACTTTCAGGTACACTAAT | 1 | 396 |
| | | | CAAACGTAGGTTTGGTGTATTCACACAGTCC | | |
| | | | CATATTTCTTGGAGG | | |
| chr7 | 42864035 | 42864112 | CCATTCTTCCTGTCACTTTCAGGTATACCAAT | 1 | 397 |
| | | | CAAACGTAGGTTTGGTCTTTTCACATAGTCC | | |
| | | | CATGTTTCTTGGAGG | | |
| chr7 | 46498923 | 46499000 | CCTCCAAGAAATATGAGACTATATGAAAAT | 1 | 398 |
| | | | ACCAAACCTACGTTTGATTGGTGTACCTGAA | | |
| | | | AGAGACAGGGAGAATGG | | |
| chr7 | 51535360 | 51535437 | CCATTCTCCCTATCACTTTCAGGTACACCAA | 1 | 399 |
| | | | TCAAACGTAGGTTTGGTCTTTTCATGTAGTC | | |
| | | | CCATATTTCTTGGAGG | | |
| chr7 | 51927106 | 51927183 | CCATTCTGCCCGTCACTTTCAGGTACACCAA | 1 | 400 |
| | | | TCAAACGTAGGTTTGGTCTTTTCACATAGTC | | |
| | | | CCATATTTCTTGGAGG | | |
| chr7 | 56976942 | 56977018 | CCGTCCGATTATATATCAGAATCTACTTCTA | 3 | 401 |
| | | | AAAAAGGATGCTTTTGAAAACCATCCCATAA | | |
| | | | GGCTGGGTGTGGTGG | | |
| chr7 | 80021598 | 80021675 | CCTACAAGGAATATAGGACTATGTGAAAAT | 1 | 402 |
| | | | ACCAAACCTACGTTTCACTGCTGTACCTGAA | | |
| | | | GGTGACAGGGAGAATGG | | |
| chr7 | 89673853 | 89673930 | CCATTCTCCCCATCATTTCCAGGTAAACCAA | 1 | 403 |
| | | | TCAAAGGTAGGTTTGGTCATTTCACATAGTC | | |
| | | | CCATATTTCTTGGAGG | | |
| chr7 | 103404790 | 103404867 | CCATTCTCCCCGTCACTTTCAGGTACACCAG | 1 | 404 |
| | | | TCAAACGTAGGTTTGGTCTTTTCACACAGTC | | |
| | | | CCATATTTCCTGGAGG | | |
| chr7 | 113053651 | 113053728 | CCATTCTCCCCATCACTTTCAGGTACAGCAA | 1 | 405 |
| | | | TCAAACGTAGGTTTGGTCTTTTCACATAGTC | | |
| | | | CCATATTTCTTGGAGG | | |
| chr7 | 125765204 | 125765279 | CCACTACAGATTCTTGGGTCAAGATGTGTGC | 4 | 406 |
| | | | AAAAGGATGCTTTAGGGTGATGGATATGAG | | |
| | | | TGGGATGAAATGAGG | | |
| chr7 | 128042158 | 128042234 | CCTGAAAAAAAACCCTGCCAGCCAGCAACT | 3 | 407 |
| | | | CTGAAAGGATGCTTTGTGTGAGTGAGCAGTG | | |
| | | | TCTGAGATGGACAGGG | | |
| chr7 | 130637332 | 130637409 | CCATTCTCCCCATCACTTTCAGGTACGCCAA | 1 | 408 |
| | | | TCAAACGTAGGTTTGGTCTTTTGACATAGTC | | |
| | | | CCATATTTCTTGGAGG | | |
| chr7 | 136983050 | 136983127 | CCGTTCTCCCCATCACTTTTAGGTACACCAA | 1 | 409 |
| | | | TCAAACGTAGGTTTGGTCTTTTCACATAGTC | | |
| | | | TCATATTTCTTGGAGG | | |
| chr7 | 143579507 | 143579584 | CCATTCTCCTGGTCACTTTCAGGTATACCAA | 1 | 410 |
| | | | TCAAACGTAGGTTTGGTCTTTTCATGTAGTC | | |
| | | | CCATATTTCTTGGAGG | | |
| chr7 | 143749881 | 143749958 | CCTCCAAGAAATATGGGACTACATGAAAAG | 1 | 411 |
| | | | ACCAAACCTACGTTTGATTGGTATACCTGAA | | |
| | | | AGTGACCAGGAGAATGG | | |
| chr8 | 2338364 | 2338441 | CCTCCAAGAACTATGGGACTATGTGAAAAG | 1 | 412 |
| | | | ACCAAACCTACGTTTGATTGGTGTACCTGAA | | |
| | | | AGTGACGGGGAGAATGG | | |
| chr8 | 2383289 | 2383366 | CCATTCTCCCCGTCACTTTCAGGTACACCAA | 1 | 413 |
| | | | TCAAACGTAGGTTTGGTCTTTTCACATAGTC | | |
| | | | CCATAGTTCTTGGAGG | | |
| chr8 | 8414568 | 8414645 | CCATTCTCCCCGTCACTTTCAGGTACACCAA | 1 | 414 |
| | | | TCAAACGTAGGTTTGGTCTTTTCACAGAGTC | | |
| | | | CCATATTTCTTGGAGG | | |
| chr8 | 24163142 | 24163219 | CCATTCTCCCCGTCACTTTCATGTACACCAA | 1 | 415 |
| | | | GCAAACGTAGGTTTGATCTTTCCACATAGTC | | |
| | | | CCGTGTTTCTTGGAGG | | |
| chr8 | 34299051 | 34299128 | CCTCCAAGAAATATGGGACTATGTGAAAAG | 1 | 416 |
| | | | ACCAAACCTACGTTTGATTGGTGTACTTGAA | | |
| | | | AGTGACAGGGAGAATGG | | |
| chr8 | 40965485 | 40965562 | CCTCCAAGAAATATGGGACTATGTGAAAAG | 1 | 417 |
| | | | ACAAAACCTACGTTTCACTGGTGTACCTGAA | | |
| | | | AGTGACAGGGAGGATGG | | |
| chr8 | 48371659 | 48371735 | CCCCCACCTTTTAAAAACATGCATACATACG | 2 | 418 |
| | | | GAAACGTTGCTTTCTGCACGATTTCATTTTA | | |
| | | | ATGGAACAGAACAGG | | |
| chr8 | 82534960 | 82535037 | CCATTTCCCCTGTCACTTTCAGGTACACCAA | 1 | 419 |
| | | | TCAAACGTAGGTTTGGTCTTTTCACATAGTA | | |
| | | | TCATATTTCTTGGAGG | | |
| chr8 | 109217624 | 109217700 | CCATTCTCCCCGTCACTTTCAGGTACACCAA | 3 | 420 |
| | | | TCAAACGTAGGTTTGGTCTTTTCACATAGTC | | |
| | | | CCATATTTCTGGAGG | | |
| chr8 | 134790285 | 134790361 | CCTTTTGTTAAAGTAATAGAATTCTGCTTCTT | 2 | 421 |
| | | | AAAGGAACCTTTCAGGCAAGATGGTGGTTA | | |
| | | | GAGCACCTAAATGGG | | |
| chr8 | 134790285 | 134790360 | CCTTTTGTTAAAGTAATAGAATTCTGCTTCTT | 4 | 422 |
| | | | AAAGGAACCTTTCAGGCAAGATGGTGGTTA | | |
| | | | GAGCACCTAAATGG | | |
| chr8_KI270821v1_alt | 519635 | 519712 | CCTCCAAGAACTATGGGACTATGTGAAAAG | 1 | 423 |
| | | | ACCAAACCTACGTTTGATTGGTGTACCTGAA | | |
| | | | AGTGACGGGGAGAATGG | | |
| chr8_KI270821v1_alt | 564557 | 564634 | CCATTCTCCCCGTCACTTTCAGGTACACCAA | 1 | 424 |
| | | | TCAAACGTAGGTTTGGCCTTTTCACATAGTC | | |
| | | | CCATAGTTCTTGGAGG | | |
| chr9 | 14951207 | 14951283 | CCTCCAAGAAATATGGGACTGGTGAAAAGA | 2 | 425 |
| | | | CCAAACCTACGTTTGACTGGTGTACCTGAAA | | |
| | | | GTGACGGGGAGACTGG | | |
| chr9 | 23249218 | 23249295 | CCTCCAAGAAACATGGGAATGTGTGAAAAG | 1 | 426 |
| | | | ACCAAACCTACGTTTGATTGGCGTACCTGAA | | |
| | | | AGTGACGGGGAGTATGG | | |
| chr9 | 26278896 | 26278973 | CCTCCAAGAAATATGGGACTGTGTGAAAAG | 1 | 427 |
| | | | ACCAAACCTACGTTTGATTGGTATACCTGAA | | |
| | | | AGTGACAGAGAGAATGG | | |
| chr9 | 27323237 | 27323314 | CCATTCTCCCCTTCACTATCAGGTACACCAA | 1 | 428 |
| | | | TCAAACGTAGGTTTAGTCTTTTCACATAGTC | | |
| | | | CCATATTTCTTGGAGG | | |
| chr9 | 31517993 | 31518070 | CCATTCTCCCCGTCACTTTCAGATACACCAG | 1 | 429 |
| | | | TCAAACGTAGGTTTGGTCTTTTCACATAGTC | | |
| | | | CCATATTTCTTGGAGG | | |
| chr9 | 39694860 | 39694937 | CCATCTTACTTTGTACTACACTGTTCTTTAGA | 1 | 430 |
| | | | GAAAGCTTCCTTTTGGAGACCAACCAGGACT | | |
| | | | CCTTAGAAGCAGAGG | | |
| chr9 | 42451132 | 42451209 | CCATCTTACTTTGTACTACACTGTTCTTTAGA | 1 | 431 |
| | | | GAAAGCTTCCTTTTGGAGACCAACCAGGACT | | |
| | | | CCTTAGAAGCAGAGG | | |
| chr9 | 60776573 | 60776650 | CCTCTGCTTCTAAGGAGTCCTGGTTGGTCTC | 1 | 432 |
| | | | CAAAAGGAAGCTTTCTCTAAAGAACAGTGT | | |
| | | | AGTACAAAGTAAGATGG | | |
| chr9 | 62647482 | 62647559 | CCTCTGCTTCTAAGGAGTCCTGGTTGGTCTC | 1 | 433 |
| | | | CAAAAGGAAGCTTTCTCTAAAGAACAGTGT | | |
| | | | AGTACAAAGTAAGATGG | | |
| chr9 | 66682030 | 66682107 | CCTCTGCTTCTAAGGAGTCCTGGTTGGTCTC | 1 | 434 |
| | | | CAAAAGGAAGCTTTCTCTAAAGAACAGTGT | | |
| | | | AGTACAAAGTAAGATGG | | |
| chr9 | 82264427 | 82264503 | CCACCACTGTGCCTGGCCATTTTCACTATTCT | 3 | 435 |
| | | | TAAAGGAAGCTTTGGTTTACAAAGGTTTGCT | | |
| | | | ACTGTACTTCCAGG | | |
| chr9 | 84042684 | 84042761 | CCATTCTCCCTGTCACTTTCAGGTACACCATT | 1 | 436 |
| | | | CAAACGTAGGTTTGGTCTTTTCTCATAGTCC | | |
| | | | CATATTTCTTGGAGG | | |
| chr9 | 95256012 | 95256089 | CCTCCAAGAAATTCGGGACTATGTGAAAAG | 1 | 437 |
| | | | ACAAAACCTACGTTTAATTGGTGTGTGGTGT | | |
| | | | ACCTGAAAGTGACAAGG | | |
| chr9 | 101816988 | 101817065 | CCTCCAAGAAATATGGGACTATGTGAAAAG | 1 | 438 |
| | | | ACCAAACCTACGTTTGATTGGTGTACCTGAA | | |
| | | | AGTGACCAGAAGAATGG | | |
| chr9 | 135842327 | 135842403 | CCTCCAAGAAATATGGGACTATGTGAAAAG | 3 | 439 |
| | | | CCCAAACCTACGTTTGACTGATGTACCTAAA | | |
| | | | GTGACGGGGAGAATGG | | |
| chr9 | 136910865 | 136910940 | CCCGCACTGTGAGCTTGGCCGAGTGCTGTCT | 4 | 440 |
| | | | GAAAGCATCCTTTCCCTTCACCTGGAGACTG | | |
| | | | GAGCGCCATAGAGG | | |
| chr10 | 13710312 | 13710389 | CCTGTCTCCCCCATTCCATGCAAAATAAAAC | 1 | 441 |
| | | | ACAAACCAAGCTTTGCTTTAAGTGCTCCCTG | | |
| | | | ATGCAGTTCAGCGTGG | | |
| chr10 | 18938129 | 18938206 | CCATTCTTCCCGTCACATTCAGGTACACCAA | 1 | 442 |
| | | | TCAAACGTAGGTTTGGTCTTTTCCCATAGTC | | |
| | | | CCATATTTCTTAGAGG | | |
| chr10 | 22712838 | 22712914 | CCCCCTGCTCAGCTTGGGGAAGAAAAATAC | 2 | 443 |
| | | | AAAAACGATGCTTTTAGGCATTTTAAACAAC | | |
| | | | TTCACTACATTGAGGG | | |
| chr10 | 22712838 | 22712913 | CCCCCTGCTCAGCTTGGGGAAGAAAAATAC | 4 | 444 |
| | | | AAAAACGATGCTTTTAGGCATTTTAAACAAC | | |
| | | | TTCACTACATTGAGG | | |
| chr10 | 40160932 | 40161009 | CCTTTGTGTTGTGTGTATTCAACTCACAGAG | 1 | 445 |
| | | | TGAAACCTTCCTTTATTCAGAGCAGTTTTGA | | |
| | | | AACACTCTTTTTGTGG | | |
| chr10 | 40390136 | 40390213 | CCTTTGTGTTGTGTGTATTCAACTCACAGAG | 1 | 446 |
| | | | TGAAACCTTCCTTTATTCAGAGCAGTTTTGA | | |
| | | | AAAACACTTTTTGTGG | | |
| chr10 | 40409152 | 40409229 | CCTTTGTGTTGTGTGTATTCAACTCACAGAG | 1 | 447 |
| | | | TGAAACCTTCCTTTATTCAGAGCAGTTTTGA | | |
| | | | AAAACTCTTTTTGTGG | | |
| chr10 | 40433940 | 40434017 | CCTTTGTGTTGTGTGTATTCAACTCACAGAG | 1 | 448 |
| | | | TGAAACCTTCCTTTATTCAGAGCAGTTTTGA | | |
| | | | AACACTCTTTTTGTGG | | |
| chr10 | 40588155 | 40588232 | CCTTTGTGTTGTGTGTATTCAACTCACAGAG | 1 | 449 |
| | | | TGAAACCTTCCTTTATTCAGAGCAGTTTTGA | | |
| | | | AATACTCTTTTTGTGG | | |
| chr10 | 41146207 | 41146284 | CCTTTGTGTTGTGTGTATTCAACTCACAGAG | 1 | 450 |
| | | | TGAAACCTTCCTTTATTCAGAGCAGTTTTGA | | |
| | | | AACACTCTTTTTGTGG | | |
| chr10 | 43835183 | 43835260 | CCATTCTCCCTGTCACTTTCAAGTACACCAA | 1 | 451 |
| | | | TCAAACCTAGGTTTGGTCTTTTCACATAGTTC | | |
| | | | CATATTTCTTGGAGG | | |
| chr10 | 54913222 | 54913299 | CCCCTCCCATCACAGGCCCTGAGGTTTAAGA | 1 | 452 |
| | | | GAAAACCATGGTTTTGTGGGCCAGGCCCATG | | |
| | | | ACCCTTCTCCTCTGGG | | |
| chr10 | 54913222 | 54913298 | CCCCTCCCATCACAGGCCCTGAGGTTTAAGA | 3 | 453 |
| | | | GAAAACCATGGTTTTGTGGGCCAGGCCCATG | | |
| | | | ACCCTTCTCCTCTGG | | |
| chr10 | 54913223 | 54913299 | CCCTCCCATCACAGGCCCTGAGGTTTAAGAG | 2 | 454 |
| | | | AAAACCATGGTTTTGTGGGCCAGGCCCATGA | | |
| | | | CCCTTCTCCTCTGGG | | |
| chr10 | 54913223 | 54913298 | CCCTCCCATCACAGGCCCTGAGGTTTAAGAG | 4 | 455 |
| | | | AAAACCATGGTTTTGTGGGCCAGGCCCATGA | | |
| | | | CCCTTCTCCTCTGG | | |
| chr10 | 58035951 | 58036028 | CCATTCTCCCCATCACTTTCAGGTACACCAA | 1 | 456 |
| | | | TCAAACGTAGGTTTCATCTTTTCACATAGTC | | |
| | | | CCACGGTTTTTGGAGG | | |
| chr10 | 58677525 | 58677602 | CCTCCAAGATATATGGGACTATGTGAAAAG | 1 | 457 |
| | | | ACCAAACCTACGTTTGATTGGTGTACCTGAA | | |
| | | | ATTGATGGGGAGAATGG | | |
| chr10 | 84021390 | 84021467 | CCTCCAAGAAATATGGGACTGTGTGAAAAG | 1 | 458 |
| | | | AACAAACCTACGTTTGATTGGTGTACGTGAA | | |
| | | | AGTGATGGGGAGAATGG | | |
| chr10 | 91442692 | 91442769 | CCATTCCTCCCGTCACTTTCAGATACACCAA | 1 | 459 |
| | | | AAAAACGTAGGTTTGGTCTCTTCACATAGTC | | |
| | | | CCACATTTCTTGGAGG | | |
| chr10 | 91446848 | 91446925 | CCTCCAAGAAATGTGGGACTATGTGAAGAG | 1 | 460 |
| | | | ACCAAACCTACGTTTTTTTGGTGTATCTGAA | | |
| | | | AGTGACGGGAGGAATGG | | |
| chr10 | 116928784 | 116928860 | CCTCCAAGGGGAATCTGAGTTCTCTGAAGAC | 3 | 461 |
| | | | AAAAAGCATGGTTTCTTTTCTTCTGTATTTCT | | |
| | | | TATTGTTTCCTAGG | | |
| chr10 | 116937771 | 116937848 | CCATTCTCCCTATCACTTTCCAGTACACCAAT | 1 | 462 |
| | | | CAAACGTAGGTTTGGTCTTTTCACATAGTCC | | |
| | | | CATATTTCTTGGAGG | | |
| chr11 | 31182070 | 31182147 | CCTCCAAGAAATATGGGACTATGTGAAAAG | 1 | 463 |
| | | | ACCAAACCTACGTTTGATTGGTATACTTGAA | | |
| | | | ATTGACAAGGAGAATGG | | |
| chr11 | 34739273 | 34739350 | CCTCCAAGAAATATGGGACTATGTGGAAAG | 1 | 464 |
| | | | ACCAAACCTACGTTTGACTGGTGTACCTGAA | | |
| | | | AGTGATGGGGAGAATGG | | |
| chr11 | 86646529 | 86646606 | CCTCTAAGAAATATGGGACTATGTGAAGAG | 1 | 465 |
| | | | ATGAAACCTACGTTTGATTGGTGTACCTGAA | | |
| | | | AGTGACGAGGAGAATGG | | |
| chr11 | 90469791 | 90469867 | CCCTCGTATACTACATGCTATAGTCAAAGCA | 3 | 466 |
| | | | GTAAACCTTCCTTTCCTTAAGCAGACCACAC | | |
| | | | TCTTTCATGCCTGGG | | |
| chr11 | 90469792 | 90469867 | CCTCGTATACTACATGCTATAGTCAAAGCAG | 4 | 467 |
| | | | TAAACCTTCCTTTCCTTAAGCAGACCACACT | | |
| | | | CTTTCATGCCTGGG | | |
| chr11 | 92429985 | 92430062 | CCATTCTCCCCATCACTTTCAGGTATACTAAT | 1 | 468 |
| | | | CAAAGGTAGGTTTGGTCTTTTCACATAGTCC | | |
| | | | CATATTTCATGGAGG | | |
| chr11 | 102818498 | 102818574 | CCATTCCCCCGTCACTTTCAGGTACACCAAT | 2 | 469 |
| | | | CAAACGTAGGTTTGGTCTTTTCACATAGTCC | | |
| | | | CATATTTCTTGGAGG | | |
| chr11 | 120765065 | 120765142 | CCATTCTCCCCGTCACTTTCAGGTACACCAA | 1 | 470 |
| | | | TCAAACGTAGGTTTTGTCTTTTCTTATAGTCC | | |
| | | | CATATTTCTTGGAGG | | |
| chr11 | 123131901 | 123131978 | CCACTGCACCTGACCAAGATCCTTAATTTTT | 1 | 471 |
| | | | CTAAACCTACGTTTATCATCTATAAAATGAG | | |
| | | | CCATCTTTTCACATGG | | |
| chr11 | 129468520 | 129468597 | CCTCCGAGAAATATGGGACTATGTGAAAAG | 1 | 472 |
| | | | ACCAAACCTACGTTTGATTGTTGTACCTGAA | | |
| | | | AGTGACAGGGAGAATGG | | |
| chr11 | 131272361 | 131272438 | CCATTCTCCCCATCACTTTTAGGTACACCAA | 1 | 473 |
| | | | TCAAACGTAGGTTTGGTCCTTTTGCATAGAC | | |
| | | | CCATATTTCTTGGAGG | | |
| chr11 | 132761415 | 132761492 | CCATTTTCCCCGTCAGTTTCATATACACCTAT | 1 | 474 |
| | | | CAAACGTAGGTTTACTGTTTTCACATAGTCC | | |
| | | | CTTATTTCTTGGAGG | | |
| chr12 | 22367416 | 22367493 | CCTCCAAGAAATATGGGACTATGTGAAAAG | 1 | 475 |
| | | | ACCAAACCTACCTTTGATTGGTGTACCTGAA | | |
| | | | AGTGACGGGCAGGATGG | | |
| chr12 | 33146384 | 33146461 | CCATTCTTCTCGTCATTTTCAAGTACACCAAT | 1 | 476 |
| | | | CAAACGTAGGTTTGGTCTTTTCGCATAGTCC | | |
| | | | CATATTTCTTGGAGG | | |
| chr12 | 33198476 | 33198553 | CCATTCTTCTCGTCACTTTCAAGTACACCAAT | 1 | 477 |
| | | | CAAACGTAGGTTTGGTCTTTTCACATAGTCC | | |
| | | | CATATTTCTTGGAGG | | |
| chr12 | 46038332 | 46038409 | CCTCCAAGAAATATAGGACTATGTGAAAAG | 1 | 478 |
| | | | ACCAAACCTACGTTTGATTGGTGTACTTGAA | | |
| | | | AGTGACAGGGAGAATGG | | |
| chr12 | 60236126 | 60236203 | CCTCCAAGAAATGTGGAACTATGTGAAAAG | 1 | 479 |
| | | | ACCAAACCTACGTTTGATTGGTGTACCTGAA | | |
| | | | AGTGACAGGGAGAATGG | | |
| chr12 | 62098359 | 62098434 | CCCTGACACTGATAAACGGATATGAAGAGA | 4 | 480 |
| | | | AAAAAGCTAGGTTTTCGCTGGAATTCCTAAG | | |
| | | | CTTGGGCTGCAGTGG | | |
| chr12 | 62112591 | 62112668 | CCCTTCTCCCAGTCACTTTTAGGTACACCAA | 1 | 481 |
| | | | TGAAACGTAGGTTTGGTCTTTTCACACAGTC | | |
| | | | CCATATTTCTTGGAGG | | |
| chr12 | 62112592 | 62112668 | CCTTCTCCCAGTCACTTTTAGGTACACCAAT | 2 | 482 |
| | | | GAAACGTAGGTTTGGTCTTTTCACACAGTCC | | |
| | | | CATATTTCTTGGAGG | | |
| chr12 | 62418577 | 62418652 | CCACTCCCTCTCCCCCAAAAAGTAAAGGTAG | 4 | 483 |
| | | | AAAACCAAGGTTTACAGGCAACAAATAGCA | | |
| | | | CAATGAATGGAATGG | | |
| chr12 | 71732311 | 71732388 | CCAAACCCGCATCGCACACCCTGTGAGGGG | 1 | 484 |
| | | | GACAAAGGAACCTTTCCGTTCCAACATCAAG | | |
| | | | GTTGTTTTGACCCAAGG | | |
| chr12 | 78047816 | 78047893 | CCATTCTTTCTGTCACTTTCAGGTATACCAGT | 1 | 485 |
| | | | CAAACCTAGGTTTGGTCTTTTCACATAGTCC | | |
| | | | CATATTTCTTGGAGG | | |
| chr12 | 81480016 | 81480093 | CCATTCTCCCCATCACTTTCAGGTACACCAA | 1 | 486 |
| | | | TCAAACGTAGGTTTGGTCTTTTCACATAGTC | | |
| | | | CCATATTTCTTGGAGG | | |
| chr12 | 96840231 | 96840307 | CCACACGGTAGAGGATAAACTAGGTGGATT | 3 | 487 |
| | | | CTCAAAGCAACCTTTGAAATAATCTATGCAG | | |
| | | | TTTTTCTGGGTACTGG | | |
| chr12 | 99187165 | 99187242 | CCACCAAGAAACATGGGACTATGTGAAAAG | 1 | 488 |
| | | | ACCAAACCTACGTTTGGTTGGTGTACCTGGA | | |
| | | | AGTGACGGGGAGAGTGG | | |
| chr12 | 107860841 | 107860918 | CCTCCAAGAAATATGGGACCATGTGAAAAG | 1 | 489 |
| | | | ACCAAACCTACGTTTGATTGGTGTACCTGAA | | |
| | | | AGTGACAGGGAGAATGG | | |
| chr12 | 110882809 | 110882885 | CCTGTAAAAAGGTCACATGGTCAGGTGTGCC | 2 | 490 |
| | | | TAAACGATCCTTTTATTTATTTATTTATTTAT | | |
| | | | TTTTAAGAAACAGG | | |
| chr12 | 119063321 | 119063397 | CCAGCCCCAAAATGTCAGGGGCTTAGAACA | 2 | 491 |
| | | | ACAAAGGTTCCTTTTCATGTTTATACTACAT | | |
| | | | GTTTGTCATGGGCTGG | | |
| chr13 | 35320704 | 35320781 | CCGTTTTCCCCATCACTTTCAGGTACACCAG | 1 | 492 |
| | | | TCAAACGTAGGTTTGGTCTTTTCACATGGTC | | |
| | | | CCACATTTCTTGGAGG | | |
| chr13 | 53133477 | 53133554 | CCTGGAATAGCTTTCCTGACTGTCTGACTTC | 1 | 493 |
| | | | AAAAACCTTGGTTTGACCACTTCGTCTATAT | | |
| | | | CATGAGGAAGGACTGG | | |
| chr13 | 53184880 | 53184956 | CCCTACTCTGAACCTACCTTGATAAAGCCTA | 3 | 494 |
| | | | GAAAACCAAGCTTTGACAAGATTTGACAAG | | |
| | | | AGATGGAATTTGGAGG | | |
| chr13 | 53184881 | 53184956 | CCTACTCTGAACCTACCTTGATAAAGCCTAG | 4 | 495 |
| | | | AAAACCAAGCTTTGACAAGATTTGACAAGA | | |
| | | | GATGGAATTTGGAGG | | |
| chr13 | 57896962 | 57897038 | CCCTTATAAAACTGAAAACTTTAACCTTTTTT | 2 | 496 |
| | | | AAAGCATGCTTTTGAATAAATTCTTTTATTA | | |
| | | | CAAAAAAGACCAGG | | |
| chr13 | 62610100 | 62610177 | CCATTCTCCCTGTCACTTTCAGGTACACCAA | 1 | 497 |
| | | | TCAAACGTAGGTTTGGTCTTTTCACGTAGTC | | |
| | | | CCATATTTCTTGGAGG | | |
| chr13 | 77004382 | 77004458 | CCCTTTATTATCCAAGTGGTTTCCTGCTCTTC | 2 | 498 |
| | | | AAACCTTCCTTTCAAAATTTTGTCTCCTACTT | | |
| | | | AAAACAAGTTAGG | | |
| chr13 | 81646075 | 81646151 | CCTTCTGTTGAGACCTACTGCTAAGAAAACA | 3 | 499 |
| | | | AAAAAGGTTCCTTTCAAATATTATTGTGAAT | | |
| | | | CAATAATGTACCTGG | | |
| chr13 | 83755854 | 83755931 | CCTCCAAGAAATATGGGACTATGTGAAAAG | 1 | 500 |
| | | | ACCAAACCTACGTTTCATTGATGGACCTGAA | | |
| | | | AGTGATGGGGAGAATGG | | |
| chr13 | 89719199 | 89719275 | CCATTCTCCCTTCACTTTCAGTTACACCAATC | 2 | 501 |
| | | | AAACGTAGGTTTGGTCTTTTCACATAGTCCC | | |
| | | | ATATTTCTTGGAGG | | |
| chr13 | 102010574 | 102010650 | CCTAGGGAAGTGATCATAGCTGAGTTTCTGG | 3 | 502 |
| | | | AAAAACCTAGGTTTTAAAGTTGAGGAGACTT | | |
| | | | AAGTCCAAAACCTGG | | |
| chr13_KI270841v1_alt | 124240 | 124316 | CCATTCTCCCTTCACTTTCAGTTACACCAATC | 2 | 503 |
| | | | AAACGTAGGTTTGGTCTTTTCACATAGTCCC | | |
| | | | ATATTTCTTGGAGG | | |
| chr14 | 25980646 | 25980723 | CCTCCAAGAAATATGGGACTATGTGAAAAG | 1 | 504 |
| | | | ACTAAACCTACGTTTGATTGGTGTACCTGAA | | |
| | | | AGTGACAGGGAGAATGG | | |
| chr14 | 35842786 | 35842863 | CCATTCTCCCTGTCACTTTCAGGTATGCCAGT | 1 | 505 |
| | | | CAAACGTAGGTTTGGTCTTTTCACATAGTCC | | |
| | | | CATATTCCTTGGAGG | | |
| chr14 | 42646400 | 42646477 | CCTCCAAGAAATATGGGACTATGTAAAAAG | 1 | 506 |
| | | | ACGAAACCTACGTTTGATTGGTGTACTTAAA | | |
| | | | AGTGACGAGGAGAATGG | | |
| chr14 | 49063242 | 49063319 | CCTCCAAGAAATATGGGACTATGTGAAAAG | 1 | 507 |
| | | | ACCAAACCTACGTTTGATTTGTGTACCTGAA | | |
| | | | AGTGATGGGGAGAATGG | | |
| chr14 | 49130379 | 49130456 | CCATTCTCCCCGTCACTTTCAGGCACACCAA | 1 | 508 |
| | | | TCAAACGTAGGTTTAGTCTTTTCACATAGTC | | |
| | | | CCATATTTCTTAGAGG | | |
| chr14 | 51352342 | 51352418 | CCTTAATGCATTCATATTTCATATTTTAAATA | 2 | 509 |
| | | | AAACCATGGTTTCCCACAGAGTGACTTCTAC | | |
| | | | TCTAAGAAATGGGG | | |
| chr14 | 51352342 | 51352417 | CCTTAATGCATTCATATTTCATATTTTAAATA | 4 | 510 |
| | | | AAACCATGGTTTCCCACAGAGTGACTTCTAC | | |
| | | | TCTAAGAAATGGG | | |
| chr14 | 60835842 | 60835919 | CCGTTCTTTCCGTCACTTTCAGGTACACCAGT | 1 | 511 |
| | | | CAAACGTAGGTTTGGTCTTTTCACATAGTCC | | |
| | | | CATATTTCTTGGAGG | | |
| chr14 | 66529072 | 66529148 | CCATTCTCCCCATCACTTTCATGTACACCAAT | 3 | 512 |
| | | | CAAACGTAGGTTTGGTCTTTGTTAACATAGT | | |
| | | | CCCATATTTCTTGG | | |
| chr14 | 79210873 | 79210949 | CCCTATAAAGCTTAGAGAAACACAGGGCTCT | 3 | 513 |
| | | | TTAAACGATCCTTTTTCTCTTTTCTGTTTTAA | | |
| | | | ATTTCATCACTTGG | | |
| chr14 | 79210874 | 79210949 | CCTATAAAGCTTAGAGAAACACAGGGCTCTT | 4 | 514 |
| | | | TAAACGATCCTTTTTCTCTTTTCTGTTTTAAA | | |
| | | | TTTCATCACTTGG | | |
| chr14 | 85371541 | 85371618 | CCATTCTCCCCATCACTTTCAGGTACACTAA | 1 | 515 |
| | | | TCAAAGGTAGGTTTGGTCTTTTCACATGGTC | | |
| | | | CTATATTTCTTGGAGG | | |
| chr14 | 92918713 | 92918790 | CCCCATAGCACGATCACATGGGACATTCAGG | 1 | 516 |
| | | | GGAAAGCAACCTTTTCCAGGAAGGAAAACC | | |
| | | | CAATGCTGGGACCCAGG | | |
| chr14 | 92918714 | 92918790 | CCCATAGCACGATCACATGGGACATTCAGG | 2 | 517 |
| | | | GGAAAGCAACCTTTTCCAGGAAGGAAAACC | | |
| | | | CAATGCTGGGACCCAGG | | |
| chr14 | 103386821 | 103386897 | CCCTTTCAGCGCTCACAGGCTATGGTTTTAT | 2 | 518 |
| | | | AAAAGGAACCTTTGATTTTGTTCATGTGAAA | | |
| | | | CTACAAAATGCCAGG | | |
| chr14_KI270847v1_alt | 33275 | 33352 | CCCCATAGCACGATCACATGGGACATTCAGG | 1 | 519 |
| | | | GGAAAGCAACCTTTTCCAGGAAGGAAAACC | | |
| | | | CAATGCTGGGACCCAGG | | |
| chr14_KI270847v1_alt | 33276 | 33352 | CCCATAGCACGATCACATGGGACATTCAGG | 2 | 520 |
| | | | GGAAAGCAACCTTTTCCAGGAAGGAAAACC | | |
| | | | CAATGCTGGGACCCAGG | | |
| chr15 | 20630566 | 20630643 | CCTCCAAGAAATATTGGAGTATGTGATAAGA | 1 | 521 |
| | | | CCAAACCTTCGTTTGACTGGTGTACCTGAAA | | |
| | | | GTGATGGGGAGAATGG | | |
| chr15 | 21675103 | 21675180 | CCATTCTCCCCGTCACTTTCAGGTACACCAA | 1 | 522 |
| | | | TCAAACGTAGGTTTGGTCTTTTCACATAGTC | | |
| | | | CCATATTTCTTGGAGG | | |
| chr15 | 22117571 | 22117648 | CCATTCTCCCCGTCACTTTCAGGTACACCAA | 1 | 523 |
| | | | TCAAACGTAGGTTTGGTCTTTTCACATAGTC | | |
| | | | CCATATTTCTTGGAGG | | |
| chr15 | 22369744 | 22369821 | CCATTCTCCCCATCACTTTCAGGTACACCAG | 1 | 524 |
| | | | TCAAACGAAGGTTTGGTCTTATCACATACTC | | |
| | | | CAATATTTCTTGGAGG | | |
| chr15 | 42302832 | 42302909 | CCTCCAAGATATATGGGACTATGTGAAAAG | 1 | 525 |
| | | | GCCAAACCTACCTTTGATTGATACACCTGAA | | |
| | | | AATGACAGGGAGAATGG | | |
| chr15 | 49967601 | 49967678 | CCTCCAAGAAATATGCGACTATGTGAAAAG | 1 | 526 |
| | | | ACCAAACCTACGTTTCATTGGTGTACCTGAA | | |
| | | | AGTGATGGGGAGAATGG | | |
| chr15 | 83964501 | 83964577 | CCTCCAAGAAATATGGGACTATGTGGAAAG | 3 | 527 |
| | | | ACCAAACCTACGTTTGTTTGGTGTACCTGAA | | |
| | | | AGTGAGGGGAGAATGG | | |
| chr15 | 87261388 | 87261465 | CCATTCTCCTCATCACTTTCAAGTACACCAA | 1 | 528 |
| | | | TCAAACGTAGGTTTGGTCTTTTCACATAGTC | | |
| | | | TTATATTTCTTGGAGG | | |
| chr15_KI270727v1_random | 409348 | 409425 | CCATTCTCCCCGTCACTTTCAGGTACACCAA | 1 | 529 |
| | | | TCAAACGTAGGTTTGGTCTTTTCACATAGTC | | |
| | | | CCATATTTCTTGGAGG | | |
| chr15_KI270851v1_alt | 14235 | 14312 | CCATTCTCCCCATCACTTTCAGGTACACCAG | 1 | 530 |
| | | | TCAAACGAAGGTTTGGTCTTATCACATACTC | | |
| | | | CAATATTTCTTGGAGG | | |
| chr15_KI270852v1_alt | 440099 | 440176 | CCATTCTCCCCGTCACTTTCAGGTACACCAA | 1 | 531 |
| | | | TCAAACGTAGGTTTGGTCTTTTCACATAGTC | | |
| | | | CCATATTTCTTGGAGG | | |
| chr16 | 22123671 | 22123748 | CCAGCAGAAGAATCTGGGGCACAGTCTGTG | 1 | 532 |
| | | | AAAAAAGGTACCTTTCTTAAGCAGGGTTCTT | | |
| | | | ATCCTTCATGGGTCTGG | | |
| chr16 | 25557623 | 25557700 | CCTCCAAGAAATATGGGACTATGTGAAAAG | 1 | 533 |
| | | | ACCAAACCTACGTTTGATTGTTGTACCTGAA | | |
| | | | AGTGAGGGGGAGAATGG | | |
| chr16 | 36427179 | 36427255 | CCTTGTGTTGTGTGTATTCAACTCACAGAGT | 2 | 534 |
| | | | TAAACGATCCTTTACACAGAGCAGATTTGAA | | |
| | | | ACACTGTTTTTCTGG | | |
| chr16 | 36476450 | 36476526 | CCTTGTGTTGTGTGTATTCAACTCACAGAGT | 2 | 535 |
| | | | TAAACGATCCTTTACACAGAGCAGATTTGAA | | |
| | | | ACACTGTTTTTCTGG | | |
| chr16 | 36512469 | 36512545 | CCTTGTGTTGTGTGTATTCAACTCACAGAGT | 2 | 536 |
| | | | TAAACGATCCTTTACACAGAGCAGATTTGAA | | |
| | | | ACACTGTTTTTCTGG | | |
| chr16 | 36520964 | 36521040 | CCTTGTGTTGTGTGTATTCAACTCACAGAGT | 2 | 537 |
| | | | TAAACGATCCTTTACACACAGCAGATTTGAA | | |
| | | | ACACTGTTTTTCTGG | | |
| chr16 | 36524704 | 36524780 | CCTTGTGTTGTGTGTATTCAACTCACAGAGT | 2 | 538 |
| | | | TAAACGATCCTTTACACAGAGCAGATTTGAA | | |
| | | | ACACTGTTTTTCTGG | | |
| chr16 | 36566812 | 36566888 | CCTTGTGTTGTGTGTATTCAACTCACAGAGT | 2 | 539 |
| | | | TAAACGATCCTTTACACAGAGCAGATTTGAA | | |
| | | | ACACTGTTTTTCTGG | | |
| chr16 | 36573603 | 36573679 | CCTTGTGTTGTGTGTATTCAACTCACAGAGT | 2 | 540 |
| | | | TAAACGATCCTTTACACAGAGCAGATTTGAA | | |
| | | | ACACTGTTTTTCTGG | | |
| chr16 | 36667694 | 36667770 | CCTTGTGTTGTGTGTATTCAACTCACAGAGT | 2 | 541 |
| | | | TAAACGATCCTTTACACAGAGCAGATTTGAA | | |
| | | | ACACTGTTTTTCTGG | | |
| chr16 | 36677320 | 36677396 | CCTTGTGTTGTGTGTATTCAACTCACAGAGT | 2 | 542 |
| | | | TAAACGATCCTTTACACAGAGCAGATTTGAA | | |
| | | | ACACTGTTTTTCTGG | | |
| chr16 | 36683096 | 36683172 | CCTTGTGTTGTGTGTATTCAACTCACAGAGT | 2 | 543 |
| | | | TAAACGATCCTTTACACAGAGCAGATTTGAA | | |
| | | | ACACTGTTTTTCTGG | | |
| chr16 | 36691251 | 36691327 | CCTTGTGTTGTGTGTATTCAACTCACAGAGT | 2 | 544 |
| | | | TAAACGATCCTTTACACAGAGCAGATTTGAA | | |
| | | | ACACTGTTTTTCTGG | | |
| chr16 | 36710951 | 36711027 | CCTTGTGTTGTGTGTATTCAACTCACAGAGT | 2 | 545 |
| | | | TAAACGATCCTTTACACAGAGCAGATTTGAA | | |
| | | | ACACTGTTTTTCTGG | | |
| chr16 | 36750364 | 36750440 | CCTTGTGTTGTGTGTATTCAACTCACAGAGT | 2 | 546 |
| | | | TAAACGATCCTTTACACAGAGCAGATTTGAA | | |
| | | | ACACTGTTTTTCTGG | | |
| chr16 | 36791455 | 36791531 | CCTTGTGTTGTGTGTATTCAACTCACAGAGT | 2 | 547 |
| | | | TAAACGATCCTTTACACACAGCAGATTTGAA | | |
| | | | ACACTGTTTTTCTGG | | |
| chr16 | 36856683 | 36856759 | CCTTGTGTTGTGTGTATTCAACTCACAGAGT | 2 | 548 |
| | | | TAAACGATCCTTTACACAGAGCAGATTTGAA | | |
| | | | ACACTGTTTTTCTGG | | |
| chr16 | 36926655 | 36926731 | CCTTGTGTTGTGTGTATTCAACTCACAGAGT | 2 | 549 |
| | | | TAAACGATCCTTTACACAGAGCAGATTTGAA | | |
| | | | ACACTGTTTTTCTGG | | |
| chr16 | 36931752 | 36931828 | CCTTGTGTTGTGTGTATTCAACTCACAGAGT | 2 | 550 |
| | | | TAAACGATCCTTTACACAGAGCAGATTTGAA | | |
| | | | ACACTGTTTTTCTGG | | |
| chr16 | 36948058 | 36948134 | CCTTGTGTTGTGTGTATTCAACTCACCGAGTT | 2 | 551 |
| | | | AAACGATCCTTTACACAGAGCAGATTTGAAA | | |
| | | | CACTGTTTTTCTGG | | |
| chr16 | 36974541 | 36974617 | CCTTGTGTTGTGTGTATTCAACTCACAGAGT | 2 | 552 |
| | | | TAAACGATCCTTTACACAGAGCAGATTTGAA | | |
| | | | ACACTGTTTTTCTGG | | |
| chr16 | 36981331 | 36981407 | CCTTGTGTTGTGTGTATTCAACTCACAGAGT | 2 | 553 |
| | | | TAAACGATCCTTTACACAGAGCAGATTTGAA | | |
| | | | ACACTGTTTTTCTGG | | |
| chr16 | 36990839 | 36990915 | CCTTGTGTTGTGTGTATTCAACTCACAGAGT | 2 | 554 |
| | | | TAAACGATCCTTTACACAGAGCAGATTTGAA | | |
| | | | ACACTGTTTTTCTGG | | |
| chr16 | 37021075 | 37021151 | CCTTGTGTTGTGTGTATTCAACTCACAGAGT | 2 | 555 |
| | | | TAAACGATCCTTTACACAGAGCAGATTTGAA | | |
| | | | ACACTGTTTTTCTGG | | |
| chr16 | 37042812 | 37042888 | CCTTGTGTTGTGTGTATTCAACTCACAGAGT | 2 | 556 |
| | | | TAAACGATCCTTTACACAGAGCAGATTTGAA | | |
| | | | ACACTGTTTTTCTGG | | |
| chr16 | 37085971 | 37086047 | CCTTGTGTTGTGTGTATTCAACTCACAGAGT | 2 | 557 |
| | | | TAAACGATCCTTTACACAGAGCAGATTTGAA | | |
| | | | ACACTGTTTTTCTGG | | |
| chr16 | 37129462 | 37129538 | CCTTGTGTTGTGTGTATTCAACTCACAGAGT | 2 | 558 |
| | | | TAAACGATCCTTTACACAGAGCAGATTTGAA | | |
| | | | ACACTGTTTTTCTGG | | |
| chr16 | 37146110 | 37146186 | CCTTGTGTTGTGTGTATTCAACTCACAGAGT | 2 | 559 |
| | | | TAAACGATCCTTTACACACAGCAGATTTGAA | | |
| | | | ACACTGTTTTTCTGG | | |
| chr16 | 37157309 | 37157385 | CCTTGTGTTGTGTGTATTCAACTCACAGAGT | 2 | 560 |
| | | | TAAACGATCCTTTACACAGAGCAGATTTGAA | | |
| | | | ACACTGTTTTTCTGG | | |
| chr16 | 37183118 | 37183194 | CCTTGTGTTGTGTGTATTCAACTCACAGAGT | 2 | 561 |
| | | | TAAACGATCCTTTACACAGAGCAGATTTGAA | | |
| | | | ACACTGTTTTTCTGG | | |
| chr16 | 37190924 | 37191000 | CCTTGTGTTGTGTGTATTCAACTCACAGAGT | 2 | 562 |
| | | | TAAACGATCCTTTACACAGAGCAGATTTGAA | | |
| | | | ACACTGTTTTTCTGG | | |
| chr16 | 37221808 | 37221884 | CCTTGTGTTGTGTGTATTCAACTCACAGAGT | 2 | 563 |
| | | | TAAACGATCCTTTACACAGAGCAGATTTGAA | | |
| | | | ACACTGTTTTTCTGG | | |
| chr16 | 37259501 | 37259577 | CCTTGTGTTGTGTGTATTCAACTCACAGAGT | 2 | 564 |
| | | | TAAACGATCCTTTACACAGAGCAGATTTGAA | | |
| | | | ACACTGTTTTTCTGG | | |
| chr16 | 37272409 | 37272485 | CCTTGTGTTGTGTGTATTCAACTCACAGAGT | 2 | 565 |
| | | | TAAACGATCCTTTACACAGAGCAGATTTGAA | | |
| | | | ACACTGTTTTTCTGG | | |
| chr16 | 37281923 | 37281999 | CCTTGTGTTGTGTGTATTCAACTCACAGAGT | 2 | 566 |
| | | | TAAACGATCCTTTACACAGAGCAGATTTGTA | | |
| | | | ACACTGTTTTTCTGG | | |
| chr16 | 37346472 | 37346548 | CCTTGTGTTGTGTGTATTCAACTCACAGAGT | 2 | 567 |
| | | | TAAACGATCCTTTACACAGAGCAGATTTGAA | | |
| | | | ACACTGTTTTTCTGG | | |
| chr16 | 37357000 | 37357076 | CCTTGTGTTGTGTGTATTCAACTCACAGAGT | 2 | 568 |
| | | | TAAACGATCCTTTACACAGAGCAGATTTGAA | | |
| | | | ACACTGTTTTTCTGG | | |
| chr16 | 37373301 | 37373377 | CCTTGTGTTGTGTGTATTCAACTCACAGAGT | 2 | 569 |
| | | | TAAACGATCCTTTACACAGAGCAGATTTGAA | | |
| | | | ACACTGTTTTTCTGG | | |
| chr16 | 37419498 | 37419574 | CCTTGTGTTGTGTGTATTCAACTCACAGAGT | 2 | 570 |
| | | | TAAACGATCCTTTACACAGAGCAGATTTGAA | | |
| | | | ACACTGTTTTTCTGG | | |
| chr16 | 37430714 | 37430790 | CCTTGTGTTGTGTGTATTCAACTCACAGAGT | 2 | 571 |
| | | | TAAACGATCCTTTACACAGAGCAGATTTGAA | | |
| | | | ACACTGTTTTTCTGG | | |
| chr16 | 37455845 | 37455921 | CCTTGTGTTGTGTGTATTCAACTCACAGAGT | 2 | 572 |
| | | | TAAACGATCCTTTACACAGAGCAGATTTGAA | | |
| | | | ACACTGTTTTTCTGG | | |
| chr16 | 37458558 | 37458634 | CCTTGTGTTGTGTGTATTCAACTCACAGAGT | 2 | 573 |
| | | | TAAACGATCCTTTACACAGAGCAGATTTGAA | | |
| | | | ACACTGTTTTTCTGG | | |
| chr16 | 37486127 | 37486203 | CCTTGTGTTGTGTGTATTCAACTCACAGAGT | 2 | 574 |
| | | | TAAACGATCCTTTACACAGAGCAGATTTGAA | | |
| | | | ACACTGTTTTTCTGG | | |
| chr16 | 37525183 | 37525259 | CCTTGTGTTGTGTGTATTCAACTCACAGAGT | 2 | 575 |
| | | | TAAACGATCCTTTACACAGAGCAGATTTGAA | | |
| | | | ACACTGTTTTTGTGG | | |
| chr16 | 37536735 | 37536811 | CCTTGTGTTGTGTGTATTCAACTCACAGAGT | 2 | 576 |
| | | | TAAACGATCCTTTACACAGAGCAGATTTGAA | | |
| | | | ACACTGTTTTTCTGG | | |
| chr16 | 37554730 | 37554806 | CCTTGTGTTGTGTGTATTCAACTCACAGAGT | 2 | 577 |
| | | | TAAACGATCCTTTACACAGAGCAGATTTGAA | | |
| | | | ACACTGTTTTTCTGG | | |
| chr16 | 37575784 | 37575860 | CCTTGTGTTGTGTGTATTCAACTCACAGAGT | 2 | 578 |
| | | | TAAACGATCCTTTACACAGAGCAGATTTGAA | | |
| | | | ACACTGTTTTTCTGG | | |
| chr16 | 37577483 | 37577559 | CCTTGTGTTGTGTGTATTCAACTCACAGAGT | 2 | 579 |
| | | | TAAACGATCCTTTACACAGAGCAGATTTGAA | | |
| | | | ACACTGTTTTTCTGG | | |
| chr16 | 37583598 | 37583674 | CCTTGTGTTGTGTGTATTCAACTCACAGAGT | 2 | 580 |
| | | | TAAACGATCCTTTACACAGAGCAGATTTGAA | | |
| | | | ACACTGTTTTTCTGG | | |
| chr16 | 37696368 | 37696444 | CCTTGTGTTGTGTGTATTCAACTCACAGAGT | 2 | 581 |
| | | | TAAACGATCCTTTCCACAGAGCAGATTTGAA | | |
| | | | ACACTGTTTTTCTGG | | |
| chr16 | 37704524 | 37704600 | CCTTGTGTTGTGTGTATTCAACTCACAGAGT | 2 | 582 |
| | | | TAAACGATCCTTTACACAGAGCAGATTTGAA | | |
| | | | ACACTGTTTTTCTGG | | |
| chr16 | 37706223 | 37706299 | CCTTGTGTTGTGTGTATTCAACTCACAGAGT | 2 | 583 |
| | | | TAAACGATCCTTTACACAGAGCAGATTTGAA | | |
| | | | ACACTGTTTTTCTGG | | |
| chr16 | 37708941 | 37709017 | CCTTGTGTTGTGTGTATTCAACTCACAGAGT | 2 | 584 |
| | | | TAAACGATCCTTTACACAGAGCAGATTTGAA | | |
| | | | ACACTGTTTTTCTGG | | |
| chr16 | 37763622 | 37763698 | CCTTGTGTTGTGTGTATTCAACTCACAGAGT | 2 | 585 |
| | | | TAAACGATCCTTTACACAGAGCAGATTTGAA | | |
| | | | ACACTGTTTTTCTGG | | |
| chr16 | 37772115 | 37772191 | CCTTGTGTTGTGTGTATTCAACTCACAGAGT | 2 | 586 |
| | | | TAAACGATCCTTTACACAGAGCAGATTTGAA | | |
| | | | ACACTGTTTTTCTGG | | |
| chr16 | 37791815 | 37791891 | CCTTGTGTTGTGTGTATTCAACTCACAGAGT | 2 | 587 |
| | | | TAAACGATCCTTTACACAGAGCAGATTTGAA | | |
| | | | ACACTGTTTTTCTGG | | |
| chr16 | 37796229 | 37796305 | CCTTGTGTTGTGTGTATTCAACTCACAGAGT | 2 | 588 |
| | | | TAAACGATCCTTTACACAGAGCAGATTTGAA | | |
| | | | ACACTGTTTTTCTGG | | |
| chr16 | 37797928 | 37798004 | CCTTGTGTTGTGTGTATTCAACTCACAGAGT | 2 | 589 |
| | | | TAAACGATCCTTTACACAGAGCAGATTTGAA | | |
| | | | ACACTGTTTTTCTGG | | |
| chr16 | 37843453 | 37843529 | CCTTGTGTTGTGTGTATTCAACTCACAGAGT | 2 | 590 |
| | | | TAAACGATCCTTTACACAGAGCAGATTTGAA | | |
| | | | ACACTGTTTTTCTGG | | |
| chr16 | 37848548 | 37848624 | CCTTGTGTTGTGTGTATTCAACTCACAGAGT | 2 | 591 |
| | | | TAAACGATCCTTTACACAGAGCAGATTTGAA | | |
| | | | ACACTGTTTTTCTGG | | |
| chr16 | 37864846 | 37864922 | CCTTGTGTTGTGTGTATTCAACTCACCGAGTT | 2 | 592 |
| | | | AAACGATCCTTTACACAGAGCAGATTTGAAA | | |
| | | | CACTGTTTTTCTGG | | |
| chr16 | 37902550 | 37902626 | CCTTGTGTTGTGTGTATTCAACTCACAGAGT | 2 | 593 |
| | | | TAAACGATCCTTTACACAGAGCAGATTTGAA | | |
| | | | ACACTGTTTTTCTGG | | |
| chr16 | 37907307 | 37907383 | CCTTGTGTTGTGTGTATTCAACTCACAGAGT | 2 | 594 |
| | | | TAAACGATCCTTTACACAGAGCAGATTTGAA | | |
| | | | ACACTGTTTTTCTGG | | |
| chr16 | 37928033 | 37928109 | CCTTGTGTTGTGTGTATTCAACTCACAGAGT | 2 | 595 |
| | | | TAAACGATCCTTTACACAGAGCAGATTTGAA | | |
| | | | ACACTGTTTTTCTGG | | |
| chr16 | 37959262 | 37959338 | CCTTGTGTTGTGTGTATTCAACTCACAGAGT | 2 | 596 |
| | | | TAAACGATCCTTTACACAGAGCAGATTTGAA | | |
| | | | ACACTGTTTTTCTGG | | |
| chr16 | 37964355 | 37964431 | CCTTGTGTTGTGTGTATTCAACTCACAGAGT | 2 | 597 |
| | | | TAAACGATCCTTTACACAGAGCAGATTTGAA | | |
| | | | ACACTGTTTTTCTGG | | |
| chr16 | 37974881 | 37974957 | CCTTGTGTTGTGTGTATTCAACTCACAGAGT | 2 | 598 |
| | | | TAAACGATCCTTTACACAGAGCAGATTTGAA | | |
| | | | AAACTGTTTTTCTGG | | |
| chr16 | 37987789 | 37987865 | CCTTGTGTTGTGTGTATTCAACTCACAGAGT | 2 | 599 |
| | | | AAAACGATCCTTTACACAGAGCAGATTTGAA | | |
| | | | ACACTGTTTTTCTGG | | |
| chr16 | 37994586 | 37994662 | CCTTGTGTTGTGTGTATTCAACTCACAGAGT | 2 | 600 |
| | | | TAAACGATCCTTTACACAGAGCAGATTTGAA | | |
| | | | ACACTGTTTTTGTGG | | |
| chr16 | 38006479 | 38006555 | CCTTGTGTTGTGTGTATTCAACTCACAGAGT | 2 | 601 |
| | | | TAAACGATCCTTTACACAGAGCAGATTTGAA | | |
| | | | ACACTGTTTTTCTGG | | |
| chr16 | 38011567 | 38011643 | CCTTGTGTTGTGTGTATTTAACTCACAGAGTT | 2 | 602 |
| | | | AAACGATCCTTTACACAGAGCAGATTTGAAA | | |
| | | | CACTGTTTTTCTGG | | |
| chr16 | 38040096 | 38040172 | CCTTGTGTTGTGTGTATTCAACTCACAGAGT | 2 | 603 |
| | | | TAAACGATCCTTTACACAGAGCAGATTTGAA | | |
| | | | ACACTGTTTTTCTGG | | |
| chr16 | 38041456 | 38041532 | CCTTGTGTTGTGTGTATTCAACTCACAGAGT | 2 | 604 |
| | | | TAAACGATCCTTTACACAGAGCAGATTTGAA | | |
| | | | ACACTGTTTTTCTGG | | |
| chr16 | 38062179 | 38062255 | CCTTGTGTTGTGTGTATTCAACTCACAGAGT | 2 | 605 |
| | | | TAAACGATCCTTTACACAGAGCAGATTTGAA | | |
| | | | ACACTGTTTTTCTGG | | |
| chr16 | 38102937 | 38103013 | CCTTGTGTTGTGTGTATTCAACTCACAGAGT | 2 | 606 |
| | | | TAAACGATCCTTTACACAGAGCAGATTTGAA | | |
| | | | ACACTGTTTTTCTGG | | |
| chr16 | 38128412 | 38128488 | CCTTGTGTTGTGTGTATTCAACTCACAGAGT | 2 | 607 |
| | | | TAAACGATCCTTTACACAGAGCAGATTTGAA | | |
| | | | ACACTGTTTTTCTGG | | |
| chr16 | 38131809 | 38131885 | CCTTGTGTTGTGTGTATTCAACTCACAGAGT | 2 | 608 |
| | | | TAAACGATCCTTTACACAGAGCAGATTTGAA | | |
| | | | ACACTGTTTTTCTGG | | |
| chr16 | 38144723 | 38144799 | CCTTGTGTTGTGTGTATTCAACTCACAGAGT | 2 | 609 |
| | | | TAAACGATCCTTTACACAGAGCAGATTTGAA | | |
| | | | ACACTGTTTTTCTGG | | |
| chr16 | 38168845 | 38168921 | CCTTGTGTTGTGTGTATTCAACTCACAGAGT | 2 | 610 |
| | | | TAAACGATCCTTTACACAGAGCAGATTTGAA | | |
| | | | ACACTGTTTTTCTGG | | |
| chr16 | 38209287 | 38209363 | CCTTGTGTTGTGTGTATTCAACTCACAGAGT | 2 | 611 |
| | | | TAAACGATCCTTTACACAGAGCAGATTTGAA | | |
| | | | ACACTGTTTTTCTGG | | |
| chr16 | 38210986 | 38211062 | CCTTGTGTTGTGTGTATTCAACTCACAGAGT | 2 | 612 |
| | | | TAAACGATCCTTTACACAGAGCAGATTTGAA | | |
| | | | ACACTGTTTTTCTGG | | |
| chr16 | 38229667 | 38229743 | CCTTGTGTTGTGTGTATTCAACTCACAGAGT | 2 | 613 |
| | | | TAAACGATCCTTTACACAGAGCAGATTTGAA | | |
| | | | ACACTGTTTTTCTGG | | |
| chr16 | 47424037 | 47424114 | CCATTCTCCCTATCACTTTCAGGTACACCAA | 1 | 614 |
| | | | TCAAACGTAGGTTTGGTCTTTTCACATAGTC | | |
| | | | CCATATTTCTTGGAGG | | |
| chr16 | 60730549 | 60730625 | CCTCGTCACTGCCAGATTTTGTGGCTACCAG | 2 | 615 |
| | | | CAAAGGATCGTTTTAAGCTGCAACTCAGGAA | | |
| | | | ATTGAGAAAATATGG | | |
| chr16 | 72545014 | 72545091 | CCTCCAAGAAATATGGGACTATGTGAAAAA | 1 | 616 |
| | | | ACCAAACCTACGTTTGATTGGTGTACCTGAA | | |
| | | | AGTGACAGGGAGAATGG | | |
| chr16 | 81945503 | 81945579 | CCCTGTGTTCTTTTATACTAAAACAAGCCAG | 2 | 617 |
| | | | CAAACCAACCTTTGAGATGTGTTGCCTTAAA | | |
| | | | CATTACTGAATGGGG | | |
| chr16 | 81945503 | 81945578 | CCCTGTGTTCTTTTATACTAAAACAAGCCAG | 4 | 618 |
| | | | CAAACCAACCTTTGAGATGTGTTGCCTTAAA | | |
| | | | CATTACTGAATGGG | | |
| chr17 | 16474024 | 16474100 | CCGAGAAACGGCTTTAGCAACAAATAAATA | 3 | 619 |
| | | | TCAAAAGGATGCTTTCTCTTCAGAATAATCT | | |
| | | | AAAGTAAGTTGGGAGG | | |
| chr17 | 34438512 | 34438589 | CCATGTTACTCCGGATAAGGACAGCAAAGG | 1 | 620 |
| | | | AGGAAAGGAACCTTTTCTGGGCCACCAGAA | | |
| | | | GGATGAGCTTGGGCTTGG | | |
| chr17 | 43690782 | 43690859 | CCCAGGGATATGCTGGCCACGGGGAGGAGC | 1 | 621 |
| | | | CGGAAACCAACCTTTGTGTCACTGTGTAGTG | | |
| | | | ACAAGTGCCTTTGGAGG | | |
| chr17 | 43690783 | 43690859 | CCAGGGATATGCTGGCCACGGGGAGGAGCC | 2 | 622 |
| | | | GGAAACCAACCTTTGTGTCACTGTGTAGTGA | | |
| | | | CAAGTGCCTTTGGAGG | | |
| chr17 | 69156298 | 69156375 | CCTTAGGGACCCATAATGGCCACAACCAGG | 1 | 623 |
| | | | AGAAAAGCAAGCTTTGATGCTTAAACACTAC | | |
| | | | TTACAGACATGTACAGG | | |
| chr17 | 74595228 | 74595305 | CCTGCCTCTGTTCCTCCTTCCTGATGGTGGCG | 1 | 624 |
| | | | GAAAGGATGCTTTTGCCAGATCAACAGTCAC | | |
| | | | ACACAACACACCAGG | | |
| chr17 | 83191644 | 83191721 | CCTGACTCCAGCCCTCCTTGACAAGGTCTCC | 1 | 625 |
| | | | GTAAAGCATGCTTTCTCTTAGGGACCCTCAG | | |
| | | | AGGGAGGCTTGGTGGG | | |
| chr17 | 83191644 | 83191720 | CCTGACTCCAGCCCTCCTTGACAAGGTCTCC | 3 | 626 |
| | | | GTAAAGCATGCTTTCTCTTAGGGACCCTCAG | | |
| | | | AGGGAGGCTTGGTGG | | |
| chr18 | 35135224 | 35135300 | CCTTATTTGGAATGTGACAAGACCCATTTGT | 3 | 627 |
| | | | TTAAACCTTGGTTTTTATGCAGAAAGAAAAG | | |
| | | | GAAGGCTGCAGTGGG | | |
| chr18 | 38918861 | 38918938 | CCATTCTCCCTGTCACTTTCAGGTACACTAAT | 1 | 628 |
| | | | CAAACGTAGGTTTGCTGTTTTTACATAGGCT | | |
| | | | CATATTTCTTGGAGG | | |
| chr18 | 45476589 | 45476666 | CCATTCTCCCCATCACTTTCAGGTACACCAG | 1 | 629 |
| | | | TCAAACGTAGGTTTGGTCTTTTCACATAGTC | | |
| | | | CCATATTTCTTGGAGG | | |
| chr18 | 48640821 | 48640896 | CCTGTTTGTTATTTTAGCTAATGTCAAAAAG | 4 | 630 |
| | | | AAAACCTTGCTTTTTCTGAACCCTTTCAGAG | | |
| | | | GCAGAAAGTGGGGG | | |
| chr18 | 71096732 | 71096808 | CCATTTTCCCCACCACTTTCACGTACAGCAA | 3 | 631 |
| | | | TCAAACGTAGGTTTGGTCTTTTCACTAGTCC | | |
| | | | CATATTTCTTGGAGG | | |
| chr19 | 24957844 | 24957920 | CCTTGTAGTGTGTGTATTCAACTCACAGAGT | 2 | 632 |
| | | | TAAACGATCCTTTACACAGAGCAGACTTGAA | | |
| | | | ACACTCTTGTTGTGG | | |
| chr19 | 25015316 | 25015392 | CCTTGTAGTGTGTGTATTCAACTCACAGAGT | 2 | 633 |
| | | | TAAACGATCCTTTACACAGAGCATACTTGAA | | |
| | | | ACACTCTTTTTGTGG | | |
| chr19 | 25074119 | 25074195 | CCTTGTGTTGTGTGTATTCAACTCACAGAGT | 2 | 634 |
| | | | TAAACGATCCTTTACACAGAGCAGACTTGAA | | |
| | | | ACACTCTTTTTGTGG | | |
| chr19 | 25827861 | 25827937 | CCTTGTGTTGTGTTTATTCAACTCACAGAGTT | 2 | 635 |
| | | | AAACGATCCTTTACACAGAGCAGACTTGAA | | |
| | | | ATACTCTTTTTGTGG | | |
| chr19 | 26054056 | 26054132 | CCTTGTAGTGTGTGTATTCAACTCACAGAGT | 2 | 636 |
| | | | TAAACGATCCTTTACACAGAGCATACTTGAA | | |
| | | | ACACTCTTTTTGTGG | | |
| chr19 | 26211777 | 26211853 | CCTTGTATTGTGAGTATTCAACTCACAGAGT | 2 | 637 |
| | | | TAAACGATCCTTTACACAGAGCAGACTTGAA | | |
| | | | ACACTCTTTTTGTGG | | |
| chr19 | 26483670 | 26483746 | CCTTGTGTTGTGTGTCTTCAACTCACAGAGTT | 2 | 638 |
| | | | AAACGATGCTTTACACAGAGTAGACTTGAA | | |
| | | | ACACTCTTTTTCTGG | | |
| chr19 | 26636516 | 26636592 | CCTTGTGTTGTGTGTATTCAACTCACAGAGT | 2 | 639 |
| | | | TAAACGATCCTTTACACAGAGCAGACTTGTA | | |
| | | | ACACTCTTTTTGTGG | | |
| chr19 | 26637877 | 26637953 | CCTTGTGTTGTGTGTATTCAACTCACAGAGT | 2 | 640 |
| | | | TAAACGATCCTTTACACAGAGCAGACGTGA | | |
| | | | AACACTCTTTTTGTGG | | |
| chr19 | 26750223 | 26750299 | CCTTGTGTTGTGTGTATTCAACTCACAGAGT | 2 | 641 |
| | | | TAAACGATCCTTTACACAGAGCAGACTTGAA | | |
| | | | ACACTCTTTTTGTGG | | |
| chr19 | 26841158 | 26841234 | CCTTGTGTTGTGTGTATTCAACTCACAGAGT | 2 | 642 |
| | | | TAAACGATCCTTTACACAGAGGAGACTTGTA | | |
| | | | ACACTCTTTTTGTGG | | |
| chr19 | 28517220 | 28517297 | CCAGGAAAAAATTTAAACTTTCTTAACTTGA | 1 | 643 |
| | | | TAAAAGGTAGCTTTCAAAACCTACAATAAAT | | |
| | | | AACATACTTAGAGTGG | | |
| chr19 | 34566821 | 34566898 | CCATTCTCCTCGTCACTTTCAGGTACACCAA | 1 | 644 |
| | | | ACAAACGTAGGTTTGGTCTTTTTACGTAGTC | | |
| | | | CCATATTTCTTGGAGG | | |
| chr19 | 52261770 | 52261847 | CCCTCTTGAAGTTAGGGAAGTAGCATTTAAG | 1 | 645 |
| | | | GGAAACGTAGCTTTACTATTAAGAATTTCAA | | |
| | | | ACAGCACTTGTCAGGG | | |
| chr19 | 52261770 | 52261846 | CCCTCTTGAAGTTAGGGAAGTAGCATTTAAG | 3 | 646 |
| | | | GGAAACGTAGCTTTACTATTAAGAATTTCAA | | |
| | | | ACAGCACTTGTCAGG | | |
| chr19 | 52261771 | 52261847 | CCTCTTGAAGTTAGGGAAGTAGCATTTAAGG | 2 | 647 |
| | | | GAAACGTAGCTTTACTATTAAGAATTTCAAA | | |
| | | | CAGCACTTGTCAGGG | | |
| chr19 | 52261771 | 52261846 | CCTCTTGAAGTTAGGGAAGTAGCATTTAAGG | 4 | 648 |
| | | | GAAACGTAGCTTTACTATTAAGAATTTCAAA | | |
| | | | CAGCACTTGTCAGG | | |
| chr20 | 11151392 | 11151469 | CCATTCTCCCCGTCACTTTCAGGTACACCAA | 1 | 649 |
| | | | TCAAACGTAGGTTTGGTCTTTTCACATATTCC | | |
| | | | CATATTTCTTGGAGG | | |
| chr20 | 14027067 | 14027143 | CCATTCTCCCTTCACTTTCAGGTACACCAATC | 2 | 650 |
| | | | AAACGTAGGTTTGGTCTTTTCACATAGTCCC | | |
| | | | ATATTTTTTGGAGG | | |
| chr20 | 50615399 | 50615476 | CCTATAGTCTCAGTTACTTGGGAGGCTGAGG | 1 | 651 |
| | | | TAAAAGGATCGTTTGAGCCCAGGAGGTGGA | | |
| | | | GGTTGCAGTGAGCCGGG | | |
| chr20 | 50615399 | 50615475 | CCTATAGTCTCAGTTACTTGGGAGGCTGAGG | 3 | 652 |
| | | | TAAAAGGATCGTTTGAGCCCAGGAGGTGGA | | |
| | | | GGTTGCAGTGAGCCGG | | |
| chr20 | 60909414 | 60909490 | CCTTTCCCAACTCTGCTATTGCCCCCACATCC | 3 | 653 |
| | | | TAAAGGAACCTTTCTTTTTTTATATATTTTAT | | |
| | | | TTTAAGTTCCAGG | | |
| chr21 | 16226086 | 16226163 | CCTCCAAGAAATATGGAACTATGTGAAAAG | 1 | 654 |
| | | | ACCAAACCTACGTTTGATTGACGTACCTGAA | | |
| | | | AGTGACAGGGAGAATGG | | |
| chr21 | 17835234 | 17835309 | CCTCTTCTGAAAGCATTGATAATCAACATTT | 4 | 655 |
| | | | TAAACGTAGCTTTTCCCCATATTGCTAGGAA | | |
| | | | GGCTCATTCCCGGG | | |
| chr21 | 19425636 | 19425713 | CCTCCAAGAAATATGGGACTATGTGAAAAG | 1 | 656 |
| | | | GCCAAACCTACGTTTGATTGCTGTACCCGAG | | |
| | | | AGTGACGGGGAGAATGG | | |
| chr21 | 32220958 | 32221035 | CCTCCAAGAAATATGGGACTATGTGAAAAG | 1 | 657 |
| | | | ACCAAACCTACGTTTGATTGGTGTACCTGAA | | |
| | | | AGTGATGGGGAGAATGG | | |
| chr21 | 34335877 | 34335953 | CCCGGGGCCTGGGTGCCCAGTGCCAGTGGTC | 3 | 658 |
| | | | AGAAAGGTTGCTTTGGTGTTTTTCATTGTTA | | |
| | | | GTGAGACAGAGATGG | | |
| chr21 | 34335878 | 34335953 | CCGGGGCCTGGGTGCCCAGTGCCAGTGGTCA | 4 | 659 |
| | | | GAAAGGTTGCTTTGGTGTTTTTCATTGTTAGT | | |
| | | | GAGACAGAGATGG | | |
| chr21 | 36315276 | 36315353 | CCATTCTCCCCATCATTTTCAGGTACACCAA | 1 | 660 |
| | | | TCAAACGTAGGTTTGATCTTTTCACATAGCC | | |
| | | | CCATATTTCTTGGAGG | | |
| chr21 | 41547952 | 41548028 | CCACCAGCACTTCTGTTAGAAGTTGCAGCAG | 3 | 661 |
| | | | AGAAAGGATCCTTTAGGCACATCTCCCAGAT | | |
| | | | CCTTGCGAAGAGGGG | | |
| chr22 | 18973194 | 18973271 | CCTGTGCCAGGGTCCTTCCACTGGGACTGGC | 1 | 662 |
| | | | AGAAACGTAGGTTTGCATGGAGTGAGAAGC | | |
| | | | AGGGGAGAGGTTGAGGG | | |
| chr22 | 18973194 | 18973270 | CCTGTGCCAGGGTCCTTCCACTGGGACTGGC | 3 | 663 |
| | | | AGAAACGTAGGTTTGCATGGAGTGAGAAGC | | |
| | | | AGGGGAGAGGTTGAGG | | |
| chr22 | 20265462 | 20265539 | CCCTCAGCCTCTCCCCTGCTTCTCACTCCATG | 1 | 664 |
| | | | CAAACCTACGTTTCTGCCAGTCCCAGCAGAA | | |
| | | | GGACCCTGGCACGGG | | |
| chr22 | 20265462 | 20265538 | CCCTCAGCCTCTCCCCTGCTTCTCACTCCATG | 3 | 665 |
| | | | CAAACCTACGTTTCTGCCAGTCCCAGCAGAA | | |
| | | | GGACCCTGGCACGG | | |
| chr22 | 20265463 | 20265539 | CCTCAGCCTCTCCCCTGCTTCTCACTCCATGC | 2 | 666 |
| | | | AAACCTACGTTTCTGCCAGTCCCAGCAGAAG | | |
| | | | GACCCTGGCACGGG | | |
| chr22 | 20265463 | 20265538 | CCTCAGCCTCTCCCCTGCTTCTCACTCCATGC | 4 | 667 |
| | | | AAACCTACGTTTCTGCCAGTCCCAGCAGAAG | | |
| | | | GACCCTGGCACGG | | |
| chrX | 27300998 | 27301075 | CCTCCAAGAAATATGGGGCTATGTGAAAAG | 1 | 668 |
| | | | ACCAAACCTACCTTTGATTGGTGTATCTGAA | | |
| | | | AGTGACGGGGAGAATGG | | |
| chrX | 28456666 | 28456743 | CCTCCAAGAAATATGGGACTATGTGAAAAG | 1 | 669 |
| | | | ACCAAACCTACGTTTGATTTGTGTACCTGAA | | |
| | | | AGTGATGGGGAGAATGG | | |
| chrX | 35634985 | 35635062 | CCATTCTCCCCGTCACTTTCAGGTACACCAA | 1 | 670 |
| | | | TCAAACGTAGGTTTGGTCTTTTCTCATTGTCC | | |
| | | | CATATTTCTTGGAGG | | |
| chrX | 39460148 | 39460223 | CCCATCAAGAGCGGTTGTGCATGGCAACAGT | 4 | 671 |
| | | | AAAAGGATGGTTTGTTACACTAGTACAAAA | | |
| | | | AGAGGTGGCCAGAGG | | |
| chrX | 43926403 | 43926480 | CCATTCTCTCTGTCACTTTCAGGTACACCAAT | 1 | 672 |
| | | | CAAACGTAGGTTTGGTCTTTTCACATAGTCC | | |
| | | | CATATTTCTTGGAGG | | |
| chrX | 44254600 | 44254677 | CCTCCAAGAAATACGGGACTATGTGAAAAG | 1 | 673 |
| | | | ACCAAACGTACGTTTGATTGGTGTACCTGAA | | |
| | | | AGTGATAGGGAGAATGG | | |
| chrX | 46088602 | 46088679 | CCTCCAAGAAATATGGGACTATGTGAAAAG | 1 | 674 |
| | | | ACCAAACCTACGTTTGATTGGTGTACCTGAA | | |
| | | | AGTGACTGGGAGAATGG | | |
| chrX | 50222874 | 50222951 | CCATTCTCCCTGTCACTTTCAGGTACACGAA | 1 | 675 |
| | | | TCAAACGTAGGTTTCATCTTTTCACATAGTC | | |
| | | | CCATATTTCTTAGAGG | | |
| chrX | 57416835 | 57416911 | CCATTCTCTCTGTCACTTTCTGGTACACCAAT | 3 | 676 |
| | | | CAAACGTAGGTTTGGTCTTTTCACATAGTTT | | |
| | | | CACATATTTCTTGG | | |
| chrX | 57856466 | 57856543 | CCTCCAAGAAATATGGGACTATGTGAAAAG | 1 | 677 |
| | | | ACCAAACCTACGTTTGATTGGTGTACCTGAA | | |
| | | | AGTGACAAGGAAAATGG | | |
| chrX | 62702479 | 62702556 | CCTGAAAAACATTGTTTCCAACCTGGTAAAT | 1 | 678 |
| | | | CAAAAGGAAGGTTTAACTTTGTTAGATAAGT | | |
| | | | CCACATATCACCAAGG | | |
| chrX | 63067129 | 63067206 | CCTCCAAGAAATGTGGGACTATGGGAAAAG | 1 | 679 |
| | | | ACCAAACCTACCTTTGTTTGGTGTACCTGAA | | |
| | | | AGTGACGGGGAGAAAGG | | |
| chrX | 64936250 | 64936327 | CCTCCAAGAAATATGGGACTATGTGAAAAG | 1 | 680 |
| | | | ACCAAACCTACGTTTCATTGGTGTACCTGAA | | |
| | | | AGTGATGGGTAGAATGG | | |
| chrX | 66720099 | 66720176 | CCTACAAGAAATATGGGACTATGGGAAAAG | 1 | 681 |
| | | | ACCAAACCTACGTTTGATTGGTACACTGGAA | | |
| | | | AGTGACAGGGATAATGG | | |
| chrX | 68529086 | 68529163 | CCATTCTCCCTGTCACTTTCTGGTACACCAAT | 1 | 682 |
| | | | CAAAGGTAGGTTTGGTCTTTTCACATAGTCC | | |
| | | | CATATTTCTTGGAGG | | |
| chrX | 73893994 | 73894071 | CCTCCAAGAAATATGGGACTATGTGAAAAG | 1 | 683 |
| | | | ACCAAACCTACGTTTGATTGGTGTACCTGAA | | |
| | | | AGTGATGGGGAGAATGG | | |
| chrX | 75723201 | 75723278 | CCATTCTCTTTGTCACTTTCAGGTATACCAAT | 1 | 684 |
| | | | CAAACGTTGGTTTGGTCTTTTTGCATAGTCCC | | |
| | | | ATATTTTGTGGAGG | | |
| chrX | 75815659 | 75815736 | CCTCCAAGAAATATGAGACTATGTGAAAAG | 1 | 685 |
| | | | ACCAAACCTACGTTTGATTAGTGTACCTGAA | | |
| | | | AATGATGGGGAGAATGG | | |
| chrX | 80967103 | 80967180 | CCATTCTTTCTGTCACTTTCAGGTACACCAAT | 1 | 686 |
| | | | CAAACGTAGGTTTGGTCTTTTCACATAGTCC | | |
| | | | CATATTTCTTGGAGG | | |
| chrX | 89936425 | 89936502 | CCATTCTCCCTGTCACTTTCAGGTACACCAA | 1 | 687 |
| | | | TCAAACGTAGGTTTGTTCTTTTCACATAGTCC | | |
| | | | CATATTTCTTGGAGG | | |
| chrX | 91038768 | 91038845 | CCATTATCCCCATCACTTTCAGGTACACCAA | 1 | 688 |
| | | | TCAAACGTAGGTTTGGTTTTTTCACATAGTTC | | |
| | | | AATATTTCTTTGAGG | | |
| chrX | 91471271 | 91471348 | CCTCCAAGAAATATGGGACTATCTGAAAAG | 1 | 689 |
| | | | ATCAAACCTACGTTTGATTGGTGTACCTGAA | | |
| | | | AGTGACAGGGAGAATGG | | |
| chrX | 96428180 | 96428257 | CCTTTCTCCCCATCACTTTCAGGTACACCAAT | 1 | 690 |
| | | | CAAACGTAGGTTTGGTCTTTTCATATAGTCC | | |
| | | | CATATTTCTTGGAGG | | |
| chrX | 100268291 | 100268368 | CCTCCAAGAAATATGGGACTATGTGCAAAG | 1 | 691 |
| | | | ATCAAACCTACGTTTGATTGCTGTACCTGAA | | |
| | | | AGTGATGGGGAGAATGG | | |
| chrX | 105811046 | 105811123 | CCATTCTCCCCATCACTTTCAGGTACACCAG | 1 | 692 |
| | | | TCAAACGTAGGTTTGGTCTTTTCACATAATC | | |
| | | | CCATATTTCTTGGAGG | | |
| chrX | 115673065 | 115673141 | CCTCCAAGAAGTATGGGACCATGGAAAAGA | 2 | 693 |
| | | | TCAAACCTACGTTTGACTGGTGTACCTGAAA | | |
| | | | GTGACTGGGAGAATGG | | |
| chrX | 117269846 | 117269923 | CCTCCAAGAAATATGGGACTATGTGAAAAG | 1 | 694 |
| | | | ACCAAACCTACGTTTGATTGGAGTACTTGAA | | |
| | | | AATGACAGGGATAATGG | | |
| chrX | 139191369 | 139191445 | CCTTTAAAGACATGCTCTTTGTGCCAGAAAT | 3 | 695 |
| | | | TCAAAGGTTGCTTTTATGTCCAGTGGGGTGG | | |
| | | | AGGGAGGAAGCTCGG | | |
| chrX | 147988614 | 147988691 | CCATTCTCCCCGTCACTTTCAGGGACCTCAA | 1 | 696 |
| | | | TCAAACGTAGGTTTTGTCTTTTCACATAGTCC | | |
| | | | CATATTTCTTGGAGG | | |
| chrX | 155321041 | 155321118 | CCTCCAAGAAATATAGGACTATGTGAAAAG | 1 | 697 |
| | | | ACCAAACCTACGTTTGACTGGTGTACCTGAA | | |
| | | | AGTGACAGGGAGAATGG | | |
| chrY | 15109391 | 15109468 | CCATTCTCCCCATCACTTTCAGGTACACCAA | 1 | 698 |
| | | | TCAAAGGTAGGTTTGGTCTTTTCACATAGTC | | |
| | | | CGATATTTCCTGCAGG | | |

Chromosomal sites were identified by searching for CCX₍₃₀₋₃₁₎-AAASSWWSSTTT-X₍₃₀₋₃₁₎-GG (SEQ ID NO: 699) where W is T or A and S is G or C. Pattern 1 is CCX₍₃₁₎-AAASSWWSSTTT-X₍₃₁₎-GG (SEQ ID NO: 699), 2 is CCX₍₃₀₎-AAASSWWSSTTT-X₍₃₁₎-GG (SEQ ID NO: 699), 3 is CCX₍₃₁₎-AAASSWWSSTTT-X₍₃₀₎-GG (SEQ ID NO: 699), and 4 is CCX₍₃₀₎-AAASSWWSSTTT-X₍₃₀₎-GG (SEQ ID NO: 699). Only the + strand is shown and the start and end corresponds to the first and last base pair in the chromosome (GRCh38) or alternate assembly when applicable.

### DNA Sequencing

Transfections of 293T cells were performed as above in sextuplet and incubated for 72 hours. Cells were harvested and replicates were combined. Episomal DNA was extracted using a modified HIRT extraction involving alkaline lysis and spin column purification essentially as described (Quan et al., Circular polymerase extension cloning of complex gene libraries and pathways. PloS one 4, e6441 (2009); and Hillson (2010), vol. 2015, pp. CPEC protocol; the entire contents of each of which are hereby incorporated by reference). Briefly, after harvesting, HEK293T cells were washed in 500 μL of ice cold PBS, resuspended in 250 μL GTE Buffer (50 mM glucose, 25 mM Tris-HCl, 10 mM EDTA and pH 8.0), incubated at room temperature for 5 minutes, and lysed on ice for 5 minutes with 200 μL lysis buffer (200 mM NaOH, 1% sodium dodecyl sulfate). Lysis was neutralized with 150 μL of a potassium acetate solution (5 M acetate, 3 M potassium, pH 6.7). Cell debris were pelleted by centrifugation at 21,130 g for 15 minutes and lysate was applied to Econospin Spin columns (Epoch Life Science, Missouri City, TX). Columns were washed twice with 750 μL wash buffer (Omega Bio-tek, Norcross, GA) and eluted in 45 μL TE buffer, pH 8.0.

Isolated episomal DNA was digested for 2 hours at 37° C. with RecBCD (10 U) following the manufacturer's instructions and purified into 10 μL EB with a MinElute Reaction Cleanup Kit (Qiagen, Valencia, CA). Mach1-T1 chemically competent cells were transformed with 5 μL of episomal extractions and plated on agarose plates selecting for carbenicillin resistance (containing 50 μg/mL carbenicillin). Individual colonies were sequenced with primer pCALNL-for-1 to determine the rate of recombination. Sequencing reads revealed either the ‘left’ intact non-recombined recCas9 site, the expected recombined product, rare instances of ‘left’ non-recombined site with small indels, or one instance of a large deletion product.

Analysis of recCas9 Catalyzed Genomic Deletions

HEK293T cells were seeded at a density of 6×10⁵ cells per well in 24 well collagen-treated plates and grown overnight (Corning, Corning, NY). Transfections reactions were brought to a final volume of 100 μL in Opti-MEM (ThermoFisher Scientific, Waltham, MA). For each transfection, 90 ng of each guide RNA expression vector, 20 ng of pma×GFP (Lonza, Allendale, NJ) and 320 ng of recCas9 expression vector were combined with 2 μL Lipofectamine 2000 in Opti-MEM (ThermoFisher Scientific, Waltham, MA) and added to individual wells. After 48 hours, cells were harvested and sorted for the GFP transfection control on a BD FACS AriaIIIu cell sorter. Cells were sorted on purity mode using a 100 m nozzle and background fluorescence was determined by comparison with untransfected cells. Sorted cells were collected on ice in PBS, pelleted and washed twice with cold PBS. Genomic DNA was harvested using the E.Z.N.A. Tissue DNA Kit (Omega Bio-Tek, Norcross, GA) and eluted in 100 μL EB. Genomic DNA was quantified using the Quant-iT PicoGreen dsDNA kit (ThermoFisher Scientific, Waltham, MA) measured on a Tecan Infinite M1000 Pro fluorescence plate reader.

Nested PCR was carried out using Q5 Hot-Start Polymerase 2× Master Mix supplemented with 3% DMSO and diluted with HyClone water, molecular biology grade (GE Life Sciences, Logan, UT). Primary PCRs were carried out at 25 uL scale with 20 ng of genomic DNA as template using the primer pair FAM19A2-F1 and FAM19A2-R1 (Table 5). The primary PCR conditions were as follows: 98° C. for 1 minute, 35 cycles of (98° C. for 10 seconds, 59° C. for 30 seconds, 72° C. for 30 seconds), 72° C. for 1 minute. A 1:50 dilution of the primary PCR served as template for the secondary PCR, using primers FAM19A2-F2 and FAM19A2-R2. The secondary PCR conditions were as follows: 98° C. for 1 minute, 30 cycles of (98° C. for 10 seconds, 59° C. for 20 seconds, 72° C. for 20 seconds), 72° C. for 1 minute. DNA was analyzed by electrophoresis on a 1% agarose gel in TAE alongside a 1 Kb Plus DNA ladder (ThermoFisher Scientific, Waltham, MA). Material to be Sanger sequenced was purified on a Qiagen Minelute column (Valencia, CA) using the manufacturer's protocol. Template DNA from 3 biological replicates was used for three independent genomic nested PCRs.

The limit of detection was calculated given that one complete set of human chromosomes weighs approximately 3.6 pg (3.3-10⁹ bp×1·10⁻²¹ g/bp). Therefore, a PCR reaction seeded with 20 ng of genomic DNA template contains approximately 5500 sets of chromosomes.

For quantification of genomic deletion, nested PCR was carried out using the above conditions in triplicate for each of the 3 biological replicates. A two-fold dilution series of genomic DNA was used as template, beginning with the undiluted stock (for sample 1, 47.17 ng/uL; for sample 2, 75.96 ng/uL; and for sample 3, 22.83 ng/uL) to reduce potential sources of pipetting error. The lowest DNA concentration for which a deletion PCR product could be observed was assumed to contain a single deletion product per total genomic DNA.

The number of genomes present in a given amount of template DNA can be inferred, and thus an estimate a minimum deletion efficiency for recCas9 at the FAM19A2 locus can be determined. For example, take the case of a two-fold dilution series, beginning with 20 ng genomic DNA template. After nested PCR, only the well seeded with 20 ng yielded the correct PCR product. At 3.6 pg per genome, that PCR contained approximately 5500 genomes, and since at least one recombined genome must have been present, the minimum deletion efficiency is 1 in 5500 or 0.018%.

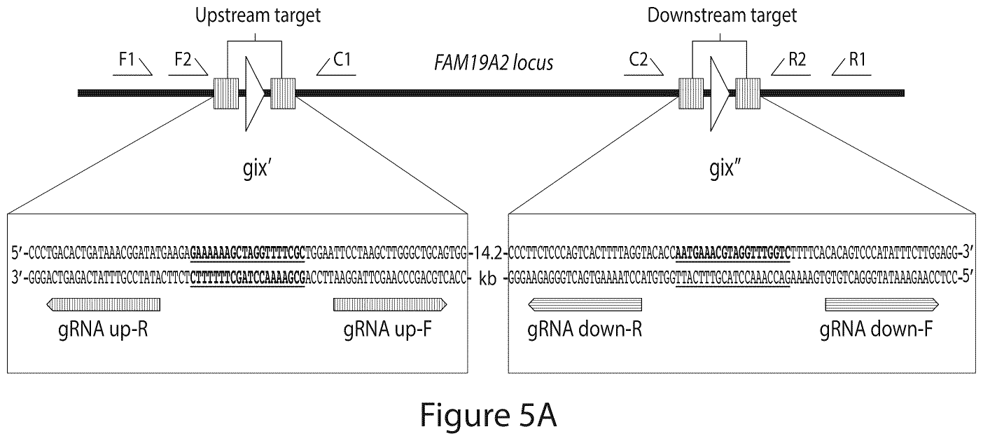

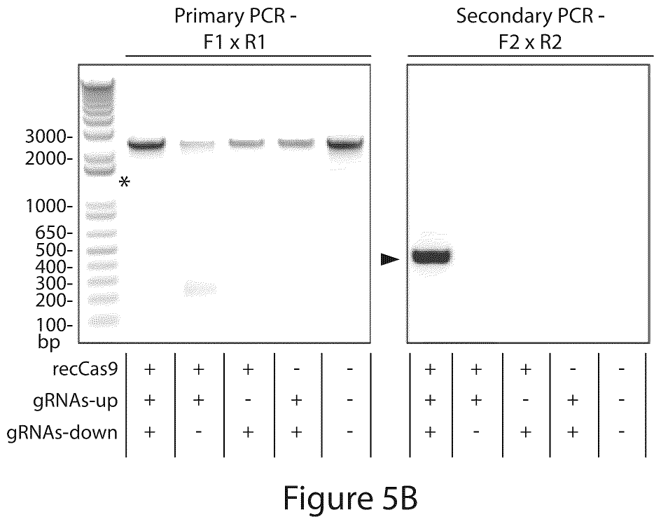

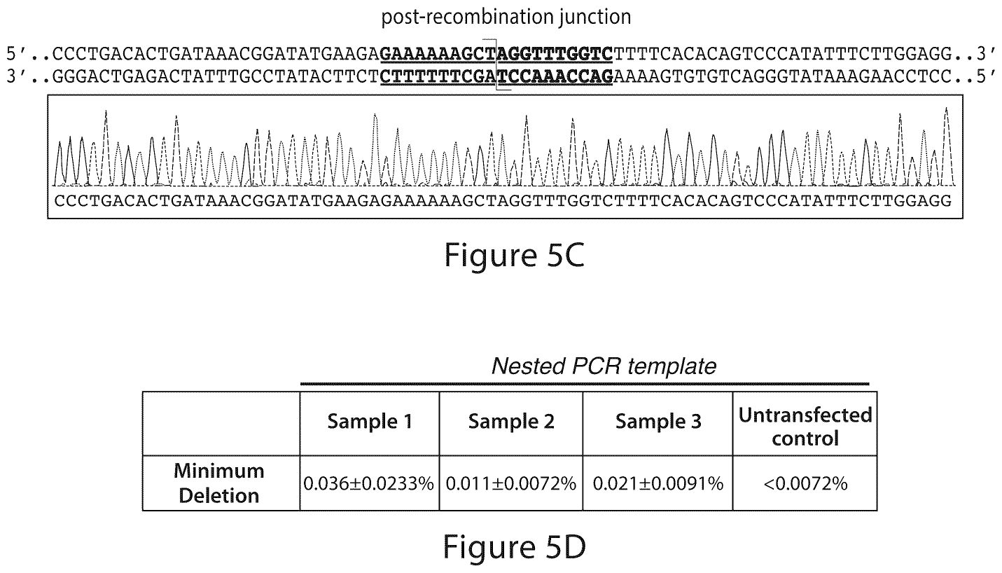

The levels of genomic DNA were quantified using a limiting dilution of genomic template because using quantitative PCR (qPCR) to determine the absolute level of genome editing would require a set of PCR conditions that unambiguously and specifically amplify only from post-recombined genomic DNA. As shown in FIG. 5B, primary PCR using genomic DNA as a template results in a roughly 2.5 kb off-target band as the dominant species; a second round of PCR using nested primers is required to reveal guide RNA- and recCas9-dependent genome editing.

### Results

Fusing Gin Recombinase to dCas9

It has been recently demonstrated that the N-terminus of dCas9 may be fused to the FokI nuclease catalytic domain, resulting in a dimeric dCas9-FokI fusion that cleaved DNA sites flanked by two guide RNA-specified sequences (see, e.g., Guilinger et al., Fusion of catalytically inactive Cas9 to FokI nuclease improves the specificity of genome modification. Nature biotechnology, (2014); Tsai et al., Dimeric CRISPR RNA-guided FokI nucleases for highly specific genome editing. Nature biotechnology, (2014); the entire contents of each of which are hereby incorporated by reference). The same fusion orientation was used to connect dCas9 to Ginβ, a highly active catalytic domain of dimeric Gin invertase previously evolved by Barbas and co-workers (Gaj et al., A comprehensive approach to zinc-finger recombinase customization enables genomic targeting in human cells. Nucleic acids research 41, 3937-3946 (2013), the entire contents of which is hereby incorporated by reference). Ginβ promiscuously recombines several 20-bp core “gix” sequences related to the native core sequence CTGTAAACCGAGGTTTTGGA (SEQ ID NO: 700) (Gaj et al., A comprehensive approach to zinc-finger recombinase customization enables genomic targeting in human cells. Nucleic acids research 41, 3937-3946 (2013); Klippel et al., The DNA Invertase Gin of Phage Mu—Formation of a Covalent Complex with DNA Via a Phosphoserine at Amino-Acid Position-9. Embo Journal 7, 1229-1237 (1988); Mertens et al., Site-specific recombination in bacteriophage Mu: characterization of binding sites for the DNA invertase Gin. The EMBO journal 7, 1219-1227 (1988); Plasterk et al., DNA inversions in the chromosome of Escherichia coli and in bacteriophage Mu: relationship to other site-specific recombination systems. Proceedings of the National Academy of Sciences of the United States of America 80, 5355-5358 (1983); the entire contents of each of which are hereby incorporated by reference). The guide RNAs localize a recCas9 dimer to a gix site flanked by two guide-RNA specified sequences, enabling the Ginβ domain to catalyze DNA recombination in a guide RNA-programmed manner (FIG. 1D).

To assay the resulting dCas9-Ginβ (recCas9) fusions, a reporter plasmid containing two recCas9 target sites flanking a poly-A terminator that blocks EGFP transcription was constructed (FIGS. 1A-1C). Each recCas9 target site consisted of a gix core pseudo-site flanked by sites matching a guide RNA protospacer sequence. Recombinase-mediated deletion removed the terminator, restoring transcription of EGFP. HEK293T cells were cotransfected with this reporter plasmid, a plasmid transcribing a guide RNA(s), and a plasmid producing candidate dCas9-Ginβ fusion proteins, and the fraction of cells exhibiting EGFP fluorescence was used to assess the relative activity of each fusion construct.

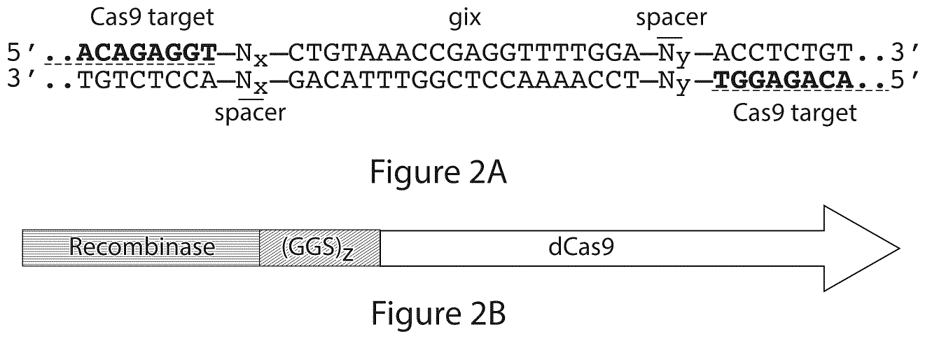

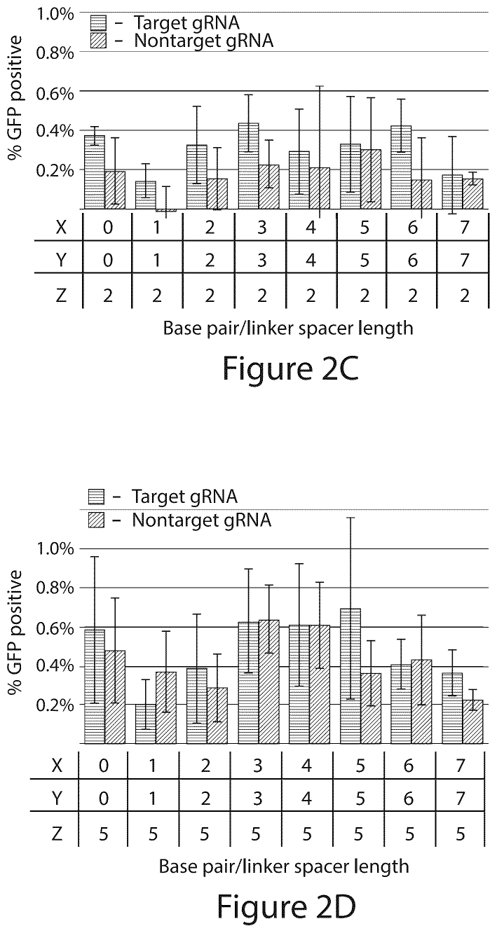

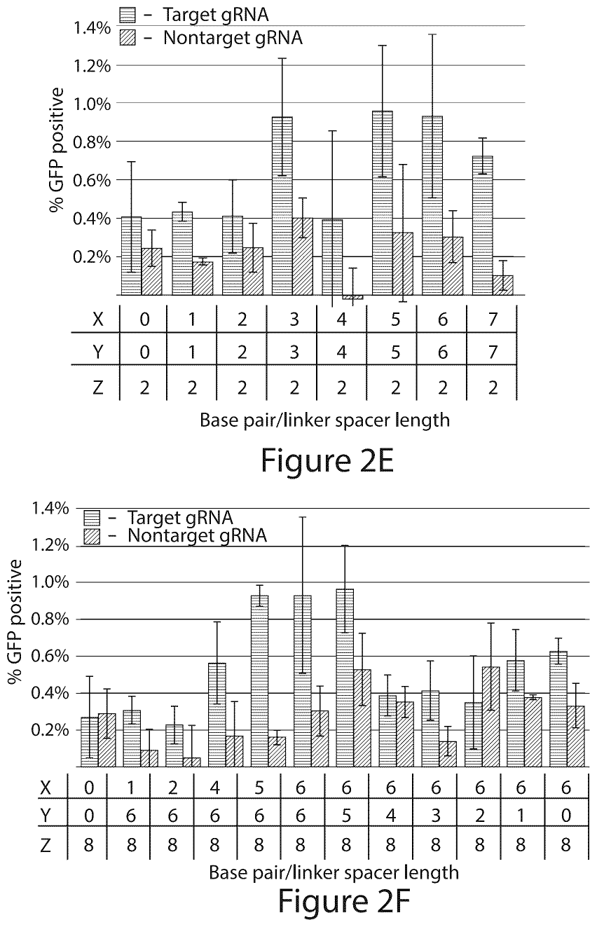

Parameters influencing the architecture of the recCas9 components, including the spacing between the core gix site and the guide RNA-binding site (from 0 to 7 bp), as well as linker length between the dCas9 and Ginβ moieties ((GGS)₂ (SEQ ID NO: 182), (GGS)₅ (SEQ ID NO: 701), or (GGS)₈ (SEQ ID NO: 183)) were varied (FIGS. 2A-2F). Most fusion architectures resulted in no observable guide RNA-dependent EGFP expression (FIGS. 1C-1D). However, one fusion construct containing a linker of eight GGS repeats and 3- to 6-base pair spacers resulted in approximately 1% recombination when a matched, but not mismatched, guide RNA was present (FIGS. 2E-2F). Recombination activity was consistently higher when 5-6 base pairs separated the dCas9 binding sites from the core (FIG. 2F). These results collectively reveal that specific fusion architectures between dCas9 and Ginβ can result in guide RNA-dependent recombination activity at spacer-flanked gix-related core sites in human cells. The 8×GGS linker fusion construct is referred to as “recCas9”.

Targeting DNA Sequences Found in the Human Genome with recCas9

Low levels of observed activity may be caused by a suboptimal guide RNA sequence or core gix sequence, consistent with previous reports showing that the efficiency of guide RNA:Cas9 binding is sequence-dependent (see, e.g., Xu et al., Sequence determinants of improved CRISPR sgRNA design. Genome research 25, 1147-1157 (2015), the entire contents of which is hereby incorporated by reference). Moreover, although the present optimization was conducted with the native gix core sequence (see, e.g., Klippel et al., The DNA Invertase Gin of Phage Mu—Formation of a Covalent Complex with DNA Via a Phosphoserine at Amino-Acid Position-9. Embo Journal 7, 1229-1237 (1988); Mertens et al., Site-specific recombination in bacteriophage Mu: characterization of binding sites for the DNA invertase Gin. The EMBO journal 7, 1219-1227 (1988); Plasterk et al., DNA inversions in the chromosome of Escherichia coli and in bacteriophage Mu: relationship to other site-specific recombination systems. Proceedings of the National Academy of Sciences of the United States of America 80, 5355-5358 (1983); the entire contents of each of which are hereby incorporated by reference), several studies have shown that zinc finger-Gin or TALE-Gin fusions are active, and in some cases more active, on slightly altered core sites. See, e.g., Gordley et al., 3rd, Synthesis of programmable integrases. Proceedings of the National Academy of Sciences of the United States of America 106, 5053-5058 (2009); Gersbach et al., Targeted plasmid integration into the human genome by an engineered zinc-finger recombinase. Nucleic acids research 39, 7868-7878 (2011); Mercer et al., Chimeric TALE recombinases with programmable DNA sequence specificity. Nucleic acids research 40, 11163-11172 (2012); Gaj et al., A comprehensive approach to zinc-finger recombinase customization enables genomic targeting in human cells. Nucleic acids research 41, 3937-3946 (2013); Gordley et al., 3rd, Evolution of programmable zinc finger-recombinases with activity in human cells. J Mol Biol 367, 802-813 (2007); Gersbach et al., 3rd, Directed evolution of recombinase specificity by split gene reassembly. Nucleic acids research 38, 4198-4206 (2010); and Gaj et al., Structure-guided reprogramming of serine recombinase DNA sequence specificity. Proceedings of the National Academy of Sciences of the United States of America 108, 498-503 (2011); the entire contents of each of which are hereby incorporated by reference). Thus, sequences found within the human genome were targeted in order to test if unmodified human genomic sequences were capable of being targeted by recCas9 and to test if varying the guide RNA and core sequences would increase recCas9 activity.

To identify potential target sites, previous findings that characterized evolved Gin variants (see, e.g., Gaj et al., A comprehensive approach to zinc-finger recombinase customization enables genomic targeting in human cells. Nucleic acids research 41, 3937-3946 (2013), the entire contents of which is hereby incorporated by reference) as well as the observations above were used. Using this information, the human genome was searched for sites that contained CCN₍₃₀₋₃₁₎-AAASSWWSSTTT-N₍₃₀₋₃₁₎-GG (SEQ ID NO: 699), where W is A or T, S is G or C, and N is any nucleotide. The N₍₃₀₋₃₁₎ includes the N of the NGG protospacer adjacent motif (PAM), the 20-base pair Cas9 binding site, a 5- to 6-base pair spacing between the Cas9 and gix sites, and the four outermost base pairs of the gix core site. The internal 12 base pairs of the gix core site (AAASSWWSSTTT, SEQ ID NO: 699) were previously determined to be important for Ginβ activity (see, e.g., Gaj et al., Nucleic acids research 41, 3937-3946 (2013).

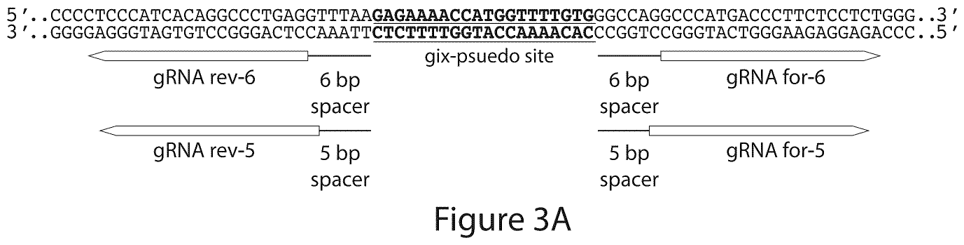

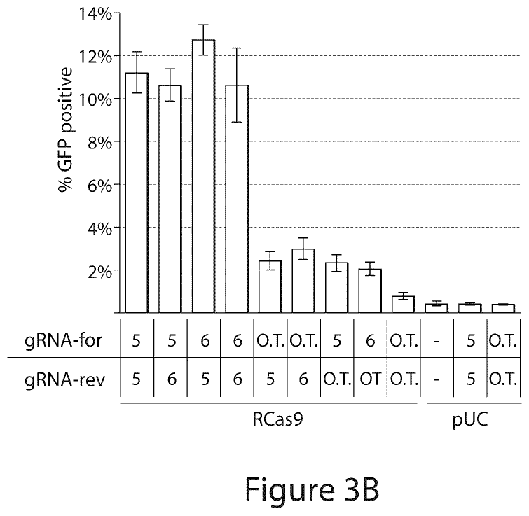

The search revealed approximately 450 such loci in the human genome (Table 9). A reporter construct was created, containing the sequence identical to one of these genomic loci, found in PCDH15, and then guide RNA expression vectors were constructed to direct recCas9 to this sequence (FIG. 3A). These vectors encoded two pairs of guide RNAs, each of which contain spacer sequences that match the 5′ and 3′ regions flanking the PCDH15 pseudo gix sites. Co-transfection of the reporter plasmid, combinations of these flanking guide RNA expression vectors, and the recCas9 expression vector resulted in EGFP expression in 11%-13% of transfected cells (FIG. 3B), representing a >10-fold improvement in activity over the results shown in FIG. 2. These findings demonstrate that a more judicious choice of recCas9 target sequences can result in substantially improved recombination efficiency at DNA sequences matching those found in the human genome.

Next, whether both guide RNA sequences were required to cause recCas9-mediated deletion was determined. HEK293T cells were co-transfected with just one of the guide RNA vectors targeting the 5′ or 3′ flanking sequences of the PCDH15 pseudo-gix core site, the PCDH15 reporter plasmid, and a recCas9 expression vector. These co-transfections resulted in 2.5-3% EGFP expression (FIG. 3B). The low levels of activity observed upon expression of just one of the targeting guide RNAs and recCas9 may be caused by the propensity of hyperactivated gix monomers to form dimers (see, e.g., Gaj et al., Enhancing the Specificity of Recombinase-Mediated Genome Engineering through Dimer Interface Redesign. J Am Chem Soc 136, 5047-5056 (2014), the entire contents of which is hereby incorporated by reference); transient dimerization may occasionally allow a single protospacer sequence to localize the dimer to a target site. No activity was detected above background when using off-target guide RNA vectors or when the recCas9 vector was replaced by pUC (FIG. 3B).

These findings demonstrate that recCas9 activity can be increased substantially over the modest activity observed in the initial experiments by choosing different target sites and matching guide RNA sequences. A greater than 10-fold increase in activity on the PCDH15 site compared to the original target sequences was observed (compare FIG. 3B with FIG. 2F). Further, maximal recombination activity is dependent on the presence of both guide RNAs and recCas9.

Orthogonality of recCas9

Next, whether recCas9 could target multiple, separate loci matching sequences found in the human genome in an orthogonal manner was tested. A subset of the recCas9 target sites in the human genome based on their potential use as a safe-harbor loci for genomic integration, or in one case, based on their location within a gene implicated in genetic disease, were selected.

To identify these sites, ENSEMBL (release 81) was searched to identify which predicted recCas9 target sites fall within annotated genes (see, e.g., Cunningham et al., Ensembl 2015. Nucleic acids research 43, D662-669 (2015), the entire contents of which is hereby incorporated by reference). One such site fell within an intronic region of FGF14. Mutations within FGF14 are believed to cause spinocerebellar ataxia 27 (SCA 27) (see, e.g., van Swieten et al., A mutation in the fibroblast growth factor 14 gene is associated with autosomal dominant cerebellar ataxia [corrected]. Am J Hum Genet 72, 191-199 (2003); Brusse et al., Spinocerebellar ataxia associated with a mutation in the fibroblast growth factor 14 gene (SCA27): A new phenotype. Mov Disord 21, 396-401 (2006); Choquet et al., A novel frameshift mutation in FGF14 causes an autosomal dominant episodic ataxia. Neurogenetics 16, 233-236 (2015); Coebergh et al., A new variable phenotype in spinocerebellar ataxia 27 (SCA 27) caused by a deletion in the FGF14 gene. Eur J Paediatr Neurol 18, 413-415 (2014); Shimojima et al., Spinocerebellar ataxias type 27 derived from a disruption of the fibroblast growth factor 14 gene with mimicking phenotype of paroxysmal non-kinesigenic dyskinesia. Brain Dev 34, 230-233 (2012); the entire contents of each of which are incorporated herein by reference). Finally, a fraction of the predicted recCas9 target sites that did not fall within genes were manually interrogated to determine if some sequences fell within safe harbor loci. Using annotations in ENSEMBL genomic targets that matched most of the five criteria for safe harbor loci described by Bushman and coworkers were identified (Cunningham et al., Ensembl 2015. Nucleic acids research 43, D662-669 (2015); and Sadelain et al., Safe harbours for the integration of new DNA in the human genome. Nat Rev Cancer 12, 51-58 (2012); the entire contents of each of which are incorporated herein by reference). Five reporters and corresponding guide RNA vector pairs containing sequences identical to those in the genome were constructed. To evaluate the orthogonality of recCas9 when programmed with different guide RNAs, all combinations of five guide RNA pairs with five reporters were tested.

Cotransfection of reporter, guide RNA plasmids, and recCas9 expression vectors revealed that three of the five reporters tested resulted in substantial levels of EGFP-positive cells consistent with recCas9-mediated recombination. This EGFP expression was strictly dependent upon cotransfection with a recCas9 expression vector and guide RNA plasmids matching the target site sequences on the reporter construct (FIG. 4A). The same guide RNA pairs that caused recombination when cotransfected with cognate reporter plasmids and a recCas9 vector were unable to mediate recombination when cotransfected with non-cognate reporter plasmids (FIG. 4A). These results demonstrate that recCas9 activity is orthogonal and will only catalyze recombination at a gix related core sites when programmed with a pair of guide RNAs matching the flanking sequences. No recombinase activity above the background level of the assay was observed when reporter plasmids were transfected without vectors expressing recCas9 and guide RNAs.

Characterization of recCas9 Products

The products of recCas9-mediated recombination of the reporter plasmids were characterized to confirm that EGFP expression was a result of recCas9-mediated removal of the poly-A terminator sequence. Reporter plasmids were sequenced for chromosome 5-site 1, chromosome 12, and chromosome 13 (FGF14 locus) after cotransfection with recCas9 expression vectors and with plasmids producing cognate or non-cognate guide RNA pairs. After incubation for 72 hours, episomal DNA was extracted (as described above) and transformed into E. coli to isolate reporter plasmids. Single colonies containing reporter plasmids were sequenced (FIG. 4B).

Individual colonies were expected to contain either an unmodified or a recombined reporter plasmid (FIG. 4C). For each biological replicate, an average of 97 colonies transformed with reporter plasmid isolated from each transfection condition were sequenced. Recombined plasmids were only observed if reporter plasmids were previously cotransfected with cognate guide RNA plasmids and recCas9 expression vectors (FIG. 4D). In two separate experiments, the percent of recombined plasmid ranged from 12% for site 1 in chromosome 5 to an average of 32% for the FGF14 locus in chromosome 13. The sequencing data therefore were consistent with the earlier flow cytometry analysis in FIG. 4A. The absolute levels of recombined plasmid were somewhat higher than the percent of EGFP-positive cells (FIG. 4). This difference likely arises because the flow cytometry assay does not report on multiple recombination events that can occur when multiple copies of the reporter plasmid are present in a single cell; even a single recombination event may result in EGFP fluorescence. As a result, the percentage of EGFP-positive cells may correspond to a lower limit on the actual percentage of recombined reporter plasmids. Alternatively, the difference may reflect the negative correlation between plasmid size and transformation efficiency (see, e.g., Hanahan, Studies on transformation of Escherichia coli with plasmids. J Mol Biol 166, 557-580 (1983), the entire contents of which is hereby incorporated by reference); the recombined plasmid is approximately 5,700 base pairs and may transform slightly better than the intact plasmid, which is approximately 6,900 base pairs.

Since zinc finger-recombinases have been reported to cause mutations at recombinase core-site junctions (see, e.g., Gaj et al., A comprehensive approach to zinc-finger recombinase customization enables genomic targeting in human cells. Nucleic acids research 41, 3937-3946 (2013), the entire contents of which is hereby incorporated by reference), whether such mutagenesis occurs from recCas9 treatment was tested. In the reporter construct, recCas9 should delete kanR and the poly-A terminator by first cleaving the central dinucleotide of both gix core sites and then religating the two cores to each other (FIG. 4C). Thus, the recombination product should be a single recombination site consisting of the first half of the ‘left’ target site and the second half of the ‘right’ target site. Erroneous or incomplete reactions could result in other products. Strikingly, all of the 134 recombined sequences examined contained the expected recombination products. Further, a total of 2,317 sequencing reads from two separate sets of transfection experiments revealed only three sequencing reads containing potential deletion products at otherwise non-recombined plasmids.

One of these deletion-containing reads was observed in a chromosome 12 reporter plasmid that was transfected with the pUC control and lacked both recCas9 target sites as well as the polyA terminator. This product was attributed to DNA damage that occurred during the transfection, isolation, or subsequent manipulation. Because recCas9 may only localize to sequences when cotransfected with reporter and cognate guide RNA expression vectors, a more relevant metric may be to measure the total number of deletion products observed when reporter plasmids are cotransfected with cognate guide RNA vectors and recCas9 expression vectors. A single indel was observed out of a total of 185 plasmids sequenced from cotransfections with the chromosome 5-site 1 reporter and cognate guide RNA. Similarly, one indel was observed out of 204 plasmids from the chromosome 12 reporter following transfection with cognate guide RNA and recCas9 expression vectors. Notably, out of 202 sequencing reads, no indels were observed from the chromosome 13 reporter following cognate guide RNA and recCas9 cotransfection, despite resulting in the highest observed levels of recombination. These observations collectively suggest that recCas9 mediates predominantly error-free recombination.

Taken together, these results establish that recCas9 can target multiple sites found within the human genome with minimal cross-reactivity or byproduct formation. Substrates undergo efficient recombination only in the presence of cognate guide RNA sequences and recCas9, give clean recombination products in human cells, and generally do not result in mutations at the core-site junctions or products such as indels that arise from cellular DNA repair.

### RecCas9-Mediated Genomic Deletion

Finally, whether recCas9 is capable of operating directly on the genomic DNA of cultured human cells was investigated. Using the list of potential recCas9 recognition sites in the human genome (Table 9), pairs of sites that, if targeted by recCas9, would yield chromosomal deletion events detectable by PCR, were sought. Guide RNA expression vectors were designed to direct recCas9 to those recCas9 sites closest to the chromosome 5-site 1 or chromosome 13 (FGF14 locus), sites which were both shown to be recombined in transient transfection assays (FIG. 4). The new target sites ranged from approximately 3 to 23 Mbp upstream and 7 to 10 Mbp downstream of chromosome 5-site 1, and 12 to 44 Mbp upstream of the chromosome 13-FGF14 site. The recCas9 expression vector was cotransfected with each of these new guide RNA pairs and the validated guide RNA pairs used for chromosome 5-site 1 or chromosome 13-FGF14, but evidence of chromosomal deletions by genomic PCR was not observed.

It was thought that genomic deletion might be more efficient if the recCas9 target sites were closer to each other on the genome. Two recCas9 sites separated by 14.2 kb within an intronic region of FAM19A2 were identified; these sites also contained identical dinucleotide cores which should facilitate deletion. FAM19A2 is one of five closely related TAFA-family genes encoding small, secreted proteins that are thought to have a regulatory role in immune and nerve cells (see, e.g., Parker et al., Admixture mapping identifies a quantitative trait locus associated with FEV1/FVC in the COPDGene Study. Genet Epidemiol 38, 652-659 (2014), the entire contents of which is hereby incorporated by reference). Small nucleotide polymorphisms located in intronic sequences of FAM19A2 have been associated with elevated risk for systemic lupus erythematosus (SLE) and chronic obstructive pulmonary disease (COPD) in genome-wide association studies (see, e.g., Parker et al., Admixture mapping identifies a quantitative trait locus associated with FEV1/FVC in the COPDGene Study. Genet Epidemiol 38, 652-659 (2014), the entire contents of which is hereby incorporated by reference); deletion of the intronic regions of this gene might therefore provide insights into the causes of these diseases. Four guide RNA sequences were cloned in expression vectors designed to mediate recCas9 deletion between these two FAM19A2 sites. Vectors expressing these guide RNAs were cotransfected with the recCas9 expression vector (FIG. 5A). RecCas9-mediated recombination between the two sites should result in deletion of the 14.2 kb intervening region. Indeed, this deletion event was detected by nested PCR using gene-specific primers that flank the two FAM19A2 recCas9 targets. The expected PCR product that is consistent with recCas9-mediated deletion was observed only in genomic DNA isolated from cells cotransfected with the recCas9 and all four guide RNA expression vectors (FIG. 5B). The deletion PCR product was not detected in the genomic DNA of cells transfected without either the upstream or downstream pair of guide RNA expression vectors alone, without the recCas9 expression plasmid, or for the genomic DNA of untransfected control cells (FIG. 5B). The estimated limit of detection for these nested PCR products was approximately 1 deletion event per 5,500 chromosomal copies. The 415-bp PCR product corresponding to the predicted genomic deletion was isolated and sequenced. Sequencing confirmed that the PCR product matched the predicted junction expected from the recombinase-mediated genomic deletion and did not contain any insertions or deletions suggestive of NHEJ (FIG. 5C).

A lower limit on the minimum genomic deletion efficiency was estimated using nested PCR on the serial dilutions of genomic template (see above or, e.g., Sykes et al., Quantitation of targets for PCR by use of limiting dilution. Biotechniques 13, 444-449 (1992), the entire contents of which is hereby incorporated by reference, for greater detail). A given amount of genomic DNA that yields the recCas9-specific nested PCR product must contain at least one edited chromosome. To establish a lower limit on this recCas9-mediated genomic deletion event, nested PCR was performed on serial dilutions of genomic DNA (isolated from cells transfected with recCas9 and the four FAM19A2 guide RNA expression vectors) to determine the lowest concentration of genomic template DNA that results in a detectable deletion product. These experiments revealed a lower limit of deletion efficiency of 0.023±0.017% (average of three biological replicates) (FIG. 5D), suggesting that recCas9-mediated genomic deletion proceeds with at least this efficiency. Nested PCR of the genomic DNA of untransfected cells resulted in no product, with an estimated limit of detection of <0.0072% recombination.

### Use of Other Alternative Recombinases

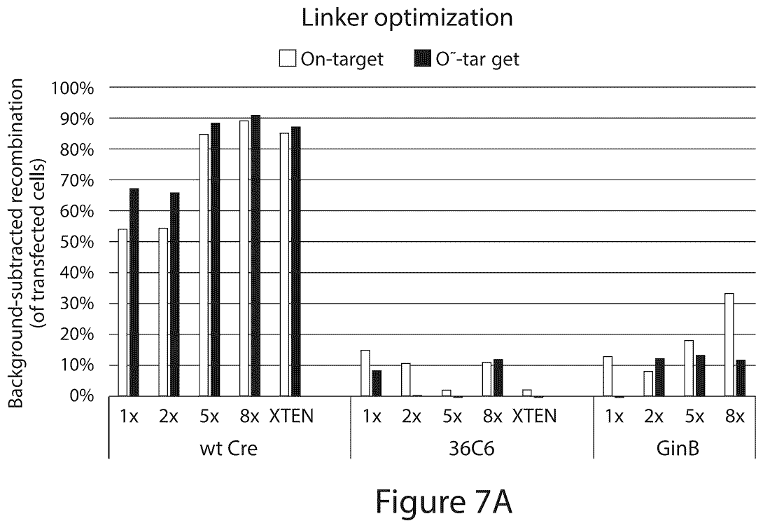

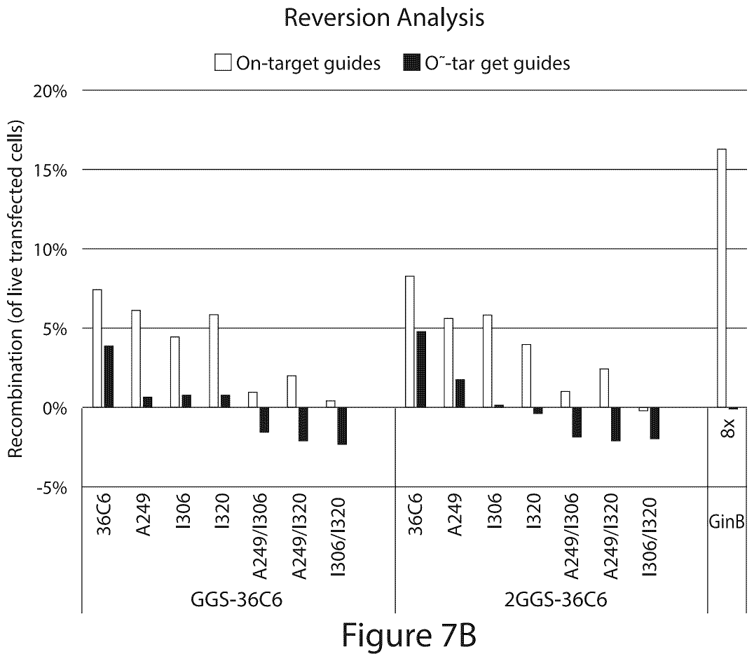

A Cre recombinase evolved to target a site in the Rosa locus of the human genome called “36C6” was fused to dCas9. This fusion was then used to recombine a plasmid-based reporter containing the Rosa target site in a guide-RNA dependent fashion. FIG. 7A demonstrates the results of linker optimization using wild-type Cre and 36C6. The 1× 2×, 5×, and 8× linkers shown are the number of GGS repeats in the linker. Reversion analysis demonstrated that making mutations to 36C6 fused to dCas9 could impact the relative guide dependence of the chimeric fusion (FIG. 7B). Reversions are labeled with their non-mutated amino acids. For example, position 306, which had been mutated to an M, was reverted to an I before the assay was performed. A GinB construct, targeting its cognate reporter, was used as a control for the experimental data shown in FIGS. 7A and 7B. The on-target guides were the chr13-102010574 guides (plasmids BC165 and 166). Abbreviations shown are GGS-36C6: dCas9-GGS-36C6; 2GGS-36C6 (using linker SEQ ID NO: 182): sdCas9-GGSGGS-36C6 (using linker SEQ ID NO: 182).

The target sequence used for 36C6 and all variant transfections is shown below: (guides—italics; Rosa site—bold):

(SEQ ID NO: 760)

CCTAGGGAAGTGATCATAGCTGAGTTTCTATCTCATGGTTTATGCTAAA
CTATATGTTGACATGTTGAGGAGACTTAAGTCCAAAACCTGG

In FIGS. 7A, 7B, 8, 9A, and 9B, the on-target guides for GinB were the chr13-102010574 guides (plasmids BC165 and 166). All off-target guides in FIGS. 7A, 7B, 8, 9A, and 9B were composed of the chr12-62418577 guides (BC163 and BC164).

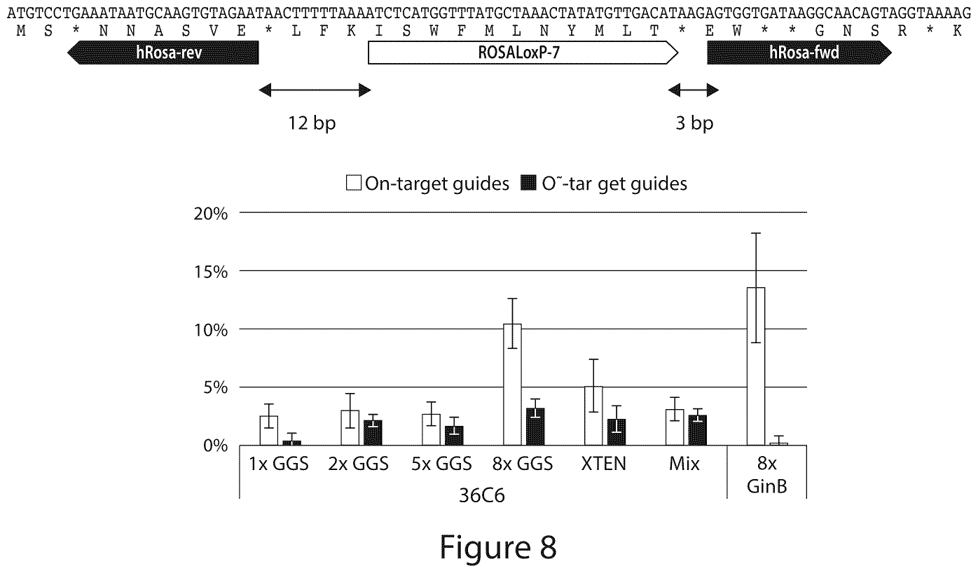

PAMs were identified flanking the Rosa26 site in the human genome that could support dCas9 binding (FIG. 8, top). Guide RNAs and a plasmid reporter were then designed to test whether the endogenous protospacers could support dCas9-36C6 activity. A GinB construct, targeting its cognate reporter, was used as a control. See FIG. 8. Mix: equal parts mixture of all 5 linker variants between Cas9 and 36C6. For hRosa, the target sequence, including guide RNA tagets, are below: (guides—italics; Rosa site—bold)

(SEQ ID NO: 767)

CCTGAAATAATGCAAGTGTAGAATAACTTTTTAAAATCTCATGGTTTAT
GCTAAACTATATGTTGACATAAGAGTGGTGATAAGGCAACAGTAGG

The on target guide plasmids for hRosa are identical to the other gRNA expression plasmids, except the protospacers are replaced with those shown above (FIG. 8).

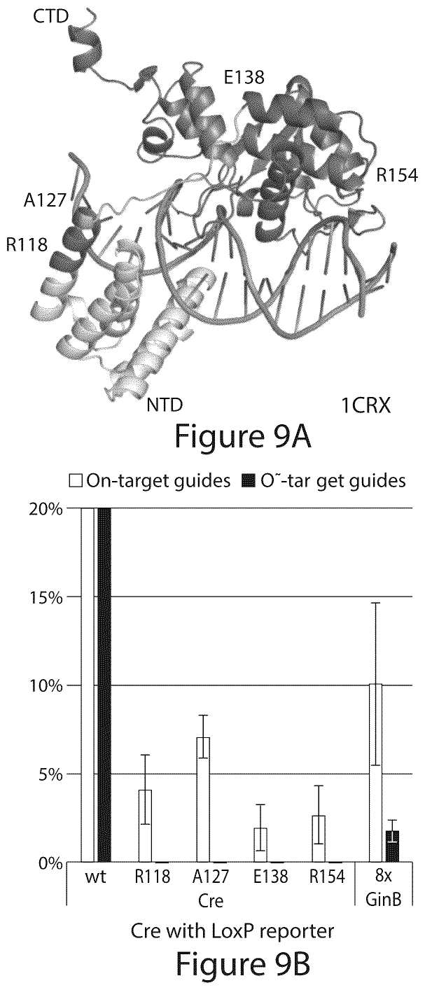

Several tested Cre truncations of dCas9-Cre recombinase fusions are shown in FIG. 9A. Truncated variants of Cre recombinase fused to dCas9 showed both appreciable recombinase activity as well as a strict reliance on the presence of guide RNA in a Lox plasmid reporter system (FIG. 9B). Truncated variants are labeled with the residue at which the truncated Cre begins. The linker for all fusion proteins shown in FIGS. 9A and 9B is 8×GGS. Wild type Cre fused to dCas9 was used as a positive control. The target sequence used for 36C6 and all variant transfections is shown below: (guides—italics; Rosa site—bold):

(SEQ ID NO: 768)

CCTAGGGAAGTGATCATAGCTGAGTTTCTATCTCATGGTTTATGCTAAA
CTATATGTTGACATGTTGAGGAGACTTAAGTCCAAAACCTGG

The on-target guides used were the chr13-102010574 guides (plasmids BC165 and 166) and the off-target guides were the chr12-62418577 guide (BC163 and BC164).

### REFERENCES

1. J. A. Doudna, E. Charpentier, Genome editing. The new frontier of genome engineering with CRISPR-Cas9. Science 346, 1258096 (2014).
2. M. R. Capecchi, Altering the genome by homologous recombination. Science 244, 1288-1292 (1989).
3. K. R. Thomas, K. R. Folger, M. R. Capecchi, High frequency targeting of genes to specific sites in the mammalian genome. Cell 44, 419-428 (1986).
4. A. Choulika, A. Perrin, B. Dujon, J. F. Nicolas, Induction of homologous recombination in mammalian chromosomes by using the I-SceI system of Saccharomyces cerevisiae. Mol Cell Biol 15, 1968-1973 (1995).
5. D. Carroll, Progress and prospects: zinc-finger nucleases as gene therapy agents. Gene Ther 15, 1463-1468 (2008).
6. J. C. Miller et al., A TALE nuclease architecture for efficient genome editing. Nature biotechnology 29, 143-U149 (2011).
7. J. K. Joung, J. D. Sander, TALENs: a widely applicable technology for targeted genome editing. Nat Rev Mol Cell Biol 14, 49-55 (2013).
8. P. Mali et al., RNA-guided human genome engineering via Cas9. Science 339, 823-826 (2013).
9. L. Cong et al., Multiplex genome engineering using CRISPR/Cas systems. Science 339, 819-823 (2013).
10. J. P. Guilinger, D. B. Thompson, D. R. Liu, Fusion of catalytically inactive Cas9 to FokI nuclease improves the specificity of genome modification. Nature biotechnology, (2014).
11. S. Q. Tsai et al., Dimeric CRISPR RNA-guided FokI nucleases for highly specific genome editing. Nature biotechnology, (2014).
12. H. Fung, D. M. Weinstock, Repair at single targeted DNA double-strand breaks in pluripotent and differentiated human cells. PloS one 6, e20514 (2011).
13. W. D. Heyer, K. T. Ehmsen, J. Liu, Regulation of homologous recombination in eukaryotes. Annu Rev Genet 44, 113-139 (2010).
14. D. Branzei, M. Foiani, Regulation of DNA repair throughout the cell cycle. Nat Rev Mol Cell Bio 9, 297-308 (2008).
15. V. T. Chu et al., Increasing the efficiency of homology-directed repair for CRISPR-Cas9-induced precise gene editing in mammalian cells. Nature biotechnology, (2015).
16. T. Maruyama et al., Increasing the efficiency of precise genome editing with CRISPR-Cas9 by inhibition of nonhomologous end joining. Nature biotechnology, (2015).
17. S. Lin, B. T. Staahl, R. K. Alla, J. A. Doudna, Enhanced homology-directed human genome engineering by controlled timing of CRISPR/Cas9 delivery. eLife 3, e04766 (2014).
18. S. Turan, C. Zehe, J. Kuehle, J. H. Qiao, J. Bode, Recombinase-mediated cassette exchange (RMCE)—A rapidly-expanding toolbox for targeted genomic modifications. Gene 515, 1-27 (2013).
19. T. Gaj, S. J. Sirk, C. F. Barbas, Expanding the Scope of Site-Specific Recombinases for Genetic and Metabolic Engineering. Biotechnology and bioengineering 111, 1-15 (2014).
20. N. D. F. Grindley, K. L. Whiteson, P. A. Rice, Mechanisms of site-specific recombination. Annu Rev Biochem 75, 567-605 (2006).
21. C. R. Sclimenti, B. Thyagarajan, M. P. Calos, Directed evolution of a recombinase for improved genomic integration at a native human sequence. Nucleic acids research 29, 5044-5051 (2001).
22. R. Shah, F. Li, E. Voziyanova, Y. Voziyanov, Target-specific variants of Flp recombinase mediate genome engineering reactions in mammalian cells. The FEBS journal 282, 3323-3333 (2015).
23. J. Karpinski et al., Directed evolution of a recombinase that excises the provirus of most HIV-1 primary isolates with high specificity. Nature biotechnology, (2016).
24. F. Buchholz, A. F. Stewart, Alteration of Cre recombinase site specificity by substrate-linked protein evolution. Nature biotechnology 19, 1047-1052 (2001).
25. B. Thyagarajan, E. C. Olivares, R. P. Hollis, D. S. Ginsburg, M. P. Calos, Site-specific genomic integration in mammalian cells mediated by phage phiC31 integrase. Mol Cell Biol 21, 3926-3934 (2001).
26. B. Thyagarajan, M. J. Guimaraes, A. C. Groth, M. P. Calos, Mammalian genomes contain active recombinase recognition sites. Gene 244, 47-54 (2000).
27. A. Akopian, J. He, M. R. Boocock, W. M. Stark, Chimeric recombinases with designed DNA sequence recognition. Proceedings of the National Academy of Sciences of the United States of America 100, 8688-8691 (2003).
28. R. M. Gordley, C. A. Gersbach, C. F. Barbas, 3rd, Synthesis of programmable integrases. Proceedings of the National Academy of Sciences of the United States of America 106, 5053-5058 (2009).
29. M. M. Prorocic et al., Zinc-finger recombinase activities in vitro. Nucleic acids research 39, 9316-9328 (2011).
30. C. A. Gersbach, T. Gaj, R. M. Gordley, A. C. Mercer, C. F. Barbas, Targeted plasmid integration into the human genome by an engineered zinc-finger recombinase. Nucleic acids research 39, 7868-7878 (2011).
31. A. C. Mercer, T. Gaj, R. P. Fuller, C. F. Barbas, Chimeric TALE recombinases with programmable DNA sequence specificity. Nucleic acids research 40, 11163-11172 (2012).
32. T. Matsuda, C. L. Cepko, Controlled expression of transgenes introduced by in vivo electroporation. Proceedings of the National Academy of Sciences of the United States of America 104, 1027-1032 (2007).
33. N. E. Sanjana et al., A transcription activator-like effector toolbox for genome engineering. Nature protocols 7, 171-192 (2012).
34. T. Gaj, A. C. Mercer, S. J. Sirk, H. L. Smith, C. F. Barbas, A comprehensive approach to zinc-finger recombinase customization enables genomic targeting in human cells. Nucleic acids research 41, 3937-3946 (2013).
35. Y. Fu et al., High-frequency off-target mutagenesis induced by CRISPR-Cas nucleases in human cells. Nature biotechnology 31, 822-826 (2013).
36. J. Quan, J. Tian, Circular polymerase extension cloning of complex gene libraries and pathways. PloS one 4, e6441 (2009).
37. N. Hillson. (2010), vol. 2015, pp. CPEC protocol.
38. R. C. Gentleman et al., Bioconductor: open software development for computational biology and bioinformatics. Genome Biol 5, R80 (2004).
39. K. Motmans, S. Thirion, J. Raus, C. Vandevyver, Isolation and quantification of episomal expression vectors in human T cells. Biotechniques 23, 1044-1046 (1997).
40. B. Hirt, Selective extraction of polyoma DNA from infected mouse cell cultures. J Mol Biol 26, 365-369 (1967).
41. A. Klippel, G. Mertens, T. Patschinsky, R. Kahmann, The DNA Invertase Gin of Phage Mu—Formation of a Covalent Complex with DNA Via a Phosphoserine at Amino-Acid Position-9. Embo Journal 7, 1229-1237 (1988).
42. G. Mertens et al., Site-specific recombination in bacteriophage Mu: characterization of binding sites for the DNA invertase Gin. The EMBO journal 7, 1219-1227 (1988).
43. R. H. Plasterk, A. Brinkman, P. van de Putte, DNA inversions in the chromosome of Escherichia coli and in bacteriophage Mu: relationship to other site-specific recombination systems. Proceedings of the National Academy of Sciences of the United States of America 80, 5355-5358 (1983).
44. H. Xu et al., Sequence determinants of improved CRISPR sgRNA design. Genome research 25, 1147-1157 (2015).
45. R. M. Gordley, J. D. Smith, T. Graslund, C. F. Barbas, 3rd, Evolution of programmable zinc finger-recombinases with activity in human cells. J Mol Biol 367, 802-813 (2007).
46. C. A. Gersbach, T. Gaj, R. M. Gordley, C. F. Barbas, 3rd, Directed evolution of recombinase specificity by split gene reassembly. Nucleic acids research 38, 4198-4206 (2010).
47. T. Gaj, A. C. Mercer, C. A. Gersbach, R. M. Gordley, C. F. Barbas, Structure-guided reprogramming of serine recombinase DNA sequence specificity. Proceedings of the National Academy of Sciences of the United States of America 108, 498-503 (2011).
48. T. Gaj et al., Enhancing the Specificity of Recombinase-Mediated Genome Engineering through Dimer Interface Redesign. J Am Chem Soc 136, 5047-5056 (2014).
49. F. Cunningham et al., Ensembl 2015. Nucleic acids research 43, D662-669 (2015).
50. J. C. van Swieten et al., A mutation in the fibroblast growth factor 14 gene is associated with autosomal dominant cerebellar ataxia [corrected]. Am J Hum Genet 72, 191-199 (2003).
51. E. Brusse et al., Spinocerebellar ataxia associated with a mutation in the fibroblast growth factor 14 gene (SCA27): A new phenotype. Mov Disord 21, 396-401 (2006).
52. K. Choquet, R. La Piana, B. Brais, A novel frameshift mutation in FGF14 causes an autosomal dominant episodic ataxia. Neurogenetics 16, 233-236 (2015).
53. J. A. Coebergh et al., A new variable phenotype in spinocerebellar ataxia 27 (SCA 27) caused by a deletion in the FGF14 gene. Eur J Paediatr Neurol 18, 413-415 (2014).
54. K. Shimojima et al., Spinocerebellar ataxias type 27 derived from a disruption of the fibroblast growth factor 14 gene with mimicking phenotype of paroxysmal non-kinesigenic dyskinesia. Brain Dev 34, 230-233 (2012).
55. M. Sadelain, E. P. Papapetrou, F. D. Bushman, Safe harbours for the integration of new DNA in the human genome. Nat Rev Cancer 12, 51-58 (2012).
56. D. Hanahan, Studies on transformation of Escherichia coli with plasmids. J Mol Biol 166, 557-580 (1983).
57. M. M. Parker et al., Admixture mapping identifies a quantitative trait locus associated with FEV1/FVC in the COPDGene Study. Genet Epidemiol 38, 652-659 (2014).
58. P. J. Sykes et al., Quantitation of targets for PCR by use of limiting dilution. Biotechniques 13, 444-449 (1992).
59. A. Rath, R. Hromas, A. De Benedetti, Fidelity of end joining in mammalian episomes and the impact of Metnase on joint processing. BMC Mol Biol 15, 6 (2014).
60. P. Rebuzzini et al., New mammalian cellular systems to study mutations introduced at the break site by non-homologous end-joining. DNA Repair (Amst) 4, 546-555 (2005).
61. J. Smith, C. Baldeyron, I. De Oliveira, M. Sala-Trepat, D. Papadopoulo, The influence of DNA double-strand break structure on end-joining in human cells. Nucleic acids research 29, 4783-4792 (2001).
62. S. Turan et al., Recombinase-mediated cassette exchange (RMCE): traditional concepts and current challenges. J Mol Biol 407, 193-221 (2011).
63. S. J. Sirk, T. Gaj, A. Jonsson, A. C. Mercer, C. F. Barbas, Expanding the zinc-finger recombinase repertoire: directed evolution and mutational analysis of serine recombinase specificity determinants. Nucleic acids research 42, 4755-4766 (2014).
64. B. P. Kleinstiver et al., Broadening the targeting range of Staphylococcus aureus CRISPR-Cas9 by modifying PAM recognition. Nature biotechnology 33, 1293-1298 (2015).
65. B. P. Kleinstiver et al., Engineered CRISPR-Cas9 nucleases with altered PAM specificities. Nature 523, 481-U249 (2015).
66. K. M. Esvelt et al., Orthogonal Cas9 proteins for RNA-guided gene regulation and editing. Nature methods 10, 1116-1121 (2013).
67. B. Zetsche et al., Cpf1 Is a Single RNA-Guided Endonuclease of a Class 2 CRISPR-Cas System. Cell 163, 759-771 (2015).
68. K. Dormiani et al., Long-term and efficient expression of human beta-globin gene in a hematopoietic cell line using a new site-specific integrating non-viral system. Gene Ther 22, 663-674 (2015).
69. E. Wijnker, H. de Jong, Managing meiotic recombination in plant breeding. Trends in plant science 13, 640-646 (2008).
70. J. F. Petolino, V. Srivastava, H. Daniell, Editing Plant Genomes: a new era of crop improvement. Plant Biotechnol J 14, 435-436 (2016).

### EQUIVALENTS AND SCOPE

Those skilled in the art will recognize, or be able to ascertain using no more than routine experimentation, many equivalents to the specific embodiments of the invention described herein. The scope of the present invention is not intended to be limited to the above description, but rather is as set forth in the appended claims.

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process.

Furthermore, it is to be understood that the invention encompasses all variations, combinations, and permutations in which one or more limitations, elements, clauses, descriptive terms, etc., from one or more of the claims or from relevant portions of the description is introduced into another claim. For example, any claim that is dependent on another claim can be modified to include one or more limitations found in any other claim that is dependent on the same base claim. Furthermore, where the claims recite a composition, it is to be understood that methods of using the composition for any of the purposes disclosed herein are included, and methods of making the composition according to any of the methods of making disclosed herein or other methods known in the art are included, unless otherwise indicated or unless it would be evident to one of ordinary skill in the art that a contradiction or inconsistency would arise.

Where elements are presented as lists, e.g., in Markush group format, it is to be understood that each subgroup of the elements is also disclosed, and any element(s) can be removed from the group. It is also noted that the term “comprising” is intended to be open and permits the inclusion of additional elements or steps. It should be understood that, in general, where the invention, or aspects of the invention, is/are referred to as comprising particular elements, features, steps, etc., certain embodiments of the invention or aspects of the invention consist, or consist essentially of, such elements, features, steps, etc. For purposes of simplicity those embodiments have not been specifically set forth in haec verba herein. Thus for each embodiment of the invention that comprises one or more elements, features, steps, etc., the invention also provides embodiments that consist or consist essentially of those elements, features, steps, etc.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in different embodiments of the invention, to the tenth of the unit of the lower limit of the range, unless the context clearly dictates otherwise. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

In addition, it is to be understood that any particular embodiment of the present invention may be explicitly excluded from any one or more of the claims. Where ranges are given, any value within the range may explicitly be excluded from any one or more of the claims. Any embodiment, element, feature, application, or aspect of the compositions and/or methods of the invention, can be excluded from any one or more claims. For purposes of brevity, all of the embodiments in which one or more elements, features, purposes, or aspects is excluded are not set forth explicitly herein.

All publications, patents and sequence database entries mentioned herein, including those items listed above, are hereby incorporated by reference in their entirety as if each individual publication or patent was specifically and individually indicated to be incorporated by reference. In case of conflict, the present application, including any definitions herein, will control.

1-80. (canceled)

81. A method for site-specific recombination between two DNA molecules, comprising:
(a) contacting a first DNA with a first fusion protein, wherein the guide nucleotide sequence-programmable DNA binding protein domain binds a first gRNA that hybridizes to a first region of the first DNA;
(b) contacting the first DNA with a second fusion protein, wherein the guide nucleotide sequence-programmable DNA binding protein domain of the second fusion protein binds a second gRNA that hybridizes to a second region of the first DNA;
(c) contacting a second DNA with a third fusion protein, wherein the guide nucleotide sequence-programmable DNA binding protein domain of the third fusion protein binds a third gRNA that hybridizes to a first region of the second DNA; and
(d) contacting the second DNA with a fourth fusion protein, wherein the guide nucleotide sequence-programmable DNA binding protein domain of the fourth fusion protein binds a fourth gRNA that hybridizes to a second region of the second DNA;
wherein the binding of the fusion proteins in steps (a)-(d) results in the tetramerization of the recombinase catalytic domains of the fusion proteins, under conditions such that the DNAs are recombined, and wherein the first, second, third, and/or fourth fusion protein is a fusion protein comprising:
(i) a guide nucleotide sequence-programmable DNA binding protein domain;
(ii) a linker; and
(iii) a recombinase catalytic domain,
wherein the recombinase catalytic domain comprises a Gin recombinase that has at least 95% sequence identity with SEQ ID NO: 713, and wherein the amino acid sequence of Gin recombinase catalytic domain comprises one or more mutations from the group consisting of H106Y, I127L, I136R, or G137F in SEQ ID NO: 713.

82. The method of claim 81, wherein the first and second DNA molecules have different sequences.

83. The method of claim 81, wherein the first and second gRNAs of steps (a) and (b) hybridize to opposing strands of the first DNA, and the third and fourth gRNAs of steps (c) and (d) hybridize to opposing strands of the second DNA.

84. The method of claim 81, wherein the first and second gRNAs of steps (a) and (b); and/or the third and fourth gRNAs of steps (c) and (d) hybridize to regions of their respective DNAs that are no more than 100 base pairs apart.

85. (canceled)

86. The method of claim 81, wherein the gRNAs of steps (a) and (b); and/or the gRNAs of steps (c) and (d) hybridize to regions of their respective DNAs at gRNA binding sites that flank a recombinase site.

87. (canceled)

88. The method of claim 86, wherein the recombinase site comprises a gix core or gix-related core sequence.

89. The method of claim 87, wherein the distance between the gix core or gix-related core sequence and at least one gRNA binding site is from 3 to 7 base pairs.

90. (canceled)

91. A method for site-specific recombination between two regions of a single DNA molecule, comprising:
(a) contacting the DNA with a first fusion protein, wherein the guide nucleotide sequence-programmable DNA binding protein domain binds a first gRNA that hybridizes to a first region of the DNA;
(b) contacting the DNA with a second fusion protein, wherein the guide nucleotide sequence-programmable DNA binding protein domain of the second fusion protein binds a second gRNA that hybridizes to a second region of the DNA;
(c) contacting the DNA with a third fusion protein, wherein the guide nucleotide sequence-programmable DNA binding protein domain of the third fusion protein binds a third gRNA that hybridizes to a third region of the DNA; and
(d) contacting the DNA with a fourth fusion protein, wherein the guide nucleotide sequence-programmable DNA binding protein domain of the fourth fusion protein binds a fourth gRNA that hybridizes to a fourth region of the DNA;
wherein the binding of the fusion proteins in steps (a)-(d) results in the tetramerization of the recombinase catalytic domains of the fusion proteins, under conditions such that the DNA is recombined, and wherein the first, second, third, and/or fourth fusion protein is a fusion protein comprising:
(i) a guide nucleotide sequence-programmable DNA binding protein domain;
(ii) a linker; and
(iii) a recombinase catalytic domain,
wherein the recombinase catalytic domain comprises a Gin recombinase that has at least 95% sequence identity with SEQ ID NO: 713, and wherein the amino acid sequence of Gin recombinase catalytic domain comprises one or more mutations from the group consisting of H106Y, I127L, I136R, or G137F in SEQ ID NO: 713.

92. The method of claim 91, wherein the two regions of the single DNA molecule that are recombined have different sequences.

93. The method of claim 91, wherein the recombination results in the deletion of a region of the DNA molecule.

94. A method of claim 93, wherein the region of the DNA molecule that is deleted is prone to cross-over events in meiosis.

95. The method of claim 91, wherein the first and second gRNAs of steps (a)-(b) hybridize to the same strand of the DNA, and the third and fourth gRNAs of steps (c)-(d) hybridize to the opposing strand of the DNA.

96. The method of 91, wherein the gRNAs of steps (a) and (b) hybridize to regions of the DNA that are no more than 100 base pairs apart, and the gRNAs of steps (c) and (d) hybridize to regions of the DNA that are no more than 100 base pairs apart.

97. (canceled)

98. The method of claim 91, wherein the gRNAs of steps (a) and (b); and/or the gRNAs of steps (c) and (d) hybridize to gRNA binding sites flanking a recombinase site.

99. (canceled)

100. The method of claim 99, wherein the recombinase site comprises a gix core or gix-related core sequence.

101. The method of claim 100, wherein the distance between the gix core or gix-related core sequence and at least one gRNA binding site is from 3 to 7 base pairs.

102-116. (canceled)

117. The method of claim 88, wherein the first and/or second DNA comprise any one of the sequences listed in Table 9.

118. The method of claim 100, wherein at least one of the two regions of the singled DNA molecule comprises any one of the sequences listed in Table 9.
